(19) United States
(12) Patent Application Publication
(10) Pub. No.: US 2020/0262813 A1
(43) Pub. Date: Aug. 20, 2020

(54) 1,5,7-TRISUBSTITUTED ISOQUINOLINE DERIVATIVES, PREPARATION THEREOF, AND USE THEREOF IN MEDICINES

(71) Applicants: SHANGHAI HAIYAN PHARMACEUTICAL TECHNOLOGY CO., LTD., Shanghai (CN); YANGTZE RIVER PHARMACEUTICAL GROUP CO., LTD., Jiangsu (CN)

(72) Inventors: Fusheng ZHOU, Shanghai (CN); Xiaoyong SHI, Shanghai (CN); Lixiao WANG, Shanghai (CN); Feng XU, Shanghai (CN); Jing XIE, Shanghai (CN); Ping BU, Shanghai (CN); Pengfei CHENG, Shanghai (CN); Zhiyuan ZHANG, Shanghai (CN); Nanping CAI, Shanghai (CN); Jiong LAN, Shanghai (CN)

(21) Appl. No.: 16/306,331

(22) PCT Filed: Nov. 10, 2017
(86) PCT No.: PCT/CN2017/110459

§ 371 Date: Nov. 30, 2018

(30) Foreign Application Priority Data
Nov. 11, 2016 (CN) 201610994495.3

(51) Int. Cl. C07D 401/14; C07D 405/14; C07D 401/12; C07D 413/14; C07D 409/14; C07D 471/08

(52) U.S. Cl. CPC C07D 401/14; C07D 405/14; C07D 401/12; A61K 45/06; C07D 409/14; C07D 471/08; C07D 413/14

(57) ABSTRACT

The present disclosure relates to 1,5,7-trisubstituted isoquinoline derivatives, their preparation and pharmaceutical use. In particular, the present disclosure discloses a compound of formula (I) or a pharmaceutically acceptable salt, stereoisomer, solvate or prodrug thereof, and a preparation method and use thereof. The definitions of the groups in the formula can be found in the specification and claims.

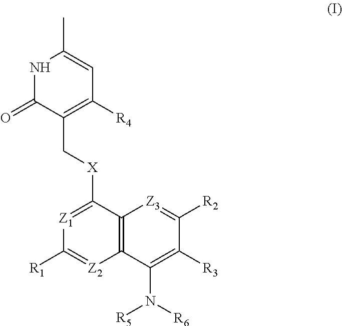

### TECHNICAL FIELD

The disclosure belongs to the field of medical technology. In particular, the present disclosure particularly relates to a 1,5,7-trisubstituted isoquinoline derivative, its preparation method and use as an EZH2 inhibitor, and a pharmaceutical composition prepared therefrom.

### BACKGROUND

The histone-lysine-N-methyltransferase EZH2 is involved in DNA methylation and DNA final transcription inhibition; and the methyl group at lysine position 27 is catalyzed to be transferred to histone H3 by the cofactor S-adenosyl-L-methionine. This methylation promotes the formation of heterochromatin, and therefore triggers gene silencing. EZH2 is part of PRC2 functional enzymes, and its gene maintains, controls and regulates the development and differentiation of an embryo through epigenetics, thus ensuring a healthy development of the embryo. The mutation or overexpression of EZH2 is associated with the formation of many cancers. EZH2 controlling gene controls the development of a tumor, and inhibiting EZH2 activity will slow the growth of the tumor. As a target inhibitor, EZH2 can regulate a variety of cancers including breast cancer, prostate cancer, melanoma and bladder cancer. PCT applications WO2011140324A1 and WO2012075080A1 disclose use of indole compounds as EZH2 inhibitors for treating cancer. PCT application WO2012118812A2 discloses use of bicyclic heterocyclic compounds as EZH2 inhibitors for treating cancer.

Therefore, the inhibition of EZH2 activity will effectively reduce cell proliferation and invasion, thereby providing a beneficial treatment for EZH2-mediated diseases or conditions. The compounds of the present disclosure, as EZH2 inhibitors, provide solutions for treatment of diseases or EZH2-mediated tumors.

### SUMMARY

The object of the present disclosure is to provide a class of novel compounds which can act as EZH2 inhibitors.

The present disclosure provides a compound represented by formula (I), or a pharmaceutically acceptable salt, stereoisomer, solvate or prodrug thereof:

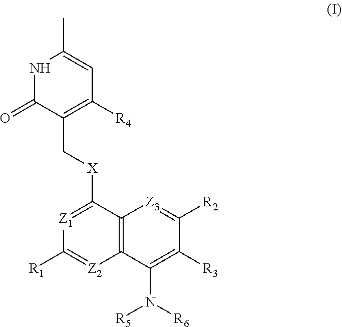

wherein, X is NH or O;

R₁, R₃ are each independently hydrogen, halogen (preferably fluorine, chlorine, bromine), C₁₋₈ alkyl (preferably C₁₋₆ alkyl, more preferably C₁₋₃ alkyl), halogenated C₁₋₈ alkyl (preferably halogenated C₁₋₆ alkyl, more preferably halogenated C₁₋₃ alkyl), C₁₋₈ alkoxy (preferably C₁₋₆ alkoxy, more preferably C₁₋₃ alkoxy), C₃₋₈ cycloalkyl (preferably C₃₋₆ cycloalkyl) or C₃₋₈ cycloalkoxy (preferably C₃₋₆ cycloalkoxy);

R₄ is a hydrogen, halogen (preferably fluorine, chlorine, bromine), hydroxy, CN, C₁₋₈ alkyl (preferably C₁₋₆ alkyl, more preferably C₁₋₃ alkyl), halogenated C₁₋₈ alkyl (preferably halogenated C₁₋₆ alkyl, more preferably halogenated C₁₋₃ alkyl), C₃₋₈ cycloalkyl (preferably C₃₋₆ cycloalkyl), C₁₋₈ alkoxy (preferably C₁₋₆ alkoxy, more preferably C₁₋₃ alkoxy), halogenated C₁₋₈ alkoxy (preferably halogenated C₁₋₆ alkoxy, more preferably halogenated C₁₋₃ alkoxy), C₃₋₈ cycloalkoxy (preferably C₃₋₆ cycloalkoxy), C₆₋₁₀ aryl (preferably phenyl), C(O)C₁₋₈ alkyl (preferably C(O)C₁₋₆ alkyl, more preferably C(O)C₁₋₃ alkyl), C(O)OC₁₋₈ alkyl (preferably C(O)OC₁₋₆ alkyl, more preferably C(O)OC₁₋₃ alkyl);

Z₁ is N or CR₈; Z₂ is N or CR₉; Z₃ is N or CR₁₀;

R₈, R₉, R₁₀ are each independently hydrogen, halogen (preferably fluorine, chlorine, bromine), C₁₋₈ alkyl (preferably C₁₋₆ alkyl, more preferably C₁₋₃ alkyl);

R₂ is a hydrogen, halogen (preferably fluorine, chlorine, bromine), CN, C₁₋₈ alkyl (preferably C₁₋₆ alkyl, more preferably C₁₋₃ alkyl), C₂₋₈ alkynyl (preferably C₂₋₆ alkynyl, more preferably C₂₋₃ alkynyl), halogenated C₁₋₈ alkyl (preferably halogenated C₁₋₆ alkyl, more preferably halogenated C₁₋₃ alkyl), C₃₋₈ cycloalkyl (preferably C₃₋₆ cycloalkyl), C₁₋₈ alkoxy (preferably C₁₋₆ alkoxy, more preferably C₁₋₃ alkoxy), C(O)NRₐ₁R_{b1}, NRₐ₂R_{b2}, NCORₐ₃, ORₐ₄, C₆₋₁₀ aryl (preferably phenyl), 4 to 6 membered saturated or unsaturated single heterocycle, 5 to 6 membered monocyclic heteroaryl ring;

Rₐ₁, R_{b1}, Rₐ₂, R_{b2}, Rₐ₃, Rₐ₄ are each independently hydrogen, C₁₋₈ alkyl (preferably C₁₋₆ alkyl, more preferably C₁₋₃ alkyl), 4 to 6 membered saturated single heterocycle, 5 to 6 membered monocyclic heteroaryl ring, or C₆₋₁₀ aryl; or

Rₐ₁, R_{b1} and the nitrogen atom attached thereto form a 5 to 6 membered saturated single heterocycle;

R₅ is a hydrogen, C₁₋₈ alkyl (preferably C₁₋₆ alkyl, more preferably C₁₋₃ alkyl) or C₁₋₈ alkyl substituted with C₁₋₈ alkoxy (preferably C₁₋₆ alkyl substituted with C₁₋₆ alkoxy, more preferably C₁₋₃ alkyl substituted with C₁₋₃ alkoxy);

R₆ is a C₁₋₈ alkyl (preferably C₁₋₆ alkyl, more preferably C₁₋₃ alkyl), C₃₋₈ cycloalkyl (preferably C₃₋₆ cycloalkyl), 4 to 6 membered saturated single heterocycle (preferably azetidine, oxetane, tetrahydrofuran, tetrahydrothiophene, pyrrolidine, piperidine, piperazine, morpholine or tetrahydropyrane, more preferably piperidine or tetrahydropyrane), spiro, spiroheterocycle, bridged ring, bridged heterocycle;

the alkyl, cycloalkyl, alkoxy, alkynyl, aryl, 4 to 6 membered saturated or unsaturated single heterocycle, 5 to 6 membered monocyclic heteroaryl ring, spiro, spiroheterocycle, bridged ring, bridged heterocycle are unsubstituted or substituted with 1, 2 or 3 substituents selected from the group consisting of NRₐ₀R_{b0}, hydroxymethyl, hydroxyethyl, carboxyl, —C(O)OC₁₋₆ alkyl, halogen, acetyl, hydroxy, C₁₋₈ alkyl (preferably C₁₋₆ alkyl, more preferably C₁₋₃ alkyl), C₁₋₈ alkoxy (preferably C₁₋₆ alkoxy, more preferably C₁₋₃ alkoxy), halogenated C₁₋₈ alkyl (preferably halogenated C₁₋₆ alkyl, more preferably halogenated C₁₋₃ alkyl), C₃₋₈ cycloalkyl (preferably C₃₋₆ cycloalkyl), halogenated C₁₋₈ alkoxy (preferably halogenated C₁₋₆ alkoxy, more preferably halogenated C₁₋₃ alkoxy), —SO₂C₁₋₈ alkyl (preferably —SO₂C₁₋₆ alkyl, more preferably —SO₂C₁₋₃ alkyl), C₆₋₁₀ aryl, 4 to 6 membered saturated single heterocycle, 5 to 6 membered monocyclic heteroaryl ring, O(CH₂)ₙOC₁₋₈ alkyl or —Y-L; wherein Y is a bond, CH₂, NH, O, CON or C(O); L is a C₆₋₁₀ aryl, 5 to 6 membered monocyclic heteroaryl ring, 4 to 6 membered saturated single heterocycle or NRₐ₀R_{b0}; Rₐ₀, R_{b0} are each independently hydrogen, acetyl, C₁₋₈ alkyl (preferably C₁₋₆ alkyl, more preferably C₁₋₃ alkyl), C₁₋₈ alkyl substituted with C₁₋₈ alkoxy (preferably C₁₋₆ alkyl substituted with C₁₋₆ alkoxy, more preferably C₁₋₃ alkyl substituted with C₁₋₃ alkoxy).

In another preferred example, alkynyl in R₂ is substituted with 4 to 6 membered saturated single heterocycle (preferably piperidine, piperazine, morpholine or tetrahydropyrane); the 4 to 6 membered saturated single heterocycle is unsubstituted or substituted with 1, 2 or 3 groups selected from the group consisting of NRₐ₀R_{b0}, hydroxymethyl, hydroxyethyl, carboxyl, —C(O)OC₁₋₆ alkyl, acetyl, hydroxy, C₁₋₃ alkyl, halogenated C₁₋₃ alkyl, C₃₋₆ cycloalkyl, azetidine, oxetane, tetrahydrofuran, tetrahydrothiophene, pyrrolidine, piperidine, oxazolidine, piperazine, dioxolane, dioxane, morpholine, thiomorpholine, thiomorpholine-1,1-dioxide or tetrahydropyrane; Rₐ₀, R_{b0} are each independently hydrogen or C₁₋₃ alkyl.

In another preferred example, Rₐ₁ is a hydrogen or C₁₋₈ alkyl (preferably C₁₋₆ alkyl, more preferably C₁₋₃ alkyl); R_{b1} is a hydrogen, C₁₋₈ alkyl (preferably C₁₋₆ alkyl, more preferably C₁₋₃ alkyl), 4 to 6 membered saturated single heterocycle (preferably piperidine, piperazine, morpholine or tetrahydropyrane), 5 to 6 membered monocyclic heteroaryl ring (preferably pyridine) or phenyl; or Rₐ₁, R_{b1} and the nitrogen atom attached thereto form a 4 to 6 membered saturated single heterocycle (preferably piperidine, piperazine, morpholine or tetrahydropyrane);

the 4 to 6 membered saturated single heterocycle, 5 to 6 membered monocyclic heteroaryl ring or phenyl is unsubstituted or substituted with 1, 2 or 3 groups selected from the group consisting of NRₐ₀R_{b0}, hydroxymethyl, hydroxyethyl, carboxyl, —C(O)OC₁₋₆ alkyl, acetyl, hydroxy, C₁₋₃ alkyl, halogenated C₁₋₃ alkyl, C₃₋₆ cycloalkyl, azetidine, oxetane, tetrahydrofuran, tetrahydrothiophene, pyrrolidine, piperidine, oxazolidine, piperazine, dioxolane, dioxane, morpholine, thiomorpholine, thiomorpholine-1,1-dioxide or tetrahydropyrane; Rₐ₀, R_{b0} are each independently hydrogen or C₁₋₃ alkyl.

In another preferred example, Rₐ₂ is a hydrogen or C₁₋₈ alkyl (preferably C₁₋₆ alkyl, more preferably C₁₋₃ alkyl); R_{b2} is a hydrogen, C₁₋₈ alkyl (preferably C₁₋₆ alkyl, more preferably C₁₋₃ alkyl), 4 to 6 membered saturated single heterocycle, 5 to 6 membered monocyclic heteroaryl ring, C₆₋₁₀ aryl (R_{b2} is preferably 5 to 6 membered monocyclic heteroaryl ring, more preferably pyridine);

the 4 to 6 membered saturated single heterocycle, 5 to 6 membered monocyclic heteroaryl ring or aryl is unsubstituted or substituted with 1, 2 or 3 groups selected from the group consisting of NRₐ₀R_{b0}, hydroxymethyl, hydroxyethyl, carboxyl, —C(O)OC₁₋₆ alkyl, acetyl, hydroxy, C₁₋₃ alkyl, halogenated C₁₋₃ alkyl, C₃₋₆ cycloalkyl, azetidine, oxetane, tetrahydrofuran, tetrahydrothiophene, pyrrolidine, piperidine, oxazolidine, piperazine, dioxolane, dioxane, morpholine, thiomorpholine, thiomorpholine-1,1-dioxide or tetrahydropyrane; Rₐ₀, R_{b0} are each independently hydrogen or C₁₋₃ alkyl.

In another preferred example, Rₐ₃ is a C₁₋₈ alkyl (preferably C₁₋₆ alkyl, more preferably C₁₋₃ alkyl).

In another preferred example, Rₐ₄ is a C₁₋₈ alkyl substituted with C₁₋₈ alkoxy (preferably C₁₋₆ alkyl substituted with C₁₋₆ alkoxy, more preferably C₁₋₃ alkyl substituted with C₁₋₃ alkoxy), 5 to 6 membered monocyclic heteroaryl ring (preferably pyridine), or C₆₋₁₀ aryl (preferably phenyl);

the 5 to 6 membered monocyclic heteroaryl ring or aryl is unsubstituted or substituted with 1, 2 or 3 groups selected from the group consisting of NRₐ₀R_{b0}, acetyl, hydroxy, C₁₋₃ alkyl, halogenated C₁₋₃ alkyl, C₃₋₆ cycloalkyl, azetidine, oxetane, tetrahydrofuran, tetrahydrothiophene, pyrrolidine, piperidine, oxazolidine, piperazine, dioxolane, dioxane, morpholine, thiomorpholine, thiomorpholine-1,1-dioxide or tetrahydropyrane; Rₐ₀, R_{b0} are each independently hydrogen or C₁₋₃ alkyl.

In another preferred example, aryl in R₂ (preferably phenyl) is unsubstituted or substituted with 1, 2 or 3 groups selected from the group consisting of C₁₋₈ alkyl (preferably C₁₋₆ alkyl, more preferably C₁₋₃ alkyl), halogenated C₁₋₈ alkyl (preferably halogenated C₁₋₆ alkyl, more preferably halogenated C₁₋₃ alkyl), C₁₋₈ alkoxy (preferably C₁₋₆ alkoxy, more preferably C₁₋₃ alkoxy), halogen, —O(CH₂)ₙOC₁₋₈ alkyl or —Y₁-L₁; wherein Y₁ is a bond, CH₂, NH, O or CON; L₁ is a C₆₋₁₀ aryl (preferably phenyl), 5 to 6 membered monocyclic heteroaryl ring (preferably pyridine), 4 to 6 membered saturated single heterocycle (preferably piperidine, piperazine, morpholine or tetrahydropyrane) or NRₐ₀R_{b0}; Rₐ₀, R_{b0} are each independently hydrogen or C₁₋₈ alkyl (preferably C₁₋₆ alkyl, more preferably C₁₋₃ alkyl);

the aryl, 4 to 6 membered saturated single heterocycle, 5 to 6 membered monocyclic heteroaryl ring in L₁ is unsubstituted or substituted with 1, 2 or 3 groups selected from the group consisting of NRₐ₀R_{b0}, hydroxymethyl, hydroxyethyl, carboxyl, —C(O)OC₁₋₆ alkyl, acetyl, hydroxy, C₁₋₃ alkyl, halogenated C₁₋₃ alkyl, C₃₋₆ cycloalkyl, azetidine, oxetane, tetrahydrofuran, tetrahydrothiophene, pyrrolidine, piperidine, oxazolidine, piperazine, dioxolane, dioxane, morpholine, thiomorpholine, thiomorpholine-1,1-dioxide or tetrahydropyrane; Rₐ₀, R_{b0} are each independently hydrogen or C₁₋₃ alkyl.

In another preferred example, the 5 to 6 membered monocyclic heteroaryl ring in R₂ is unsubstituted or substituted with 1, 2 or 3 groups selected from the group consisting of halogen, C₁₋₈ alkyl (preferably C₁₋₆ alkyl, more preferably C₁₋₃ alkyl), halogenated C₁₋₈ alkyl (preferably halogenated C₁₋₆ alkyl, more preferably halogenated C₁₋₃ alkyl), C₃₋₈ cycloalkyl (preferably C₃₋₆ cycloalkyl) or —Y₂-L₂; wherein Y₂ is a bond, CH₂ or C(O); L₂ is a hydrogen, C₃₋₆ cycloalkyl, 4 to 6 membered saturated single heterocycle or NRₐ₀R_{b0}; Rₐ₀, R_{b0} are each independently hydrogen or C₁₋₈ alkyl (preferably C₁₋₆ alkyl, more preferably C₁₋₃ alkyl);

the 4 to 6 membered saturated single heterocycle in L₂ is unsubstituted or substituted with 1, 2 or 3 groups selected from the group consisting of NRₐ₀R_{b0}, hydroxymethyl, hydroxyethyl, carboxyl, —C(O)OC₁₋₆ alkyl, acetyl, hydroxy, C₁₋₃ alkyl, halogenated C₁₋₃ alkyl, C₃₋₆ cycloalkyl, azetidine, oxetane, tetrahydrofuran, tetrahydrothiophene, pyrrolidine, piperidine, oxazolidine, piperazine, dioxolane, dioxane, morpholine, thiomorpholine, thiomorpholine-1,1-dioxide or tetrahydropyrane; Rₐ₀, R_{b0} are each independently hydrogen or C₁₋₃ alkyl.

In another preferred example, R₆ is a C₁₋₆ alkyl, CHRₐ₅R_{b5}, C₃₋₆ cycloalkyl, 4 to 6 membered saturated single heterocycle (preferably azetidine, oxetane, tetrahydrofuran, tetrahydrothiophene, pyrrolidine, piperidine, piperazine, morpholine or tetrahydropyrane, more preferably piperidine or tetrahydropyrane), spiro, spiroheterocycle, bridged ring, bridged heterocycle; wherein Ras is a hydrogen, methyl or ethyl; R_{b5} is a phenyl, 5 to 6 membered monocyclic heteroaryl ring (preferably pyridine) or 4 to 6 membered saturated single heterocycle (preferably piperidine, piperazine, morpholine or tetrahydropyrane); the alkyl, phenyl is unsubstituted or substituted with 1, 2 or 3 groups selected from the group consisting of C₁₋₃ alkoxy, NRₐ₀R_{b0} or 4 to 6 membered saturated single heterocycle (preferably piperidine, piperazine, morpholine or tetrahydropyrane); Rₐ₀, R_{b0} are each independently hydrogen, methyl or ethyl.

In another preferred example, cycloalkyl in R₆ is unsubstituted or substituted with 1, 2 or 3 groups selected from the group consisting of NRₐ₀R_{b0}, halogen, hydroxy, C₁₋₈ alkyl (preferably C₁₋₆ alkyl, more preferably C₁₋₃ alkyl), C₁₋₈ alkoxy (preferably C₁₋₆ alkoxy, more preferably C₁₋₃ alkoxy), halogenated C₁₋₈ alkyl (preferably halogenated C₁₋₆ alkyl, more preferably halogenated C₁₋₃ alkyl), C₃₋₈ cycloalkyl (preferably C₃₋₆ cycloalkyl), 4 to 6 membered saturated single heterocycle (preferably azetidine, oxetane, tetrahydrofuran, tetrahydrothiophene, pyrrolidine, piperidine, piperazine, morpholine or tetrahydropyrane); Rₐ₀, R_{b0} are each independently hydrogen, acetyl, C₁₋₈ alkyl (preferably C₁₋₆ alkyl, more preferably C₁₋₃ alkyl), C₁₋₈ alkyl substituted with C₁₋₈ alkoxy (preferably C₁₋₆ alkyl substituted with C₁₋₆ alkoxy, more preferably C₁₋₃ alkyl substituted with C₁₋₃ alkoxy); the 4 to 6 membered saturated single heterocycle is unsubstituted or substituted with C₁₋₈ alkoxy (preferably C₁₋₆ alkoxy, more preferably C₁₋₃ alkoxy).

In another preferred example, the 4 to 6 membered saturated single heterocycle in R₆ is unsubstituted or substituted with 1, 2 or 3 groups selected from the group consisting of acetyl, C₁₋₈ alkyl (preferably C₁₋₆ alkyl, more preferably C₁₋₃ alkyl), C₁₋₈ alkyl substituted with C₁₋₈ alkoxy (preferably C₁₋₆ alkyl substituted with C₁₋₆ alkoxy, more preferably C₁₋₃ alkyl substituted with C₁₋₃ alkoxy), —SO₂C₁₋₈ alkyl (preferably —SO₂C₁₋₆ alkyl, more preferably —SO₂C₁₋₃ alkyl), halogenated C₁₋₈ alkyl (preferably halogenated C₁₋₆ alkyl, more preferably halogenated C₁₋₃ alkyl), C₃₋₈ cycloalkyl (preferably C₃₋₆ cycloalkyl), 4 to 6 membered saturated single heterocycle (preferably azetidine, oxetane, tetrahydrofuran, tetrahydrothiophene, pyrrolidine, piperidine, piperazine, morpholine or tetrahydropyrane); the acetyl is unsubstituted or substituted with CN or hydroxy.

In another preferred example, X is NH.

In another preferred example, R₂ is a halogen (preferably fluorine, chlorine, bromine), CN, C₁₋₈ alkyl (preferably C₁₋₆ alkyl, more preferably C₁₋₃ alkyl), halogenated C₁₋₈ alkyl (preferably halogenated C₁₋₆ alkyl, more preferably halogenated C₁₋₃ alkyl), C₃₋₈ cycloalkyl (preferably C₃₋₆ cycloalkyl), or C₁₋₈ alkoxy (preferably C₁₋₆ alkoxy, more preferably C₁₋₃ alkoxy).

In another preferred example, R₂ is a C₂₋₈ alkynyl (preferably C₂₋₆ alkynyl, more preferably C₂₋₃ alkynyl); the alkynyl is substituted with 4 to 6 membered saturated single heterocycle (preferably piperidine, piperazine, morpholine or tetrahydropyrane);

the 4 to 6 membered saturated single heterocycle is unsubstituted or substituted with 1, 2 or 3 groups selected from the group consisting of NRₐ₀R_{b0}, hydroxymethyl, hydroxyethyl, carboxyl, —C(O)OC₁₋₆ alkyl, acetyl, hydroxy, C₁₋₃ alkyl, halogenated C₁₋₃ alkyl, C₃₋₆ cycloalkyl, azetidine, oxetane, tetrahydrofuran, tetrahydrothiophene, pyrrolidine, piperidine, oxazolidine, piperazine, dioxolane, dioxane, morpholine, thiomorpholine, thiomorpholine-1,1-dioxide or tetrahydropyrane; Rₐ₀, R_{b0} are each independently hydrogen or C₁₋₃ alkyl.

In another preferred example, R₂ is C(O)NRₐ₁R_{b1}; Rₐ₁ is a hydrogen or C₁₋₈ alkyl (preferably C₁₋₆ alkyl, more preferably C₁₋₃ alkyl); R_{b1} is a hydrogen, C₁₋₈ alkyl (preferably C₁₋₆ alkyl, more preferably C₁₋₃ alkyl), 4 to 6 membered saturated single heterocycle (preferably piperidine, piperazine, morpholine or tetrahydropyrane), 5 to 6 membered monocyclic heteroaryl ring (preferably pyridine) or phenyl; or Rₐ₁, R_{b1} and the nitrogen atom attached thereto form a 4 to 6 membered saturated single heterocycle (preferably piperidine, piperazine, morpholine or tetrahydropyrane ring);

the 4 to 6 membered saturated single heterocycle, 5 to 6 membered monocyclic heteroaryl ring or phenyl is unsubstituted or substituted with 1, 2 or 3 groups selected from the group consisting of NRₐ₀R_{b0}, hydroxymethyl, hydroxyethyl, carboxyl, —C(O)OC₁₋₆ alkyl, acetyl, hydroxy, C₁₋₃ alkyl, halogenated C₁₋₃ alkyl, C₃₋₆ cycloalkyl, azetidine, oxetane, tetrahydrofuran, tetrahydrothiophene, pyrrolidine, piperidine, oxazolidine, piperazine, dioxolane, dioxane, morpholine, thiomorpholine, thiomorpholine-1,1-dioxide or tetrahydropyrane; Rₐ₀, R_{b0} are each independently hydrogen or C₁₋₃ alkyl.

In another preferred example, R₂ is NRₐ₂R_{b2}; Rₐ₂ is hydrogen or C₁₋₈ alkyl (preferably C₁₋₆ alkyl, more preferably C₁₋₃ alkyl); R_{b2} is hydrogen, C₁₋₈ alkyl (preferably C₁₋₆ alkyl, more preferably C₁₋₃ alkyl), 4 to 6 membered saturated single heterocycle, 5 to 6 membered monocyclic heteroaryl ring, C₆₋₁₀ aryl (R_{b2} preferably 5 to 6 membered monocyclic heteroaryl ring, more preferably pyridine);

the 4 to 6 membered saturated single heterocycle, 5 to 6 membered monocyclic heteroaryl ring or aryl is unsubstituted or substituted with 1, 2 or 3 groups selected from the group consisting of NRₐ₀R_{b0}, hydroxymethyl, hydroxyethyl, carboxyl, —C(O)OC₁₋₆ alkyl, acetyl, hydroxy, C₁₋₃ alkyl, halogenated C₁₋₃ alkyl, C₃₋₆ cycloalkyl, azetidine, oxetane, tetrahydrofuran, tetrahydrothiophene, pyrrolidine, piperidine, oxazolidine, piperazine, dioxolane, dioxane, morpholine, thiomorpholine, thiomorpholine-1,1-dioxide or tetrahydropyrane; Rₐ₀, R_{b0} are each independently hydrogen or C₁₋₃ alkyl.

In another preferred example, R₂ is NCORₐ₃; Rₐ₃ is C₁₋₈ alkyl (preferably C₁₋₆ alkyl, more preferably C₁₋₃ alkyl).

In another preferred example, R₂ is ORₐ₄; Rₐ₄ is C₁₋₈ alkyl substituted with C₁₋₈ alkoxy (preferably C₁₋₆ alkyl substituted with C₁₋₆ alkoxy, more preferably C₁₋₃ alkyl substituted with C₁₋₃ alkoxy), 5 to 6 membered monocyclic heteroaryl ring (preferably pyridine), C₆₋₁₀ aryl (preferably phenyl);

the 5 to 6 membered monocyclic heteroaryl ring or aryl is unsubstituted or substituted with 1, 2 or 3 groups selected from the group consisting of NRₐ₀R_{b0}, acetyl, hydroxy, C₁₋₃ alkyl, halogenated C₁₋₃ alkyl, C₃₋₆ cycloalkyl, azetidine, oxetane, tetrahydrofuran, tetrahydrothiophene, pyrrolidine, piperidine, oxazolidine, piperazine, dioxolane, dioxane, morpholine, thiomorpholine, thiomorpholine-1,1-dioxide or tetrahydropyrane; Rₐ₀, R_{b0} are each independently hydrogen or C₁₋₃ alkyl.

In another preferred example, R₂ is phenyl; the phenyl is unsubstituted or substituted with 1, 2 or 3 groups selected from the group consisting of C₁₋₈ alkyl (preferably C₁₋₆ alkyl, more preferably C₁₋₃ alkyl), halogenated C₁₋₈ alkyl (preferably halogenated C₁₋₆ alkyl, more preferably halogenated C₁₋₃ alkyl), C₁₋₈ alkoxy (preferably C₁₋₆ alkoxy, more preferably C₁₋₃ alkoxy), halogen, —O(CH₂)ₙOC₁₋₃ alkyl or —Y₁-L₁; wherein Y₁ is a bond, CH₂, NH, O or CON; L₁ is C₆₋₁₀ aryl (preferably phenyl), 5 to 6 membered monocyclic heteroaryl ring (preferably pyridine), 4 to 6 membered saturated single heterocycle (preferably piperidine, piperazine, morpholine or tetrahydropyrane) or NRₐ₀R_{b0}; Rₐ₀, R_{b}0 are each independently hydrogen or C₁₋₈ alkyl (preferably C₁₋₆ alkyl, more preferably C₁₋₃ alkyl);

the aryl, 4 to 6 membered saturated single heterocycle, 5 to 6 membered monocyclic heteroaryl ring is unsubstituted or substituted with 1, 2 or 3 groups selected from the group consisting of NRₐ₀R_{b0}, hydroxymethyl, hydroxyethyl, carboxyl, —C(O)OC₁₋₆ alkyl, acetyl, hydroxy, C₁₋₃ alkyl, halogenated C₁₋₃ alkyl, C₃₋₆ cycloalkyl, azetidine, oxetane, tetrahydrofuran, tetrahydrothiophene, pyrrolidine, piperidine, oxazolidine, piperazine, dioxolane, dioxane, morpholine, thiomorpholine, thiomorpholine-1,1-dioxide or tetrahydropyrane; Rₐ₀, R_{b0} are each independently hydrogen or C₁₋₃ alkyl.

In another preferred example, R₂ is a structure shown by formula A:

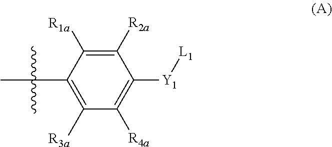

wherein R₁ₐ, R₂ₐ, R₃ₐ, R₄ₐ are each independently hydrogen, C₁₋₈ alkyl (preferably C₁₋₆ alkyl, more preferably C₁₋₃ alkyl), halogenated C₁₋₈ alkyl (preferably halogenated C₁₋₆ alkyl, more preferably halogenated C₁₋₃ alkyl), C₁₋₈ alkoxy (preferably C₁₋₆ alkoxy, more preferably C₁₋₃ alkoxy) or halogen; Y₁, L₁ are as defined above.

In another preferred example, R₁ₐ, R₃ₐ, R₄ₐ are hydrogen.

In another preferred example, R₂ is 5 to 6 membered monocyclic heteroaryl ring (preferably pyridine, pyrimidine or pyrazole); the 5 to 6 membered monocyclic heteroaryl ring is unsubstituted or substituted with 1, 2 or 3 groups selected from the group consisting of halogen, C₁₋₈ alkyl (preferably C₁₋₆ alkyl, more preferably C₁₋₃ alkyl), halogenated C₁₋₈ alkyl (preferably halogenated C₁₋₆ alkyl, more preferably halogenated C₁₋₃ alkyl), C₃₋₈ cycloalkyl (preferably C₃₋₆ cycloalkyl) or —Y₂-L₂; wherein Y₂ is a bond, CH₂ or C(O); L₂ is a hydrogen, C₃₋₆ cycloalkyl, 4 to 6 membered saturated single heterocycle or NRₐ₀R_{b0}; Rₐ₀, R_{b0} are each independently hydrogen or C₁₋₈ alkyl (preferably C₁₋₆ alkyl, more preferably C₁₋₃ alkyl);

the 4 to 6 membered saturated single heterocycle is unsubstituted or substituted with 1, 2 or 3 groups selected from the group consisting of NRₐ₀R_{b0}, hydroxymethyl, hydroxyethyl, carboxyl, —C(O)OC₁₋₆ alkyl, acetyl, hydroxy, C₁₋₃ alkyl, halogenated C₁₋₃ alkyl, C₃₋₆ cycloalkyl, azetidine, oxetane, tetrahydrofuran, tetrahydrothiophene, pyrrolidine, piperidine, oxazolidine, piperazine, dioxolane, dioxane, morpholine, thiomorpholine, thiomorpholine-1,1-dioxide or tetrahydropyrane; Rₐ₀, R_{b0} are each independently hydrogen or C₁₋₃ alkyl.

In another preferred example, R₂ is a structure shown by formula B or C:

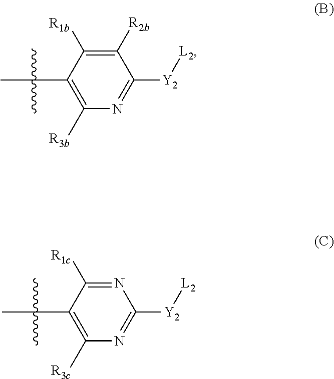

wherein R_{1b}, R_{2b}, R_{3b}, R_{1c}, R_{3c} are each independently hydrogen, halogen, C₁₋₈ alkyl (preferably C₁₋₆ alkyl, more preferably C₁₋₃ alkyl), halogenated C₁₋₈ alkyl (preferably halogenated C₁₋₆ alkyl, more preferably halogenated C₁₋₃ alkyl), C₃₋₈ cycloalkyl (preferably C₃₋₆ cycloalkyl); Y₂, L₂ are as defined above.

In another preferred example, R_{1b}, R_{3b} are hydrogen.

In another preferred example, R_{1c}, R_{3c} are hydrogen.

In another preferred example, R₂ is 4 to 6 membered saturated single heterocycle (preferably piperidine, piperazine, morpholine or tetrahydropyrane); the 4 to 6 membered saturated single heterocycle is unsubstituted or substituted with NRₐ₀R_{b0}, hydroxymethyl, hydroxyethyl, carboxyl, —C(O)OC₁₋₆ alkyl, acetyl, hydroxy, C₁₋₃ alkyl, halogenated C₁₋₃ alkyl, C₃₋₆ cycloalkyl, azetidine, oxetane, tetrahydrofuran, tetrahydrothiophene, pyrrolidine, piperidine, oxazolidine, piperazine, dioxolane, dioxane, morpholine, thiomorpholine, thiomorpholine-1,1-dioxide or tetrahydropyrane; Rₐ₀, R_{b0} are each independently hydrogen or C₁₋₃ alkyl.

In another preferred example, R₂ is fluorine, chlorine, bromine, CN, methyl, ethyl, n-propyl, isopropyl, cyclopropyl, methoxy, ethoxy or trifluoromethyl, or R₂ is the following structure:

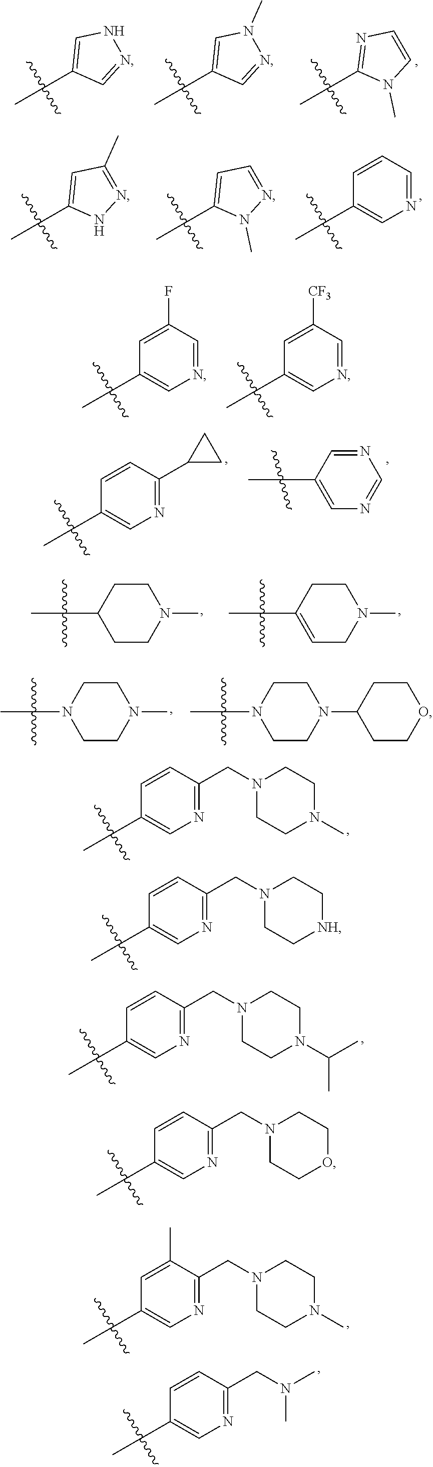

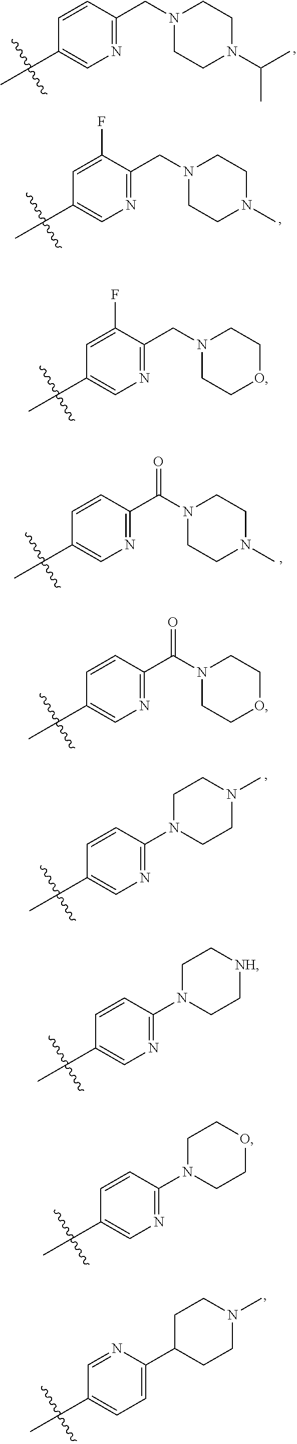

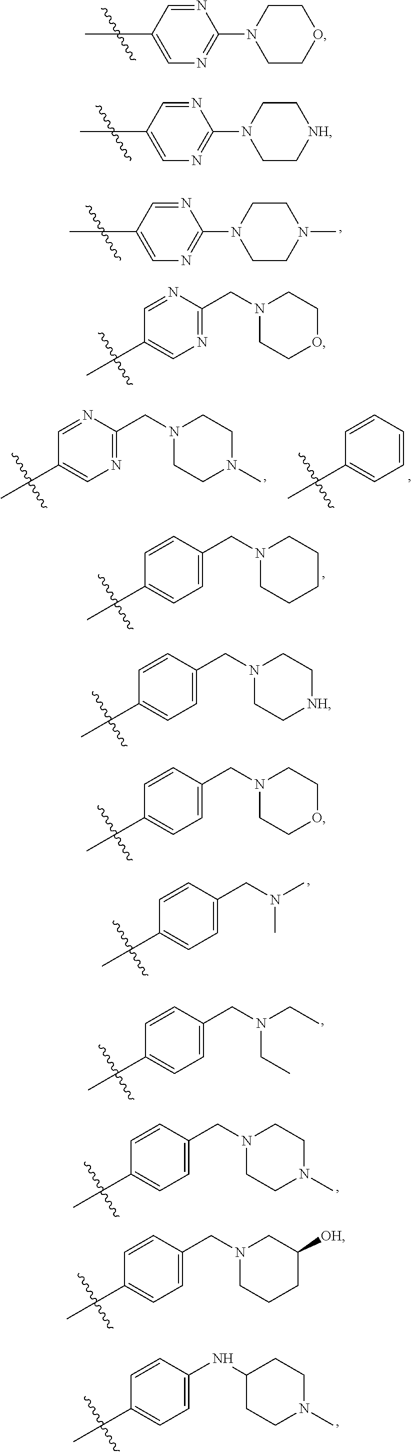

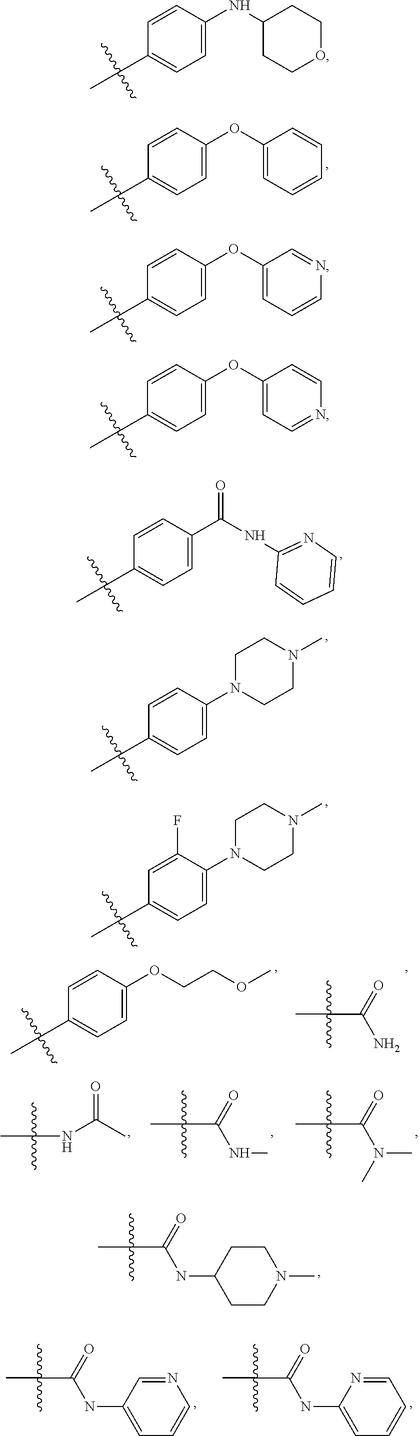

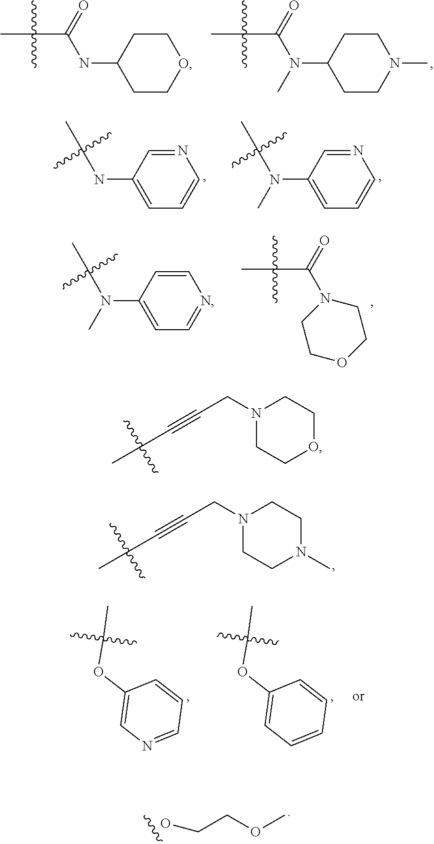

In another preferred example, R₅ is hydrogen, methyl, ethyl, or methoxy substituted ethyl.

In another preferred example, R₆ is C₁₋₆ alkyl or CHRₐ₅R_{b5}; wherein Rₐ₅ is hydrogen, methyl or ethyl; R_{b5} is phenyl, 5 to 6 membered monocyclic heteroaryl ring (preferably pyridine) or 4 to 6 membered saturated single heterocycle (preferably piperidine, piperazine, morpholine or tetrahydropyrane); the alkyl, phenyl are unsubstituted or substituted with 1, 2 or 3 groups selected from the group consisting of C₁₋₃ alkoxy, NRₐ₀R_{b0} or 4 to 6 membered saturated single heterocycle (preferably piperidine, piperazine, morpholine or tetrahydropyrane); Rₐ₀, R_{b0} are each independently hydrogen, methyl or ethyl.

In another preferred example, R₆ is C₃₋₆ cycloalkyl (preferably cyclohexyl); the cycloalkyl is unsubstituted or substituted with 1, 2 or 3 groups selected from the group consisting of NRₐ₀R_{b0}, halogen, hydroxy, C₁₋₈ alkyl (preferably C₁₋₆ alkyl, more preferably C₁₋₃ alkyl), C₁₋₈ alkoxy (preferably C₁₋₆ alkoxy, more preferably C₁₋₃ alkoxy), halogenated C₁₋₈ alkyl (preferably halogenated C₁₋₆ alkyl, more preferably halogenated C₁₋₃ alkyl), C₃₋₈ cycloalkyl (preferably C₃₋₆ cycloalkyl), 4 to 6 membered saturated single heterocycle (preferably azetidine, oxetane, tetrahydrofuran, tetrahydrothiophene, pyrrolidine, piperidine, piperazine, morpholine or tetrahydropyrane); Rₐ₀, R_{b0} are each independently hydrogen, acetyl, C₁₋₈ alkyl (preferably C₁₋₆ alkyl, more preferably C₁₋₃ alkyl), C₁₋₈ alkyl substituted with C₁₋₈ alkoxy (preferably C₁₋₆ alkyl substituted with C₁₋₆ alkoxy, more preferably C₁₋₃ alkyl substituted with C₁₋₃ alkoxy); the 4 to 6 membered saturated single heterocycle is unsubstituted or substituted with C₁₋₈ alkoxy (preferably C₁₋₆ alkoxy, more preferably C₁₋₃ alkoxy).

In another preferred example, R₆ is a structure shown by formula D:

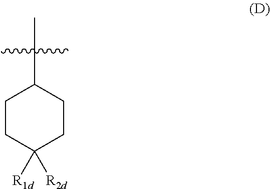

wherein R_{1d} is hydrogen, halogen or C₁₋₈ alkyl (preferably C₁₋₆ alkyl, more preferably C₁₋₃ alkyl); R_{2d} is hydrogen, NRₐ₀R_{b0}, halogen, hydroxy, C₁₋₈ alkyl (preferably C₁₋₆ alkyl, more preferably C₁₋₃ alkyl), C₁₋₈ alkoxy (preferably C₁₋₆ alkoxy, more preferably C₁₋₃ alkoxy), 4 to 6 membered saturated single heterocycle (preferably azetidine, oxetane, tetrahydrofuran, tetrahydrothiophene, pyrrolidine, piperidine, piperazine, morpholine or tetrahydropyrane); Rₐ₀, R_{b0} are each independently hydrogen, acetyl, C₁₋₈ alkyl (preferably C₁₋₆ alkyl, more preferably C₁₋₃ alkyl), C₁₋₈ alkyl substituted with C₁₋₈ alkoxy (preferably C₁₋₆ alkyl substituted with C₁₋₆ alkoxy, more preferably C₁₋₃ alkyl substituted with C₁₋₃ alkoxy); the 4 to 6 membered saturated single heterocycle is unsubstituted or substituted with C₁₋₈ alkoxy (preferably C₁₋₆ alkoxy, more preferably C₁₋₃ alkoxy).

In another preferred example, R₆ is 4 to 6 membered saturated single heterocycle (preferably azetidine, oxetane, tetrahydrofuran, tetrahydrothiophene, pyrrolidine, piperidine, piperazine, morpholine or tetrahydropyrane, more preferably piperidine or tetrahydropyrane); the 4 to 6 membered saturated single heterocycle is unsubstituted or substituted with 1, 2 or 3 groups selected from the group consisting of acetyl, C₁₋₈ alkyl (preferably C₁₋₆ alkyl, more preferably C₁₋₃ alkyl), C₁₋₈ alkyl substituted with C₁₋₈ alkoxy (preferably C₁₋₆ alkyl substituted with C₁₋₆ alkoxy, more preferably C₁₋₃ alkyl substituted with C₁₋₃ alkoxy), —SO₂C₁₋₈ alkyl (preferably —SO₂C₁₋₆ alkyl, more preferably —SO₂C₁₋₃ alkyl), halogenated C₁₋₈ alkyl (preferably halogenated C₁₋₆ alkyl, more preferably halogenated C₁₋₃ alkyl), C₃₋₈ cycloalkyl (preferably C₃₋₆ cycloalkyl), 4 to 6 membered saturated single heterocycle (preferably azetidine, oxetane, tetrahydrofuran, tetrahydrothiophene, pyrrolidine, piperidine, piperazine, morpholine or tetrahydropyrane); the acetyl is unsubstituted or substituted with CN or hydroxy.

In another preferred example, R₆ is tetrahydropyran or a structure of formula E:

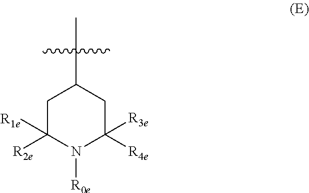

wherein R₁ₑ, R₂ₑ, R₃ₑ, R₄ₑ are each independently hydrogen or methyl; R₀ₑ is hydrogen, acetyl, C₁₋₈ alkyl (preferably C₁₋₆ alkyl, more preferably C₁₋₃ alkyl), C₁₋₈ alkyl substituted with C₁₋₈ alkoxy (preferably C₁₋₆ alkyl substituted with C₁₋₆ alkoxy, more preferably C₁₋₃ alkyl substituted with C₁₋₃ alkoxy), —SO₂C₁₋₈ alkyl (preferably —SO₂C₁₋₆ alkyl, more preferably —SO₂C₁₋₃ alkyl), halogenated C₁₋₈ alkyl (preferably halogenated C₁₋₆ alkyl, more preferably halogenated C₁₋₃ alkyl), C₃₋₈ cycloalkyl (preferably C₃₋₆ cycloalkyl), 4 to 6 membered saturated single heterocycle (preferably azetidine, oxetane, tetrahydrofuran, tetrahydrothiophene, pyrrolidine, piperidine, piperazine, morpholine or tetrahydropyrane); the acetyl is unsubstituted or substituted with CN or hydroxy.

In another preferred example, R₆ is spiro, spiroheterocycle, bridged ring, or bridged heterocycle.

In another preferred example, R₆ is a bicyclic spiroheterocycle containing 1-2 nitrogen or oxygen atoms.

In another preferred example, R₆ is a bicyclic bridged heterocycle containing 1-2 nitrogen or oxygen atoms.

In another preferred example, R₆ is the following structure:

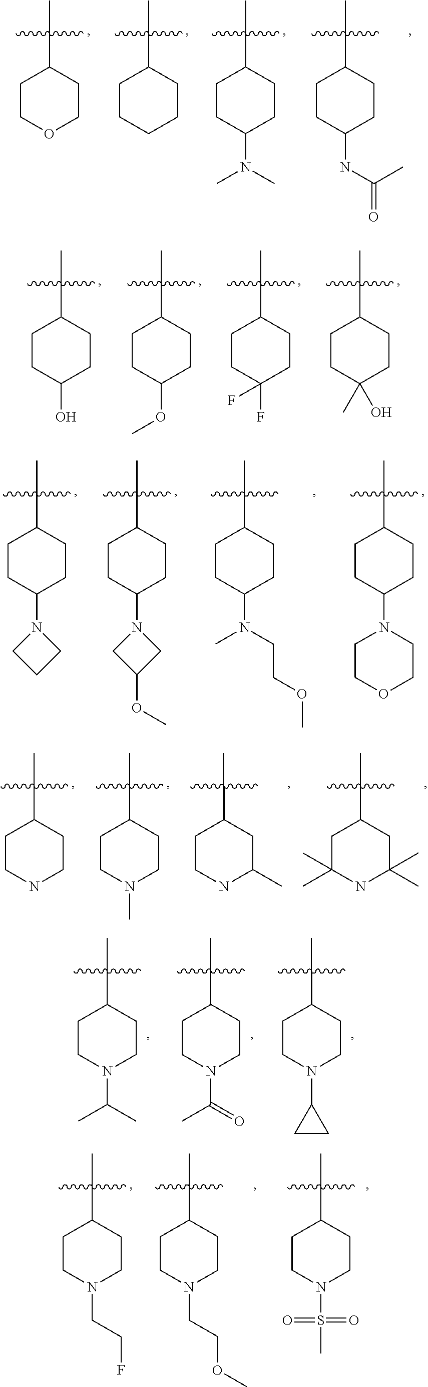

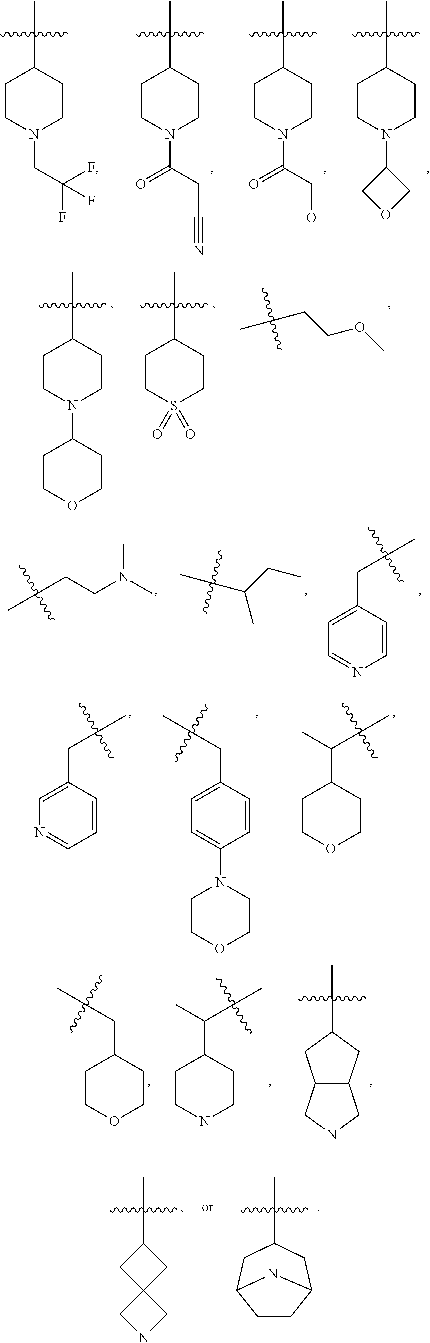

In another preferred example, R₁ is hydrogen or halogen (preferably fluorine, chlorine, bromine).

In another preferred example, R₃ is hydrogen or halogen (preferably fluorine, chlorine, bromine).

In another preferred example, R₄ is hydroxy, C₁₋₃ alkyl (preferably methyl or ethyl) or C₁₋₃ alkoxy (preferably methoxy).

In another preferred example, Z₁ is N; Z₂ is CR₉; Z₃ is CR₁₀; R₉, R₁₀ are each independently hydrogen, halogen (preferably fluorine, chlorine, bromine), C₁₋₈ alkyl (preferably C₁₋₆ alkyl, more preferably C₁₋₃ alkyl), preferably R₉, R₁₀ are hydrogen.

In another preferred example, Z₁ is N; Z₂, Z₃ are CH; X is NH; R₁ is hydrogen; R₃ is hydrogen or fluorine; R₂, R₄, R₅, R₆ are as defined in the present specification.

In another preferred example, Z₁ is N; Z₂ is N; Z₃ is CR₁₀; R₁₀ is hydrogen, halogen (preferably fluorine, chlorine, bromine), C₁₋₈ alkyl (preferably C₁₋₆ alkyl, more preferably C₁₋₃ alkyl), preferably R₁₀ is hydrogen.

In another preferred example, Z₁, Z₂ are N; Z₃ is CH; X is NH; R₁, R₃ are hydrogen; R₂, R₄, R₅, R₆ are as defined in the present specification.

In another preferred example, Z₁ is N; Z₂ is CR₉; Z₃ is N; R₉ is hydrogen, halogen (preferably fluorine, chlorine, bromine), C₁₋₈ alkyl (preferably C₁₋₆ alkyl, more preferably C₁₋₃ alkyl), preferably R₉ is hydrogen.

In another preferred example, Z₁, Z₃ are N; Z₂ is CH; X is NH; R₁, R₃ are hydrogen; R₂, R₄, R₅, R₆ are as defined in the present specification.

In another preferred example, Z₁ is CR₈; Z₂ is N; Z₃ is CR₁₀; R₈, R₁₀ are each independently hydrogen, halogen (preferably fluorine, chlorine, bromine), C₁₋₈ alkyl (preferably C₁₋₆ alkyl, more preferably C₁₋₃ alkyl), preferably R₈, R₁₀ are hydrogen.

In another preferred example, Z₂ is N; Z₁, Z₃ are CH; X is NH; R₁, R₃ are hydrogen; R₂, R₄, R₅, R₆ are as defined in the present specification.

In another preferred example, the compound is selected from the following Table A

TABLE A

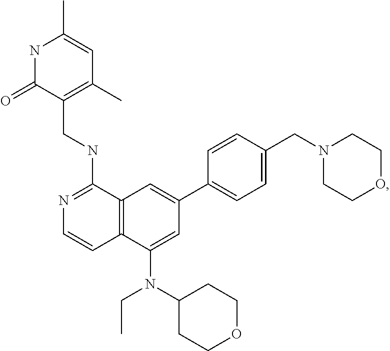 [SMILES: CCN(c1cc(-c2ccc(CN3CCOCC3)cc2)cc2c(NCc3c(C)cc(C)[nH]c3=O)nccc12)C1CCOCC1]

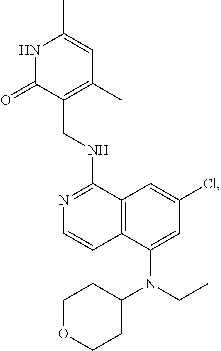 [SMILES: CCN(c1cc(Cl)cc2c(NCc3c(C)cc(C)[nH]c3=O)nccc12)C1CCOCC1]

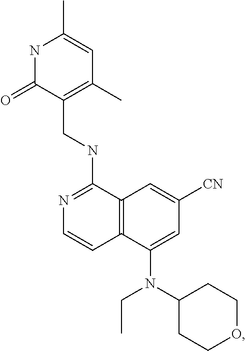 [SMILES: CCN(c1cc(C#N)cc2c(NCc3c(C)cc(C)[nH]c3=O)nccc12)C1CCOCC1]

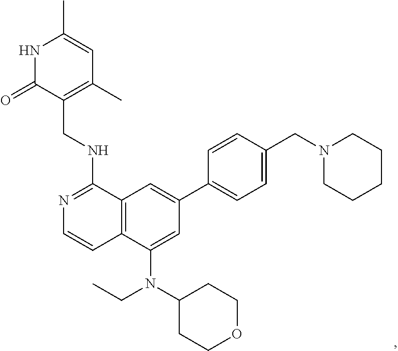 [SMILES: CCN(c1cc(-c2ccc(CN3CCCCC3)cc2)cc2c(NCc3c(C)cc(C)[nH]c3=O)nccc12)C1CCOCC1]

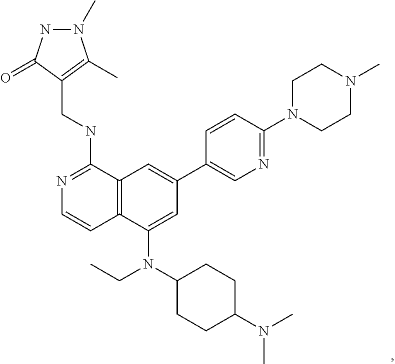 [SMILES: CCN(c1cc(-c2ccc(N3CCN(C)CC3)nc2)cc2c(NCc3c(C)n(C)[nH]c3=O)nccc12)C1CCC(N(C)C)CC1]

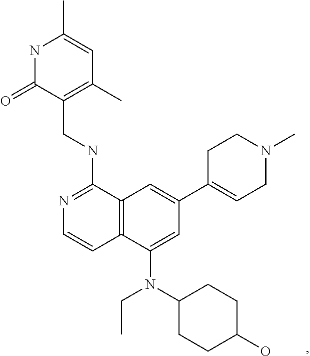 [SMILES: CCN(c1cc(C2=CCN(C)CC2)cc2c(NCc3c(C)cc(C)[nH]c3=O)nccc12)C1CCC(O)CC1]

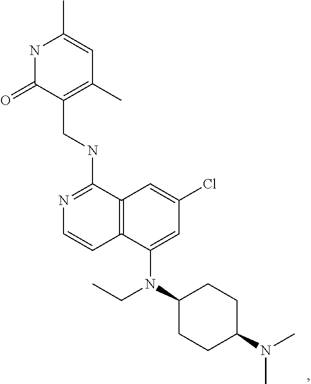 [SMILES: CCN(c1cc(Cl)cc2c(NCc3c(C)cc(C)[nH]c3=O)nccc12)[C@H]1CC[C@@H](N(C)C)CC1]

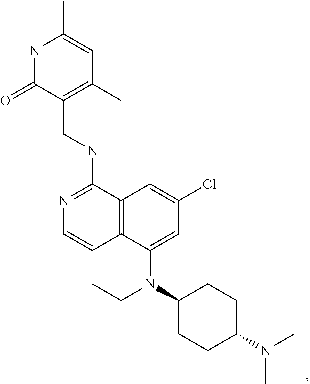 [SMILES: CCN(c1cc(Cl)cc2c(NCc3c(C)cc(C)[nH]c3=O)nccc12)[C@H]1CC[C@H](N(C)C)CC1]

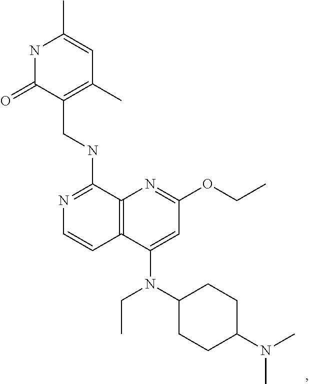 [SMILES: CCOc1cc(N(CC)C2CCC(N(C)C)CC2)c2ccnc(NCc3c(C)cc(C)[nH]c3=O)c2n1]

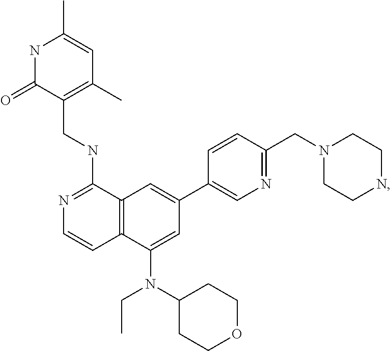

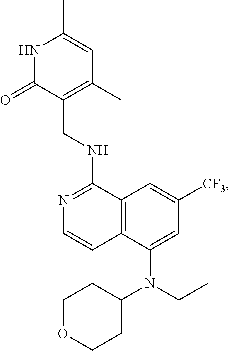 [SMILES: CCN(c1cc(C(F)(F)F)cc2c(NCc3c(C)cc(C)[nH]c3=O)nccc12)C1CCOCC1]

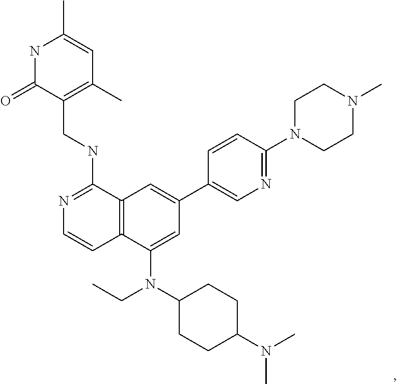 [SMILES: CCN(c1cc(-c2ccc(N3CCN(C)CC3)nc2)cc2c(NCc3c(C)cc(C)[nH]c3=O)nccc12)C1CCC(N(C)C)CC1]

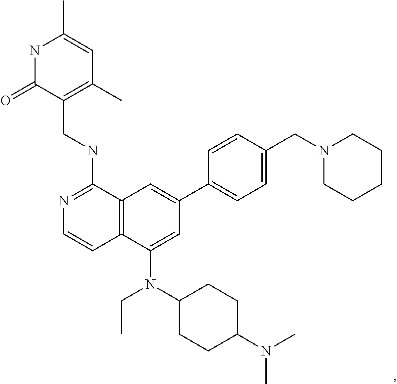 [SMILES: CCN(c1cc(-c2ccc(CN3CCCCC3)cc2)cc2c(NCc3c(C)cc(C)[nH]c3=O)nccc12)C1CCC(N(C)C)CC1]

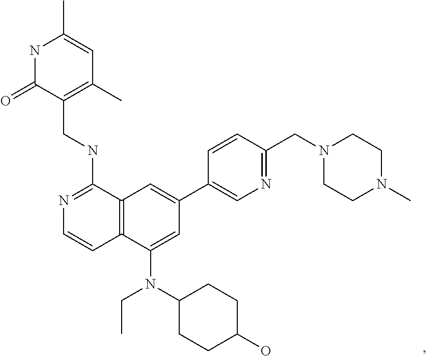 [SMILES: CCN(c1cc(-c2ccc(CN3CCN(C)CC3)nc2)cc2c(NCc3c(C)cc(C)[nH]c3=O)nccc12)C1CCC(O)CC1]

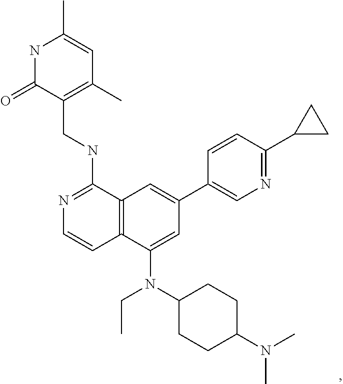 [SMILES: CCN(c1cc(-c2ccc(C3CC3)nc2)cc2c(NCc3c(C)cc(C)[nH]c3=O)nccc12)C1CCC(N(C)C)CC1]

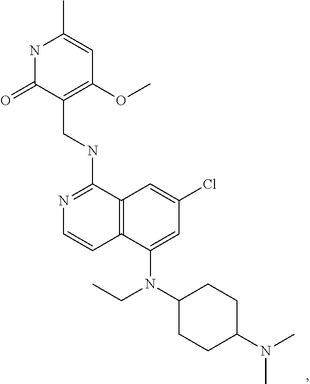 [SMILES: CCN(c1cc(Cl)cc2c(NCc3c(OC)cc(C)[nH]c3=O)nccc12)C1CCC(N(C)C)CC1]

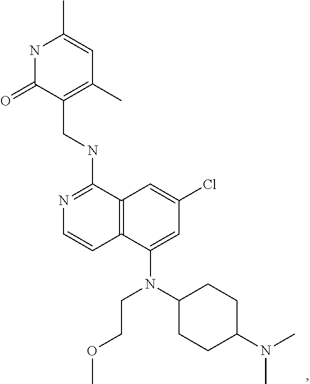 [SMILES: COCCN(c1cc(Cl)cc2c(NCc3c(C)cc(C)[nH]c3=O)nccc12)C1CCC(N(C)C)CC1]

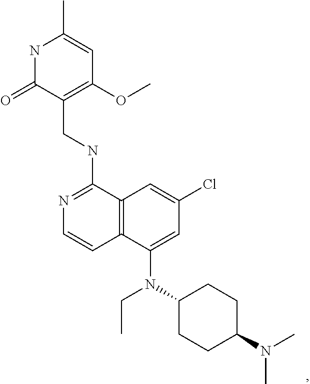 [SMILES: CCN(c1cc(Cl)cc2c(NCc3c(OC)cc(C)[nH]c3=O)nccc12)[C@H]1CC[C@H](N(C)C)CC1]

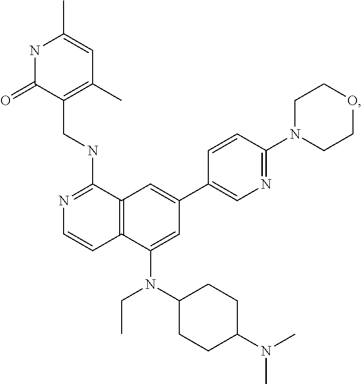 [SMILES: CCN(c1cc(-c2ccc(N3CCOCC3)nc2)cc2c(NCc3c(C)cc(C)[nH]c3=O)nccc12)C1CCC(N(C)C)CC1]

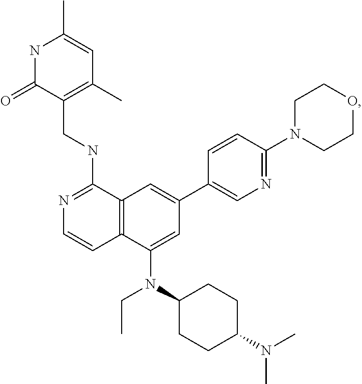 [SMILES: CCN(c1cc(-c2ccc(N3CCOCC3)nc2)cc2c(NCc3c(C)cc(C)[nH]c3=O)nccc12)[C@H]1CC[C@H](N(C)C)CC1]

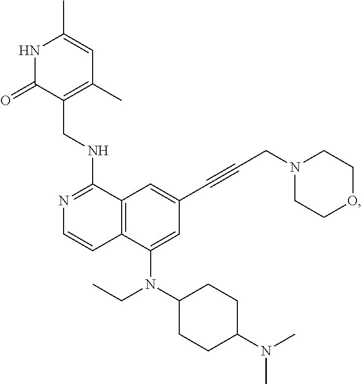 [SMILES: CCN(c1cc(C#CCN2CCOCC2)cc2c(NCc3c(C)cc(C)[nH]c3=O)nccc12)C1CCC(N(C)C)CC1]

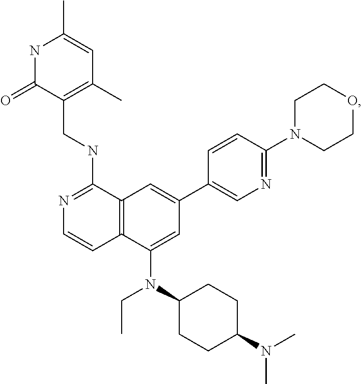 [SMILES: CCN(c1cc(-c2ccc(N3CCOCC3)nc2)cc2c(NCc3c(C)cc(C)[nH]c3=O)nccc12)[C@H]1CC[C@@H](N(C)C)CC1]

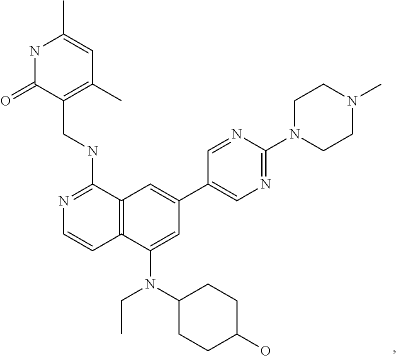 [SMILES: CCN(c1cc(-c2cnc(N3CCN(C)CC3)nc2)cc2c(NCc3c(C)cc(C)[nH]c3=O)nccc12)C1CCC(O)CC1]

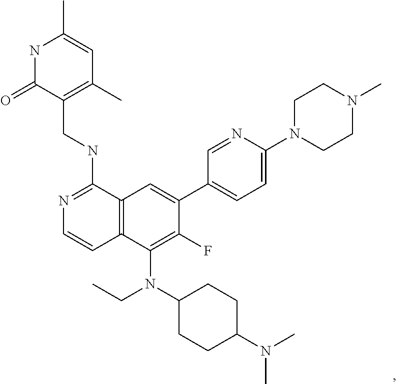 [SMILES: CCN(c1c(F)c(-c2ccc(N3CCN(C)CC3)nc2)cc2c(NCc3c(C)cc(C)[nH]c3=O)nccc12)C1CCC(N(C)C)CC1]

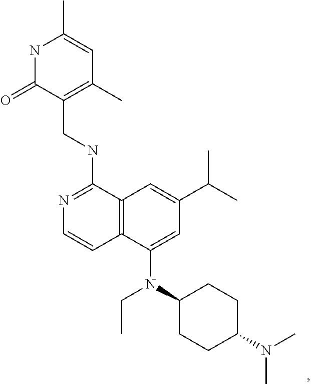 [SMILES: CCN(c1cc(C(C)C)cc2c(NCc3c(C)cc(C)[nH]c3=O)nccc12)[C@H]1CC[C@H](N(C)C)CC1]

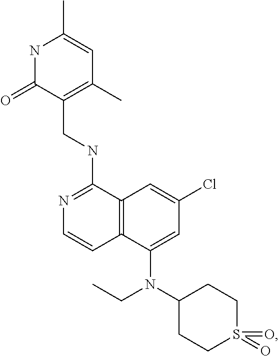 [SMILES: CCN(c1cc(Cl)cc2c(NCc3c(C)cc(C)[nH]c3=O)nccc12)C1CCS(=O)(=O)CC1]

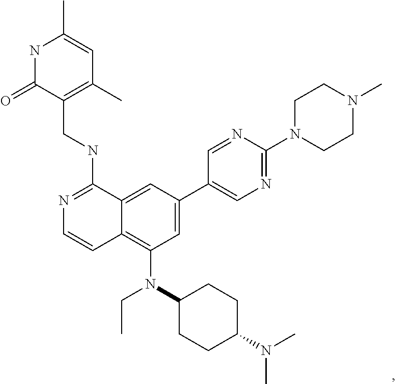

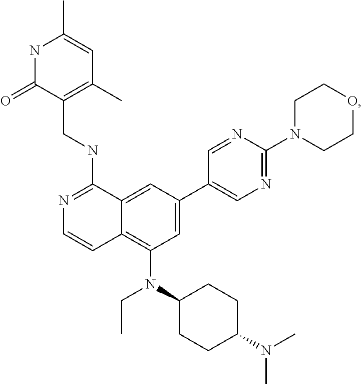 [SMILES: CCN(c1cc(-c2cnc(N3CCOCC3)nc2)cc2c(NCc3c(C)cc(C)[nH]c3=O)nccc12)[C@H]1CC[C@H](N(C)C)CC1]

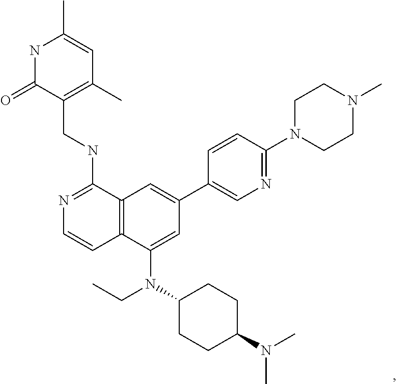 [SMILES: CCN(c1cc(-c2ccc(N3CCN(C)CC3)nc2)cc2c(NCc3c(C)cc(C)[nH]c3=O)nccc12)[C@H]1CC[C@H](N(C)C)CC1]

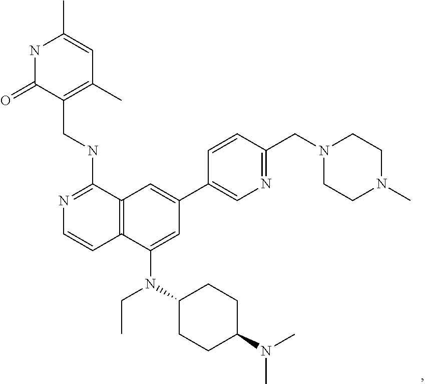 [SMILES: CCN(c1cc(-c2ccc(CN3CCN(C)CC3)nc2)cc2c(NCc3c(C)cc(C)[nH]c3=O)nccc12)[C@H]1CC[C@H](N(C)C)CC1]

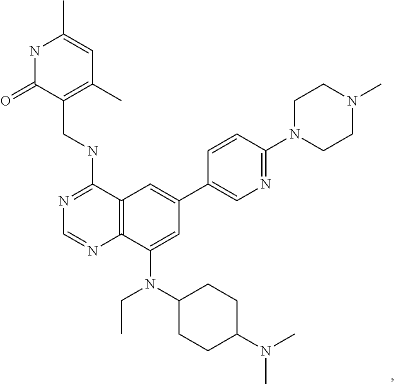 [SMILES: CCN(c1cc(-c2ccc(N3CCN(C)CC3)nc2)cc2c(NCc3c(C)cc(C)[nH]c3=O)ncnc12)C1CCC(N(C)C)CC1]

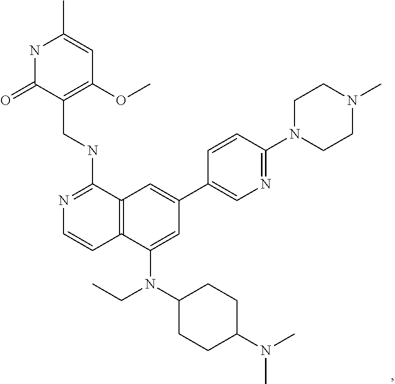 [SMILES: CCN(c1cc(-c2ccc(N3CCN(C)CC3)nc2)cc2c(NCc3c(OC)cc(C)[nH]c3=O)nccc12)C1CCC(N(C)C)CC1]

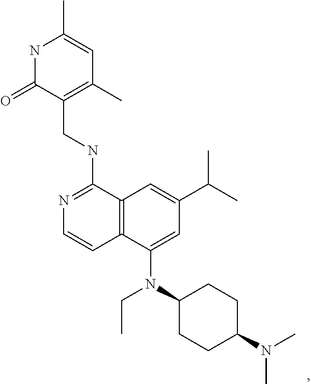 [SMILES: CCN(c1cc(C(C)C)cc2c(NCc3c(C)cc(C)[nH]c3=O)nccc12)[C@H]1CC[C@@H](N(C)C)CC1]

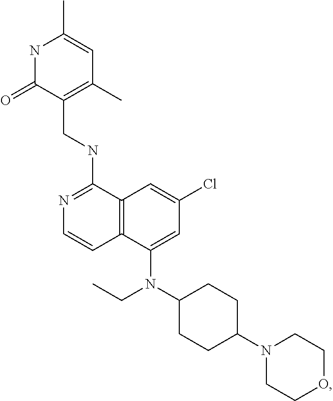 [SMILES: CCN(c1cc(Cl)cc2c(NCc3c(C)cc(C)[nH]c3=O)nccc12)C1CCC(N2CCOCC2)CC1]

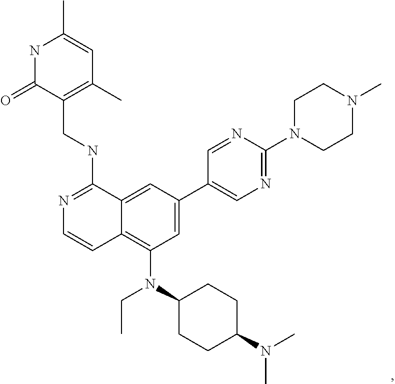 [SMILES: CCN(c1cc(-c2cnc(N3CCN(C)CC3)nc2)cc2c(NCc3c(C)cc(C)[nH]c3=O)nccc12)[C@H]1CC[C@@H](N(C)C)CC1]

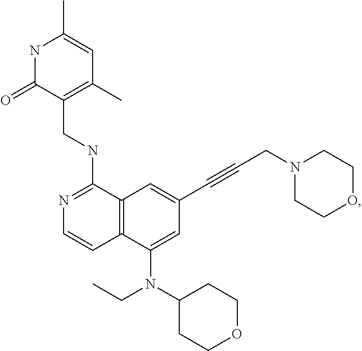 [SMILES: CCN(c1cc(C#CCN2CCOCC2)cc2c(NCc3c(C)cc(C)[nH]c3=O)nccc12)C1CCOCC1]

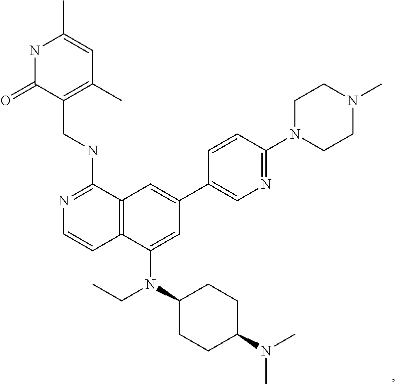 [SMILES: CCN(c1cc(-c2ccc(N3CCN(C)CC3)nc2)cc2c(NCc3c(C)cc(C)[nH]c3=O)nccc12)[C@H]1CC[C@@H](N(C)C)CC1]

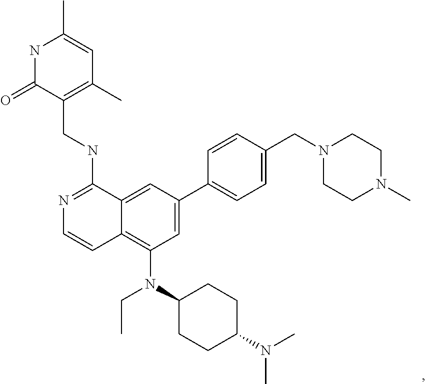 [SMILES: CCN(c1cc(-c2ccc(CN3CCN(C)CC3)cc2)cc2c(NCc3c(C)cc(C)[nH]c3=O)nccc12)[C@H]1CC[C@H](N(C)C)CC1]

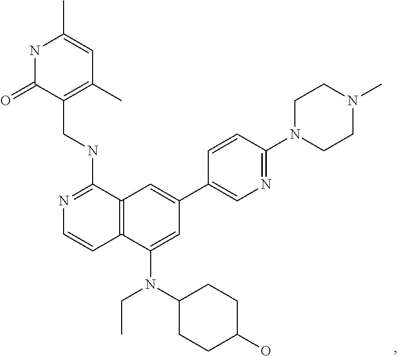 [SMILES: CCN(c1cc(-c2ccc(N3CCN(C)CC3)nc2)cc2c(NCc3c(C)cc(C)[nH]c3=O)nccc12)C1CCC(O)CC1]

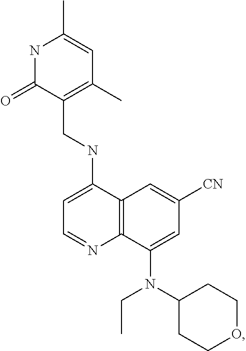 [SMILES: CCN(c1cc(C#N)cc2c(NCc3c(C)cc(C)[nH]c3=O)ccnc12)C1CCOCC1]

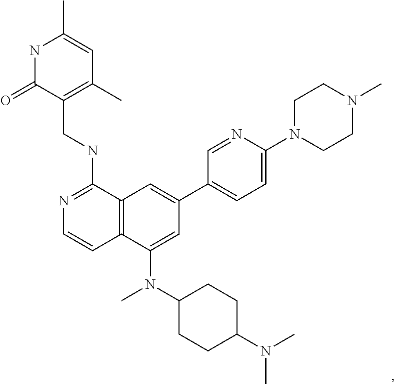 [SMILES: Cc1cc(C)c(CNc2nccc3c(N(C)C4CCC(N(C)C)CC4)cc(-c4ccc(N5CCN(C)CC5)nc4)cc23)c(=O)[nH]1]

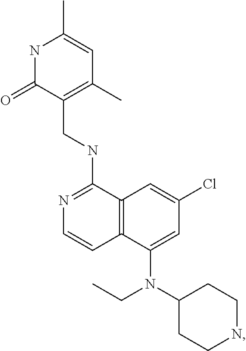

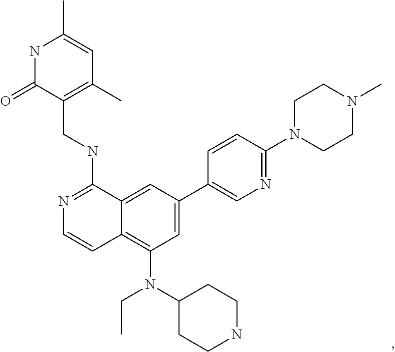 [SMILES: CCN(c1cc(-c2ccc(N3CCN(C)CC3)nc2)cc2c(NCc3c(C)cc(C)[nH]c3=O)nccc12)C1CCNCC1]

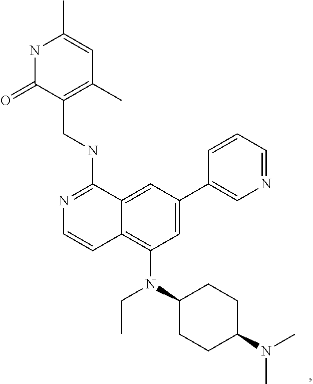 [SMILES: CCN(c1cc(-c2cccnc2)cc2c(NCc3c(C)cc(C)[nH]c3=O)nccc12)[C@H]1CC[C@@H](N(C)C)CC1]

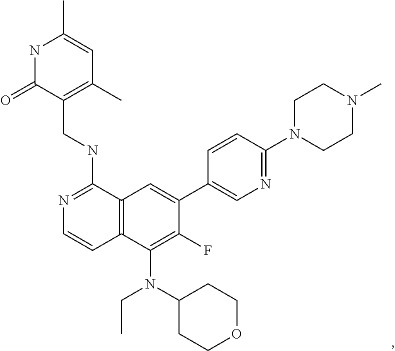 [SMILES: CCN(c1c(F)c(-c2ccc(N3CCN(C)CC3)nc2)cc2c(NCc3c(C)cc(C)[nH]c3=O)nccc12)C1CCOCC1]

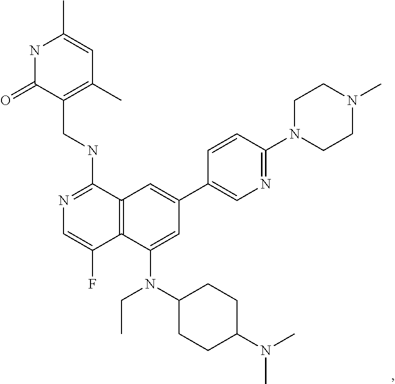 [SMILES: CCN(c1cc(-c2ccc(N3CCN(C)CC3)nc2)cc2c(NCc3c(C)cc(C)[nH]c3=O)ncc(F)c12)C1CCC(N(C)C)CC1]

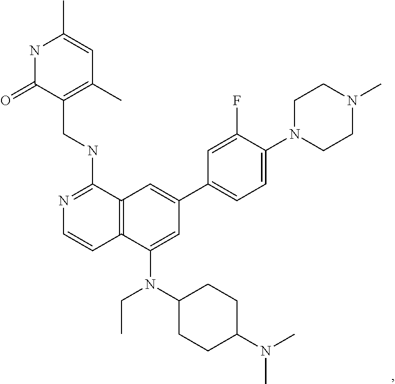 [SMILES: CCN(c1cc(-c2ccc(N3CCN(C)CC3)c(F)c2)cc2c(NCc3c(C)cc(C)[nH]c3=O)nccc12)C1CCC(N(C)C)CC1]

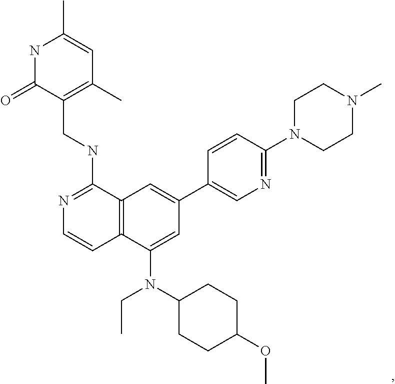 [SMILES: CCN(c1cc(-c2ccc(N3CCN(C)CC3)nc2)cc2c(NCc3c(C)cc(C)[nH]c3=O)nccc12)C1CCC(OC)CC1]

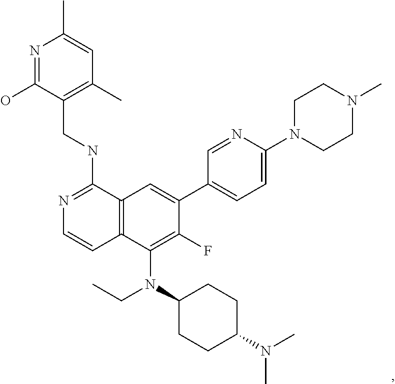 [SMILES: CCN(c1c(F)c(-c2ccc(N3CCN(C)CC3)nc2)cc2c(NCc3c(C)cc(C)nc3O)nccc12)[C@H]1CC[C@H](N(C)C)CC1]

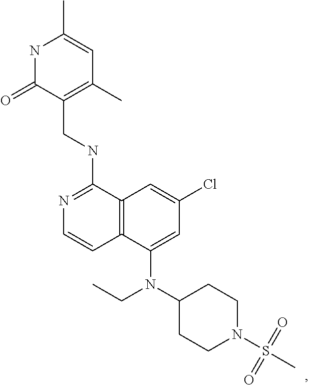 [SMILES: CCN(c1cc(Cl)cc2c(NCc3c(C)cc(C)[nH]c3=O)nccc12)C1CCN(S(C)(=O)=O)CC1]

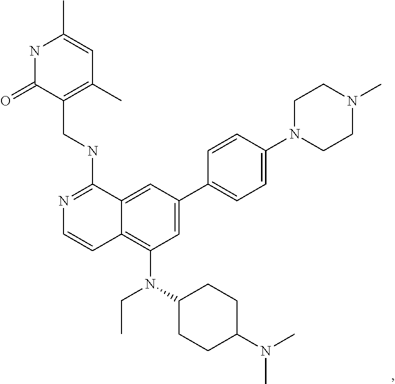

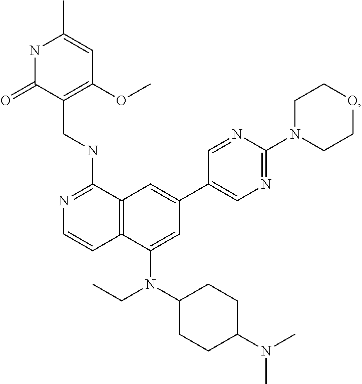 [SMILES: CCN(c1cc(-c2cnc(N3CCOCC3)nc2)cc2c(NCc3c(OC)cc(C)[nH]c3=O)nccc12)C1CCC(N(C)C)CC1]

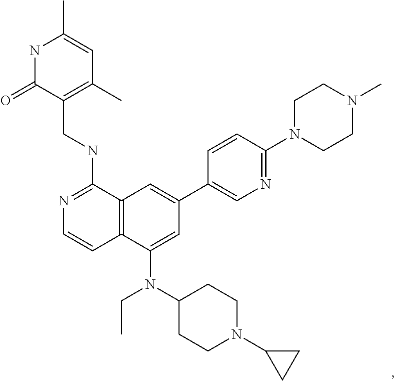 [SMILES: CCN(c1cc(-c2ccc(N3CCN(C)CC3)nc2)cc2c(NCc3c(C)cc(C)[nH]c3=O)nccc12)C1CCN(C2CC2)CC1]

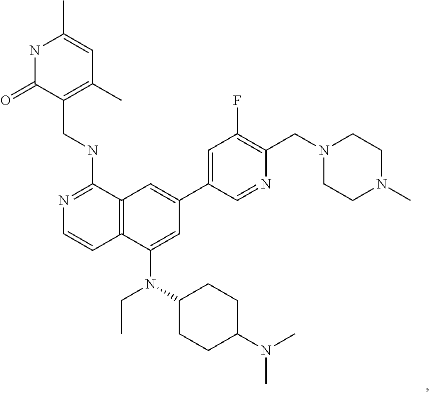

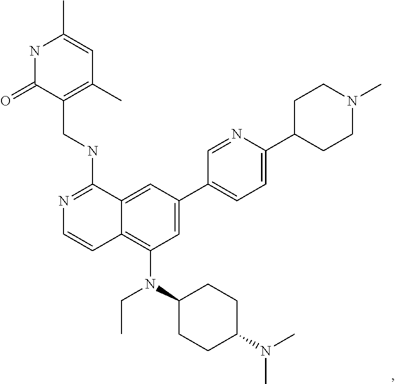 [SMILES: CCN(c1cc(-c2ccc(C3CCN(C)CC3)nc2)cc2c(NCc3c(C)cc(C)[nH]c3=O)nccc12)[C@H]1CC[C@H](N(C)C)CC1]

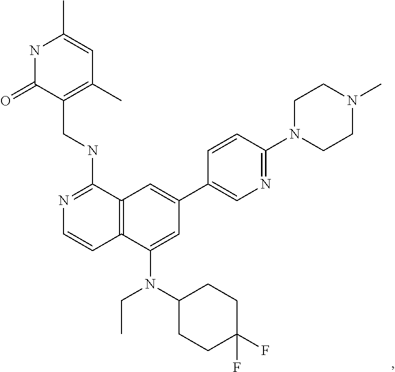 [SMILES: CCN(c1cc(-c2ccc(N3CCN(C)CC3)nc2)cc2c(NCc3c(C)cc(C)[nH]c3=O)nccc12)C1CCC(F)(F)CC1]

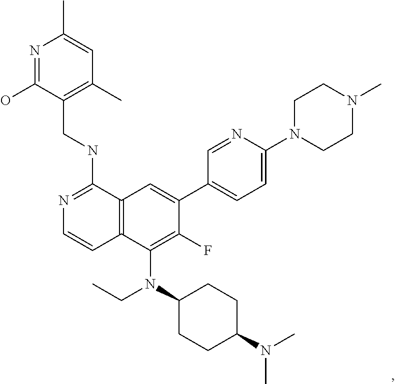 [SMILES: CCN(c1c(F)c(-c2ccc(N3CCN(C)CC3)nc2)cc2c(NCc3c(C)cc(C)nc3O)nccc12)[C@H]1CC[C@@H](N(C)C)CC1]

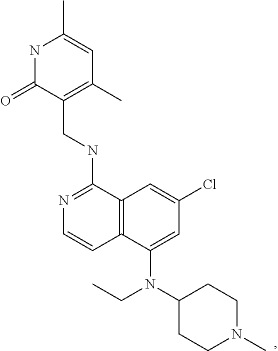 [SMILES: CCN(c1cc(Cl)cc2c(NCc3c(C)cc(C)[nH]c3=O)nccc12)C1CCN(C)CC1]

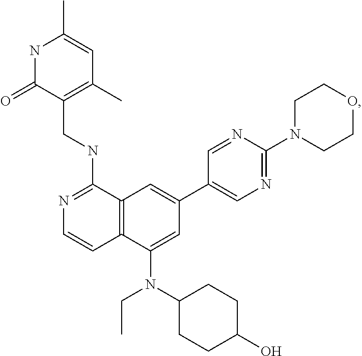 [SMILES: CCN(c1cc(-c2cnc(N3CCOCC3)nc2)cc2c(NCc3c(C)cc(C)[nH]c3=O)nccc12)C1CCC(O)CC1]

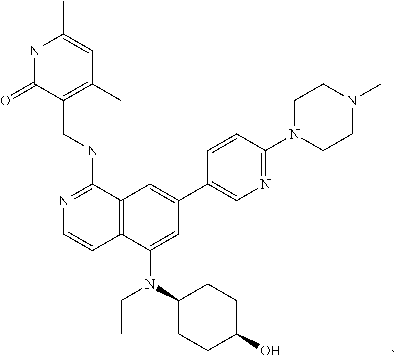 [SMILES: CCN(c1cc(-c2ccc(N3CCN(C)CC3)nc2)cc2c(NCc3c(C)cc(C)[nH]c3=O)nccc12)[C@H]1CC[C@@H](O)CC1]

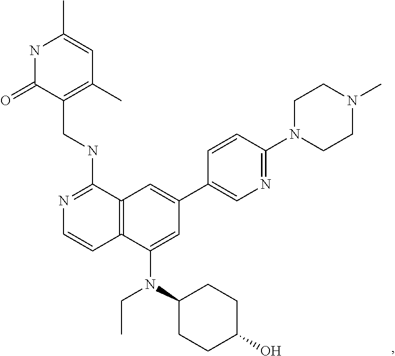 [SMILES: CCN(c1cc(-c2ccc(N3CCN(C)CC3)nc2)cc2c(NCc3c(C)cc(C)[nH]c3=O)nccc12)[C@H]1CC[C@H](O)CC1]

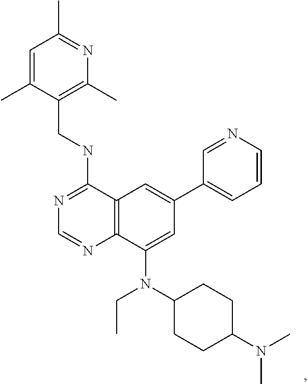

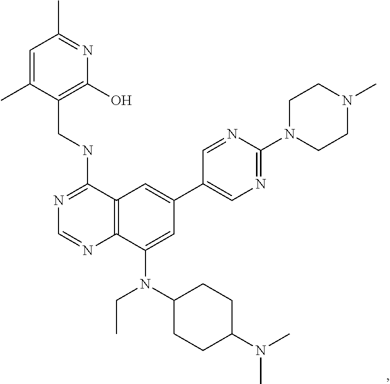

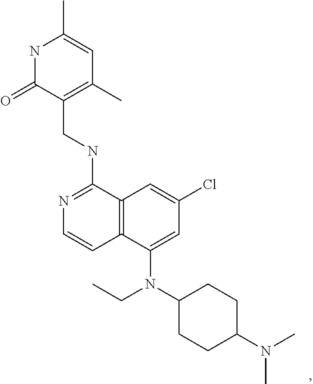 [SMILES: CCN(c1cc(Cl)cc2c(NCc3c(C)cc(C)[nH]c3=O)nccc12)C1CCC(N(C)C)CC1]

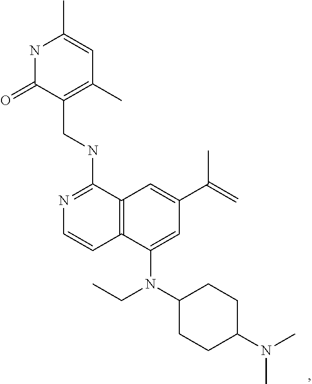 [SMILES: C=C(C)c1cc(N(CC)C2CCC(N(C)C)CC2)c2ccnc(NCc3c(C)cc(C)[nH]c3=O)c2c1]

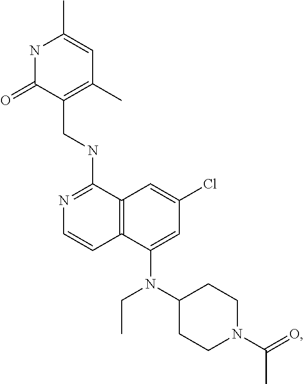 [SMILES: CCN(c1cc(Cl)cc2c(NCc3c(C)cc(C)[nH]c3=O)nccc12)C1CCN(C(C)=O)CC1]

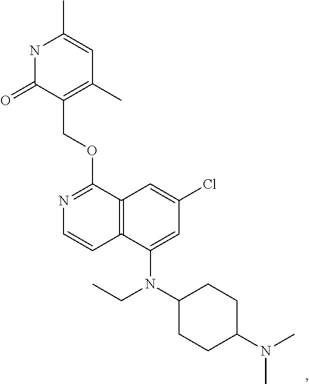 [SMILES: CCN(c1cc(Cl)cc2c(OCc3c(C)cc(C)[nH]c3=O)nccc12)C1CCC(N(C)C)CC1]

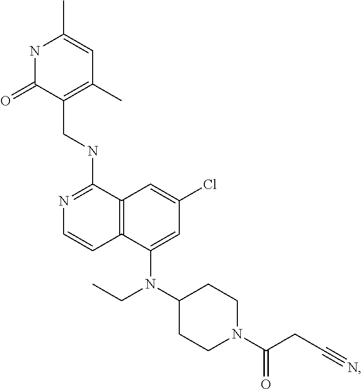 [SMILES: CCN(c1cc(Cl)cc2c(NCc3c(C)cc(C)[nH]c3=O)nccc12)C1CCN(C(=O)CC#N)CC1]

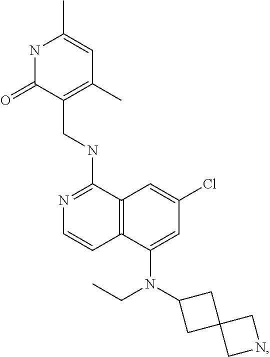

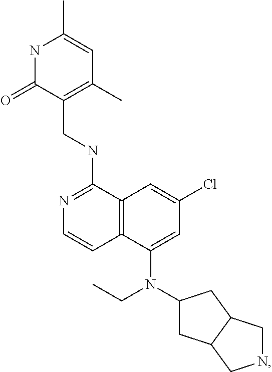

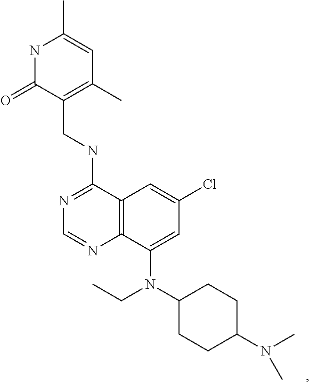 [SMILES: CCN(c1cc(Cl)cc2c(NCc3c(C)cc(C)[nH]c3=O)ncnc12)C1CCC(N(C)C)CC1]

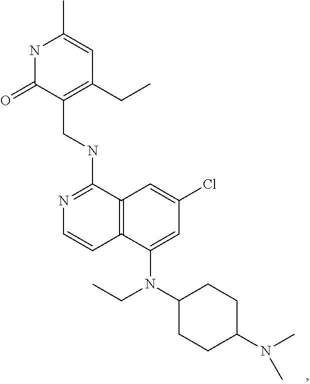 [SMILES: CCc1cc(C)[nH]c(=O)c1CNc1nccc2c(N(CC)C3CCC(N(C)C)CC3)cc(Cl)cc12]

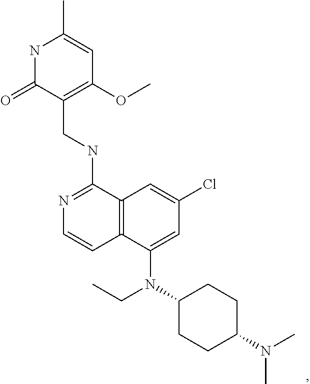 [SMILES: CCN(c1cc(Cl)cc2c(NCc3c(OC)cc(C)[nH]c3=O)nccc12)[C@H]1CC[C@@H](N(C)C)CC1]

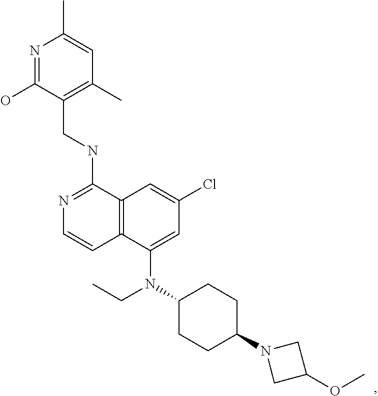 [SMILES: CCN(c1cc(Cl)cc2c(NCc3c(C)cc(C)nc3O)nccc12)[C@H]1CC[C@H](N2CC(OC)C2)CC1]

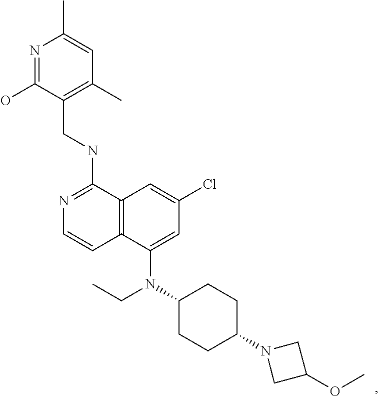 [SMILES: CCN(c1cc(Cl)cc2c(NCc3c(C)cc(C)nc3O)nccc12)[C@H]1CC[C@@H](N2CC(OC)C2)CC1]

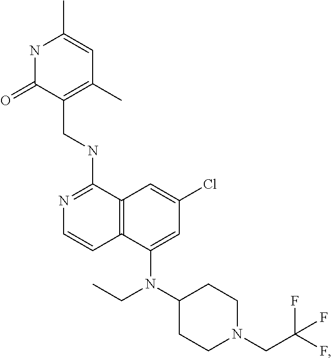 [SMILES: CCN(c1cc(Cl)cc2c(NCc3c(C)cc(C)[nH]c3=O)nccc12)C1CCN(CC(F)(F)F)CC1]

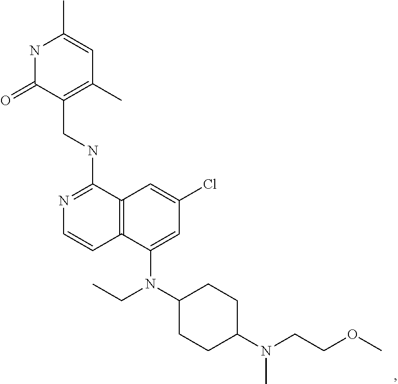 [SMILES: CCN(c1cc(Cl)cc2c(NCc3c(C)cc(C)[nH]c3=O)nccc12)C1CCC(N(C)CCOC)CC1]

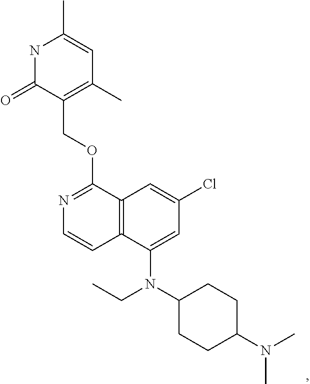 [SMILES: CCN(c1cc(Cl)cc2c(OCc3c(C)cc(C)[nH]c3=O)nccc12)C1CCC(N(C)C)CC1]

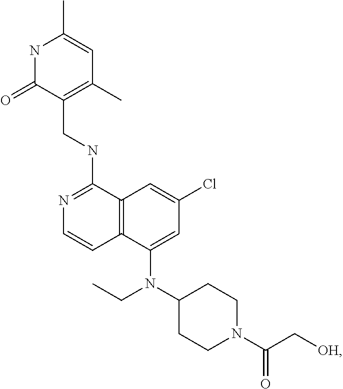 [SMILES: CCN(c1cc(Cl)cc2c(NCc3c(C)cc(C)[nH]c3=O)nccc12)C1CCN(C(=O)CO)CC1]

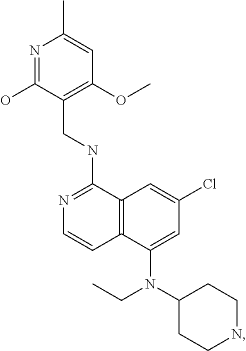

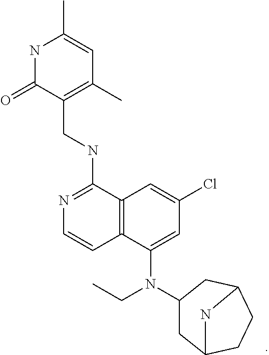 [SMILES: CCN(c1cc(Cl)cc2c(NCc3c(C)cc(C)[nH]c3=O)nccc12)C1CC2CCC(C1)N2]

According to a second aspect of the present disclosure, a pharmaceutical composition is provided. The pharmaceutical composition includes the compound of the first aspect of the disclosure, or a pharmaceutically acceptable salt, solvate, stereoisomer or prodrug thereof; and a pharmaceutically acceptable carrier.

According to a third aspect of the present disclosure, there is provided use of the compound of the first aspect of the disclosure, or a pharmaceutically acceptable salt, solvate, stereoisomer or prodrug thereof, or the pharmaceutical composition of the second aspect of the disclosure in the preparation of EZH2 inhibitors.

According to a fourth aspect of the present disclosure, there is provided use of the compound of the first aspect of the disclosure, or a pharmaceutically acceptable salt, solvate, stereoisomer or prodrug thereof, or the pharmaceutical composition of the second aspect of the disclosure in the preparation of a drug for the treatment of EZH2-mediated diseases or conditions.

According to a fifth aspect of the present disclosure, there is provided a method of treating a disease or condition mediated by EZH2, wherein the method includes administering to a patient in need thereof a therapeutically effective amount of the compound of the first aspect of the present disclosure, or a pharmaceutically acceptable salt, solvate, stereoisomer or prodrug thereof, or the pharmaceutical composition of the second aspect of the present disclosure.

According to a sixth aspect of the present disclosure, there is provided a method of treating a disease or condition mediated by EZH2, wherein the method includes administering to a patient in need thereof a therapeutically effective amount of the compound of the first aspect of the present disclosure, or a pharmaceutically acceptable salt, solvate, stereoisomer or prodrug thereof, and another therapeutically active agent.

In another preferred example, the disease or condition mediated by EZH2 is selected from: cancer, pulmonary arterial hypertension, myelofibrosis, human immunodeficiency virus (HIV) disease, graft versus host disease (GVHD), Weaver syndrome, psoriasis vulgaris or liver fibrosis.

In another preferred example, the disease or condition mediated by EZH2 is cancer.

In another preferred example, the cancers mediated by EZH2 include, but are not limited to, thyroid cancer, cardiac sarcoma, lung cancer, gastrointestinal cancer, genitourinary tract tumor, liver cancer, mantle cell lymphoma, osteosarcoma, nervous system sarcoma, gynecological cancer, hematological system tumor, adrenal neuroblastoma, skin cancer, astrocytic tumor, breast cancer, colorectal cancer, endometrial cancer, head and neck cancer, oral cavity cancer.

It should be understood that each of the above technical features of the disclosure and each technical feature specifically described below (such as in Examples) can be combined with each other within the scope of the present disclosure so as to constitute new or preferred technical solutions which need not be specified again herein.

### DETAILED DESCRIPTION OF THE EMBODIMENTS

The inventors have conducted extensive and intensive studies and have unexpectedly found that such 1,5,7-trisubstituted isoquinoline derivatives have high inhibitory activities against enzymes such as EZH2 Y641F and cells such as SU-DHL-6 and SU-DHL-10. Therefore, this series of compounds are hopefully developed as a drug for the treatment of tumors. Based on this, the inventors completed the present invention.

### Definition of Terms

As used herein, “alkyl” refers to straight and branched saturated aliphatic hydrocarbon groups; C₁₋₈ alkyl is an alkyl containing 1 to 8 carbon atoms, and may preferably be a C₁₋₆ alkyl, more preferably a C₁₋₃ alkyl, wherein the C₁₋₆ alkyl and C₁₋₃ alkyl have similar definitions; non-limiting examples of alkyl include: methyl, ethyl, n-propyl, isopropyl, n-butyl, isobutyl, tert-butyl, sec-butyl, n-pentane, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-decyl, 2-methyl-2-ethylhexyl, 2-methyl-3-ethylhexyl, 2,2-diethylpentyl, n-decyl, 3,3-diethylhexyl, 2,2-diethylhexyl, and various of their branched isomers.

As used herein, “cycloalkyl” refers to a saturated or partially unsaturated monocyclic cyclic hydrocarbon group; “C₃₋₈ cycloalkyl” refers to a cyclic hydrocarbon group containing 3 to 8 carbon atoms, and may preferably be a C₃₋₆ cycloalkyl with similar definition; non-limiting examples of cycloalkyl include cyclopropyl, cyclobutyl, cyclopentyl, cyclopentenyl, cyclohexyl, cyclohexenyl, cyclohexadienyl, cycloheptyl, cycloheptatrienyl. cyclooctyl and the like, preferably cyclopropyl, cyclopentyl, cyclohexenyl.

As used herein, “spiro” refers to a polycyclic group in which two single rings share one carbon atom (spiro atom), wherein these polycyclic groups may contain one or more double bonds, but none of the rings has a completely conjugated it electron system. According to the number of rings therein, the spiros are divided into bicyclic spiros or polycyclic spiros, wherein the bicyclic spiros are preferable. And 4 membered/5 membered, 5 membered/5 membered, or 5 membered/6 membered bicyclic spiros are more preferable. For example:

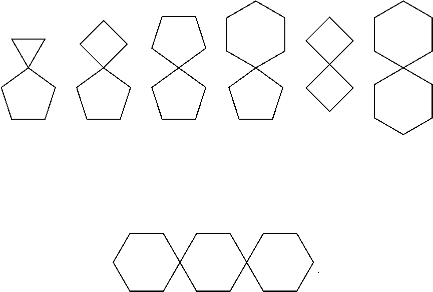

As used herein, “spiroheterocycle” refers to a polycyclic hydrocarbon in which two single rings share one atom (spiro atom), wherein one or two ring atoms are selected from heteroatoms such as nitrogen, oxygen, or S(O)ₙ (wherein n is an integer from 0 to 2), the remaining ring atoms are carbon atoms. These spiroheterocycles may contain one or more double bonds, but none of the rings has a completely conjugated π-electron system. According to the number of rings, the spiroheterocycles are divided into bicyclic spiroheterocycles or polycyclic spiroheterocycles, wherein bicyclic spiroheterocycles are preferable. And 4 membered/5 membered, 5 membered/5 membered, or 5 membered/6 membered bicyclic spiroheterocycles are more preferable. For example:

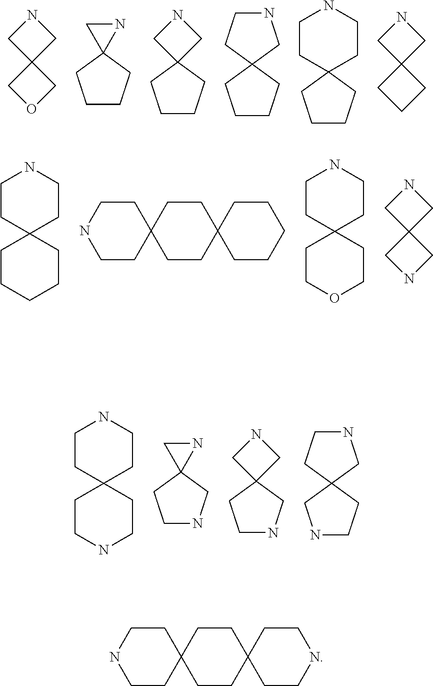

As used herein, “bridged ring” refers to a group containing multiple rings which share two or more carbon atoms. The shared carbon atoms are known as bridgehead carbons. Between two bridgehead carbons there may be a carbon chain or a bond, which is called as a bridge. These bridged rings may contain one or more double bonds, but none of the rings has a completely conjugated i-electron system. Bicyclic or tricyclic bridged rings are preferred. For example:

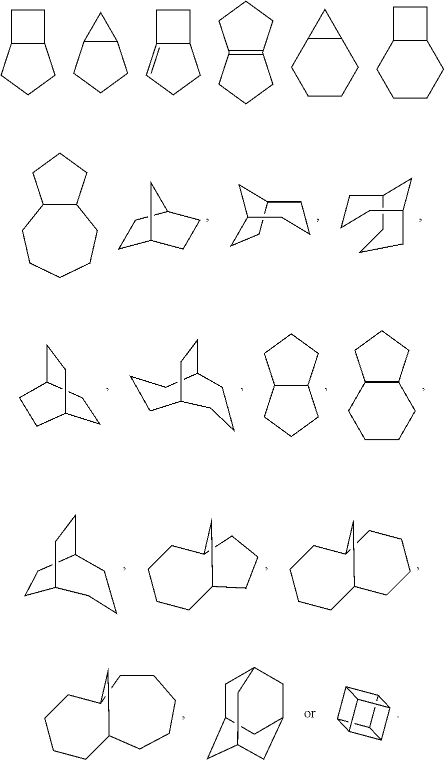

As used herein, “bridged heterocycle” refers to a group containing multiple rings which share two or more atoms and have one or more ring atoms selected from heteroatoms such as nitrogen, oxygen, or S(O)ₙ (wherein n is an integer from 0 to 2) and the remaining ring atoms being carbon atoms. These bridged heterocycles may contain one or more double bonds, but none of the rings has a completely conjugated i-electron system. Bicyclic or tricyclic bridged heterocycles are preferred. For example:

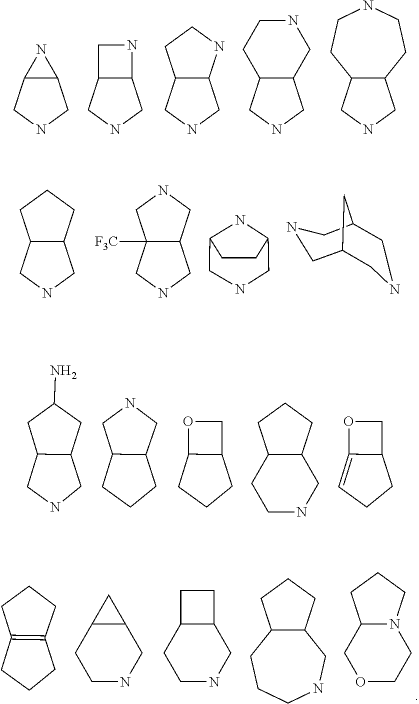

As used herein, “8 to 10 membered bicyclic ring” refers to a two-ring-containing bridged ring having 8 to 10 ring atoms. The bicyclic ring may be a saturated full-carbon bicyclic or partially unsaturated full-carbon bicyclic ring. Examples of bicyclic ring include (but not limited to):

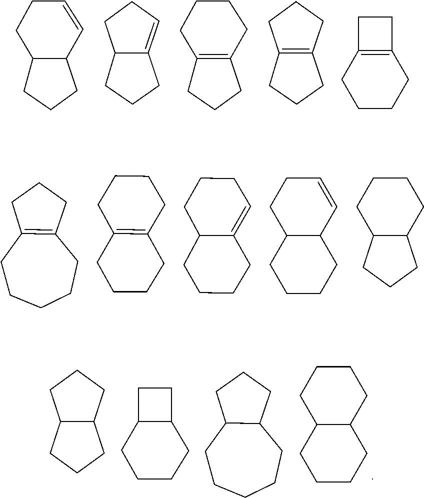

As used herein, “8 to 10 membered bis-heterocycle” refers to a two-ring-containing bridged heterocycle having 8 to 10 ring atoms, wherein 1, 2, 3, 4 or 5 carbon ring atoms are replaced by heteroatoms selected from nitrogen, oxygen or sulfur. Examples of bis-heterocycles include, but are not limited to, tetrahydroquinoline ring, tetrahydroisoquinoline ring, decahydroquinoline ring, and the like.

As used herein, “C₁₋₈ alkoxy” refers to —O—(C₁₋₈ alkyl), wherein the alkyl is as defined above. C₁₋₆ alkoxy is preferred, and C₁₋₃ alkoxy is more preferred. Non-limiting examples include methoxy, ethoxy, propoxy, isopropoxy, butoxy, tert-butoxy, isobutoxy, pentoxy, and the like.

As used herein, “C₃₋₈ cycloalkoxy” refers to —O—(C₃₋₈ cycloalkyl), wherein the cycloalkyl is as defined above. C₃₋₆ cycloalkoxy is preferred. Non-limiting examples include cyclopropyloxy, cyclobutyloxy, cyclopentoxy, cyclohexyloxy, etc.

As used herein, “C₆₋₁₀ aryl” refers to a full-carbon monocyclic or fused polycyclic (ie, rings that share an adjacent pair of carbon atoms) group having a conjugated xi-electron system, and refers to an aryl containing 6 to 10 carbon atoms; phenyl and naphthyl are preferred, and phenyl is more preferred.

As used herein, “a bond” means that two groups connected thereby are connected by a covalent bond.

As used herein, “halogen” refers to fluorine, chlorine, bromine, or iodine.

As used herein, “halogenated” means that one or more (e.g., 1, 2, 3, 4 or 5) hydrogens in a group are substituted by halogen(s).

For example, “halogenated C₁₋₈ alkyl” means that the alkyl is substituted with one or more (e.g., 1, 2, 3, 4 or 5) halogens, wherein the alkyl is as defined above. Halogenated C₁₋₆ alkyl is preferred, and halogenated C₁₋₃ alkyl is more preferred. Examples of halogenated C₁₋₈ alkyl include, but not limited to, monochloromethyl, dichloromethyl, trichloromethyl, monochloroethyl, 1,2-dichloroethyl, trichloroethyl, monobromoethyl, monofluoromethyl, difluoromethyl, trifluoromethyl, monofluoroethyl, difluoroethyl, trifluoroethyl, and the like.

For another example, “halogenated C₁₋₈ alkoxy” means that the alkoxy is substituted with one or more (e.g., 1, 2, 3, 4 or 5) halogens, wherein the alkoxy is as defined above. Halogenated C₁₋₆ alkoxy is preferred, and halogenated C₁₋₃ alkoxy is more preferred. Examples of the halogenated C₁₋₈ alkoxy include, but are not limited to, trifluoromethoxy, trifluoroethoxy, monofluoromethoxy, monofluoroethoxy, difluoromethoxy, difluoroethoxy, and the like.

For another example, “halogenated C₃₋₈ cycloalkyl” means that a cycloalkyl is substituted with one or more (e.g., 1, 2, 3, 4 or 5) halogens, wherein the cycloalkyl is as defined above. Halogenated C₃₋₆ cycloalkyl is preferred. Examples of the halogenated C₃₋₈ cycloalkyl include, but not limited to, trifluorocyclopropyl, monofluorocyclopropyl, monofluorocyclohexyl, difluorocyclopropyl, difluorocyclohexyl and the like.

As used herein, “deuterated C₁₋₈ alkyl” means that an alkyl is substituted with one or more (e.g., 1, 2, 3, 4, or 5) deuterium atoms, wherein the alkyl is as defined above. Deuterated C₁₋₆ alkyl is preferred, and deuterated C₁₋₃ alkyl is more preferred. Examples of the deuterated C₁₋₈ alkyl include, but not limited to, monodeuterated methyl, monodeuterated ethyl, dideuterated methyl, dideuterated ethyl, trideuterated methyl, trideuterated ethyl and the like.

As used herein, “amino” refers to NH₂, “cyano” refers to CN, “nitro” refers to NO₂, “benzyl” refers to —CH₂-phenyl, “oxo” refers to ═O, “carboxyl” refers to —C(O)OH, “acetyl” refers to —C(O)CH₃, “hydroxymethyl” refers to —CH₂OH, “hydroxyethyl” refers to —CH₂CH₂OH, “hydroxy” refers to —OH, “thiol” refers to SH, the structure of “cyclopropylidene” is:

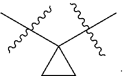 [SMILES: *C1(*)CC1]

As used herein, “heteroaryl ring” and “heteroaryl” can be used interchangeably, and refer to having 5-10 carbon atoms, wherein 5 or 6 membered monocyclic heteroaryls or 8 to 10 membered bicyclic heteroaryls are preferred; 6, 10 or 14 it electrons are shared in the ring array; and other than carbon atoms, the heteroaryl also has 1 to 5 heteroatoms. “Heteroatom” refers to nitrogen, oxygen or sulfur.

As used herein, “3 to 6 membered saturated or partially unsaturated monocyclic ring” refers to a saturated or partially unsaturated full-carbon monocyclic ring containing 3 to 6 ring atoms. Examples of the 3 to 6 membered saturated or partially unsaturated monocyclic rings include, but not limited to, cyclopropyl ring, cyclobutyl ring, cyclopentyl ring, cyclopentenyl ring, cyclohexyl ring, cyclohexenyl ring, cyclohexadienyl ring, cycloheptyl ring, cycloheptatrienyl ring, cyclooctyl ring and the like.

As used herein, “3 to 6 membered saturated single heterocycle” means a 3 to 6 membered monocycle in which 1, 2 or 3 carbon atoms are substituted by heteroatom(s) selected from nitrogen, oxygen, or S(O)ₜ (wherein t is an integer from 0 to 2), but which does not include a ring portion of —O—O—, —O—S— or —S—S—, and the remaining ring atoms of which are carbons; 4 to 6 membered is preferred, and 5 to 6 membered is more preferred. Examples of 3 to 6 membered saturated single heterocycles include, but not limited to, epoxypropane, azetidine, oxetane, tetrahydrofuran, tetrahydrothiophene, pyrrolidine, piperidine, pyrroline, oxazolidine, piperazine, dioxolane, dioxane, morpholine, thiomorpholine, thiomorpholine-1,1-dioxide, tetrahydropyran and the like.

As used herein, “5 to 6 membered monocyclic heteroaryl ring” refers to a monocyclic heteroaryl ring containing 5 to 6 ring atoms, examples include but are not limited to, thiophene ring, N-alkylpyrrole ring, furan ring, thiazole ring, imidazole ring, oxazole ring, pyrrole ring, pyrazole ring, triazole ring, tetrazole ring, isoxazole ring, oxadiazole ring, thiadiazole ring, pyridine ring, pyridazine ring, pyrimidine ring, pyrazine ring and the like.

As used herein, “8 to 10 membered bicyclic heteroaryl ring” refers to a bicyclic heteroaryl ring containing 8 to 10 ring atoms, including, for example, but not limited to, benzofuran, benzothiophene, indole, isoindole, quinoline, isoquinoline, indazole, benzothiazole, benzimidazole, quinazoline, quinoxaline, cinnoline, phthalizine.

As used herein, “substituted” means that one or more hydrogen atoms, preferably 1-5 hydrogen atoms in a group are independently substituted by a corresponding number of substituents, and more preferably, 1 to 3 hydrogen atoms are independently substituted by a corresponding number of substituents. It goes without saying that the substituents are only located in their possible chemical positions, and those skilled in the art can determine (by experiment or theory) the possible or impossible substitutions without undue effort. For example, an amino or hydroxy with a free hydrogen may be unstable when combined with a carbon atom having an unsaturated (eg. olefinic) bond.

As used herein, any one of the above groups may be substituted or unsubstituted. When the above groups are substituted, the substituents are preferably 1 to 5 groups independently selected from the group consisting of CN, halogen, C₁₋₈ alkyl (preferably C₁₋₆ alkyl, more preferably C₁₋₃ alkyl), C₁₋₈ alkoxy (preferably C₁₋₆ alkoxy, more preferably C₁₋₃ alkoxy), halogenated C₁₋₈ alkyl (preferably halogenated C₁₋₆ alkyl, more preferably halogenated C₁₋₃ alkyl), C₃₋₈ cycloalkyl (preferably C₃₋₆ cycloalkyl), halogenated C₁₋₈ alkoxy (preferably halogenated C₁₋₆ alkoxy, more preferably halogenated C₁₋₃ alkoxy), amino substituted with C₁₋₈ alkyl, amino, amino substituted with halogenated C₁₋₈ alkyl, 4 to 6 membered saturated single heterocycle, 5 to 6 membered monocyclic heteroaryl ring, 8 to 10 membered bicyclic heteroaryl ring, spiro, spiroheterocycle, bridged ring or bridged heterocycle.

The above-mentioned various substituents themselves of the present disclosure can also be substituted with the groups described herein.

When 4 to 6 membered saturated single heterocycles described herein are substituted, the positions of the substituents may be at their possible chemical positions, and representative substitutions of the exemplary single heterocycles are shown below (wherine Sub represents a substituent):

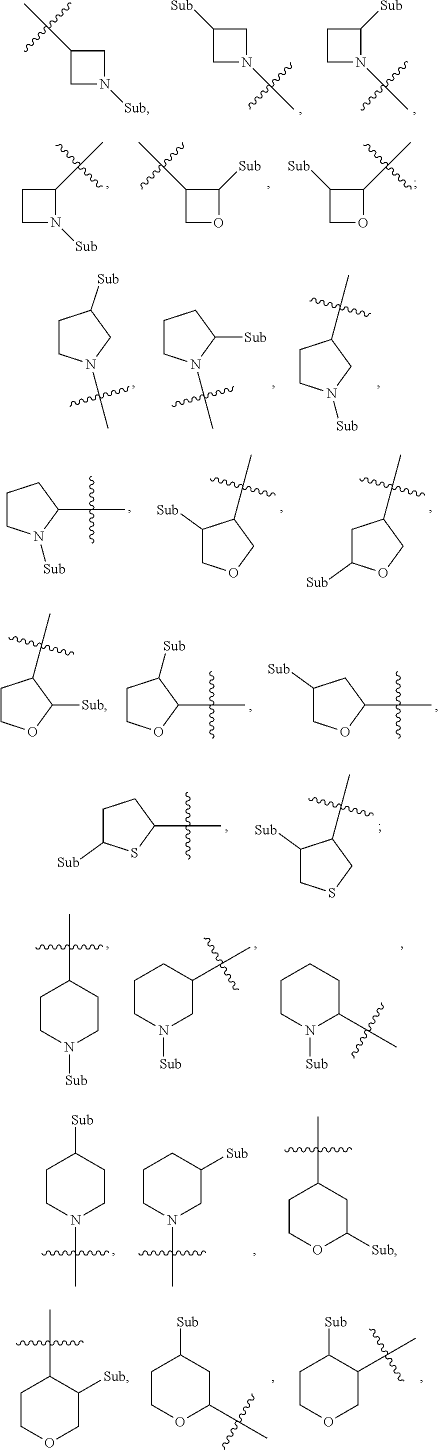

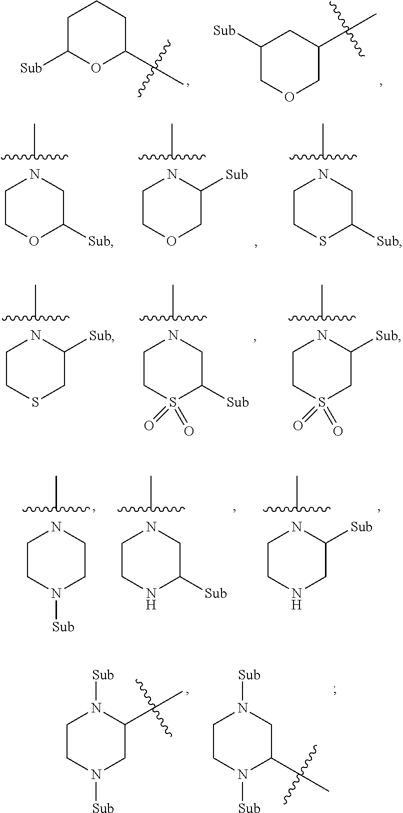

wherein “Sub” represents the various types of substituents described herein; “ [symbol] ” represents connections with other atoms.

As used herein, “EZH2 inhibitor” refers to an agent (refers to a compound of formula (I) in the present disclosure) that can inhibit increased expression of the histone lysine N-methyltransferase EZH2, is a catalytic functional subunit of PRC2, and is responsible for Lys27 methylation of specific histone H3 (H3K27) and indispensable for stem cell self-renewal.

As used herein, “disease or condition mediated by EZH2” refers to an abnormal condition in a patient resulting from an abnormal epigenetic modification caused by abnormal expression of the histone lysine N-methyltransferase EZH2.

As used herein, “therapeutically effective amount” refers to an amount of the compound of the present disclosure that will elicit the biological or medical response of an individual, for example, reduction or inhibition of an enzyme or a protein activity, or amelioration of a symptom, alleviation of a condition, slow or delay disease progression, or prevention of a disease, etc.

As used herein, “pharmaceutically acceptable carrier” refers to a non-toxic, inert, solid, semi-solid substance or liquid filler, diluent, encapsulating material or auxiliary formulation or any type of excipient that is compatible with the patient which is preferably a mammal and more preferably a human. It is suitable for delivering active agent to a target without stopping the activity of the agent.

As used herein, “patient” refers to an animal, preferably a mammal, and more preferably a human being. The term “mammal” refers to a warm-blooded vertebrate mammal, including, for example, cat, dog, rabbit, bear, fox, wolf, monkey, deer, rat, pig and human.

As used herein, “treating/treatment” refers to alleviating, delaying progression, attenuating, preventing, or maintaining an existing disease or disorder (eg. cancer). Treating/treatment also includes curing one or more symptoms of the disease or disorder, preventing its development or reducing to some extent.

Preparation Method

The present disclosure provides the preparation method of compounds of formula (I), the compounds of the present disclosure can be prepared by a variety of synthetic operations, exemplary preparation methods of these compounds may include (but not limited to) the processes described below.

Preferably, the compounds of formula (I) can be prepared through the following schemes and exemplary methods described in embodiment, as well as according to the related publications available for those skilled in the art.

The steps of the method can be expanded or combined as desired in practice.

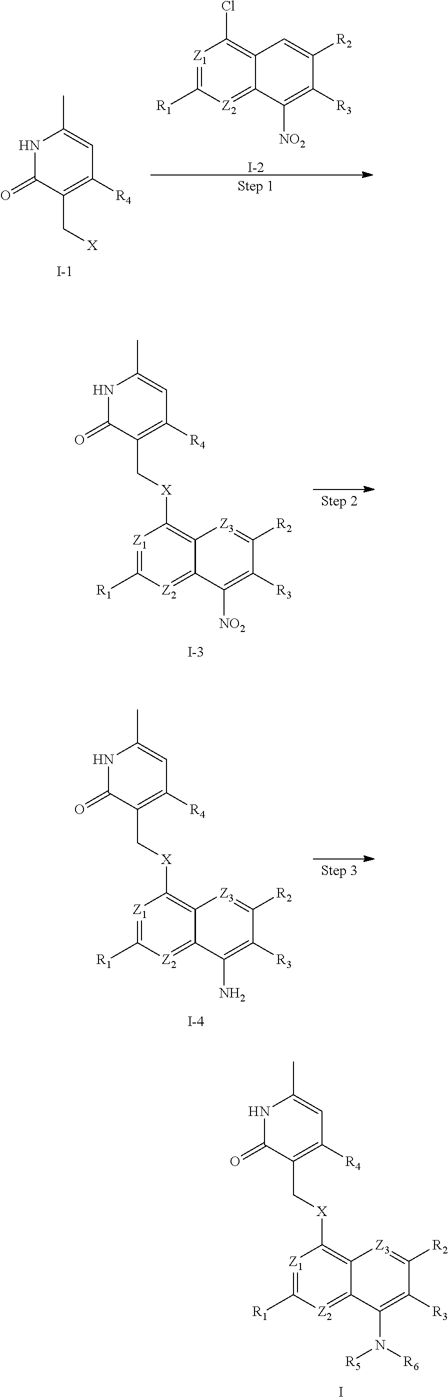

Step 1: In the presence of an alkali system, a nucleophilic reaction site or group (eg. NH, OH, etc.) in the compound of formula (I-1) undergoes a substitution reaction with a compound of formula (I-2) to form a compound of formula (I-3), wherein suitable alkali systems include potassium carbonate in DMSO, potassium carbonate in DMF, etc.

Step 2: The nitro group in formula (I-2) is reduced to an amino to obtain a compound of formula (I-4), and the reduction methods can refer to conventional methods in the field.

Step 3: Aldehyde or ketone of R₅ and R₆ undergoes a reductive amination with the compound of formula (I-4) to give a compound of formula (I). The reductive amination is performed in conditions of toluene reflux for water removal and reduction with reductant, or reduction in catalytic systems formed from acid-metal hydride, wherein the acid includes Lewis acid such as acetic acid, trifluoroacetic acid, titanium tetrachloride, or Bronsted acid, the solvent may be selected from dichloromethane, 1,2-dichloroethane, 1,4-dioxane, tetrahydrofuran, methanol, ethyl ether, acetonitrile and other solvents, and appropriate reductants include sodium triacetoxyborohydride, sodium cyanoborohydride, sodium borohydride, etc. The reaction temperature is from room temperature to 70° C.

The R₂ in formula (I) compound may be modified by methods known to those skilled in the art, such as hydrogenation or metal reduction, or alkylation, ether formation, ester formation, amide formation, carboxylic acid formation, etc. Arylation with arylboronic acids can be carried out in the presence of Pd catalysts, suitable ligands and bases, preferably carbonates or phosphates of sodium, potassium or cesium, organic bases such as triethylamine, DIPEA, etc. can also be used. Solvents can be changed between toluene, 1,4-dioxane, DMF, acetonitrile, alcohols, etc., and in some cases even water and other solvents. Common catalysts, for example, Pd(PPh₃)₄ or Pd(OAc)₂, PdCl₂-type precursors of PdO catalyst, together with more efficient ligands, promote more complex reactions.

The raw materials including the compound of formula (I-2) and formula (I-1) are known to those skilled in the art, or can be prepared by known methods.

The preferred raw material may be formula (I-2-A):

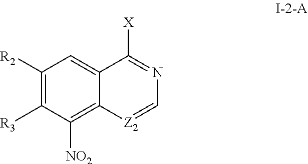

The compound of formula (I-2-A) can be prepared by the method (A) including the following steps:

method (A)

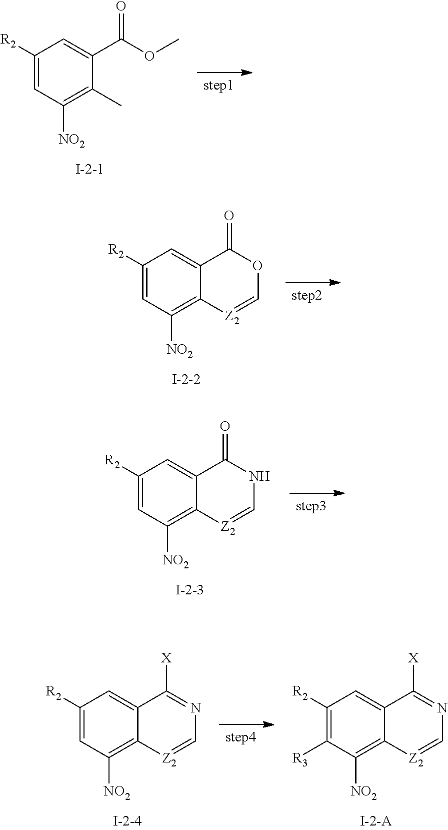

A compound of formula (I-2-2) is obtained by cyclization of compound of formula (I-2-1) with DMF-DMA, the compound of formula (I-2-2) is reacted with ammonia-methanol solution to give a compound of formula (I-2-3), halogenation of the compound of formula (I-2-3) with SOX₂, SO₂X₂, POX₃ or PX₅ (X is preferably chlorine or bromine), preferably POCl₃, is conducted to give a compound of formula (I-2-4). The compound of formula (I-2-4) can be modified by conventional methods in the art to obtain the compound of formula (I-2-A).

In another aspect of the preparation of the compound of formula (I), the preferred raw material may be formula (I-1-A):

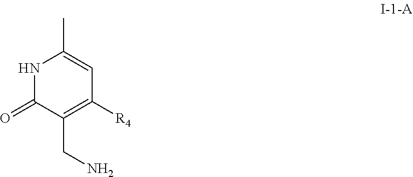

The compound of formula (I-1-A) can be prepared by the following steps:

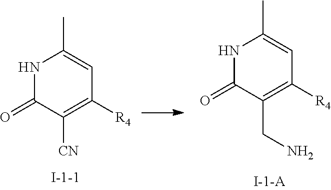

The compound of formula (I-1-1) is reduced by an appropriate reduction method to give a compound of formula (I-1-A). Suitable reduction methods include: reduction with borane in a tetrahydrofuran solution, reduction with lithium aluminum hydride in an anhydrous inert solvent, wherein the inert solvents include: methyl tert-butyl ether, tetrahydrofuran, etc.

The intermediate compound of formula (I-1-1) can be obtained in two different ways. In the first embodiment of the synthetic route, the compound of formula (I-1-1) can be prepared by method 1 comprising the following steps:

Method 1

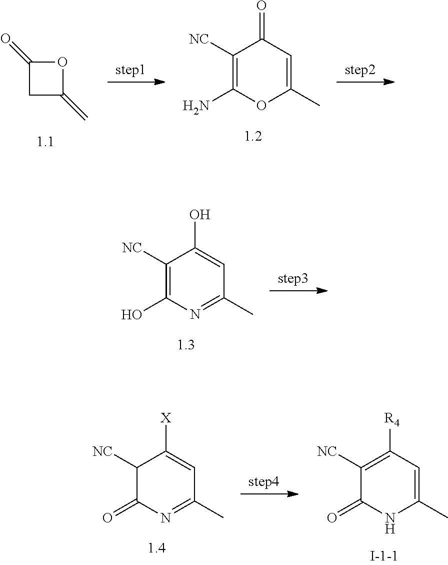

The compound of formula (1.1) is reacted with malononitrile to give the compound of formula (1.2) through ring-opening and reclosing reaction, the compound of formula (1.2) is reacted with an acid to obtain the compound of formula (1.3), and halogenation of the compound of formula (1.3) is conducted with SOX₂, SO₂X₂, POX₃ or PX₅ (X is preferably chlorine or bromine), preferably POCl₃, to give a compound of formula (1.4). The compound of formula (1.4) is reacted with an alcohol under basic conditions to give a compound of formula (I-1-1), suitable bases include alkali metal hydroxides, preferably sodium hydroxide, potassium hydroxide, alkali metal alkoxides, preferably sodium alkoxides.

In the second embodiment of the synthetic route, the compound of formula (I-1-1) can be prepared by the method 2 including the following steps:

Method 2

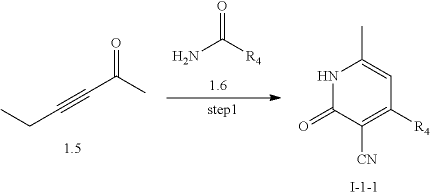

A ring-closing reaction of the compound of formula (1.5) with the compound of formula (1.6) is conducted under basic conditions to give the compound of formula (I-1-1). Suitable basic systems include potassium tert-butoxide in DMSO, potassium carbonate in DMSO, potassium carbonate in DMF, etc.

The reactions in the above steps are all conventional reactions known to those skilled in the art. Unless otherwise specified, the reagents and raw material compounds used in the synthetic route are all commercially available, or can be prepared by those skilled in the art by referring to known methods according to the different compound structures designed.

Compared with the prior art, the main advantages of the present disclosure are to provide a series of novel 1,5,7-trisubstituted isoquinoline derivatives which have high inhibitory activity against EZH2 and can be used as drugs for treating tumors.

The present invention will be further illustrated below with reference to the specific examples. It should be understood that these examples are only to illustrate the invention but not to limit the disclosure of the invention. The experimental methods without specific conditions in the following embodiments are generally carried out according to conventional conditions, or in accordance with the conditions recommended by the manufacturer. Unless indicated otherwise, parts and percentage are calculated by weight. Unless otherwise defined, terms used herein are of the same meanings that are familiar to those skilled in the art. In addition, any methods and materials similar with or equivalent to those described herein can be applied to the present invention.

As used herein, DMB refers to 2,4-dimethoxybenzyl, THF refers to tetrahydrofuran, EA refers to ethyl acetate, PE refers to petroleum ether, Ac₂O refers to acetic anhydride, NBS refers to N-bromosuccinimide, DCM refers to dichloromethane, AIBN refers to azodiisobutyronitrile, Pd(dppf)Cl₂ refers to [1,1′-bis(diphenylphosphino)ferrocene]palladium dichloride, TFA refers to trifluoroacetic acid, TBSCl refers to tert-butyldimethylchlorosilane, NCS refers to N-chlorosuccinimide, DHP refers to dihydrogenpyran, LiAlH₄ refers to lithium aluminium hydride, PMB refers to p-methoxybenzyl, LiHMDS refers to lithium bistrimethylsilylamide, Pd₂(dba)₃ refers to tris(dibenzylideneacetone)dipalladium, RuPhos refers to 2-dicyclohexylphosphoryl-2′,6′-diisopropoxy-1,1′-biphenyl, DMAP refers to 4-dimethylaminopyridine, THP refers to tetrahydropyran, n-BuLi refers to n-butyllithium, TMsOTf refers to trimethylsilyl trifluoromethanesulfonate, TEBAC refers to triethylbenzylammonium chloride, HATU refers to 2-(7-azobenzotriazole)-N,N,N′,N′-tetramethyluronium hexafluorophosphate, DMF refers to dimethylformamide, DMSO refers to dimethylsulfoxide, DIEA refers to N,N-diisopropylethylamine, BINAP refers to (2R,3 S)-2,2′-bis diphenylphosphino-1,1′-binaphthyl.

As used herein, room temperature refers to about 20-25° C.

Preparation of Intermediate 1a

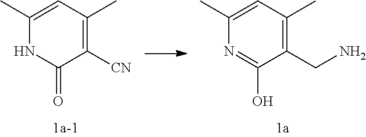

To a solution of compound 1a-1 (22.5 g, 152 mmol) in tetrahydrofuran (500 mL) was slowly added lithium aluminum hydride (11.5 g, 0.3 mol) under ice-bath, and the mixture was stirred at room temperature overnight. 15 mL of water and 30 mL of sodium hydroxide solution (15%) were added to the system respectively, filtered, and the filtrate was concentrated to give 110 g of white solid compound 1a. MS m/z (ESI): N/A.

Preparation of Intermediate 2a

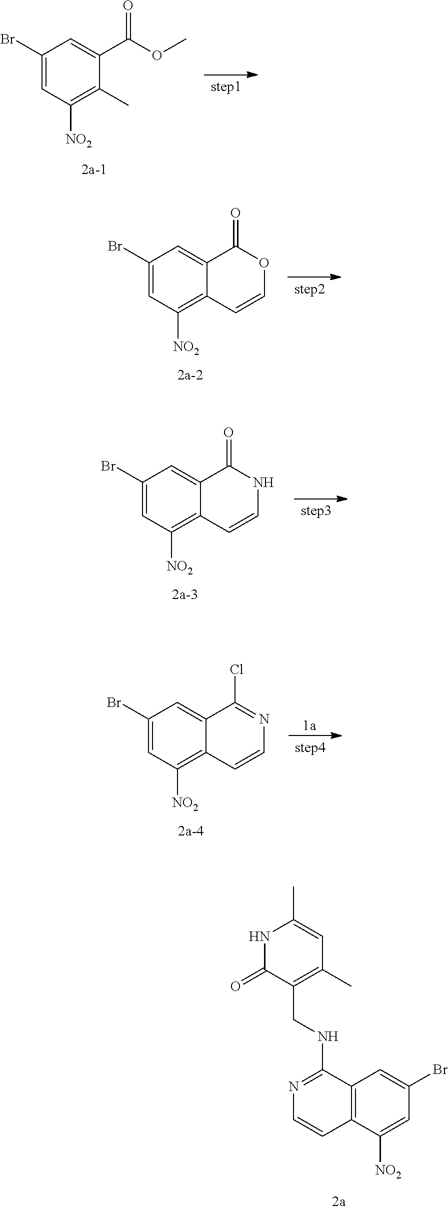 [SMILES: COC(=O)c1cc(Br)cc([N+](=O)[O-])c1C ; O=c1occc2c([N+](=O)[O-])cc(Br)cc12 ; O=c1[nH]ccc2c([N+](=O)[O-])cc(Br)cc12 ; O=[N+]([O-])c1cc(Br)cc2c(Cl)nccc12 ; Cc1cc(C)c(CNc2nccc3c([N+](=O)[O-])cc(Br)cc23)c(=O)[nH]1]

Step 1: A mixture of compound 2a-1 (500 mg, 1.82 mmol), DMF-DMA (575 mg, 5.47 mmol) and 5 mL of DMF was stirred under nitrogen at 110° C. overnight. Using LC-MS to monitor the reaction until the reaction was complete. The reaction solution was cooled and concentrated, and purified by silica gel column chromatography to give compound 2a-2 (130 mg, 26%) as a white solid. MS m/z (ESI): N/A.

Step 2: To compound 2a-2 (130 mg, 0.481 mmol) was added NH₃.MeOH (3 mL, 7M). The mixture was stirred overnight at 60° C. in a sealed tube. LC-MS was used to trace the reaction until the reaction was complete. The mixture was cooled and filtered. The filter cake was distilled under reduced pressure to give 80 mg of compound 2a-3 as a yellow solid. MS m/z (ESI): 268 [M+H]⁺.

Step 3: To compound 2a-3 (80 mg, 0.297 mmol) was added 2 mL of phosphorus oxychloride, the mixture was stirred at 100° C. for 2 hours. LC-MS was used to trace the reaction until the reaction was complete. The mixture was cooled and poured into ice water. The pH was adjusted to 8 with aqueous ammonia. Extracted with ethyl acetate and dried, concentrated to give 100 mg of white solid compound 2a-4. MS m/z (ESI): 289 [M+H]⁺.

Step 4: Compound 2a-4 (100 mg, 0.347 mmol), compound 1a (98 mg, 0.522 mmol) and potassium carbonate (240 mg, 1.74 mmol) were added to 5 mL of DMF, the mixture was stirred under argon atmosphere at 70° C. overnight. LC-MS was used to trace the reaction until the reaction was complete. The mixture was cooled and poured into water and filtered to give 150 mg of red solid compound 2a. MS m/z (ESI): 405.1 [M+H]⁺.

Preparation of Intermediate 3a

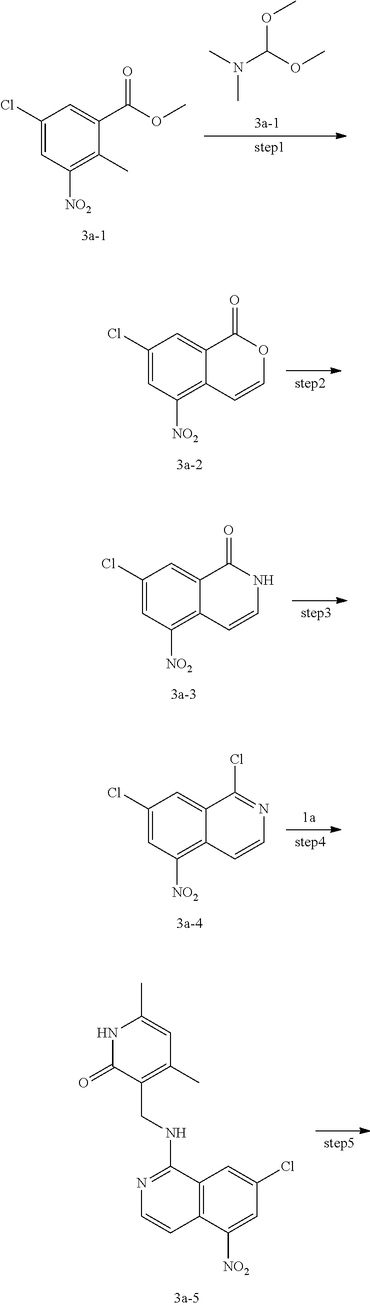

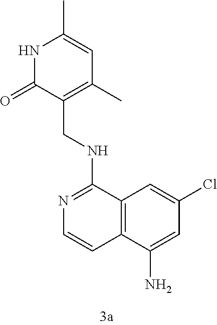 [SMILES: Cc1cc(C)c(CNc2nccc3c(N)cc(Cl)cc23)c(=O)[nH]1]

Step 1: A solution of compound 3a-1 (2 g, 9.3 mmol) and compound 3a.1 (3.1 g, 27.9 mmol) in DMF (20 mL) was stirred at 110° C. overnight. LC-MS was used to trace the reaction until the reaction was complete. The reaction solution was cooled and poured into water, extracted with ethyl acetate, dried and concentrated, and purified by combiflash to give yellow solid compound 3a-2 (1.3 g, 66.3%). MS m/z (ESI): 226 [M+H]⁺.

Step 2: The preparation method is the same as that of compound 2a-3, except that compound 2a-2 in the preparation method of 2a-3 is replaced by compound 3a-2. 900 mg crude product of compound 3a-3 is obtained as a yellow solid. MS m/z (ESI): 225[M+H]⁺.

Step 3: The preparation method is the same as that of compound 2a-4, except that compound 2a-3 in the preparation method of 2a-4 is replaced by compound 3a-3. After purification by combiflash, compound 3a-4 is obtained as a white solid (1.8 g, 72%). MS m/z (ESI): 288.9 [M+H]⁺.

Step 4: The preparation method is the same as that of compound 2a, except that compound 2a-4 in the preparation method of 2a is replaced by compound 3a-4. 700 mg of red solid compound 3a-5 is obtained. MS m/z (ESI): 359 [M+H]⁺.

Step 5: A solution of compound 3a-5 (700 mg, 1.96 mmol) and sodium bicarbonate (246 mg, 2.93 mmol) in ethanol (100 mL) was stirred at 80° C., and a solution of sodium thiosulfate (1.7 g, 9.78 mmol) in water (15 mL) was added dropwise, the mixture was stirred at 80° C. for 1 hour. LC-MS was used to trace the reaction until the reaction was complete. The reaction solution was cooled to room temperature, hydrochloric acid solution (300 mL, 37%) was added and the solution was heated to 60° C. and stirred for 1 hour. Cooled to room temperature and sodium hydroxide (4N) was added to adjust pH to 8, extracted with ethyl acetate, the organic layer was separated, dried and concentrated to give 600 mg of compound 3a as a yellow solid. MS m/z (ESI): 329 [M+H]⁺.

Preparation of Intermediate 4a

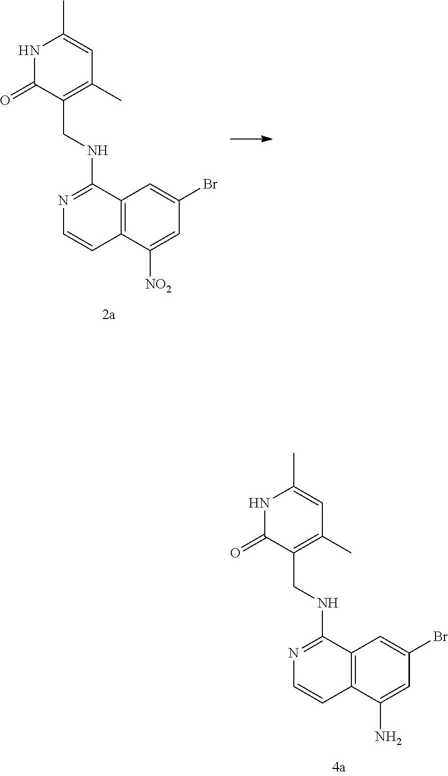 [SMILES: Cc1cc(C)c(CNc2nccc3c([N+](=O)[O-])cc(Br)cc23)c(=O)[nH]1 ; Cc1cc(C)c(CNc2nccc3c(N)cc(Br)cc23)c(=O)[nH]1]

A solution of compound 2a (150 mg, 0.372 mmol) and sodium bicarbonate (39 mg, 0.465 mmol) in ethanol (5 mL) was stirred at 80° C., and a solution of sodium thiosulfate (324 mg, 1.86 mmol) in water (0.5 mL) was added dropwise. After the addition was complete, the reaction solution was concentrated and the residue was dissolved in water (100 mL) and filtered. The filter cake was collected to give 100 mg of a pale yellow solid compound 4a. MS m/z (ESI): 375.1 [M+H]⁺.

Preparation of Intermediate 5a

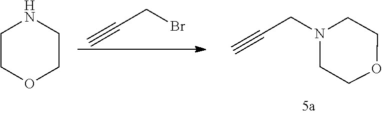

To a solution of compound 3-bromopropyne (1.64 g, 13.8 mmol) in THF (10 mL) was added potassium carbonate (3.17 g, 23 mmol) and morpholine (1 g, 11.5 mmol), and the mixture was stirred at 70° C. for 6 hours. The reaction solution was extracted with ethyl acetate/water system, the organic layer was dried and concentrated, purified by combiflash (EA/PE=1:2) to give compound 5a (450 mg, 31%). MS m/z (ESI): N/A.

Preparation of Intermediate 6a

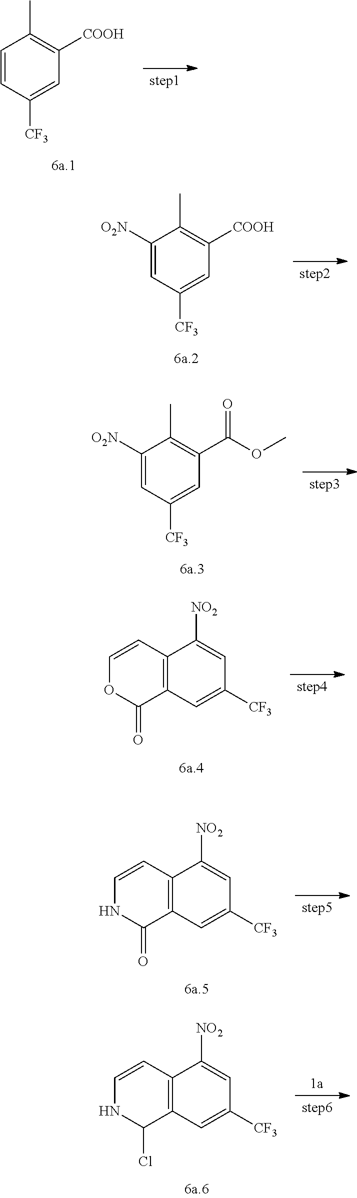 [SMILES: Cc1ccc(C(F)(F)F)cc1C(=O)O ; Cc1c(C(=O)O)cc(C(F)(F)F)cc1[N+](=O)[O-] ; COC(=O)c1cc(C(F)(F)F)cc([N+](=O)[O-])c1C ; O=c1occc2c([N+](=O)[O-])cc(C(F)(F)F)cc12 ; O=c1[nH]ccc2c([N+](=O)[O-])cc(C(F)(F)F)cc12 ; O=[N+]([O-])c1cc(C(F)(F)F)cc2c1C=CNC2Cl]

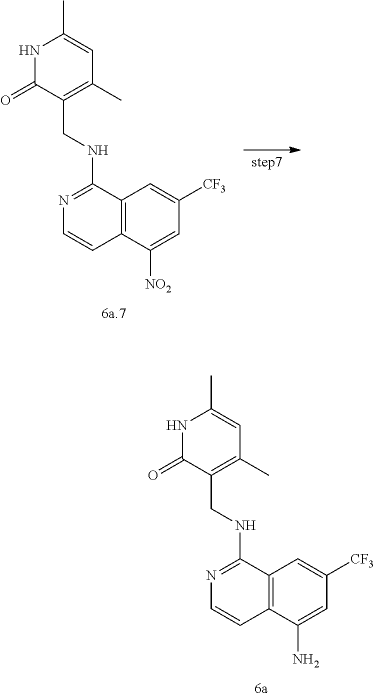 [SMILES: Cc1cc(C)c(CNc2nccc3c([N+](=O)[O-])cc(C(F)(F)F)cc23)c(=O)[nH]1 ; Cc1cc(C)c(CNc2nccc3c(N)cc(C(F)(F)F)cc23)c(=O)[nH]1]

Step 1: To a solution of compound 6a.1 (500 mg, 2.45 mmol) in sulfuric acid (5 mL, 12N) was added nitric acid (0.5 mL, 12N) in an ice bath, and the mixture was stirred at room temperature for 2 hours. Water was added to the reaction solution and the mixture was filtered. The cake was washed with water and dried to give 560 mg of solid compound 6a.2. MS m/z (ESI): 248 [MH]⁺.

Step 2: To a solution of compound 6a.2 (560 mg, 2.25 mmol) in DMF (5 mL) was added methyl iodide (639 mg, 4.5 mmol) and potassium carbonate (931 mg, 6.75 mmol), the mixture was stirred at 60° C. for 5 hours. LC-MS was used to trace the reaction until the reaction was completed. The reaction solution was extracted with ethyl acetate/water and the organic layer was concentrated and purified by combiflash (EA/PE=10:1) to give compound 6a.3 (530 mg, 89%). MS m/z (ESI): 262 [MH]⁺.

Step 3: The preparation method is the same as that of compound 2a-2, except that compound 2a-1 in the preparation method of 2a-2 is replaced by compound 6a.3, and the mixture is stirred at 110° C. for 4 hours. MS m/z (ESI): N/A.

Step 4: The preparation method is the same as that of compound 2a-3, except that compound 2a-2 in the preparation method of 2a-3 is replaced by compound 6a.4. MS m/z (ESI): 259[M+H]⁺.

Step 5: The preparation method is the same as that of compound 2a-4, except that compound 2a-3 in the preparation method of 2a-4 is replaced by compound 6a.5. MS m/z (ESI): 277 [M+H]⁺.

Step 6: The preparation method is the same as that of compound 2a, except that compound 2a-4 in the preparation method of 2a is replaced by compound 6a.6, and the mixture is stirred at 70° C. for 5 hours. MS m/z (ESI): 393 [M+H]⁺.

Step 7: The preparation method is the same as that of compound 3a, except that compound 3a-5 in the preparation method of 3a was replaced by compound 6a.7, and the mixture is stirred at 80° C. overnight. MS m/z (ESI): 363 [M+H]⁺.

Preparation of Intermediate 7a

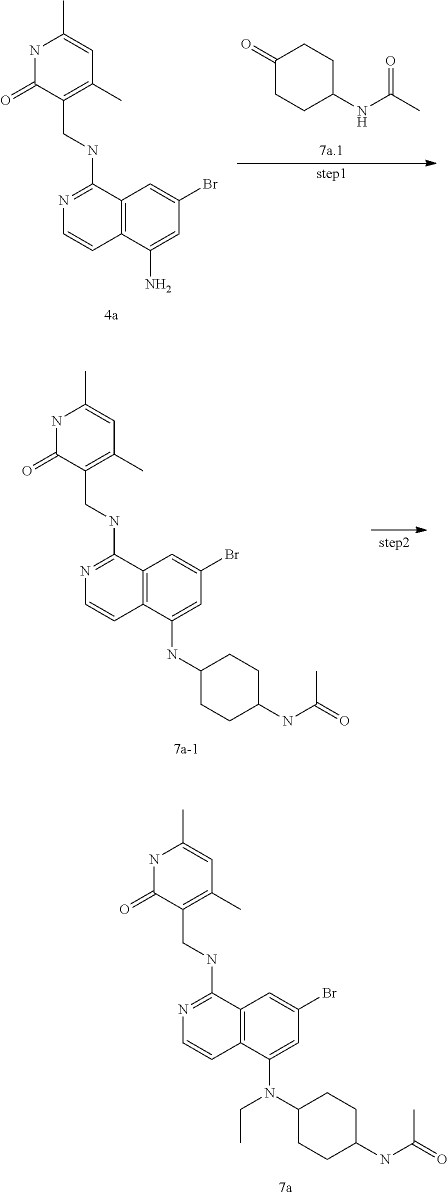 [SMILES: Cc1cc(C)c(CNc2nccc3c(N)cc(Br)cc23)c(=O)[nH]1 ; CC(=O)NC1CCC(=O)CC1 ; CC(=O)NC1CCC(Nc2cc(Br)cc3c(NCc4c(C)cc(C)[nH]c4=O)nccc23)CC1 ; CCN(c1cc(Br)cc2c(NCc3c(C)cc(C)[nH]c3=O)nccc12)C1CCC(NC(C)=O)CC1]

Step 1: Compound 4a (500 mg, 1.34 mmol), compound 7a.1 (624 mg, 4.02 mmol) and a solution of 7.5 mL trifluoroacetic acid in 1,4-dioxane (75 mL) were stirred at room temperature for 2 hours, sodium triacetoxyborohydride (852 mg, 4.02 mmol) was added in batches under an ice bath, stirred at room temperature overnight. LC-MS was used to trace the reaction until the reaction was complete. The reaction solution was poured into water, then the pH was adjusted to 8 with sodium bicarbonate solution, extracted with dichloromethane and dried, concentrated to give 700 mg of compound 7a-1. MS m/z (ESI): 512.2 [M+H]⁺.

Step 2: The preparation method is the same as that of compound 7a-1, except that compounds 4a and 7a. 1 in the preparation method of 7a-1 are replaced by compound 7a-1 and acetaldehyde. Purified by combiflash to give solid compound 7a (400 mg, 58%). MS m/z (ESI): 542.3 [M+H]⁺.

Preparation of Intermediate 8a

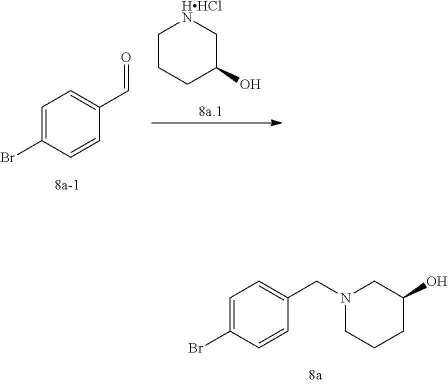

A solution of compound 8a.1 (1.02 g, 7.43 mmol) and potassium carbonate (2.65 g, 14.86 mmol) in acetonitrile (20 mL) was stirred at 50° C. for 2 hours. The reaction solution was filtered and concentrated. Methanol (50 mL) and compound 8a-1 (550 mg, 2.97 mmol) were added to the residue. Sodium triacetoxyborohydride (1.89 g, 8.92 mmol) was added in batches under an ice bath and the mixture was stirred overnight at room temperature. LC-MS was used to trace the reaction until the reaction was complete. The reaction solution was poured into water and the pH was adjusted to 1, extracted with ethyl acetate to remove impurities, the aqueous layer was adjusted to pH 7, and then extracted with ethyl acetate, the organic layers were combined and dried to give 600 mg of compound 8a. MS m/z (ESI): 272.1 [M+H]⁺.

Preparation of Intermediate 9a

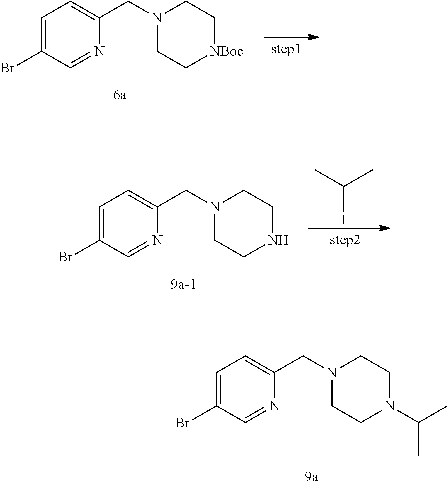

Step 1: To a solution of compound 6a (410 mg, 1.15 mmol) in dichloromethane (4 mL) was added hydrochloric acid/1,4-dioxane (4 mL, 4 M), the mixture was stirred at room temperature overnight. LC-MS was used to trace the reaction until the reaction was complete. The reaction solution was concentrated to give 336 mg of white solid compound 9a-1. MS m/z (ESI): 258 [M+H]⁺.

Step 2: To a solution of compound 9a-1 (286 mg, 0.88 mmol) and 2-iodopropane (249 mg, 1.47 mmol) in DMF (5 mL) was added potassium carbonate (338 mg, 2.44 mmol), and the mixture was stirred under argon atmosphere at 60° C. for 3 hours. LC-MS was used to trace the reaction until the reaction was complete. The reaction solution was poured into water and extracted with ethyl acetate. The organic layer was dried and concentrated to give 220 mg of compound 9a. MS m/z (ESI): 298 [M+H]⁺.

Preparation of Intermediate 10a

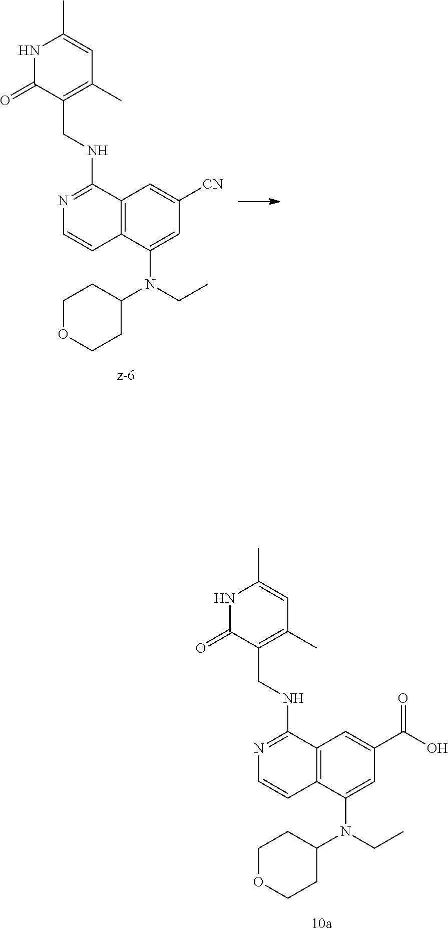 [SMILES: CCN(c1cc(C#N)cc2c(NCc3c(C)cc(C)[nH]c3=O)nccc12)C1CCOCC1 ; CCN(c1cc(C(=O)O)cc2c(NCc3c(C)cc(C)[nH]c3=O)nccc12)C1CCOCC1]

Sodium hydroxide (30 mL, 1N) was added to a solution of compound z-6 (700 mg, 1.65 mmol) in ethanol (30 mL), and the mixture was stirred at 100° C. for 20 hours. LC-MS was used to trace the reaction until the reaction was complete. The reaction solution was concentrated to remove ethanol and hydrochloric acid solution (2M) was added for neutralization, extracted with ethyl acetate, and the organic layer was dried and concentrated to give 250 mg of a yellow compound 10a. MS m/z (ESI): 451.3 [M+H]⁺.

Preparation of Intermediate 11a

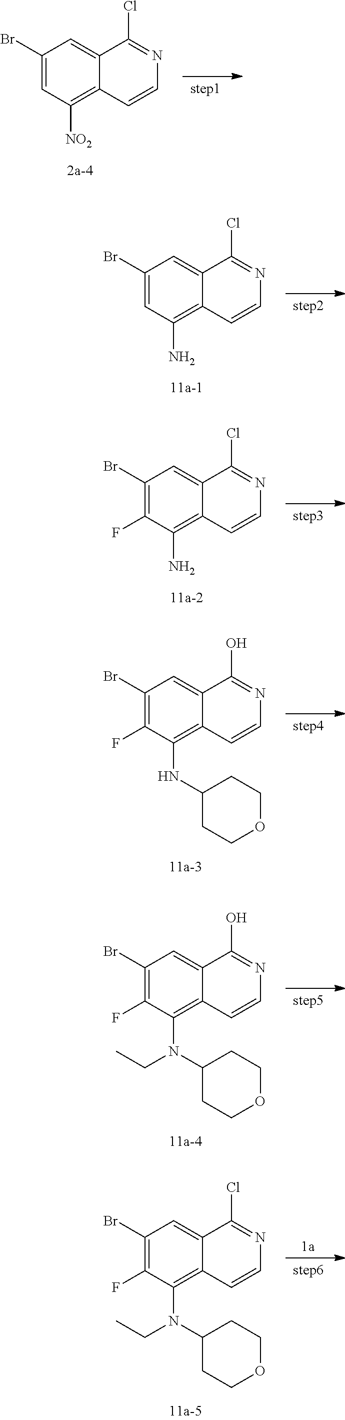 [SMILES: O=[N+]([O-])c1cc(Br)cc2c(Cl)nccc12 ; Nc1cc(Br)cc2c(Cl)nccc12 ; Nc1c(F)c(Br)cc2c(Cl)nccc12 ; Oc1nccc2c(NC3CCOCC3)c(F)c(Br)cc12 ; CCN(c1c(F)c(Br)cc2c(O)nccc12)C1CCOCC1 ; CCN(c1c(F)c(Br)cc2c(Cl)nccc12)C1CCOCC1]

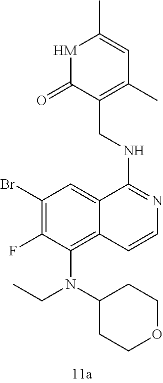

Step 1: A solution of compound 2a-4 (5 g, 6.99 mmol) in acetic acid (20 mL) was heated to 75° C., iron powder (1.96 g, 34.96 mmol) was slowly added, and the mixture was stirred at 75° C. for 1 h. LC-MS was used to trace the reaction until the reaction was complete. The solution was cooled to room temperature, filtered and the filtrate was poured into water, extracted with ethyl acetate and concentrated, purified by combiflash to give compound 11a-1 as a yellow solid (1.2 g, 67%). MS m/z (ESI): 257 [M+H]⁺.

Step 2: To a solution of compound 11a-1 (1.2 g, 4.67 mmol) in acetonitrile (200 mL) was added selectfluor (1.5 g, 4.2 mmol) and the mixture was stirred overnight at room temperature. LC-MS was used to trace the reaction until the reaction was complete. The reaction solution was poured into water, extracted with ethyl acetate and concentrated, purified by combiflash to give compound 11a-2 (260 mg, 20%) as a yellow solid. MS m/z (ESI): 275 [M+H]⁺.

Step 3: The preparation method is the same as that of compound 7a, except that compound 7a-1 and acetaldehyde in the preparation method of 7a are replaced by compound 11a-2 and tetrahydropyrone. MS m/z (ESI): 341[M+H]⁺.

Step 4: The preparation method is the same as that of compound 7a, except that compound 7a-1 in the preparation method of 7a is replaced by compound 11a-3. MS m/z (ESI): 368 [M+H]⁺.

Step 5: The preparation method is the same as that of compound 2a-4, except that compound 2a-3 in the preparation method of 2a-4 is replaced by compound 11a-4. MS m/z (ESI): 387 [M+H]⁺.

Step 6: The preparation method is the same as that of compound 2a, except that compound 2a-4 in the preparation method of 2a is replaced by compound 11a-5. MS m/z (ESI): 503 [M+H]⁺.

Preparation of Intermediate 12a

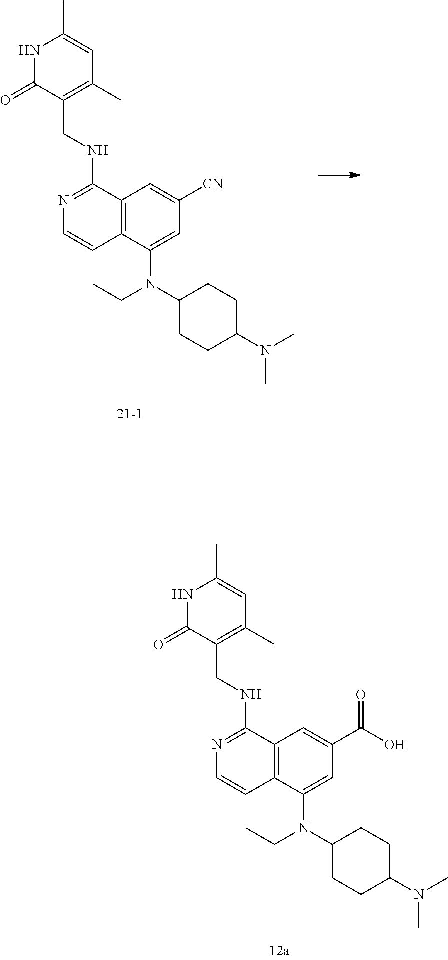 [SMILES: CCN(c1cc(C#N)cc2c(NCc3c(C)cc(C)[nH]c3=O)nccc12)C1CCC(N(C)C)CC1 ; CCN(c1cc(C(=O)O)cc2c(NCc3c(C)cc(C)[nH]c3=O)nccc12)C1CCC(N(C)C)CC1]

The preparation method is the same as that of compound 10a, except that compound z-6 in the preparation method of 10a is replaced by compound 21-1. MS m/z (ESI): 492.4 [M+H]⁺.

Preparation of Intermediate 13a

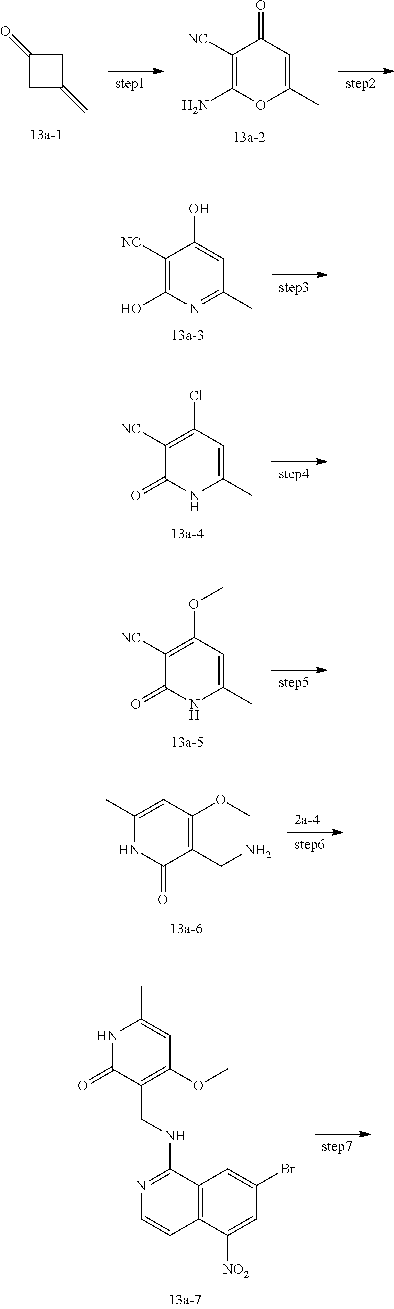

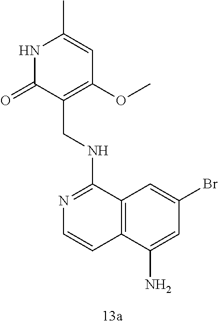 [SMILES: COc1cc(C)[nH]c(=O)c1CNc1nccc2c(N)cc(Br)cc12]

Step 1: A solution of compound malononitrile (12 g, 181.7 mmol) in dry tetrahydrofuran (225 mL) was stirred under ice bath for 1 h. NaH (4.8 g, 199.8 mmol) was added in batches and stirred for 2 h, compound 13a-1 (16.8 g, 199.8 mmol) was added dropwise, the mixture was slowly warmed to room temperature and reacted for 1 hour. The reaction solution was quenched with hydrochloric acid solution, extracted with ethyl acetate, and the organic layer was dried and concentrated to give a yellow solid compound 13a-2 which was used directly for the next step. MS m/z (ESI): 151 [M+H]⁺.

Step 2: A mixture of compound 13a-2 (28 g, 181.7 mmol), hydrochloric acid (23.2 g, 4 M, 636.4 mmol) and water (160 mL) was stirred under reflux for 5 hours. The reaction solution was filtered, and the solid residue was recrystallized from methanol to obtain 25 g of compound 13a-3. MS m/z (ESI): 151 [M+H]⁺.

Step 3: The preparation method is the same as that of compound 2a-4, except that compound 2a-3 in the preparation method of 2a-4 is replaced by compound 13a-3. MS m/z (ESI): 169 [M+H]⁺.

Step 4: A mixture of compound 13a-4 (300 mg, 1.78 mmol), sodium methoxide (481 mg, 8.9 mmol) and methanol (15 mL) was microwaved at 100° C. for 16 h. LC-MS was used to trace the reaction until the reaction was complete. Concentrated to remove the solvent, the residue was added water and the pH was adjusted to 7, filtered to give 225 mg of solid compound 13a-5. MS m/z (ESI): 419 [M+H]⁺.

Step 5: The preparation method is the same as that of compound 1a, except that compound 1a-1 in the preparation method of 1a is replaced by compound 13a-5. MS m/z (ESI): 152 [M+H]⁺.

Step 6: The preparation method is the same as that of compound 2a, except that compound 2a-4 in the preparation method of 2a is replaced by compound 13a-6. MS m/z (ESI): 152 [M+H]⁺.

Step 7: The preparation method is the same as that of compound 11a-1, except that compound 2a-4 in the preparation method of 11a-1 is replaced by compound 13a-7. MS m/z (ESI): 389 [M+H]⁺.

Preparation of Intermediate 14a

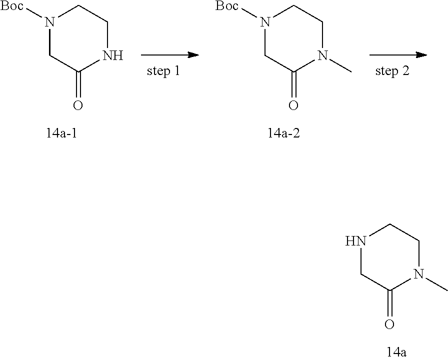

Step 1: The preparation method is the same as that of compound z-225, except that compound z-156 in the preparation method of z-225 is replaced by compound 14a-1. MS m/z (ESI): 159 [M+H]⁺.

Step 2: The preparation method is the same as that of compound 9a-1, except that compound 6a in the preparation method of 9a-1 was replaced by compound 14a-2. MS m/z (ESI): 115 [M+H]⁺.

Preparation of Intermediate 15a

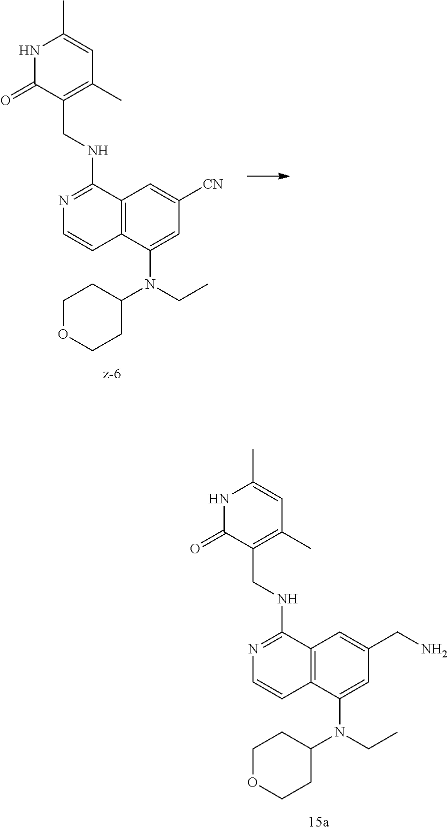 [SMILES: CCN(c1cc(C#N)cc2c(NCc3c(C)cc(C)[nH]c3=O)nccc12)C1CCOCC1 ; CCN(c1cc(CN)cc2c(NCc3c(C)cc(C)[nH]c3=O)nccc12)C1CCOCC1]

The preparation method is the same as that of compound 1a, except that compound 1a-1 in the preparation method of 1a is replaced by compound z-6. MS m/z (ESI): 436 [M+H]⁺.

Preparation of Intermediates 16a to 37a

General procedure: Compounds 16a to 37a are prepared by method similar to that of intermediate 7a with isoquinoline aldehyde ketone compounds as raw materials. Their structures are as shown in formula (I), wherein when X is NH; Z₁ is N; Z₂, Z₃ are CH; R₁, R₃ are hydrogen; R₂, R₄, NR₅R₆ are the structures shown in the following table:

| Compounds | NR₅R₆ | R₂ | R₄ | MS[M + H]⁺ |
|---|---|---|---|---|
| 16a | 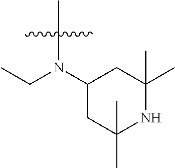 | Br | CH₃ | 541 |
| 17a | 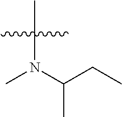 [SMILES: *N(C)C(C)CC] | Br | CH₃ | 443 |
| 18a | 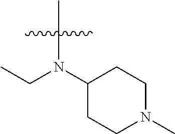 [SMILES: *N(CC)C1CCN(C)CC1] | Br | CH₃ | 498 |
| 19a | 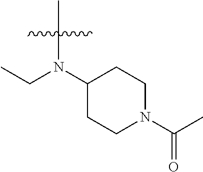 [SMILES: *N(CC)C1CCN(C(C)=O)CC1] | Br | CH₃ | 527 |
| 20a | 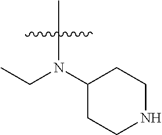 [SMILES: *N(CC)C1CCNCC1] | Br | CH₃ | 485 |
| 21a | 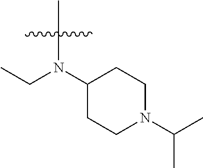 [SMILES: *N(CC)C1CCN(C(C)C)CC1] | Br | CH₃ | 527 |
| 22a | 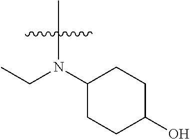 [SMILES: *N(CC)C1CCC(O)CC1] | Br | CH₃ | 500 |
| 23a | 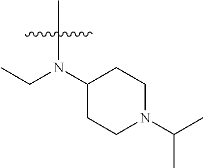 [SMILES: *N(CC)C1CCN(C(C)C)CC1] | Br | CH₃O | 542 |
| 24a | 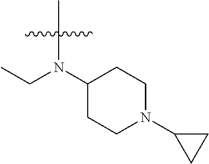 [SMILES: *N(CC)C1CCN(C2CC2)CC1] | Br | CH₃ | 525 |
| 25a | 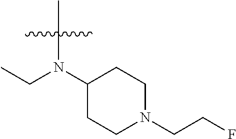 [SMILES: *N(CC)C1CCN(CCF)CC1] | Br | CH₃ | 531 |
| 26a | 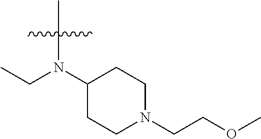 [SMILES: *N(CC)C1CCN(CCOC)CC1] | Br | CH₃ | 543 |
| 27a | 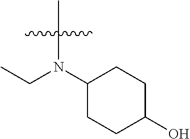 [SMILES: *N(CC)C1CCC(O)CC1] | Br | CH₃ | 613 |
| 28a | 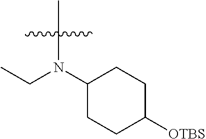 [SMILES: *N(CC)C1CCC(O[Si](C)(C)C(C)(C)C)CC1] | Br | CH₃ | 615 |
| 29a | 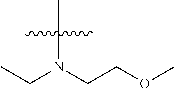 [SMILES: *N(CC)CCOC] | Br | CH₃ | 459 |
| 30a | 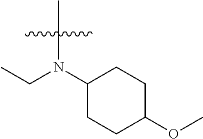 [SMILES: *N(CC)C1CCC(OC)CC1] | Br | CH₃ | 513 |
| 31a | 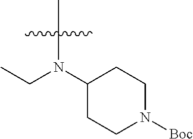 [SMILES: *N(CC)C1CCN(C(=O)OC(C)(C)C)CC1] | Br | CH₃ | 584 |
| 32a | 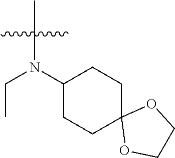 [SMILES: *N(CC)C1CCC2(CC1)OCCO2] | Br | CH₃ | 542 |
| 33a | 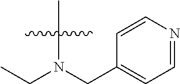 [SMILES: *N(CC)Cc1ccncc1] | Br | CH₃ | 493 |
| 34a | 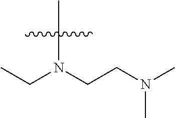 [SMILES: *N(CC)CCN(C)C] | Br | CH₃ | 472 |
| 35a | 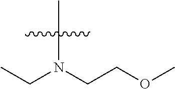 [SMILES: *N(CC)CCOC] | Br | CH₃ | 459 |
| 36a | 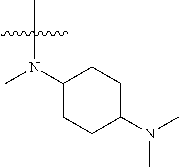 | Br | CH₃ | 513 |
| 37a | Br | 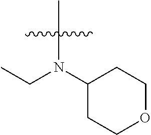 [SMILES: *N(CC)C1CCOCC1] | CH₃O | 501 |

Preparation of Intermediate 38a

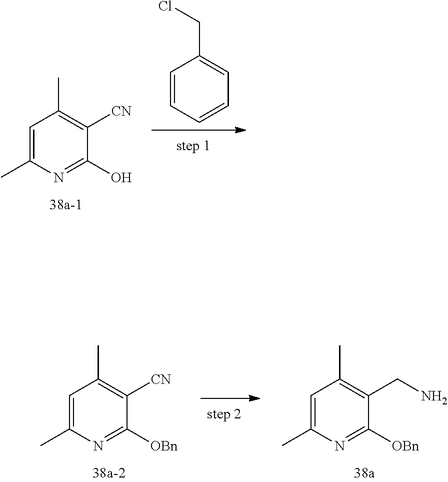 [SMILES: Cc1cc(C)c(C#N)c(O)n1 ; ClCc1ccccc1 ; Cc1cc(C)c(C#N)c(OCc2ccccc2)n1 ; Cc1cc(C)c(CN)c(OCc2ccccc2)n1]

Step 1: A solution of compound 38a-1 (10.36 g, 70 mmol), benzyl chloride (10.58 g, 84 mmol) and silver oxide (19.5 g, 84 mmol) in toluene (260 mL) was stirred at 105° C. overnight. LC-MS was used to trace the reaction until the reaction was complete. The reaction solution was cooled and filtered. The filtrate was concentrated and purified by combiflash to give 4 g of compound 38a-2. MS m/z (ESI): 239 [M+H]⁺.

Step 2: The preparation method is the same as that of compound 1a, except that compound 1a-1 in the preparation method of 1a is replaced by compound 38a-2. MS m/z (ESI): 243 [M+H]⁺.

Preparation of Intermediate 39a

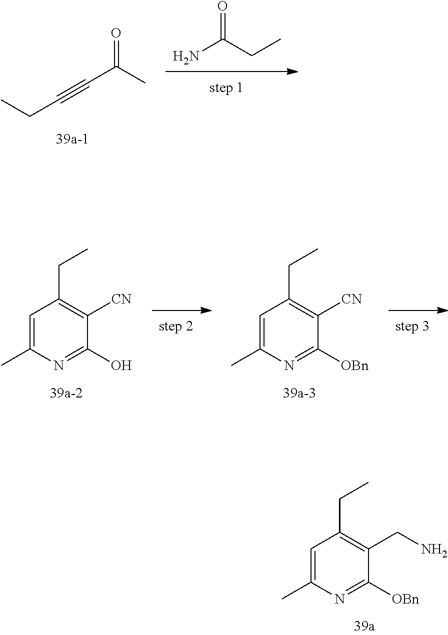

Step 1: To a solution of compound propionamide (420 mg, 5 mmol) in DMSO (10 mL) was added potassium tert-butoxide (560 mg, 5.25 mmol) and stirred for 30 minutes at room temperature. Compound 39a-1 (480 mg, 5 mmol) was added under ice bath, and the mixture was stirred at room temperature for 3 hours. LC-MS was used to trace the reaction until the reaction was complete. The reaction solution was added saturated ammonium chloride solution and water, a solid was precipitated and filtered, the filter cake was washed with water and dried under reduced pressure to give 520 mg of compound 39a-2. MS m/z (ESI): 163[M+H]⁺.

Step 2: The preparation method is the same as that of compound 38a-2, except that compound 38a-1 in the preparation method of 38a-2 is replaced by compound 39a-2. MS m/z (ESI): 253 [M+H]⁺.

Step 3: The preparation method is the same as that of compound 1a, except that compound 1a-1 in the preparation method of 1a is replaced by compound 39a-3. MS m/z (ESI): 257 [M+H]⁺.

Preparation of Intermediate 40a

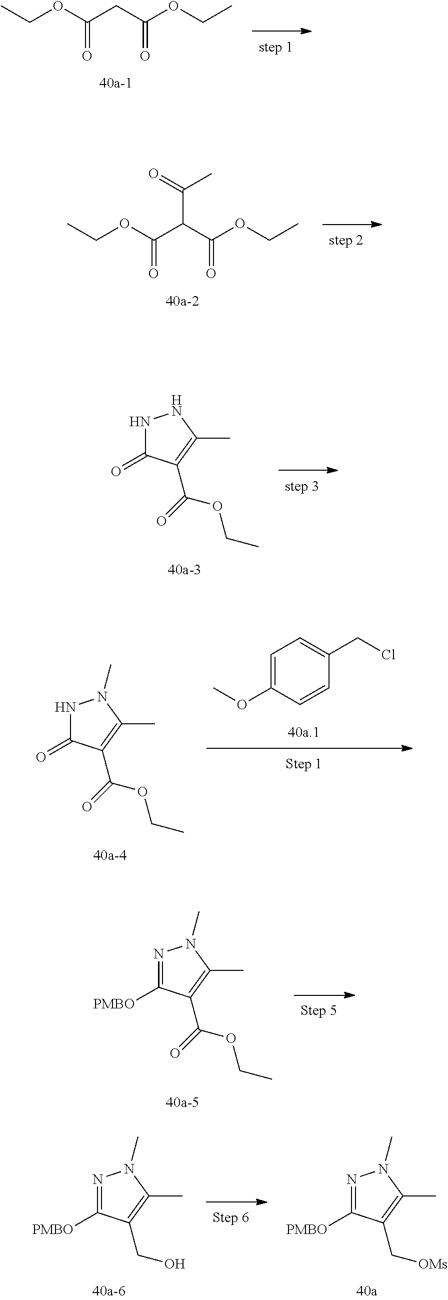

Step 1: To a solution of compound 40a-1 (120 g, 0.74 mol), magnesium chloride (63.5 g, 0.67 mol) and triethylamine (187 mL, 1.33 mol) in acetonitrile (250 mL) was slowly added acetyl chloride (52.3 g, 0.67 mol) dropwise at below 20° C., and the mixture was stirred at room temperature overnight. The reaction solution was adjusted to pH 1-2 with hydrochloric acid solution, extracted with ethyl acetate, the organic layer was dried and concentrated to give compound 40a-2 which was used directly in the next step. MS m/z (ESI): 203[M+H]⁺.

Step 2: To a solution of compound 40a-2 (154 g, 0.59 mol) in acetic acid (500 mL) was slowly added hydrazine hydrate (36 g, 0.59 mol). The mixture was heated to 100° C. and stirred for 3 hours. The reaction solution was concentrated to 200 mL and stirred after adding 200 mL of ethanol, filtered to give compound 40a-3. MS m/z (ESI): 171 [M+H]⁺.

Step 3: To a solution of sodium hydroxide (1 g, 25.6 mmol) in water (18 mL) was added compound 40a-3 (2 g, 12.8 mol) and dimethyl sulfate (3.22 g, 25.6 mmol) under ice bath, and the mixture was stirred at room temperature for 1 hour, heated to 80° C. and stirred for 3 hours. The reaction solution was filtered and the filter cake was dried under reduced pressure to give compound 40a-4. MS m/z (ESI): 185 [M+H]⁺.

Step 4: To a solution of compound 40a-4 (20 g, 108 mmol) in DMF (260 mL) was slowly added sodium hydride (6.5 g, 130 mmol) in an ice bath under argon atmosphere and stirred for 1 hour under ice bath before adding compound 40a.1 (20.34 g, 130 mmol), the mixture was stirred at room temperature for 16 hours. The reaction solution was extracted with ethyl acetate/water, the organic layer was dried, concentrated, and purified by combiflash to give compound 40a-5. MS m/z (ESI): 305.1 [M+H]⁺.

Step 5: The preparation method is the same as that of compound 1a, except that compound 1a-1 in the preparation method of 1a is replaced by compound 40a-5. MS m/z (ESI): N/A.

Step 6: The preparation method is the same as that of compound 29b-2, except that compound 29b-1 in the preparation method of 29b-2 is replaced by compound 40a-6. MS m/z (ESI): N/A.

Preparation of Intermediate 41a

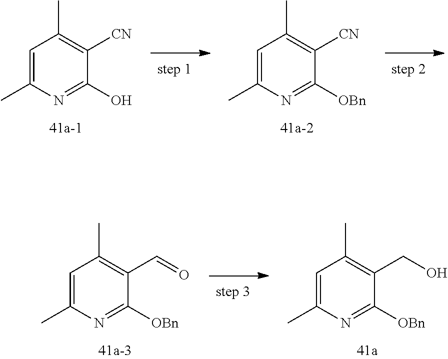 [SMILES: Cc1cc(C)c(C#N)c(O)n1 ; Cc1cc(C)c(C#N)c(OCc2ccccc2)n1 ; Cc1cc(C)c(C=O)c(OCc2ccccc2)n1 ; Cc1cc(C)c(CO)c(OCc2ccccc2)n1]

Step 1: The preparation method is the same as that of compound 38a-2, except that compound 38a-1 in the preparation method of 38a-2 is replaced by compound 41a-1. MS m/z (ESI): 239 [M+H]⁺.

Step 2: To a solution of compound 41a-2 (300 mg, 1.26 mmol) in dichloromethane (10 mL) was added DIBAL-H (2 mL, 3 mmol) under ice bath and the mixture was stirred at room temperature overnight. LC-MS was used to trace the reaction until the reaction was complete. The reaction was quenched with methanol and the system was concentrated and then purified by combiflash to give 100 mg of white solid compound 41a-3. MS m/z (ESI): 242 [M+H]⁺.

Step 3: To a solution of compound 41a-3 (60 mg, 0.249 mmol) in methanol (5 mL) was added sodium borohydride (5 mg, 0.124 mmol) under ice bath and the mixture was stirred at room temperature for 2 hours. LC-MS was used to trace the reaction until the reaction was complete. The system was quenched with water, extracted with ethyl acetate, and the organic layer was dried and concentrated to give compound 41a which was directly used in the next reaction. MS m/z (ESI): N/A.

Preparation of Intermediates 42a and 43a

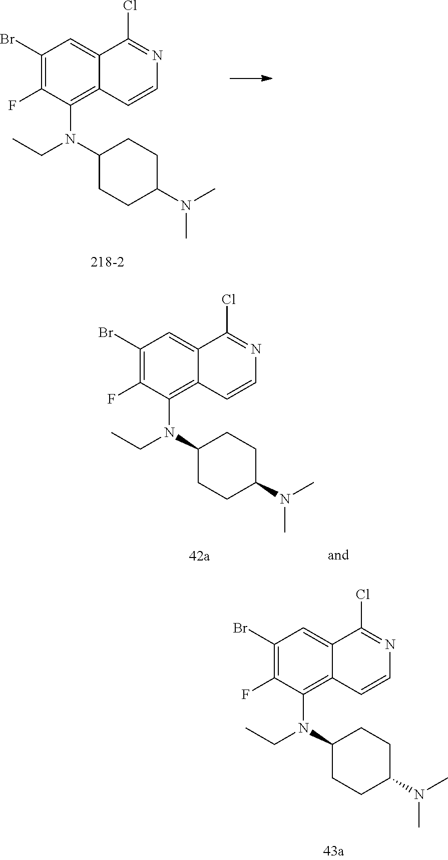 [SMILES: CCN(c1c(F)c(Br)cc2c(Cl)nccc12)C1CCC(N(C)C)CC1 ; CCN(c1c(F)c(Br)cc2c(Cl)nccc12)[C@H]1CC[C@@H](N(C)C)CC1 ; CCN(c1c(F)c(Br)cc2c(Cl)nccc12)[C@H]1CC[C@H](N(C)C)CC1]

Compounds 42a and 43a were obtained by chiral resolution. MS m/z (ESI): 428 [M+H]⁺.

Preparation of Intermediate 1b

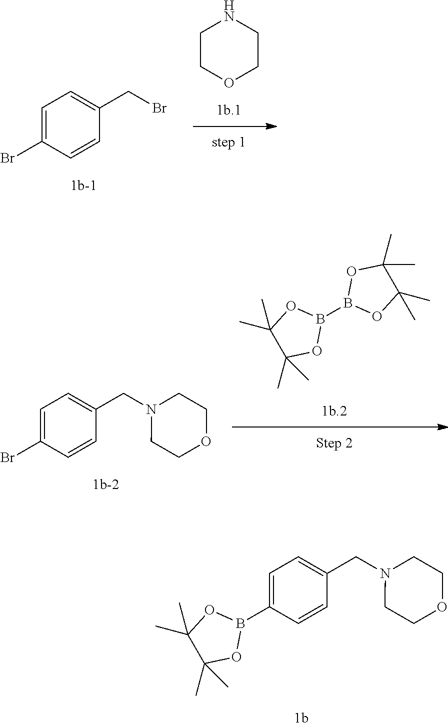

Step 1: To a solution of compound 1b-1 (1 g, 4 mmol) in acetonitrile (20 mL) was added compound 1b.1 (3.5 mL), and the mixture was heated to reflux under an argon atmosphere for 4 hours. LC-MS was used to trace the reaction until the reaction was complete. After cooling, the reaction solution was poured into 200 mL of water, extracted with ethyl acetate, combined and concentrated to give 1.1 g of crude product of compound 1b-2. MS m/z (ESI): 255 [M+H]⁺.

Step 2: To a solution of compound 1b-2 (1 g, 3.92 mmol) in DMF (20 mL) was added compound Pd(dppf)Cl₂ (144 mg, 0.19 mmol), compound 1b.2 (1.2 g, 3.33 mmol) and potassium acetate (570 mg, 5.8 mmol), the mixture was stirred under argon atmosphere at 80° C. overnight. The reaction solution was concentrated and purified by combiflash to give compound 1b (400 mg, 31%). MS m/z (ESI): 304 [M+H]⁺.

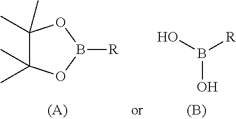

or boric acid or borate ester intermediates

The intermediate compounds are represented by formula (A) and (B), and the substituent R is shown in the following table.

General procedure: Compounds 2b to 25b were prepared from bromides as raw materials by method similar to that of intermediate 1b.

| Intermediates | | MS |
|---|---|---|
| No | R | [M + H]⁺ |
| 2b | 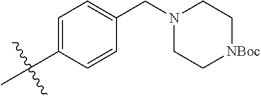 [SMILES: *c1ccc(CN2CCN(C(=O)OC(C)(C)C)CC2)cc1] | 403 |
| 3b | 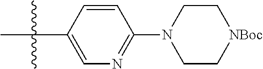 [SMILES: *c1ccc(N2CCN(C(=O)OC(C)(C)C)CC2)nc1] | 390 |
| 4b | 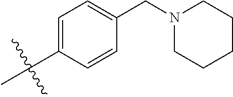 [SMILES: *c1ccc(CN2CCCCC2)cc1] | 302 |
| 5b | 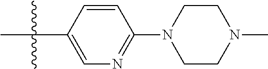 [SMILES: *c1ccc(N2CCN(C)CC2)nc1] | 304 |
| 6b | 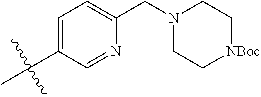 [SMILES: *c1ccc(CN2CCN(C(=O)OC(C)(C)C)CC2)nc1] | 403 |
| 7b | 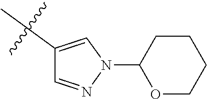 [SMILES: *c1cnn(C2CCCCO2)c1] | 279 |
| 8b | 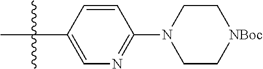 [SMILES: *c1ccc(N2CCN(C(=O)OC(C)(C)C)CC2)nc1] | 390 |
| 9b | 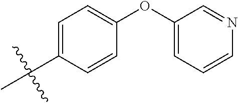 [SMILES: *c1ccc(Oc2cccnc2)cc1] | 298 |
| 10b | 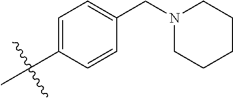 [SMILES: *c1ccc(CN2CCCCC2)cc1] | 302 |
| 11b | 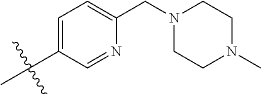 [SMILES: *c1ccc(CN2CCN(C)CC2)nc1] | 318 |
| 12b | 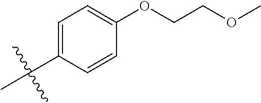 [SMILES: *c1ccc(OCCOC)cc1] | 279 |
| 13b | 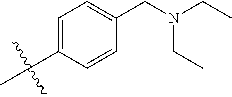 [SMILES: *c1ccc(CN(CC)CC)cc1] | 290 |
| 14b | 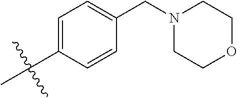 [SMILES: *c1ccc(CN2CCOCC2)cc1] | 304 |
| 15b | 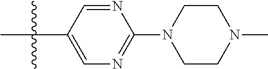 [SMILES: *c1cnc(N2CCN(C)CC2)nc1] | 305 |
| 16b | 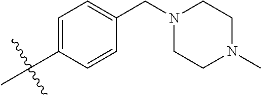 [SMILES: *c1ccc(CN2CCN(C)CC2)cc1] | 317 |
| 17b | 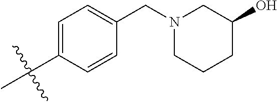 [SMILES: *c1ccc(CN2CCC[C@H](O)C2)cc1] | 318 |
| 18b | 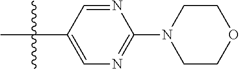 [SMILES: *c1cnc(N2CCOCC2)nc1] | 292 |
| 19b | 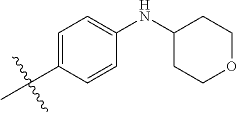 [SMILES: *c1ccc(NC2CCOCC2)cc1] | 304 |
| 20b | 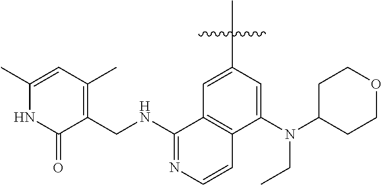 [SMILES: *c1cc(N(CC)C2CCOCC2)c2ccnc(NCc3c(C)cc(C)[nH]c3=O)c2c1] | 451 |
| 21b | 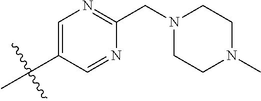 [SMILES: *c1cnc(CN2CCN(C)CC2)nc1] | 272 |
| 22b | 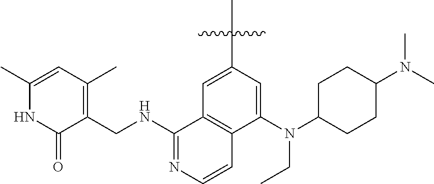 | 533 |
| 23b | 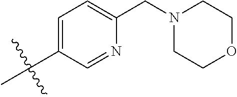 [SMILES: *c1ccc(CN2CCOCC2)nc1] | 305 |
| 24b | 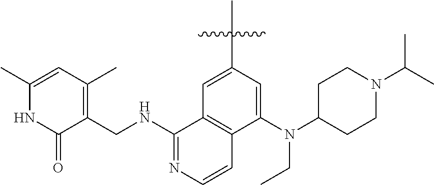 [SMILES: *c1cc(N(CC)C2CCN(C(C)C)CC2)c2ccnc(NCc3c(C)cc(C)[nH]c3=O)c2c1] | 574 |
| 25b | 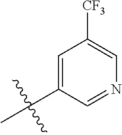 [SMILES: *c1cncc(C(F)(F)F)c1] | 274 |

Preparation of Intermediate 27b

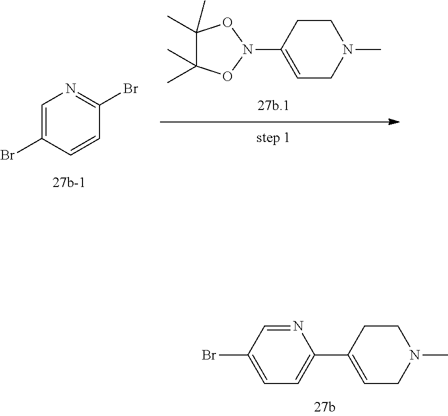

The preparation method is the same as that of compound 1b, except that compounds 1b-2 and 1b.2 in the preparation method of 1b are replaced by compounds 27b-1 and 27b.1. MS m/z (ESI): 255.1 [M+H]⁺.

Preparation of Intermediate 28b

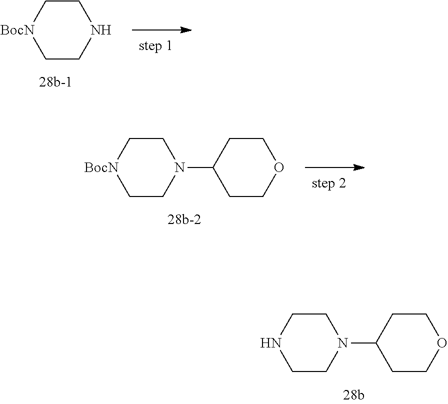 [SMILES: CC(C)(C)OC(=O)N1CCNCC1 ; CC(C)(C)OC(=O)N1CCN(C2CCOCC2)CC1 ; C1CN(C2CCOCC2)CCN1]

Step 1: The preparation method is the same as that of compound 11a-3, except that compound 11a-2 in the preparation method of 11a-3 is replaced by compound 28b-1. MS m/z (ESI): 271.2 [M+H]⁺.

Step 2: The preparation method is the same as that of compound z-9, except that compound 9-2 in the preparation method of z-9 is replaced by compound 28b-2. MS m/z (ESI): 171.2 [M+H]⁺.

Preparation of Intermediate 29b

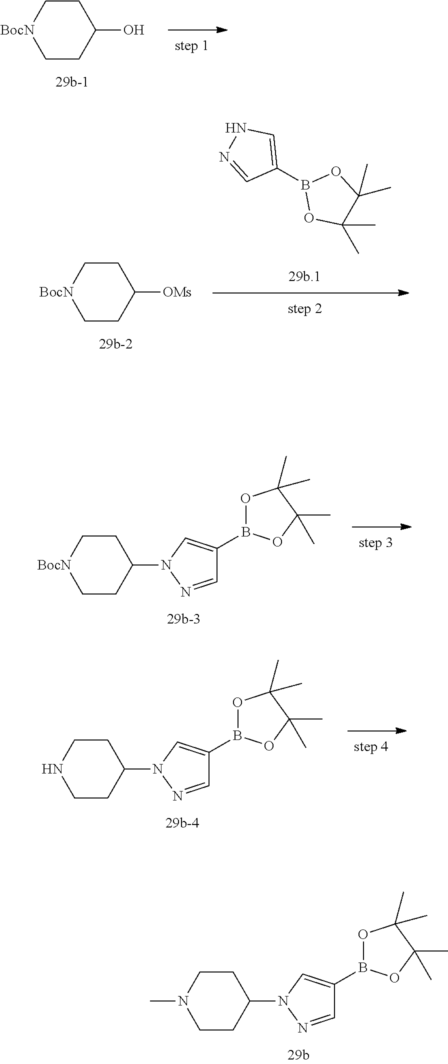

Step 1: To a solution of compound 29b-1 (500 mg, 2.48 mmol) in dichloromethane (30 mL) was added triethylamine (500 mg, 4.97 mmol), stirred under ice bath and MsCl (340 mg, 2.98 mmol) was added, the mixture was stirred at room temperature for 20 hours. The reaction solution was diluted with dichloromethane, washed with 1M hydrochloric acid and sodium bicarbonate solution, cooled to room temperature, and the organic layer was dried and concentrated to give 650 mg of white solid compound 29b-2. MS m/z (ESI): 224.1 [M+H]⁺.

Step 2: A mixture of compound 29b-2 (528 mg, 2 mmol), compound 29b.1 (427 mg, 2.2 mmol), cesium carbonate (847 mg, 2.6 mmol) and DMF (5 mL) was stirred under argon atmosphere at 100° C. overnight. The mixture was added sodium bicarbonate solution, concentrated and purified by combiflash to give 150 mg of compound 29b-3. MS m/z (ESI): 378 [M+H]⁺.

Step 3: The preparation method is the same as that of compound z-8, except that compound 8-1 in the preparation method of z-8 is replaced by compound 29b-3. MS m/z (ESI): 278 [M+H]⁺.

Step 4: The preparation method is the same as that of compound z-226, except that the compound z-201 in the preparation method of z-226 is replaced by compound 29b-4. MS m/z (ESI): 292 [M+H]⁺.

Preparation of Intermediate 30b

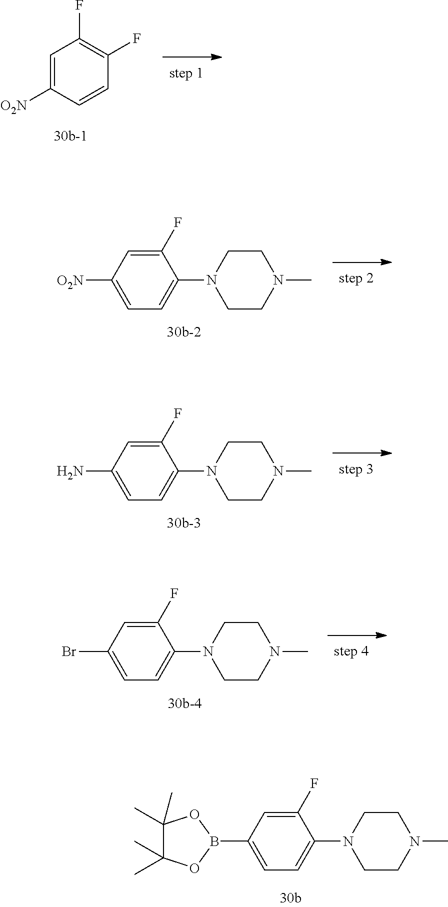

Step 1: The preparation method is the same as that of compound 9a, except that compound 9a-1 and 2-iodopropane in the preparation method of compound 9a are replaced by compound 30b-1 and N-methylpiperazine. MS m/z (ESI): 240 [M+H]⁺.

Step 2: The preparation method is the same as that of compound 1-2, except that compound 1-1 in the preparation method of 1-2 is replaced by compound 30b-2. MS m/z (ESI): 210 [M+H]⁺.

Step 3: To a solution of compound 30b-3 (700 mg, 3.35 mmol) in water (50 mL) was added 5 mL of hydrobromic acid and tert-butyl nitrite (517 mg, 5.02 mmol) under an ice bath, and the mixture was stirred for 30 minutes and then was added copper bromide (960 mg, 6.69 mmol) under an ice bath. The reaction was continued for 1 hour. LC-MS was used to trace the reaction until the reaction was complete. The reaction solution was poured into water, adjusted to pH 8 with saturated sodium bicarbonate solution, extracted with ethyl acetate, and the organic layer was dried, concentrated, and then purified by combiflash to give 410 mg of compound 30b-4. MS m/z (ESI): 275 [M+H]⁺.

Step 4: The preparation method is the same as that of compound 1b, except that compound 1b-2 in the preparation method of 1b is replaced by compound 30b-4. MS m/z (ESI): 321 [M+H]⁺.

Preparation of Intermediate 31b

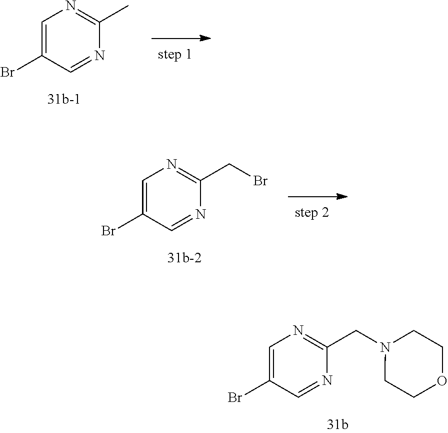

Step 1: To a solution of compound 31b-1 (500 mg, 2.9 mmol) in carbon tetrachloride (6 mL) was added NBS (540 mg, 3 mmol) and benzoyl peroxide (49 mg, 0.2 mmol) under argon atmosphere, the mixture was stirred at 80° C. for 36 hours. The reaction solution was concentrated, and purified by combiflash to give 160 mg of compound 31b-2. MS m/z (ESI): 275 [M+H]⁺.

Step 2: The preparation method is the same as that of compound 9a, except that compound 9a-1 and 2-iodopropane in the preparation method of compound 9a are replaced by compound 31b-1 and morpholine. MS m/z (ESI): 258[M+H]⁺.

Preparation of Intermediate 32b

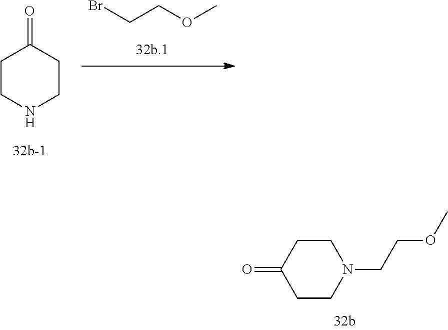

The preparation method is the same as that of compound 9a, except that compound 9a-1 and 2-iodopropane in the preparation method of compound 9a are replaced by compounds 32b-1 and 32b. 1. MS m/z (ESI): 158 [M+H]⁺.

Preparation of Intermediate 33b

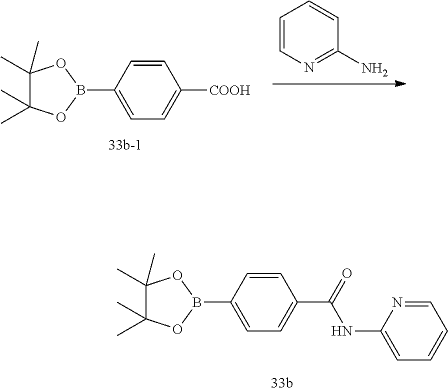 [SMILES: CC1(C)OB(c2ccc(C(=O)O)cc2)OC1(C)C ; Nc1ccccn1 ; CC1(C)OB(c2ccc(C(=O)Nc3ccccn3)cc2)OC1(C)C]

A solution of compound 33b-1 (1 g, 4 mmol), DIPEA (1 g, 8 mmol), HATU (2.45 g, 6.45 mmol) in DMF (25 mL) was stirred for 1 hour, then 2-aminopyridine (450 mg, 4.8 mmol) was added and stirred overnight. LC-MS was used to trace the reaction until the reaction was complete. The reaction solution was poured into water and extracted with ethyl acetate. The organic layer was dried and concentrated to give 1.1 g of compound 33b. MS m/z (ESI): 325 [M+H]⁺.

Preparation of Intermediate 34b

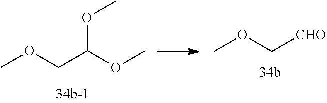

A solution of compound 34b-1 (3 g, 25 mmol) in hydrochloric acid (30 mL, 1 M) was stirred at room temperature overnight. The reaction solution was extracted with dichloromethane, and the organic layer was dried and concentrated to give 1.1 g of compound 34b which was used directly in the next reaction. MS m/z (ESI): N/A.

Preparation of Intermediate 35b

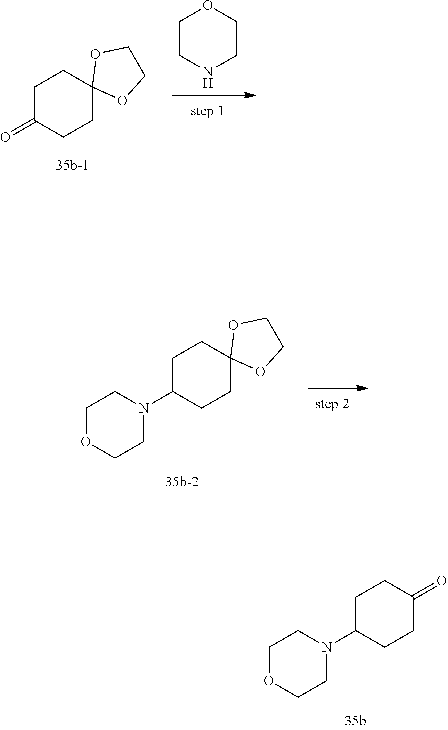

Step 1: To a solution of compound 35b-1 (7.8 g, 50 mmol) in dichloroethane (150 mL) was added morpholine (4.4 g, 50 mmol) and sodium triacetoxyborohydride (21 g, 100 mmol), and the mixture was stirred at room temperature overnight. Sodium hydroxide (20 mL, 10%) was added to the reaction solution, the organic layer was dried and concentrated to give 10 g of compound 35b-2. MS m/z (ESI): 228 [M+H]⁺.

Step 2: A solution of compound 35b-2 (10 g, 44 mmol) in hydrochloric acid (100 mL, 6 M) was stirred at 50° C. overnight. The reaction solution was cooled to room temperature, adjusted to pH 8 with potassium carbonate, extracted with ethyl acetate, and the organic layer was dried and concentrated to give 10 g of compound 35b which was used directly in the next reaction. MS m/z (ESI): 184 [M+H]⁺.

Preparation of Intermediate 36b

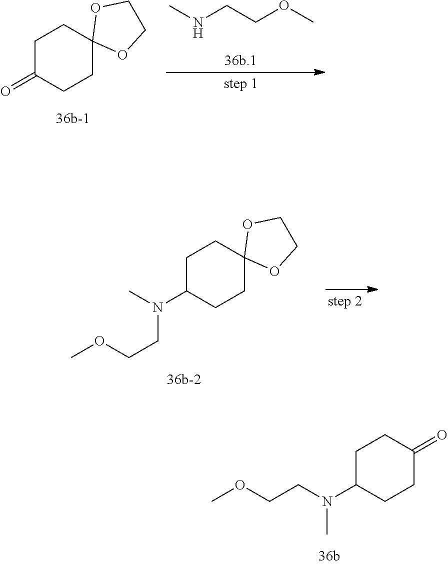

Step 1: The preparation method is the same as that of compound 35b-2, except that morpholine in the preparation method of 35b-2 is replaced by compound 36b.1. MS m/z (ESI): 230 [M+H]⁺.

Step 2: The preparation method is the same as that of compound 35b, except that compound 35b-2 in the preparation method of compound 35b is replaced by compound 36b-2. MS m/z (ESI): 186 [M+H]⁺.

Preparation of Intermediate 37b

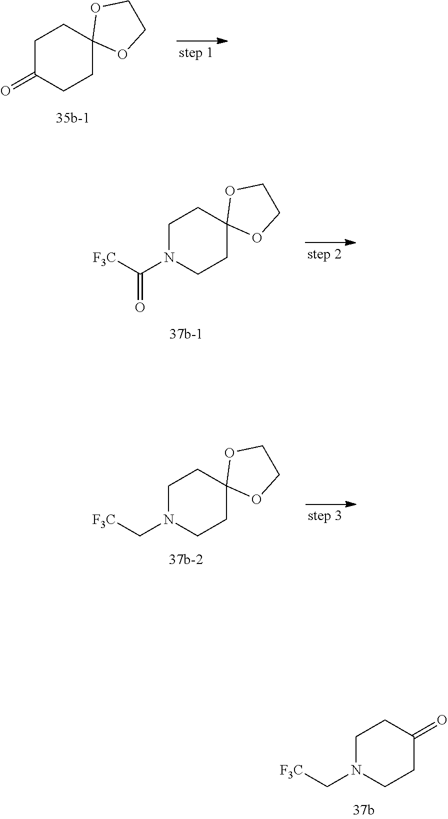 [SMILES: O=C1CCC2(CC1)OCCO2 ; O=C(N1CCC2(CC1)OCCO2)C(F)(F)F ; FC(F)(F)CN1CCC2(CC1)OCCO2 ; O=C1CCN(CC(F)(F)F)CC1]

Step 1: To a solution of compound 35b-1 (1 g, 6.98 mmol), triethylamine (1.41 g, 13.97 mmol), DMAP (128 mg, 1.05 mmol) in dichloroethane (30 mL) was added trifluoroacetic anhydride (2.2 g, 10.48 mmol) dropwise under an ice bath. The mixture was stirred at room temperature overnight. The reaction solution was poured into water and extracted with dichloromethane. The organic layer was washed with 1 M hydrochloric acid and water, dried and concentrated to give 1.3 g of compound 37b-1. MS m/z (ESI): 240 [M+H]⁺.

Step 2: To a solution of compound 37b-1 (1.6 g, 6.69 mmol) in THF (15 mL) was added dropwise borane-tetrahydrofuran (13.4 mL, 1 M) in an ice bath and the mixture was stirred at reflux overnight. The system was quenched with methanol and concentrated. The residue was dissolved in ethyl acetate and washed with saturated brine. The organic layer was dried and concentrated to give 1.2 g of compound 37b-2. MS m/z (ESI): 226 [M+H]⁺.

Step 3: The preparation method is the same as that of compound 35b, except that compound 35b-2 in the preparation method of compound 35b is replaced by compound 37b-2. MS m/z (ESI): 182 [M+H]⁺.

Preparation of Intermediate 38b

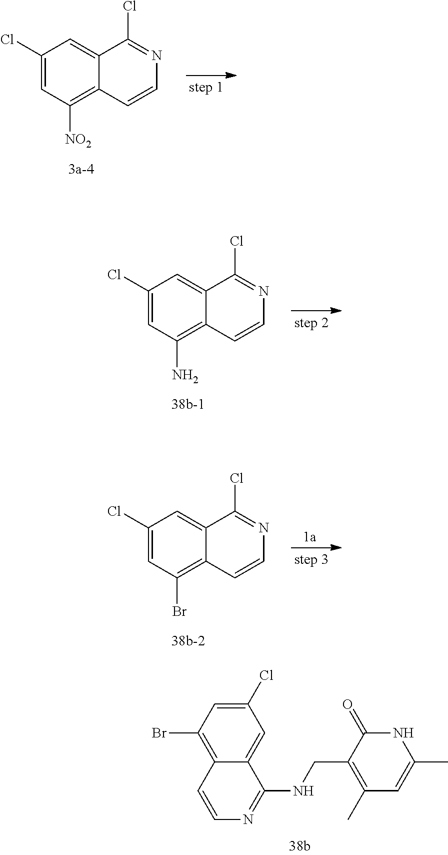 [SMILES: O=[N+]([O-])c1cc(Cl)cc2c(Cl)nccc12 ; Nc1cc(Cl)cc2c(Cl)nccc12 ; Clc1cc(Br)c2ccnc(Cl)c2c1 ; Cc1cc(C)c(CNc2nccc3c(Br)cc(Cl)cc23)c(=O)[nH]1]

Step 1: To a solution of compound 3a-4 (1 g, 4.35 mmol) in tetrahydrofuran (20 mL) and water (4 mL) was added ammonium chloride (1.06 g, 20 mmol) and iron powder (1.12 g, 20 mmol), and the mixture was stirred at 80° C. for 5 hours. LC-MS was used to trace the reaction until the reaction was complete. The reaction solution was conducted cold filtration, and the filtrate was concentrated to give compound 38b-1. MS m/z (ESI): 213 [M+H]⁺.

Step 2: To a solution of compound 38b-1 (220 mg, 1 mmol) in acetonitrile (30 mL) was added isoamyl nitrite (234 mg, 2 mmol), cuprous bromide (286 mg, 2 mmol) and copper bromide (446 mg, 2 mmol), the mixture was stirred at room temperature for 6 hours. LC-MS was used to trace the reaction until the reaction was complete. The reaction solution was concentrated, and purified by combiflash to give compound 38b-2. MS m/z (ESI): 275.9 [M+H]⁺.

Step 3: The preparation method is the same as that of compound 2a, except that compound 2a-4 in the preparation method of 2a is replaced by compound 38b-2. MS m/z (ESI): 392 [M+H]⁺.

Preparation of Intermediate 39b

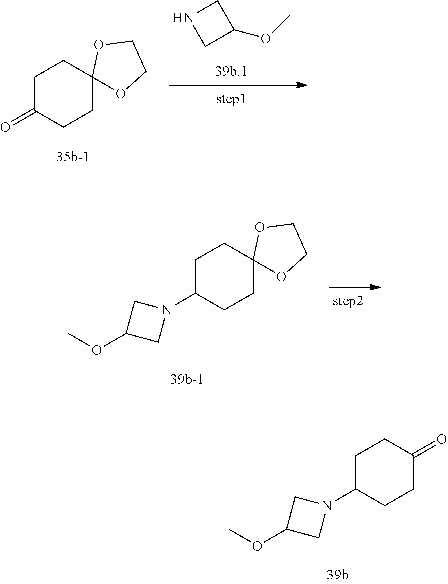

Step 1: The preparation method is the same as that of compound 35b-2, except that morpholine in the preparation method of 35b-2 is replaced by compound 39b.1. MS m/z (ESI): 228 [M+H]⁺.

Step 2: The preparation method is the same as that of compound 35b, except that compound 35b-2 in the preparation method of 35b is replaced by compound 39b-1. MS m/z (ESI): 184 [M+H]⁺.

Preparation of Intermediate 40b

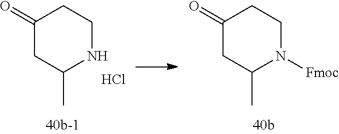

To a mixture of compound 40b-1 (450 mg, 3 mmol), sodium carbonate (636 mg, 6 mmol), 20 mL of 1,4-dioxane and 5 mL of water was added Fmoc-Cl (813 mg, 3.15 mmol) under an ice bath, and the mixture was stirred at room temperature for 16 hours. LC-MS was used to trace the reaction until the reaction was complete. The reaction solution was extracted with dichloromethane, and the organic layer was dried and concentrated, and then purified by combiflash to give compound 40b. MS m/z (ESI): 336 [M+H]⁺.

Preparation of Intermediate 41b

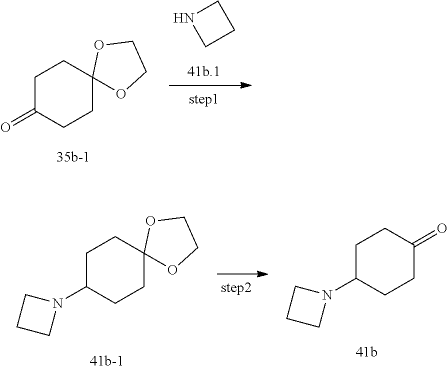

Step 1: The preparation method is the same as that of compound 35b-2, except that morpholine in the preparation method of 35b-2 is replaced by compound 41b.1. MS m/z (ESI): 197[M+H]⁺.

Step 2: The preparation method is the same as that of compound 35b, except that compound 35b-2 in the preparation method of 35b is replaced by compound 41b-1. MS m/z (ESI): 154 [M+H]⁺.

Preparation of Intermediate 42b

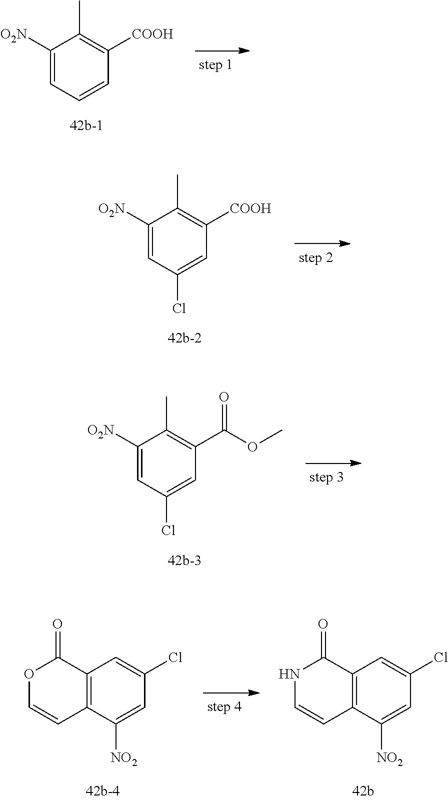

Step 1: A solution of compound 42b-1 (20 g, 110.4 mmol) and DCDMH (21.8 g, 110.4 mmol) in concentrated sulfuric acid (150 mL) was heated to 100° C. and stirred overnight. The reaction solution was cooled and poured into ice water. The solid was precipitated and filtered. The filter cake was dried under reduced pressure to give 32 g of solid compound 42b-2. MS m/z (ESI): N/A.

Step 2: To a solution of compound 42b-2 (32 g, 148.4 mmol) in methanol (150 mL) was added concentrated sulfuric acid (10 mL) dropwise and the mixture was heated to 90° C. and stirred overnight. LC-MS was used to trace the reaction until reaction was complete. 5 g of potassium carbonate in water (10 mL) was slowly added to the reaction solution. The reaction solution was then concentrated, and extracted with ethyl acetate/water. The organic layer was dried and concentrated to give 25 g of compound 42b-3. MS m/z (ESI): 230.6 [M+H]⁺.

Step 3: The preparation method is the same as that of compound 2a-2, except that compound 2a-1 in the preparation method of 2a-2 is replaced by compound 42b-3. MS m/z (ESI): 226.7 [M+H]⁺.

Step 4: The preparation method is the same as that of compound 2a-3, except that compound 2a-2 in the preparation method of 2a-3 is replaced by compound 42b-4. MS m/z (ESI): 224.9 [M+H]⁺.

Preparation of Intermediate 43b

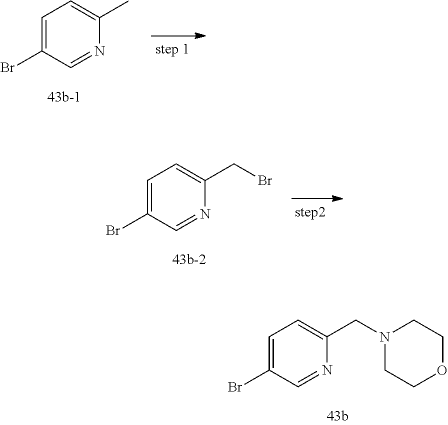

Step 1: The preparation method is the same as that of compound 31b-2, except that compound 31b-1 in the preparation method of 31b-2 is replaced by compound 43b-1. MS m/z (ESI): 249 [M+H]⁺.

Step 2: The preparation method is the same as that of compound 9a, except that compound 9a-1 and 2-iodopropane in the preparation method of compound 9a are replaced by compound 43b-2 and morpholine. MS m/z (ESI): 259 [M+H]⁺.

Preparation of Intermediate 44b

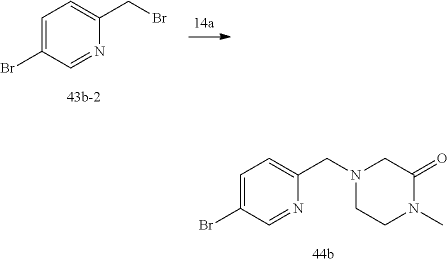 [SMILES: BrCc1ccc(Br)cn1 ; CN1CCN(Cc2ccc(Br)cn2)CC1=O]

The preparation method is the same as that of compound 9a, except that compound 9a-1 and 2-iodopropane in the preparation method of compound 9a are replaced by compound 43b-2 and 14a. MS m/z (ESI): 286 [M+H]⁺.

Preparation of Intermediate 45b

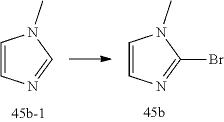

The preparation method is the same as that of compound 31b-2, except that compound 9a-1 in the preparation method of 31b-2 is replaced by compound 45b-1. MS m/z (ESI): 161 [M+H]⁺.

Preparation of Intermediate 46b

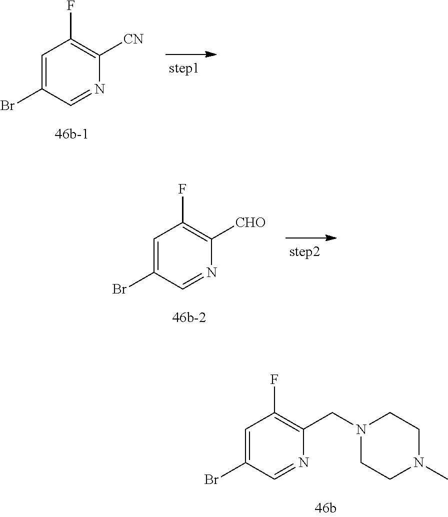 [SMILES: N#Cc1ncc(Br)cc1F ; O=Cc1ncc(Br)cc1F ; CN1CCN(Cc2ncc(Br)cc2F)CC1]

Step 1: To a solution of compound 46b-1 (500 mg, 2.49 mmol) in tetrahydrofuran (10 mL) was added diisobutylaluminium hydride (531 mg, 3.73 mmol) at −78° C. under an argon atmosphere, and the mixture was stirred at the same temperature for 3 hours. LC-MS was used to trace the reaction until the reaction was complete. The reaction solution was poured into ice water and extracted with ethyl acetate. The organic layer was dried and concentrated, and purified by combiflash to give 210 mg of compound 46b-2. MS m/z (ESI): 204 [M+H]⁺.

Step 2: To a solution of compound 46b-2 (500 mg, 2.4 mmol) in dichloromethane (10 mL) was added N-methylpiperazine (430 mg, 4.9 mmol) and sodium triacetoxyborohydride (1.56 g, 7.3 mmol) under argon atmosphere, and the mixture was stirred at room temperature for 16 hours. LC-MS was used to trace the reaction until the reaction was complete. To the reaction solution was added saturated sodium bicarbonate solution, the organic layer was separated, dried and concentrated, and purified by combiflash to give 210 mg of compound 46b. MS m/z (ESI): 288 [M+H]⁺.

Preparation of Intermediate 47b

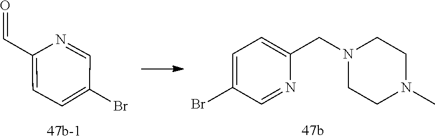

The preparation method is the same as that of compound 46b, except that compound 46b-2 in the preparation method of 46b is replaced by compound 47b-1. MS m/z (ESI): 270.1 [M+H]⁺.

Preparation of Intermediate 48b

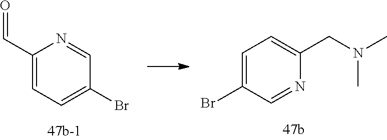

The preparation method is the same as that of compound 46b, except that compound 46b-2 and N-methylpiperazine in the preparation method of compound 46b are replaced by compound 47b-1 and dimethylamine. MS m/z (ESI): 215 [M+H]⁺.

Preparation of Intermediate 49b

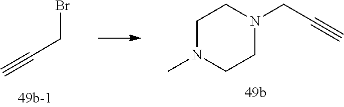

The preparation method is the same as that of compound 9a, except that compound 9a-1 and 2-iodopropane in the preparation method of compound 9a are replaced by compound 49b-1 and N-methylpiperazine. MS m/z (ESI): 139 [M+H]⁺.

Preparation of Intermediate 50b

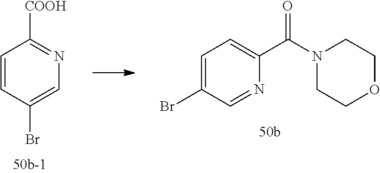 [SMILES: O=C(O)c1ccc(Br)cn1 ; O=C(c1ccc(Br)cn1)N1CCOCC1]

A solution of compound 50b-1 (1 g, 5 mmol) in dichlorosulfoxide (10 mL, 138 mmol) was stirred at 90° C. for 2 hours. The reaction solution was concentrated under reduced pressure to remove the solvent. To a solution of residue in triethylamine (1.52 g, 1.5 mmol) and dichloromethane (20 mL) was added morpholine (523 mg, 6 mmol) dropwise under an ice bath. The mixture was stirred at room temperature overnight. The reaction solution was extracted with dichloromethane/water system and the organic layer was dried and concentrated to give 1.34 g of compound 50b. MS m/z (ESI): 271[M+H]⁺.

Preparation of Intermediate 51b

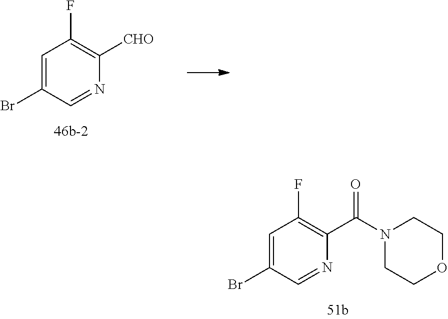 [SMILES: O=Cc1ncc(Br)cc1F ; O=C(c1ncc(Br)cc1F)N1CCOCC1]

The preparation method is the same as that of compound 46b, except that N-methylpiperazine in the preparation method of 46b is replaced by morpholine. MS m/z (ESI): 275[M+H]⁺.

Preparation of Intermediate 52b

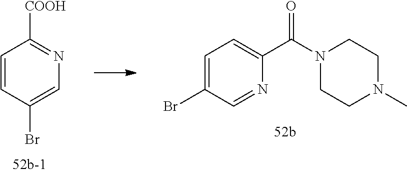 [SMILES: O=C(O)c1ccc(Br)cn1 ; CN1CCN(C(=O)c2ccc(Br)cn2)CC1]

The preparation method is the same as that of compound 50b, except that compound 50b-1 in the preparation method of 50b is replaced by N-methylpiperazine. MS m/z (ESI): 285[M+H]⁺.

Preparation of Intermediates 1c to 6c

General procedure: Compounds 1c to 6c are prepared by method similar to that of intermediate 6a, with isoquinoline compounds as raw materials. The structures thereof are as shown in formula (Ia), wherein when X is NH; Z₁ is N; Z₂, Z₃ are CH; R₃, R₅ and R₆ are hydrogen, R₀, R₁, R₂ and R₄ are the structures shown in the following table:

| | |
|---|---|
| | Ia |
| 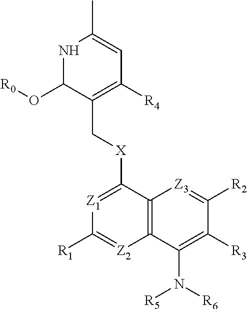 | |

| Intermediates | | | | | |
|---|---|---|---|---|---|
| No. | R₂ | R₄ | R₀ | R₁ | MS[M + H]⁺ |
| 1c | F | CH₃ | H | H | 313 |
| 2c | Cl | CH₃O | H | H | 345 |
| 3c | Cl | CH₃ | H | H | 329 |
| 4c | Cl | CH₃CH₂ | Bn | H | 435 |
| 5c | Cl | CH₃ | Bn | H | 420 |
| 6c | Br | CH₃ | H | CH₃O | 403 |

Preparation of Intermediates 1d to 5d

General procedure: Compounds 1d to 5d are prepared by a method similar to that of compound z-7, with isoquinoline compounds as raw materials. The structures thereof are as shown in formula (I), wherein when X is NH; Z₁ is N; Z₂, Z₃ are CH; R₁, R₃ are hydrogen; R₂, R₄, NR₅R₆ are the structures shown in the following table:

| Intermediates | | | | |
|---|---|---|---|---|
| No. | NR₅R₆ | R2 | R4 | MS[M + H]⁺ |
| 1d | 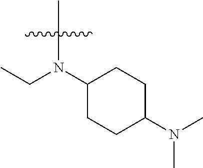 [SMILES: *N(CC)C1CCC(N(C)C)CC1] | 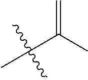 [SMILES: *C(=C)C] | CH₃ | 488 |
| 2d | 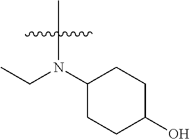 [SMILES: *N(CC)C1CCC(O)CC1] | 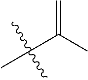 [SMILES: *C(=C)C] | CH₃ | 461 |
| 3d | 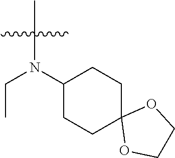 [SMILES: *N(CC)C1CCC2(CC1)OCCO2] | 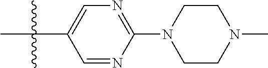 [SMILES: *c1cnc(N2CCN(C)CC2)nc1] | CH₃ | 639 |
| 4d | 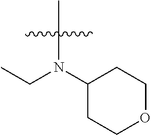 [SMILES: *N(CC)C1CCOCC1] | 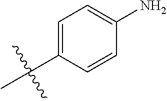 [SMILES: *c1ccc(N)cc1] | CH₃ | 498 |
| 5d | 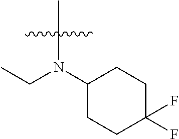 [SMILES: *N(CC)C1CCC(F)(F)CC1] | Br | CH₃ | 519 |

### Example 1: Preparation of 3-((5-(ethyl (tetrahydro-2H-pyran-4-yl) amino)-7-(4-(morpholino) phenyl) isoquinolin-1-ylamino) methyl)-4,6-dimethylpyridine-2(1H)-one (compound z-1)

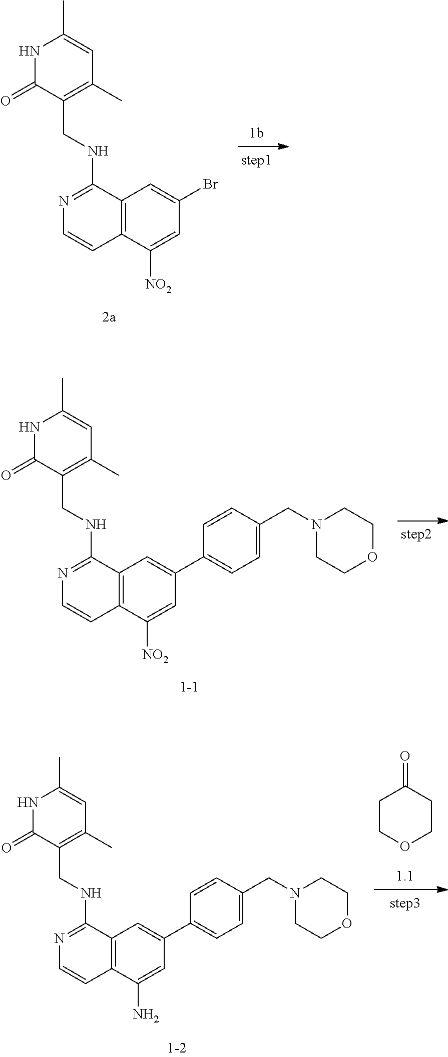 [SMILES: Cc1cc(C)c(CNc2nccc3c([N+](=O)[O-])cc(Br)cc23)c(=O)[nH]1 ; Cc1cc(C)c(CNc2nccc3c([N+](=O)[O-])cc(-c4ccc(CN5CCOCC5)cc4)cc23)c(=O)[nH]1 ; Cc1cc(C)c(CNc2nccc3c(N)cc(-c4ccc(CN5CCOCC5)cc4)cc23)c(=O)[nH]1 ; O=C1CCOCC1]

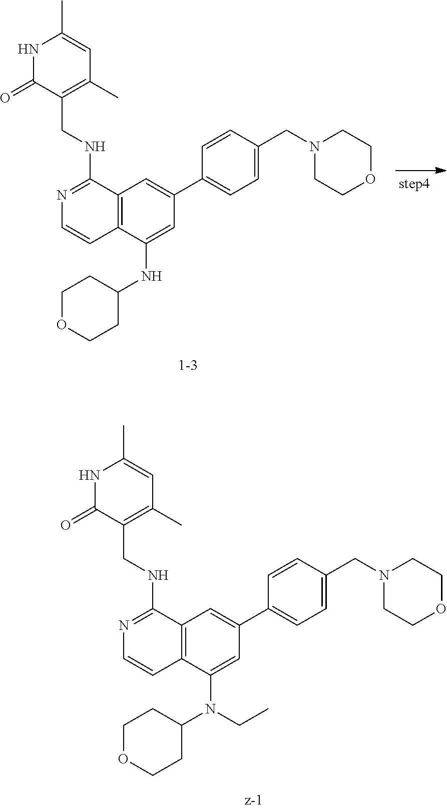 [SMILES: Cc1cc(C)c(CNc2nccc3c(NC4CCOCC4)cc(-c4ccc(CN5CCOCC5)cc4)cc23)c(=O)[nH]1 ; CCN(c1cc(-c2ccc(CN3CCOCC3)cc2)cc2c(NCc3c(C)cc(C)[nH]c3=O)nccc12)C1CCOCC1]

Step 1: A mixture of compound 2a (200 mg, 0.496 mmol), compound 1b (225 mg, 0.744 mmol), Pd(dppf)Cl₂ (1.8 mg, 0.025 mmol), sodium carbonate (105 mg, 0.992 mmol), 1,4-dioxane (4 mL) and 0.4 mL of water was stirred under argon atmosphere at 100° C. for 30 minutes. LC-MS was used to trace the reaction until the reaction was complete. The reaction solution was cooled and poured into water, and filtered to obtain 200 mg of a red solid compound 1-1. MS m/z (ESI): 500.3[M+H]⁺.

Step 2: To a solution of compound 1-1 (200 mg, 0.4 mmol) in methanol (20 mL) was added palladium on carbon (20 mg) under the protection of nitrogen and the mixture was stirred overnight under hydrogen atmosphere at room temperature. LC-MS was used to trace the reaction until the reaction was complete. The reaction solution was filtered, and the filtrate was concentrated to give 150 mg of crude compound 1-2 as an oil. MS m/z (ESI): N/A.

Step 3: A solution of compound 1-2 (130 mg, 0.277 mmol), compound 1.1 (138 mg, 1.38 mmol) and 0.5 mL acetic acid in dichloromethane (20 mL) was stirred at room temperature for 30 minutes, triacetoxy borohydride (586 mg, 2.77 mmol) was added in batches under an ice bath, and the mixture was stirred at room temperature for 7 hours. LC-MS was used to trace the reaction until the reaction was complete. The reaction solution was poured into water, adjusted to pH 8 with sodium bicarbonate solution, extracted with dichloromethane, dried and concentrated to give 200 mg of oily compound 1-3. MS m/z (ESI): 554.1 [M+H]⁺.

Step 4: A solution of compound 1-3 (200 mg, 0.361 mmol), acetaldehyde (159 mg, 3.61 mmol) and 0.5 mL acetic acid in dichloromethane (20 mL) was stirred under an ice bath for 30 minutes, sodium triacetoxyborohydride (766 mg, 3.61 mmol) was added in batches, stirred overnight at room temperature. LC-MS was used to trace the reaction until the reaction was complete. The reaction solution was poured into water, adjusted to pH 8 with sodium bicarbonate solution, extracted with dichloromethane, dried, concentrated and purified by Prep-HPLC to give compound z-1 (3 mg, 1.43%) as a white solid. MS m/z (ESI): 582.4[M+H]⁺. ¹H NMR (500 MHz, DMSO) δ 8.39 (s, 1H), 8.22 (s, 1H), 7.89 (d, 1H), 7.75 (d, 2H), 7.64 (s, 1H), 7.42 (d, 2H), 7.37 (t, 1H), 7.17 (d, 1H), 5.90 (s, 1H), 4.50 (d, 2H), 3.86-3.82 (m, 2H), 3.60-3.68 (m, 4H), 3.52 (s, 2H), 3.22-3.18 (m, 5H), 2.39-2.36 (m, 4H), 2.22 (s, 3H), 2.13 (s, 3H), 1.76-1.70 (m, 2H), 1.64-1.56 (m, 2H), 0.88 (t, 3H).

### Example 2: Preparation of 3-((7-bromo-5-(tetrahydro-2H-pyran-4-ylamino) isoquinolin-1-ylamino) methyl)-4,6-dimethyl-2 (1H)-one (compound z-2)

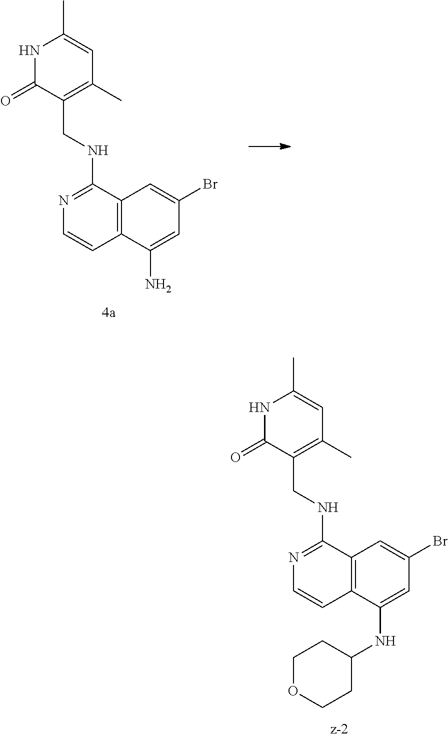 [SMILES: Cc1cc(C)c(CNc2nccc3c(N)cc(Br)cc23)c(=O)[nH]1 ; Cc1cc(C)c(CNc2nccc3c(NC4CCOCC4)cc(Br)cc23)c(=O)[nH]1]

The preparation method was the same as that of compound z-1, except that compound 1-3 and acetaldehyde in the preparation method of z-1 were replaced by compound 4a and tetrahydropyrone. After purified by Prep-HPLC, a white solid compound z-2 (57 mg, 31%) was obtained. MS m/z (ESI): 457.2 [M+H]⁺.

### Example 3: Preparation of 3-((7-bromo-5-(ethyl (tetrahydro-2H-pyran-4-yl)amino) isoquinolin-1-ylamino) methyl)-4,6-dimethyl-2(1H)-one (compound z-3)

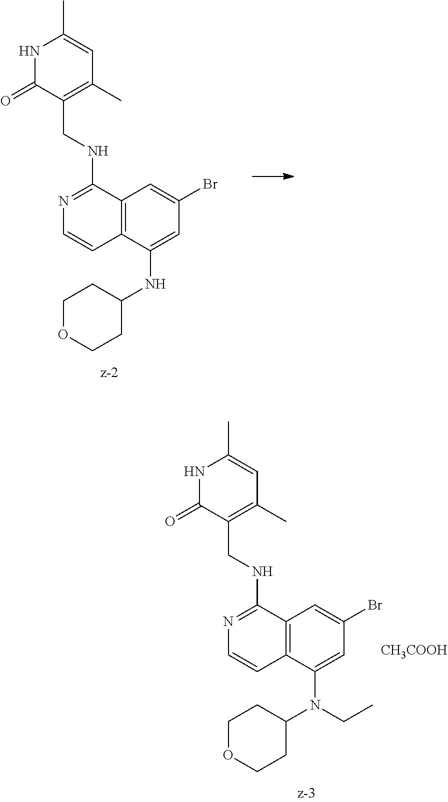

The preparation method was the same as that of compound z-1, except that compound 1-3 in the preparation method of z-1 was replaced by compound z-2. After purified by Prep-HPLC, a brown oil compound z-3 (7 mg, 4.4%) was obtained. MS m/z (ESI): 485.2 [M+H]⁺.

### Example 4: Preparation of 3-((7-chloro-5-(ethyl (tetrahydro-2H-pyran-4-yl) amino) isoquinolin-1-ylamino)methyl)-4,6-dimethyl-2(1H)-one (compound z-4)

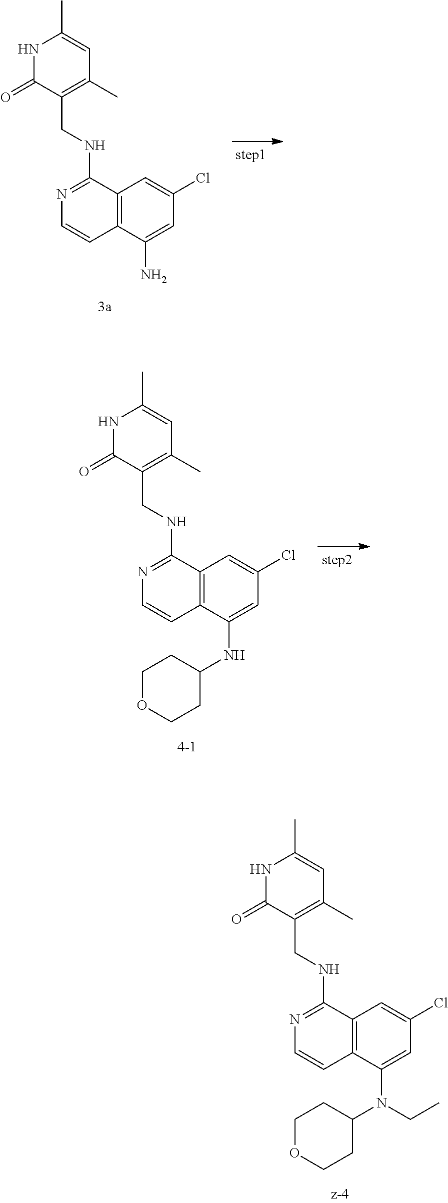 [SMILES: Cc1cc(C)c(CNc2nccc3c(N)cc(Cl)cc23)c(=O)[nH]1 ; Cc1cc(C)c(CNc2nccc3c(NC4CCOCC4)cc(Cl)cc23)c(=O)[nH]1 ; CCN(c1cc(Cl)cc2c(NCc3c(C)cc(C)[nH]c3=O)nccc12)C1CCOCC1]

Step 1: The preparation method was the same as that of compound z-1, except that compound 1-3 and acetaldehyde in the preparation method of z-1 were replaced by compound 3a and tetrahydropyrone. After purified by Prep-TLC, a yellow solid compound 4-1 (40 mg, 10.6%) was obtained. MS m/z (ESI): 413 [M+H]⁺.

Step 2: The preparation method was the same as that of compound z-1. The difference was that compound 1-3 in the preparation method of z-1 was replaced by compound 4-1, and toluene was used as a solvent. After purified by Prep-HPLC, a white solid compound z-4 (3.5 mg, 10%) was obtained. MS m/z (ESI): 441[M+H]⁺. ¹H NMR (500 MHz, DMSO-d₆): δ11.460 (s, 1H), 8.140 (s, 1H), 7.915 (d, 1H), 7.356 (s, 1H), 7.216 (s, 1H), 7.105 (d, 1H), 5.892 (s, 1H), 4.434 (d, 2H), 3.843 (d, 2H), 3.232-3.169 (m, 5H), 2.205 (s, 3H), 2.133 (s, 3H), 1.694-1.575 (m, 4H), 0.853 (t, 3H).

### Example 5: Preparation of 3-((5-(ethyl (tetrahydro-2H-pyran-4-yl) amino) isoquinolin-1-ylamino) methyl)-4,6-dimethyl-2 (1H)-one (compound z-5)

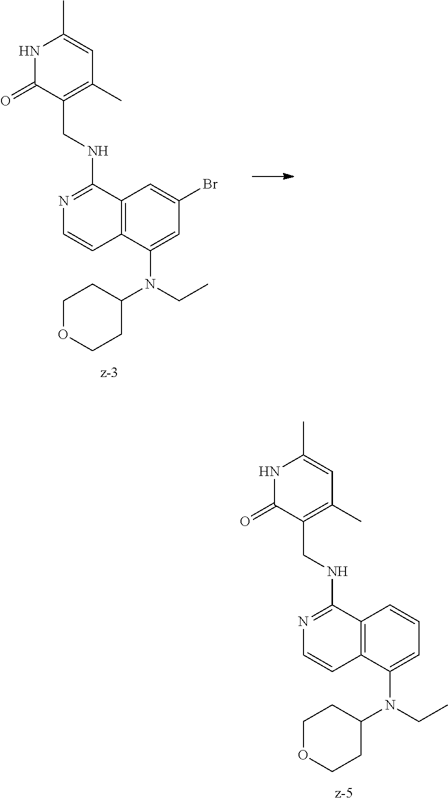 [SMILES: CCN(c1cc(Br)cc2c(NCc3c(C)cc(C)[nH]c3=O)nccc12)C1CCOCC1 ; CCN(c1cccc2c(NCc3c(C)cc(C)[nH]c3=O)nccc12)C1CCOCC1]

To a solution of compound z-3 (100 mg, 0.206 mmol) in methanol (20 mL) was added palladium on carbon (20 mg) under the protection of nitrogen and the mixture was stirred at room temperature overnight under hydrogen atmosphere. LC-MS was used to trace the reaction until the reaction was complete. The reaction solution was filtered and concentrated, and purified by Prep-HPLC to give compound z-5 (4 mg, 5%) as a white solid. MS m/z (ESI): 407.3 [M+H]⁺.

### Example 6: Preparation of 1-((4,6-dimethyl-2-oxo-1,2-dihydropyridin-3-yl) methyl amino)-5-(ethyl (tetrahydro-2H-pyran-4-yl)amino)isoquinoline-7-carbonitrile (compound z-6)

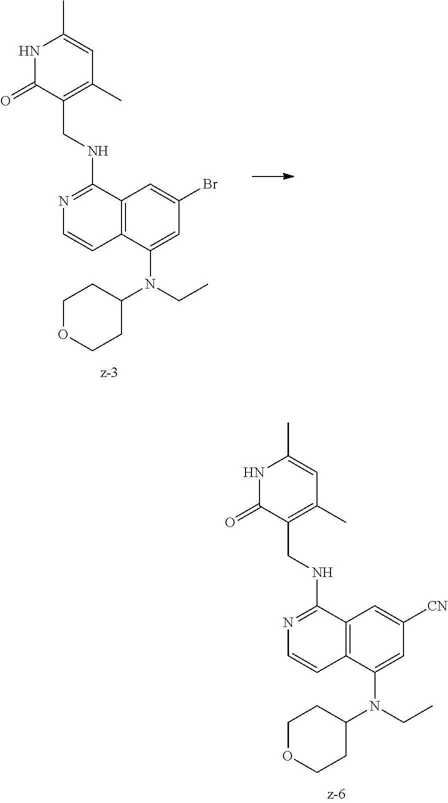 [SMILES: CCN(c1cc(Br)cc2c(NCc3c(C)cc(C)[nH]c3=O)nccc12)C1CCOCC1 ; CCN(c1cc(C#N)cc2c(NCc3c(C)cc(C)[nH]c3=O)nccc12)C1CCOCC1]

A solution of compound z-3 (170 mg, 0.35 mmol), zinc cyanide (100 mg, 0.84 mmol) and Tetrakis(triphenylphosphine)palladium (40 mg, 0.034 mmol) in DMF (6 mL) was stirred at 120° C. for 30 minutes under an argon atmosphere. LC-MS was used to trace the reaction until the reaction was complete. The reaction solution was filtered, then the filtrate was poured into water, extracted with ethyl acetate, concentrated, and purified by Prep-HPLC to give a white solid compound z-6 (11 mg, 7%). MS m/z (ESI): 432.3[M+H]⁺. ¹H NMR (500 MHz, DMSO) δ 11.39 (s, 1H), 8.56 (s, 1H), 7.98 (d, 1H), 7.56 (s, 1H), 7.39-7.35 (m, 1H), 7.07 (d, 1H), 5.83 (s, 1H), 4.38 (d, 2H), 3.79-3.75 (m, 2H), 3.18-3.07 (m, 5H), 2.13 (s, 3H), 2.06 (s, 3H), 1.64-1.58 (m, 2H), 1.55-1.47 (m, 2H), 0.76 (t, 3H).

### Example 7: Preparation of 3-((5-(ethyl (tetrahydro-2H-pyran-4-yl) amino)-7-(1-methyl-1H-pyrazol-4-yl) isoquinoline-1-yl amino) methyl)-4,6-dimethylpyridin-2(1H)-one (compound z-7)

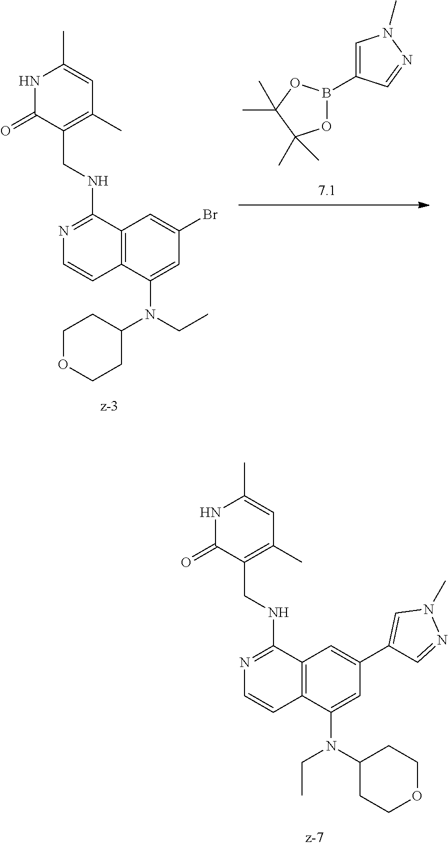 [SMILES: CCN(c1cc(Br)cc2c(NCc3c(C)cc(C)[nH]c3=O)nccc12)C1CCOCC1 ; Cn1cc(B2OC(C)(C)C(C)(C)O2)cn1 ; CCN(c1cc(-c2cnn(C)c2)cc2c(NCc3c(C)cc(C)[nH]c3=O)nccc12)C1CCOCC1]

A mixture of compound z-3 (70 mg, 0.144 mmol), compound 7.1 (60 mg, 0.288 mmol), Pd(dppf)Cl₂ (5 mg, 0.007 mmol), sodium carbonate (46 mg, 0.433 mmol), 1,4-dioxane (6 mL) and 0.6 mL of water was microwaved at 100° C. under argon atmosphere for 30 minutes. LC-MS was used to trace the reaction until the reaction was complete. The reaction solution was poured into water and extracted with dichloromethane, concentrated, and purified by Prep-HPLC to give compound z-7 (12 mg, 17.1%) as a white solid. MS m/z (ESI): 487.3 [M+H]⁺.

### Example 8: Preparation of 3-((5-(ethyl (tetrahydro-2H-pyran-4-yl) amino)-7-(4-(piperazin-1-yl methyl) phenyl) isoquinoline-1-yl amino) methyl)-4,6-dimethyl-2(1H)-one (compound z-8)

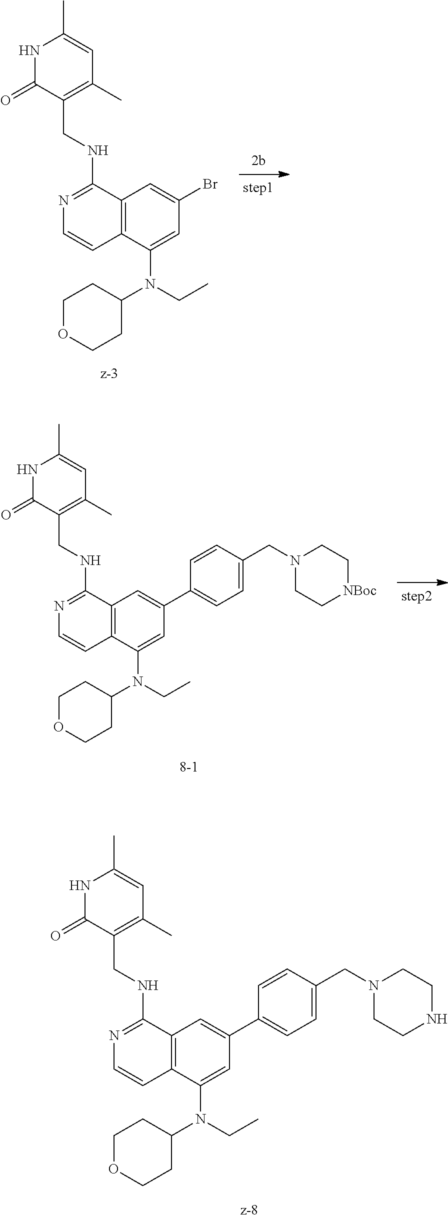 [SMILES: CCN(c1cc(Br)cc2c(NCc3c(C)cc(C)[nH]c3=O)nccc12)C1CCOCC1 ; CCN(c1cc(-c2ccc(CN3CCN(C(=O)OC(C)(C)C)CC3)cc2)cc2c(NCc3c(C)cc(C)[nH]c3=O)nccc12)C1CCOCC1 ; CCN(c1cc(-c2ccc(CN3CCNCC3)cc2)cc2c(NCc3c(C)cc(C)[nH]c3=O)nccc12)C1CCOCC1]

Step 1: The preparation method was the same as that of compound z-7, except that compound 7.1 in the preparation method of z-7 was replaced by compound 2b, and microwave reaction was performed for 15 minutes to obtain 82 mg of yellow oil compound 8-1. MS m/z (ESI): 681.4[M+H]⁺.

Step 2: To a solution of compound 8-1 (82 mg, 0.12 mmol) in dichloromethane (6 mL) was added 4 mL of trifluoroacetic acid under argon atmosphere and stirred at room temperature for 2 hours. LC-MS was used to trace the reaction until the reaction was complete. The reaction solution was concentrated and purified by Prep-HPLC to give compound z-8 (8 mg, 8.1%) as a white solid. MS m/z (ESI): N/A.

### Example 9: Preparation of 3-((7-chloro-5-(ethyl (octahydrocyclopenta[c]pyrrol-5-yl) amino) isoquinolin-1-yl amino) methyl)-4,6-dimethylpyridine-2-ol (compound z-9)

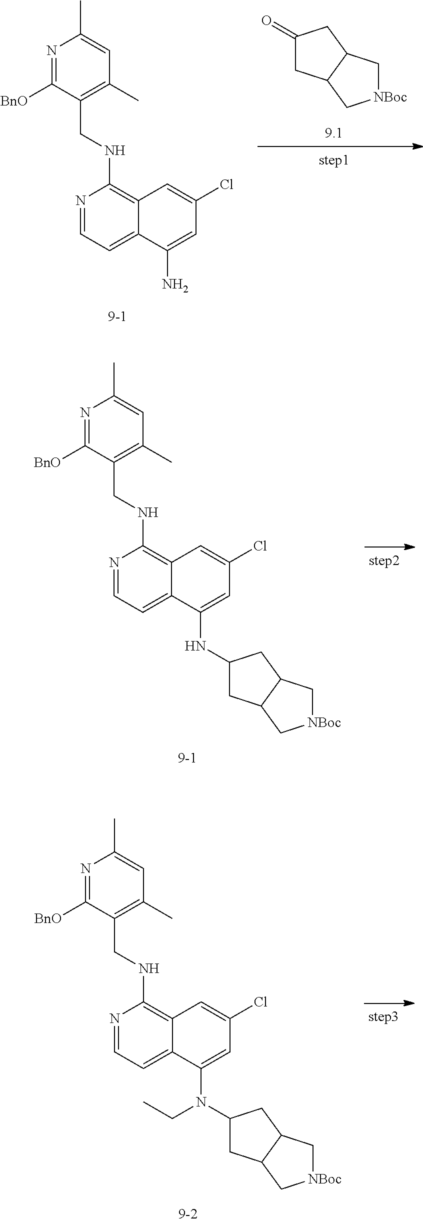 [SMILES: Cc1cc(C)c(CNc2nccc3c(N)cc(Cl)cc23)c(OCc2ccccc2)n1 ; CC(C)(C)OC(=O)N1CC2CC(=O)CC2C1 ; Cc1cc(C)c(CNc2nccc3c(NC4CC5CN(C(=O)OC(C)(C)C)CC5C4)cc(Cl)cc23)c(OCc2ccccc2)n1 ; CCN(c1cc(Cl)cc2c(NCc3c(C)cc(C)nc3OCc3ccccc3)nccc12)C1CC2CN(C(=O)OC(C)(C)C)CC2C1]

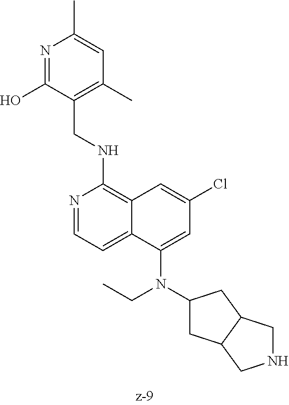 [SMILES: CCN(c1cc(Cl)cc2c(NCc3c(C)cc(C)nc3O)nccc12)C1CC2CNCC2C1]

Step 1: The preparation method was the same as that of compound 4-1, except that compound 3a and tetrahydropyrone in the preparation method of compound 4-1 were replaced by compounds 9-1 and 9.1. MS m/z (ESI): 628 [M+H]⁺.

Step 2: The preparation method was the same as that of compound z-4, except that compound 4-1 in the preparation method of compound z-4 was replaced by compound 9-1. MS m/z (ESI): 656 [M+H]⁺.

Step 3: To a solution of compound 9-2 (180 mg, 0.275 mmol) in dichloromethane (10 mL) was added 4M hydrochloric acid/1,4-dioxane (3 mL), and the mixture was stirred at room temperature for 2 hours. LC-MS was used to trace the reaction until the reaction was complete. After concentrated, the reaction solution was purified by Prep-HPLC to give a white solid compound z-9 (5.1 mg, 11%). MS m/z (ESI): 466[M+H]⁺. ¹H NMR (400 MHz, DMSO-d₆) δ 12.45 (s, 1H), 9.83 (s, 1H), 8.89-8.86 (m, 2H), 8.44 (d, 1H), 7.80-7.70 (m, 2H), 7.36 (d, 1H), 6.25 (s, 1H), 4.52 (d, 2H), 3.20-2.97 (m, 7H), 2.67-2.65 (m, 2H), 2.38 (s, 3H), 2.21 (s, 3H), 2.05-1.97 (m, 2H), 1.31-1.29 (m, 2H), 0.76 (t, 3H).

### Example 10: Preparation of 3-((5-(ethyl (tetrahydro-2H-pyran-4-yl) amino)-7-isopropyl isoquinolin-1-yl amino) methyl)-4,6-dimethyl-2(1H)-one (compound z-10)

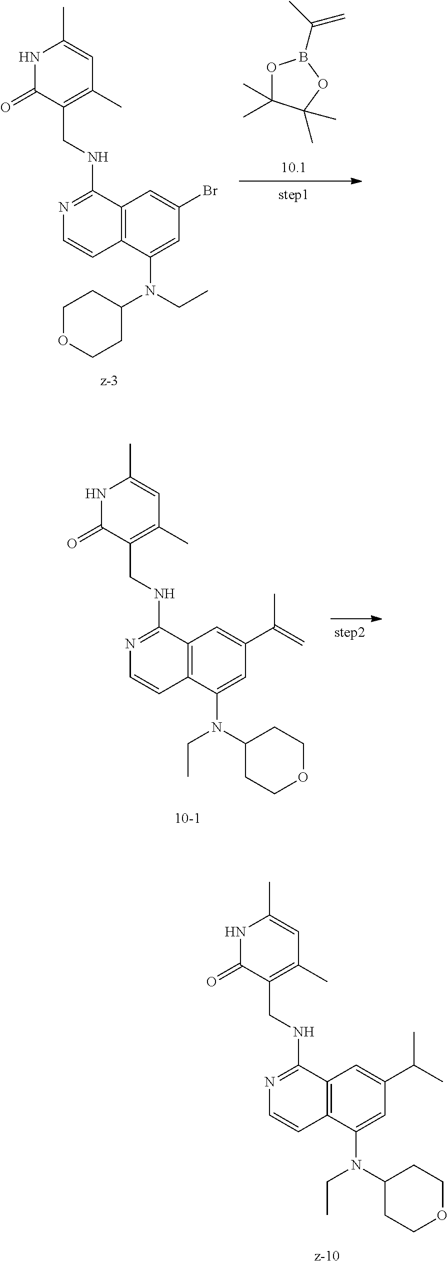 [SMILES: CCN(c1cc(Br)cc2c(NCc3c(C)cc(C)[nH]c3=O)nccc12)C1CCOCC1 ; C=C(C)B1OC(C)(C)C(C)(C)O1 ; C=C(C)c1cc(N(CC)C2CCOCC2)c2ccnc(NCc3c(C)cc(C)[nH]c3=O)c2c1 ; CCN(c1cc(C(C)C)cc2c(NCc3c(C)cc(C)[nH]c3=O)nccc12)C1CCOCC1]

Step 1: The preparation method was the same as that of compound z-7, except that compound 7.1 in the preparation method of z-7 was replaced by compound 10.1, and acetonitrile was used as the solvent. After combiflash purification, compound 10-1 (66 mg, 72%) was obtained. MS m/z (ESI): 447.4[M+H]⁺.

Step 2: A solution of compound 10-1 (66 mg, 0.188 mmol) and palladium on carbon (10 mg) in methanol (20 mL) was stirred at room temperature under hydrogen atmosphere overnight. LC-MS was used to trace the reaction until the reaction was complete. The reaction solution was filtered and the filtrate was concentrated and purified by Prep-HPLC to give compound z-10 as a white solid (19 mg, 29%). MS m/z (ESI): 449.3 [M+H]⁺.

### Example 11: Preparation of 3-((7-ethyl-5-(ethyl (tetrahydro-2H-pyran-4-yl) amino) isoquinolin-1-yl amino) methyl)-4,6-dimethyl-2(1H)-one (compound z-11)

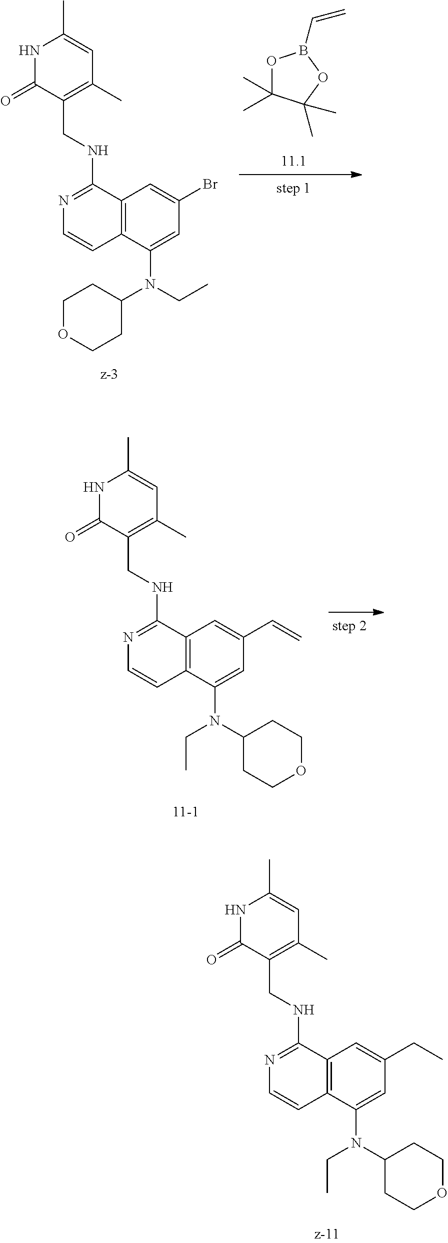 [SMILES: CCN(c1cc(Br)cc2c(NCc3c(C)cc(C)[nH]c3=O)nccc12)C1CCOCC1 ; C=CB1OC(C)(C)C(C)(C)O1 ; C=Cc1cc(N(CC)C2CCOCC2)c2ccnc(NCc3c(C)cc(C)[nH]c3=O)c2c1 ; CCc1cc(N(CC)C2CCOCC2)c2ccnc(NCc3c(C)cc(C)[nH]c3=O)c2c1]

Step 1: The preparation method was the same as that of compound z-7, except that compound 7.1 in the preparation method of compound z-7 was replaced by compound 11.1, and acetonitrile was used as the solvent. After combiflash purification, compound 11-1 (70 mg, 79%) was obtained. MS m/z (ESI): 433.4[M+H]⁺.

Step 2: The preparation method was the same as that of compound z-10, except that compound 10-1 in the preparation method of compound z-10 was replaced by compound 11-1. After purified by Prep-HPLC, compound z-11 (20 mg, 28.4%) was obtained as a white solid. MS m/z (ESI): 435.3 [M+H]⁺.

### Example 12: Preparation of 3-((5-(ethyl (tetrahydro-2H-pyran-4-yl) amino)-7-fluoro isoquinolin-1-yl amino) methyl)-4,6-dimethyl-2(1H)-one (compound z-12)

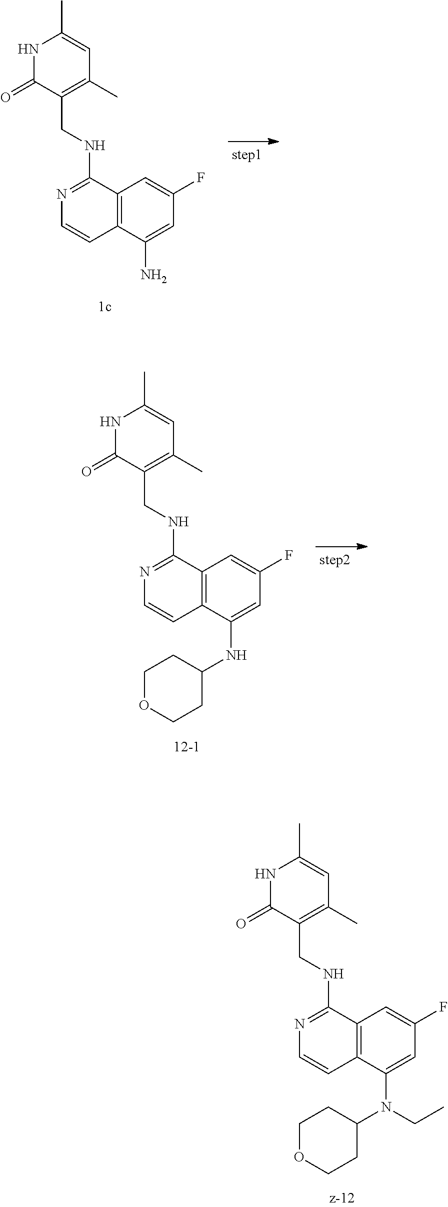 [SMILES: Cc1cc(C)c(CNc2nccc3c(N)cc(F)cc23)c(=O)[nH]1 ; Cc1cc(C)c(CNc2nccc3c(NC4CCOCC4)cc(F)cc23)c(=O)[nH]1 ; CCN(c1cc(F)cc2c(NCc3c(C)cc(C)[nH]c3=O)nccc12)C1CCOCC1]

Step 1: To a solution of compound 1c (100 mg, 0.32 mmol) in 1,4-dioxane (10 mL) was added tetrahydropyrone (70 mg, 0.64 mmol) and trifluoroacetic acid (110 mg, 0.96 mmol), the mixture was stirred at room temperature for 1 hour. After sodium triacetoxyborohydride (210 mg, 0.96 mmol) was added, the mixture was stirred at room temperature for 1 hour. The reaction solution was concentrated to give 80 mg of a yellow solid compound 12-1 which was used directly in the next step. MS m/z (ESI): 397 [M+H]⁺.

Step 2: The preparation method was the same as that of compound 12-1, except that compound 1c and tetrahydropyrone in the preparation method of compound 12-1 were replaced by compound 12-1 and acetaldehyde. After purified by Prep-HPLC, a white solid compound z-12 (0.85 mg, 1%) was obtained. MS m/z (ESI): 425.3 [M+H]⁺.

### Example 13: Preparation of 3-((5-(ethyl (piperidin-4-yl) amino)-7-(trifluoromethyl) isoquinolin-1-yl amino)methyl)-4,6-dimethylpyridine-2(1H)-one (compound z-13)

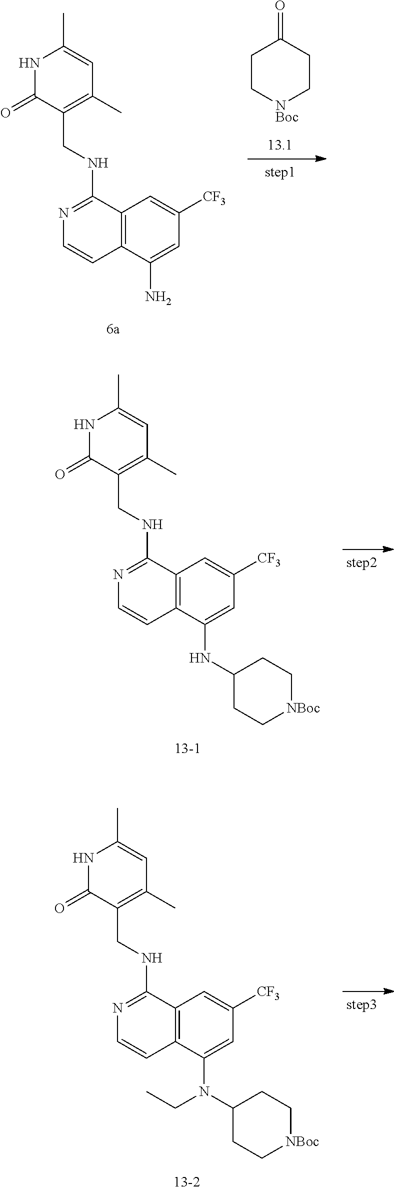 [SMILES: Cc1cc(C)c(CNc2nccc3c(N)cc(C(F)(F)F)cc23)c(=O)[nH]1 ; CC(C)(C)OC(=O)N1CCC(=O)CC1 ; Cc1cc(C)c(CNc2nccc3c(NC4CCN(C(=O)OC(C)(C)C)CC4)cc(C(F)(F)F)cc23)c(=O)[nH]1 ; CCN(c1cc(C(F)(F)F)cc2c(NCc3c(C)cc(C)[nH]c3=O)nccc12)C1CCN(C(=O)OC(C)(C)C)CC1]

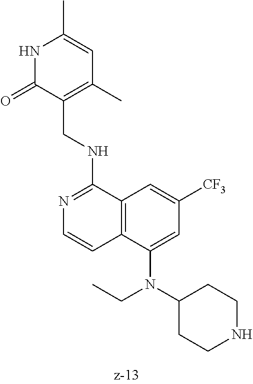 [SMILES: CCN(c1cc(C(F)(F)F)cc2c(NCc3c(C)cc(C)[nH]c3=O)nccc12)C1CCNCC1]

Step 1: The preparation method was the same as that of compound 4-1, except that compound 3a and tetrahydropyrone in the preparation method of compound 4-1 were replaced by compounds 6a and 13.1. MS m/z (ESI): 546 [M+H]⁺.

Step 2: The preparation method was the same as that of compound z-4, except that compound 4-1 in the preparation method of compound z-4 was replaced by compound 13-1. MS m/z (ESI): 574 [M+H]⁺.

Step 3: The preparation method was the same as that of compound z-8, except that compound 17-1 in the preparation method of compound z-8 was replaced by compound 13-2. After purified by HPTLC, compound z-13 (100 mg, 67.2%) was obtained as a white solid. MS m/z (ESI): 474 [M+H]⁺.

### Example 14: Preparation of 3-((5-(ethyl (tetrahydro-2H-pyran-4-yl)amino)-7-(1H-pyrazol-4-yl) isoquinolin-1-yl amino) methyl-4,6-dimethyl-2(1H)-one (compound z-14)

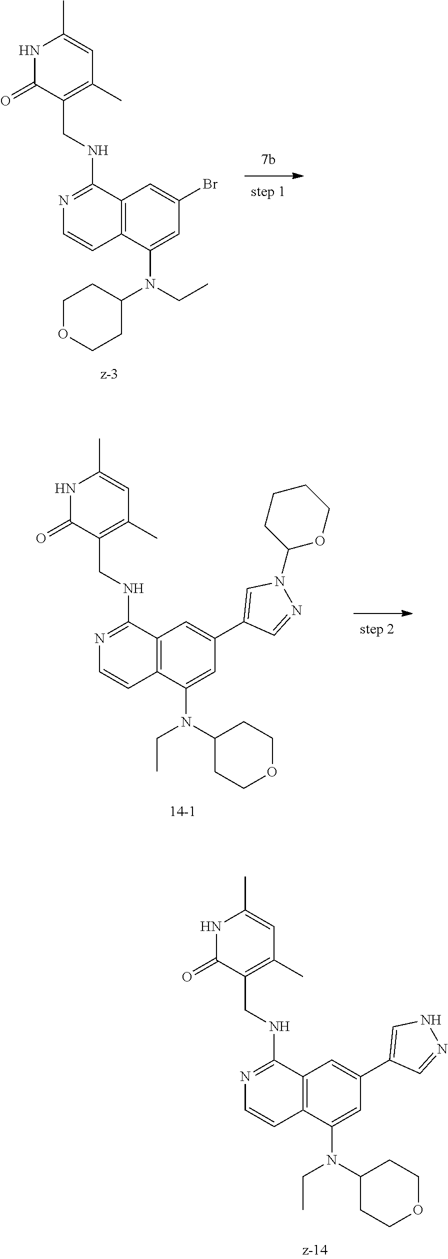 [SMILES: CCN(c1cc(Br)cc2c(NCc3c(C)cc(C)[nH]c3=O)nccc12)C1CCOCC1 ; CCN(c1cc(-c2cnn(C3CCCCO3)c2)cc2c(NCc3c(C)cc(C)[nH]c3=O)nccc12)C1CCOCC1 ; CCN(c1cc(-c2cn[nH]c2)cc2c(NCc3c(C)cc(C)[nH]c3=O)nccc12)C1CCOCC1]

Step 1: The preparation method was the same as that of compound z-7. The difference was that compound 7.1 in the preparation method of compound z-7 was replaced by compound 7b. After purified by combiflash, compound 14-1 (80 mg, 70%) was obtaine. MS m/z (ESI): 557.4[M+H]⁺.

Step 2: To compound 14-1 (80 mg, 0.144 mmol) was added hydrochloric acid/1,4-dioxane (4 mL) and dichloromethane (4 mL), and the mixture was stirred at room temperature for 2 hours. LC-MS was used to trace the reaction until the reaction was complete. The reaction solution was concentrated, the residue was washed with saturated sodium bicarbonate solution, dried and concentrated, and purified by Prep-HPLC to give compound z-14 (19 mg, 28%) as a white solid. MS m/z (ESI): 473.3 [M+H]⁺.

### Example 15: Preparation of 3-((7-bromo-5-((4-(dimethylamino) cyclohexyl) (ethyl) amino) isoquinolin-1-yl amino) methyl)-4,6-dimethyl-2(1H)-one (compound z-15)

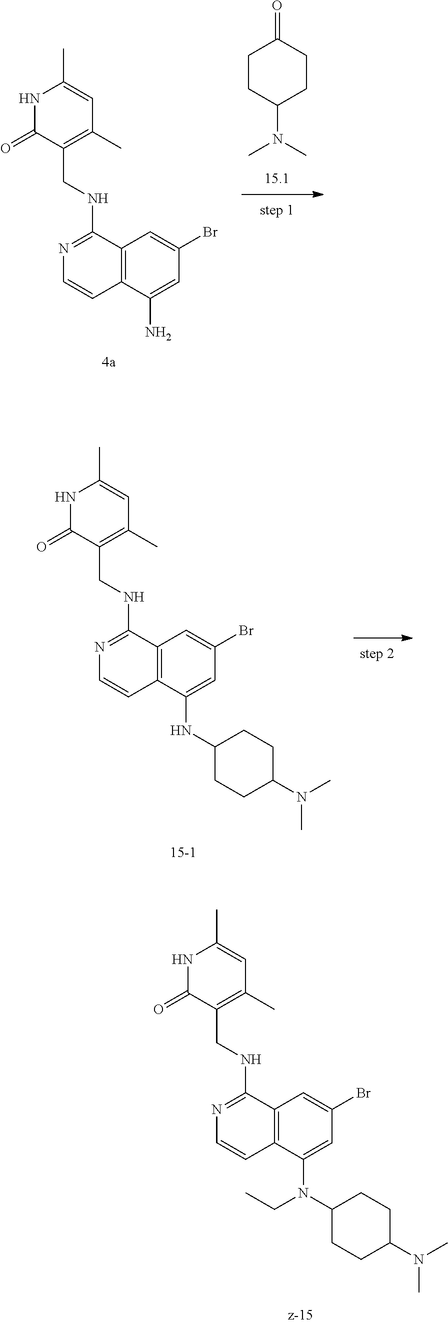

Step 1: To a solution of compound 4a (800 mg, 0.22 mmol) in 1,4-dioxane (20 mL) was added 15.1 (460 mg, 3.2 mmol) and trifluoroacetic acid (740 mg, 6.4 mmol). The mixture was stirred at room temperature for 1 hour, added sodium triacetoxyborohydride (1.34 g, 6.4 mmol), and then stirred at room temperature for 2 hours. LC-MS was used to trace the reaction until the reaction was complete. The reaction solution was adjusted to pH 8 with saturated sodium bicarbonate, extracted with dichloromethane and concentrated, and purified by silica gel column chromatography (dichloromethane/methanol=4:1) to give 150 mg of yellow solid compound 15-1. MS m/z (ESI): 498.0 [M+H]⁺.

Step 2: The preparation method was the same as that of compound 15-1, except that compounds 4a and 15.1 in the preparation method of compound 15-1 were replaced by compound 15-1 and compound acetaldehyde. After silica gel column chromatography (dichloromethane/methanol=4:1) purification, a yellow solid compound z-15 (30 mg, 19%) was obtained. MS m/z (ESI): 526.0 [M+H]⁺.

### Example 16: Preparation of 3-((5-(ethyl (tetrahydro-2H-pyran-4-yl) amino)-7-(trifluoromethyl) isoquinolin-1-ylamino)methyl)-4,6-dimethyl-2(1H)-one (compound z-16)

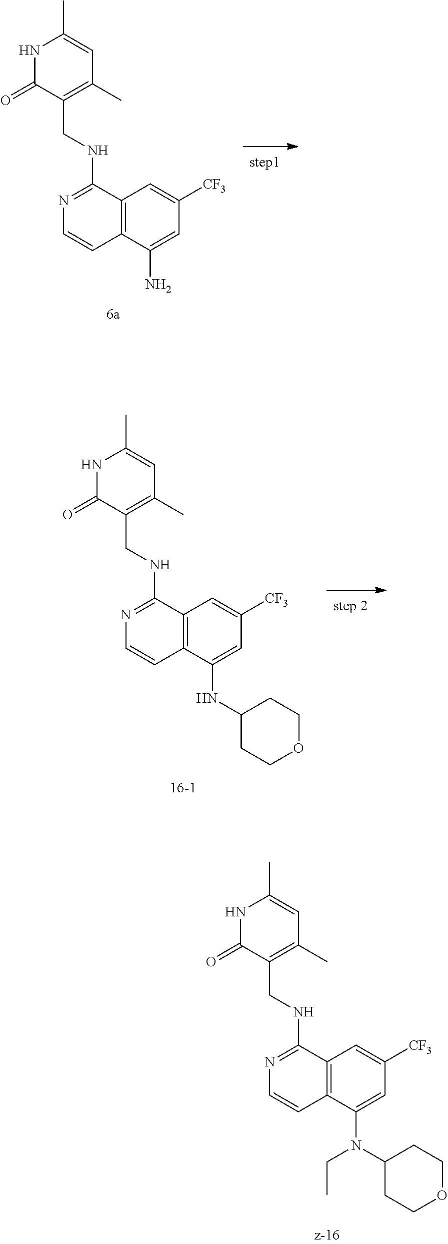 [SMILES: Cc1cc(C)c(CNc2nccc3c(N)cc(C(F)(F)F)cc23)c(=O)[nH]1 ; Cc1cc(C)c(CNc2nccc3c(NC4CCOCC4)cc(C(F)(F)F)cc23)c(=O)[nH]1 ; CCN(c1cc(C(F)(F)F)cc2c(NCc3c(C)cc(C)[nH]c3=O)nccc12)C1CCOCC1]

Step 1: The preparation method was the same as that of compound z-1, except that compound 1-3 and acetaldehyde in the preparation method of compound z-1 were replaced by compound 6a and tetrahydropyrone. MS m/z (ESI): 448 [M+H]⁺.

Step 2: The preparation method was the same as that of compound z-1, except that compound 1-3 in the preparation method of compound z-1 was replaced by compound 16-1. After purified by Prep-HPLC, compound z-16 was obtained as a white solid (9.6 mg, 4%). MS m/z (ESI): 475.3[M+H]⁺. ¹H NMR (500 MHz, DMSO-d₆): δ 11.48 (br. s., 1H), 8.45 (s, 1H), 8.02 (d, 1H), 7.57 (br. s., 1H), 7.49 (s, 1H), 7.17 (d, 1H), 5.90 (s, 1H), 4.46 (d, 2H), 3.82 (m, 2H), 3.23-3.20 (m, 5H), 2.20 (s, 3H), 2.13 (s, 3H), 1.69-1.58 (m, 4H), 0.83 (t, 3H).

### Example 17: Preparation of 3-((5-(((1R,4R)-4-(dimethylamino) cyclohexyl) (ethyl)amino)-7-(6-(1-methylpiperidine-4-yl)pyridine-3-yl)-isoquinolinylamino) methyl)-4,6-dimethylpyridine-2(1H)-one (compound z-17)

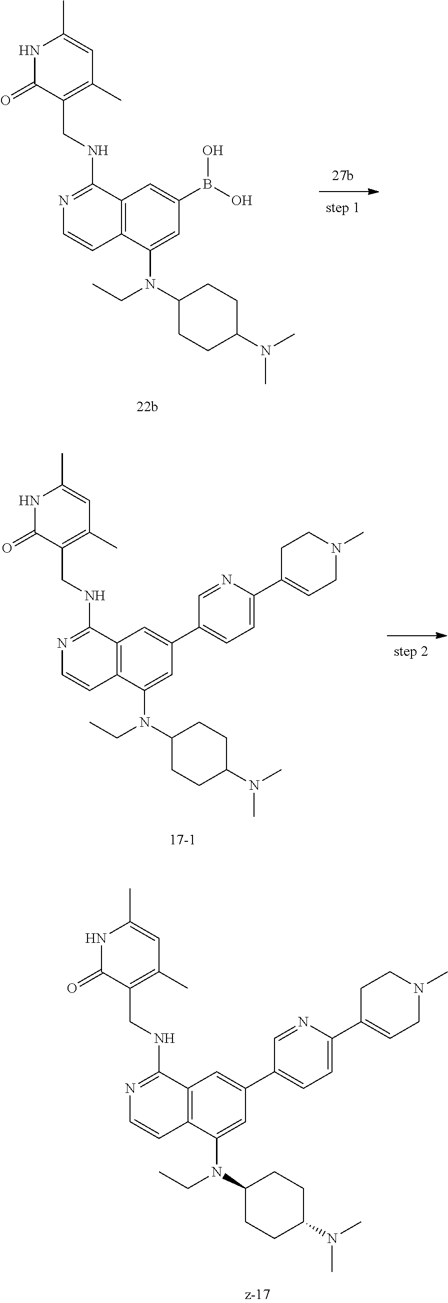 [SMILES: CCN(c1cc(B(O)O)cc2c(NCc3c(C)cc(C)[nH]c3=O)nccc12)C1CCC(N(C)C)CC1 ; CCN(c1cc(-c2ccc(C3=CCN(C)CC3)nc2)cc2c(NCc3c(C)cc(C)[nH]c3=O)nccc12)C1CCC(N(C)C)CC1 ; CCN(c1cc(-c2ccc(C3=CCN(C)CC3)nc2)cc2c(NCc3c(C)cc(C)[nH]c3=O)nccc12)[C@H]1CC[C@H](N(C)C)CC1]

Step 1: The preparation method was the same as that of compound z-7, except that compounds z-3 and 7.1 in the preparation method of compound z-7 were replaced by compounds 22b and 27b. MS m/z (ESI): 620 [M+H]⁺.

Step 2: To a solution of compound 17-1 (30 mg, 0.048 mmol) in methanol (10 mL) was added palladium on carbon (10 mg, 10%) under the protection of argon and the mixture was stirred under hydrogen atmosphere at room temperature overnight. LC-MS was used to trace the reaction until the reaction was complete. The reaction solution was filtered, and the filtrate was concentrated and purified by Prep-HPLC to give a white solid compound z-17 (3 mg, 10%). MS m/z (ESI): 622.5[M+H]⁺. ¹H NMR (400 MHz, DMSO) δ 8.90 (s, 1H), 8.22 (s, 2H), 8.07 (d, 1H), 7.86 (d, 1H), 7.58 (s, 1H), 7.36 (d, 1H), 7.29 (br. s, 1H), 7.07 (d, 1H), 5.86 (s, 1H), 4.47-4.43 (m, 2H), 3.25-3.18 (m, 2H), 3.01-2.89 (m, 3H), 2.74-2.50 (m, 3H), 2.34-2.05 (m, 15H), 1.97-1.72 (m, 8H), 1.51-1.36 (m, 3H), 1.25-1.14 (m, 2H), 0.85 (t, 3H).

### Example 18: Preparation of 1-((4,6-dimethyl-2-oxo-1,2-dihydropyridin-3-yl) methyl amino)-5-(methyl (tetrahydro-2H-pyran-4-yl) amino) isoquinoline-7-carboxamide (compound z-18)

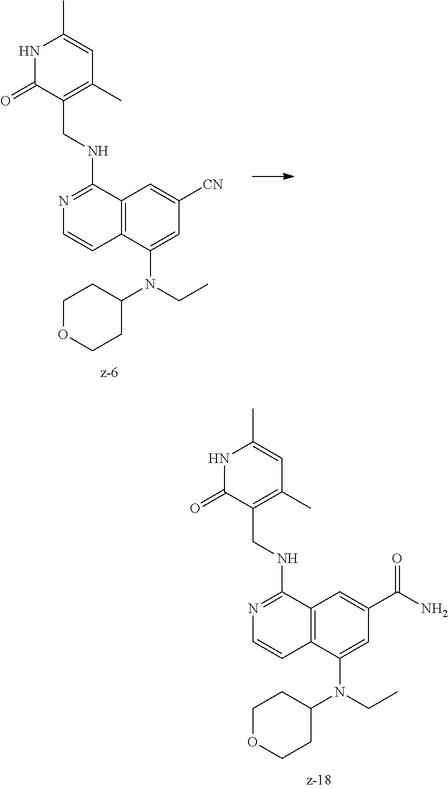 [SMILES: CCN(c1cc(C#N)cc2c(NCc3c(C)cc(C)[nH]c3=O)nccc12)C1CCOCC1 ; CCN(c1cc(C(N)=O)cc2c(NCc3c(C)cc(C)[nH]c3=O)nccc12)C1CCOCC1]

To a solution of compound z-6 (100 mg, 0.23 mmol) in hydrogen peroxide (4 mL) and DMSO (12 mL) was added potassium carbonate (138 mg, 0.92 mmol) and the mixture was stirred at room temperature for 2 hours. LC-MS was used to trace the reaction until the reaction was complete. The reaction solution was poured into water, extracted with ethyl acetate, concentrated and purified by Prep-HPLC to give compound z-18 as a white solid (20 mg, 19.2%). MS m/z (ESI): 450.3 [M+H]⁺.

### Example 19: Preparation of N-(1-((4,6-dimethyl-2-oxo-1,2-dihydropyridin-3-yl) methyl amino)-5-(ethyl (tetrahydro-2H-pyran-4-yl) amino) isoquinolin-7-yl) acetamide (compound z-19)

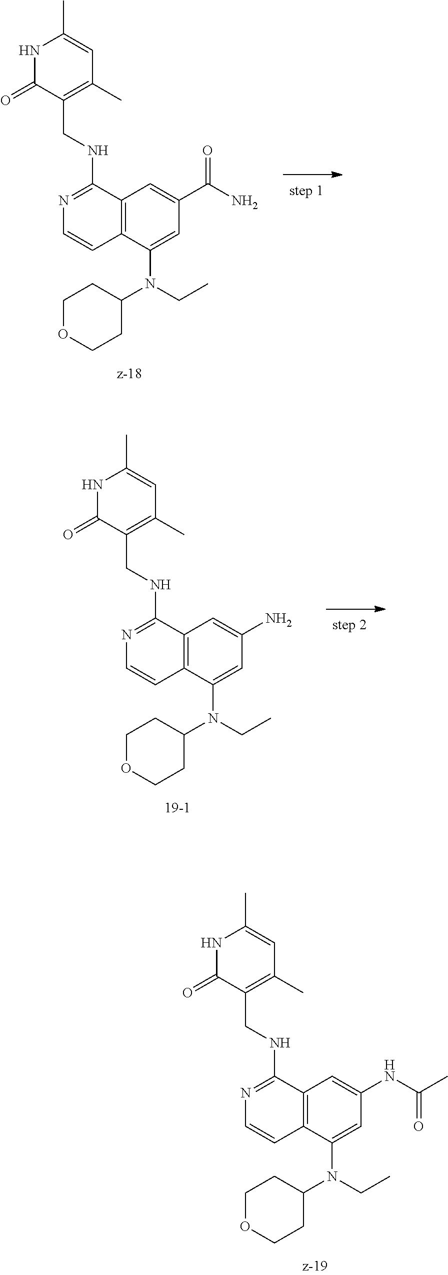 [SMILES: CCN(c1cc(C(N)=O)cc2c(NCc3c(C)cc(C)[nH]c3=O)nccc12)C1CCOCC1 ; CCN(c1cc(N)cc2c(NCc3c(C)cc(C)[nH]c3=O)nccc12)C1CCOCC1 ; CCN(c1cc(NC(C)=O)cc2c(NCc3c(C)cc(C)[nH]c3=O)nccc12)C1CCOCC1]

Step 1: To a solution of compound z-18 (245 mg, 0.54 mmol) and sodium hydroxide (108 mg, 2.7 mmol) in 1,4-dioxane (5 mL) and water (5 mL) was added sodium hypochlorite solution (201 mg, 2.7 mmol) dropwise. The mixture was stirred for 3 hours at 80° C. LC-MS was used to trace the reaction until the reaction was complete. The pH of the reaction solution was adjusted to 7-8 with 4N hydrochloric acid solution. The solvent was concentrated to remove the solvent, the residue was extracted with dichloromethane/water and the organic layer was dried and concentrated to give 250 mg of compound 19-1 as a solid which was used directly for the next reaction. MS m/z (ESI): 422 [M+H]⁺.

Step 2: To a solution of compound 19-1 (105 mg, 0.25 mmol) and triethylamine (101 mg, 1 mmol) in dichloromethane (5 mL) was added dropwise acetic anhydride (51 mg, 0.5 mmol) under an ice bath, the mixture was stirred at room temperature for 3 hours. LC-MS was used to trace the reaction until the reaction was complete. The reaction was quenched with sodium carbonate solution, extracted with ethyl acetate/water, the organic layer was dried and concentrated, and purified by Prep-HPLC to give solid compound z-19 (8.2 mg, 7%). MS m/z (ESI): 464.3 [M+H]⁺.

### Example 20: Preparation of 3-((7-bromo-5-((4-(dimethylamino) cyclohexyl)(ethyl)amino) isoquinolin-1-ylamino)methyl)-4,6-dimethylpyridine-2(1H) (compound z-20)

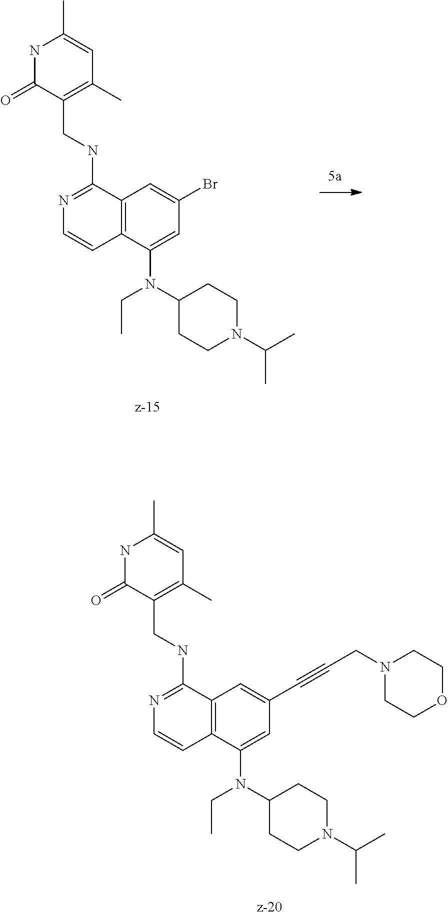 [SMILES: CCN(c1cc(Br)cc2c(NCc3c(C)cc(C)[nH]c3=O)nccc12)C1CCN(C(C)C)CC1 ; CCN(c1cc(C#CCN2CCOCC2)cc2c(NCc3c(C)cc(C)[nH]c3=O)nccc12)C1CCN(C(C)C)CC1]

To compound z-15 (50 mg, 0.1 mmol), 5a (30 mg, 0.25 mmol), copper iodide (2 mg, 0.01 mmol) and pd(Pph₃)₂Cl₂ (7 mg, 0.01 mmol) were added DMF 1 mL and triethylamine 1 mL. The mixture was sealed at 120° C. for 1 hour to conduct reaction. LC-MS was used to trace the reaction until the reaction was complete. The mixture was cooled and filtered. The filtrate was concentrated and purified by Prep-HPLC to give compound z-20 (37 mg, 65%) as a white solid. MS m/z (ESI): 571 [M+H]⁺.

### Example 21: Preparation of 1-((4,6-dimethyl-2-oxo-1,2-dihydropyridin-3-yl) methylamino)-5-((4-(dimethylamino)) cyclohexyl) (ethyl)amino) isoquinoline-7-carboxamide (compound z-21)

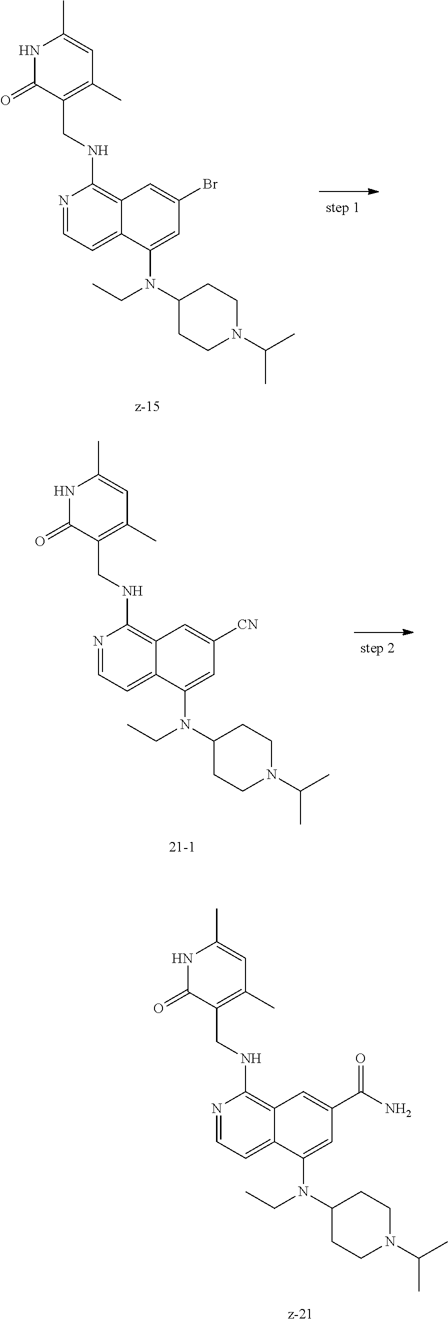 [SMILES: CCN(c1cc(Br)cc2c(NCc3c(C)cc(C)[nH]c3=O)nccc12)C1CCN(C(C)C)CC1 ; CCN(c1cc(C#N)cc2c(NCc3c(C)cc(C)[nH]c3=O)nccc12)C1CCN(C(C)C)CC1 ; CCN(c1cc(C(N)=O)cc2c(NCc3c(C)cc(C)[nH]c3=O)nccc12)C1CCN(C(C)C)CC1]

Step 1: The preparation method was the same as that of compound z-6, except that compound z-3 in the preparation method of compound z-6 was replaced by compound 12a. MS m/z (ESI): 473.3 [M+H]⁺.

Step 2: A mixture of compound 21-1 (70 mg, 0.148 mmol), sodium hydroxide (4 mL, 1 M) and ethanol (4 mL) was stirred at room temperature for 2 hours. LC-MS was used to trace the reaction until the reaction was complete. The reaction solution was poured into water. The pH was adjusted to 3, extracted with ethyl acetate, the aqueous layer was concentrated, and the residue was dissolved with ethyl acetate. The organic layers were combined, concentrated and purified by Prep-HPLC to give compound z-21 (1 mg, 1.4%) as a white solid. MS m/z (ESI): 491.4 [M+H]⁺.

### Examples 23-194, 195-215

Compounds Z-23 to Z-194 and Z-195 to Z-215 have structures as shown in formula (I), wherein when X is NH; Z₁ is N; Z₂, Z₃ are CH; R₁, R₃ are hydrogen; R₂, R₄, NR₅R₆ are the structures shown in the following table:

General procedure: compounds Z-23 to Z-131, z-191 to z-193, z-195 were prepared by a method similar to that of Example 7 using compound z-3 and different boronic acid and boronate ester intermediates as raw materials.

Compounds Z-132 to Z-150, z-158, z-197 to z-199 were prepared by a method similar to that of Example 4 using compound 3a and various aldehydes and ketones as raw materials.

Compounds Z-151 to Z-157, z-159, z-160 and z-196 were prepared by a method similar to that of Example 8 using compound z-3 and various boronic acids and boronate esters as raw materials.

Compounds Z-161 to Z-176 were prepared by a method similar to that of compound 33b using different carboxylic acids and amines as raw materials.

Compounds Z-177 to Z-190 were prepared by a method similar to that of Example 20 using isoquinoline halides as raw materials.

Compounds Z-199 to Z-209, Z-215 were prepared by a method similar to that of Example 13.

Compounds Z-210 to Z-214 were prepared by a method similar to that of Example 17.

| Example No. | Compound | NR₅R₆ | R₂ | R₄ | MS[M + H]⁺ |
|---|---|---|---|---|---|
| 23 | z-23 | 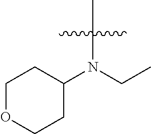 [SMILES: *N(CC)C1CCOCC1] | 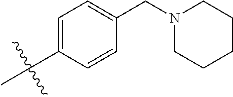 [SMILES: *c1ccc(CN2CCCCC2)cc1] | Me | 580.5 |
| 24 | z-24 | 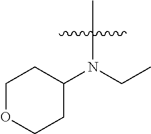 [SMILES: *N(CC)C1CCOCC1] | 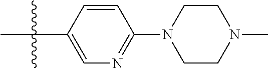 [SMILES: *c1ccc(N2CCN(C)CC2)nc1] | Me | 582 |
| 25 | z-25 | 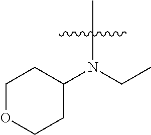 [SMILES: *N(CC)C1CCOCC1] | Me | Me | 421.2 |
| 26 | z-26 | 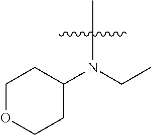 [SMILES: *N(CC)C1CCOCC1] | 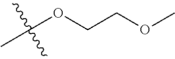 [SMILES: *OCCOC] | Me | 481 |
| 27 | z-27 | 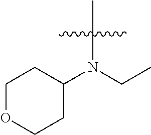 [SMILES: *N(CC)C1CCOCC1] | 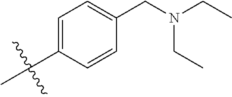 [SMILES: *c1ccc(CN(CC)CC)cc1] | Me | 568 |
| 28 | z-28 | 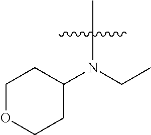 [SMILES: *N(CC)C1CCOCC1] | 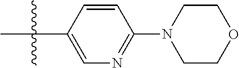 [SMILES: *c1ccc(N2CCOCC2)nc1] | Me | 569.4 |
| 29 | z-29 | 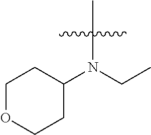 [SMILES: *N(CC)C1CCOCC1] | 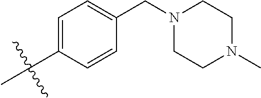 [SMILES: *c1ccc(CN2CCN(C)CC2)cc1] | Me | 595 |
| 30 | z-30 | 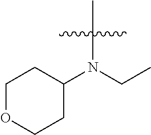 [SMILES: *N(CC)C1CCOCC1] | 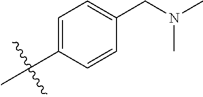 [SMILES: *c1ccc(CN(C)C)cc1] | Me | 540 |
| 31 | z-31 | 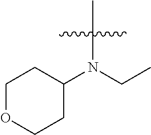 [SMILES: *N(CC)C1CCOCC1] | 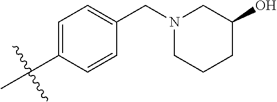 [SMILES: *c1ccc(CN2CCC[C@H](O)C2)cc1] | Me | 596 |
| 32 | z-32 | 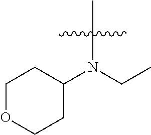 [SMILES: *N(CC)C1CCOCC1] | 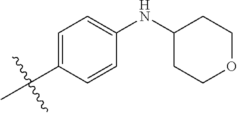 [SMILES: *c1ccc(NC2CCOCC2)cc1] | Me | 582 |
| 33 | z-33 | 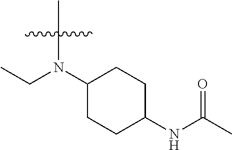 [SMILES: *N(CC)C1CCC(NC(C)=O)CC1] | 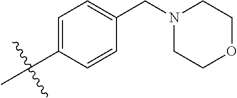 [SMILES: *c1ccc(CN2CCOCC2)cc1] | Me | 637 |
| 34 | z-34 | 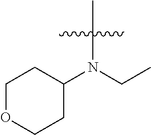 [SMILES: *N(CC)C1CCOCC1] | 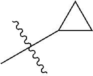 [SMILES: *C1CC1] | Me | 447 |
| 35 | z-35 | 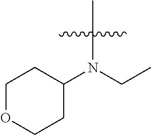 [SMILES: *N(CC)C1CCOCC1] | 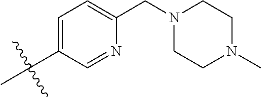 [SMILES: *c1ccc(CN2CCN(C)CC2)nc1] | Me | 596 |
| 36 | z-36 | 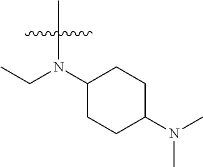 [SMILES: *N(CC)C1CCC(N(C)C)CC1] | 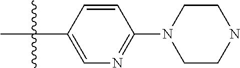 [SMILES: *c1ccc(N2CCNCC2)nc1] | Me | 624 |
| 37 | z-37 | 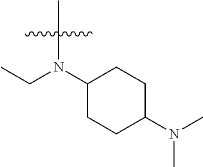 [SMILES: *N(CC)C1CCC(N(C)C)CC1] | 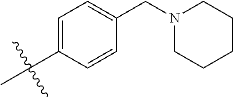 [SMILES: *c1ccc(CN2CCCCC2)cc1] | Me | 621.3 |
| 38 | z-38 | 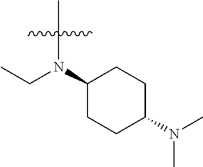 [SMILES: *N(CC)[C@H]1CC[C@H](N(C)C)CC1] | 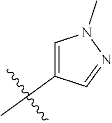 [SMILES: *c1cnn(C)c1] | Me | 528.4 |
| 39 | z-39 | 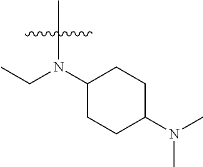 [SMILES: *N(CC)C1CCC(N(C)C)CC1] | 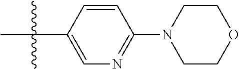 [SMILES: *c1ccc(N2CCOCC2)nc1] | Me | 610.5 |
| 40 | z-40 | 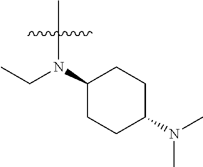 [SMILES: *N(CC)[C@H]1CC[C@H](N(C)C)CC1] | 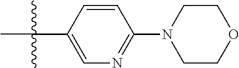 [SMILES: *c1ccc(N2CCOCC2)nc1] | Me | 610.5 |
| 41 | z-41 | 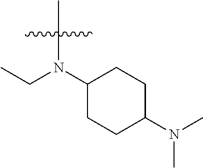 [SMILES: *N(CC)C1CCC(N(C)C)CC1] | 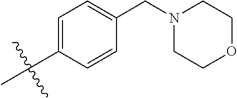 [SMILES: *c1ccc(CN2CCOCC2)cc1] | Me | 623.5 |
| 42 | z-42 | 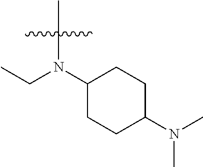 [SMILES: *N(CC)C1CCC(N(C)C)CC1] | 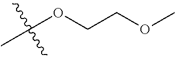 [SMILES: *OCCOC] | Me | 598.4 |
| 43 | z-43 | 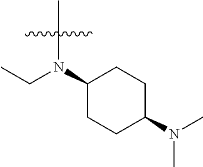 [SMILES: *N(CC)[C@H]1CC[C@@H](N(C)C)CC1] | 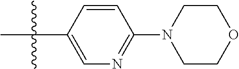 [SMILES: *c1ccc(N2CCOCC2)nc1] | Me | 610.5 |
| 44 | z-44 | 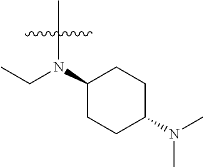 [SMILES: *N(CC)[C@H]1CC[C@H](N(C)C)CC1] | 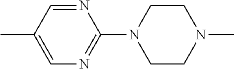 | Me | 624.5 |
| 45 | z-45 | 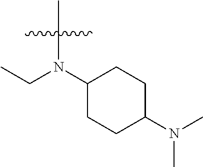 [SMILES: *N(CC)C1CCC(N(C)C)CC1] | 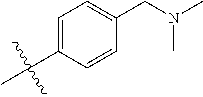 [SMILES: *c1ccc(CN(C)C)cc1] | Me | 581.5 |
| 46 | z-46 | 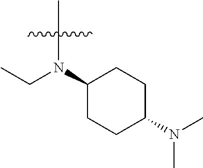 [SMILES: *N(CC)[C@H]1CC[C@H](N(C)C)CC1] | 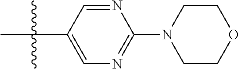 [SMILES: *c1cnc(N2CCOCC2)nc1] | Me | 611.4 |
| 47 | z-47 | 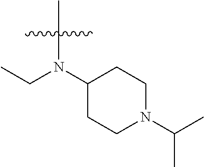 [SMILES: *N(CC)C1CCN(C(C)C)CC1] | 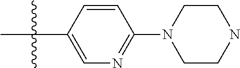 [SMILES: *c1ccc(N2CCNCC2)nc1] | Me | 623 |
| 48 | z-48 | 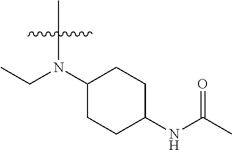 [SMILES: *N(CC)C1CCC(NC(C)=O)CC1] | 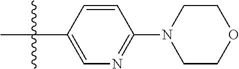 [SMILES: *c1ccc(N2CCOCC2)nc1] | Me | 624.5 |
| 49 | z-49 | 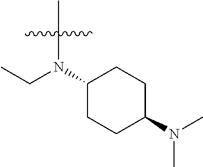 [SMILES: *N(CC)[C@H]1CC[C@H](N(C)C)CC1] | 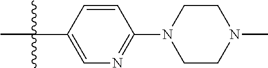 [SMILES: *c1ccc(N2CCN(C)CC2)nc1] | Me | 623.5 |
| 50 | z-50 | 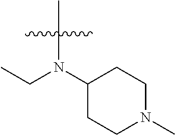 [SMILES: *N(CC)C1CCN(C)CC1] | 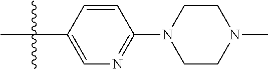 [SMILES: *c1ccc(N2CCN(C)CC2)nc1] | Me | 595 |
| 51 | z-51 | 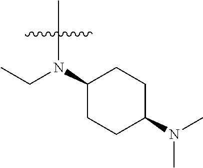 [SMILES: *N(CC)[C@H]1CC[C@@H](N(C)C)CC1] | 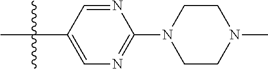 [SMILES: *c1cnc(N2CCN(C)CC2)nc1] | Me | 624.5 |
| 52 | z-52 | 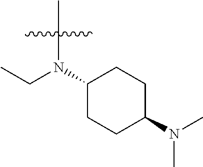 [SMILES: *N(CC)[C@H]1CC[C@H](N(C)C)CC1] | 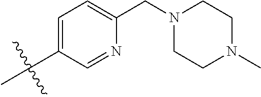 [SMILES: *c1ccc(CN2CCN(C)CC2)nc1] | Me | 637.5 |
| 53 | z-53 | 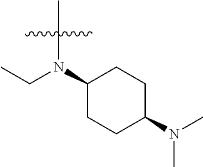 [SMILES: *N(CC)[C@H]1CC[C@@H](N(C)C)CC1] | 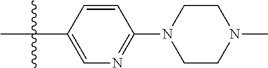 [SMILES: *c1ccc(N2CCN(C)CC2)nc1] | Me | 623.5 |
| 54 | z-54 | 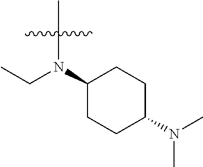 [SMILES: *N(CC)[C@H]1CC[C@H](N(C)C)CC1] | 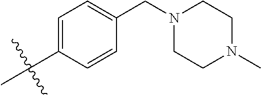 [SMILES: *c1ccc(CN2CCN(C)CC2)cc1] | Me | 636.5 |
| 55 | z-55 | 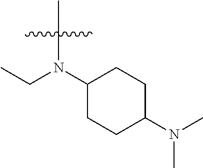 [SMILES: *N(CC)C1CCC(N(C)C)CC1] | 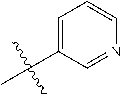 [SMILES: *c1cccnc1] | Me | 525 |
| 56 | z-56 | 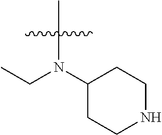 [SMILES: *N(CC)C1CCNCC1] | 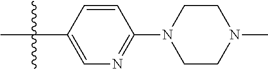 [SMILES: *c1ccc(N2CCN(C)CC2)nc1] | Me | 581.4 |
| 57 | z-57 | 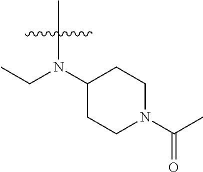 [SMILES: *N(CC)C1CCN(C(C)=O)CC1] | 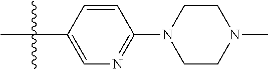 [SMILES: *c1ccc(N2CCN(C)CC2)nc1] | Me | 623 |
| 58 | z-58 | 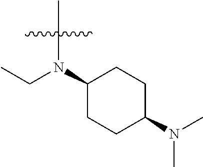 [SMILES: *N(CC)[C@H]1CC[C@@H](N(C)C)CC1] | 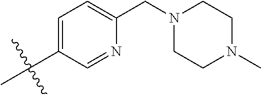 [SMILES: *c1ccc(CN2CCN(C)CC2)nc1] | Me | 637.4 |
| 59 | z-59 | 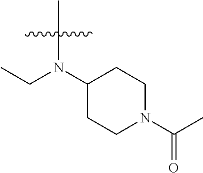 [SMILES: *N(CC)C1CCN(C(C)=O)CC1] | 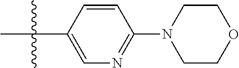 [SMILES: *c1ccc(N2CCOCC2)nc1] | Me | 610 |
| 60 | z-60 | 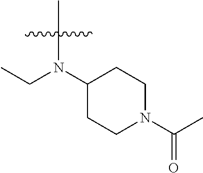 [SMILES: *N(CC)C1CCN(C(C)=O)CC1] | 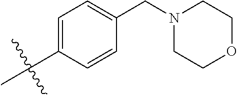 [SMILES: *c1ccc(CN2CCOCC2)cc1] | Me | 623 |
| 61 | z-61 | 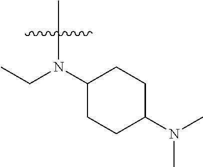 [SMILES: *N(CC)C1CCC(N(C)C)CC1] | 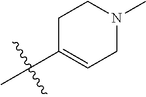 [SMILES: *C1=CCN(C)CC1] | Me | 544 |
| 62 | z-62 | 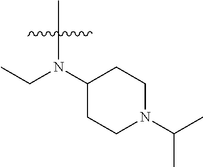 [SMILES: *N(CC)C1CCN(C(C)C)CC1] | 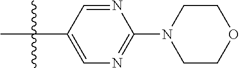 [SMILES: *c1cnc(N2CCOCC2)nc1] | Me | 611 |
| 63 | z-63 | 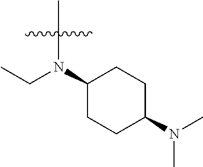 [SMILES: *N(CC)[C@H]1CC[C@@H](N(C)C)CC1] | 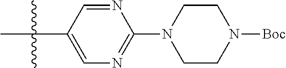 [SMILES: *c1cnc(N2CCN(C(=O)OC(C)(C)C)CC2)nc1] | Me | 723 |
| 64 | z-64 | 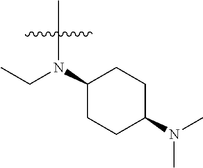 [SMILES: *N(CC)[C@H]1CC[C@@H](N(C)C)CC1] | 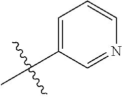 [SMILES: *c1cccnc1] | Me | 525.4 |
| 65 | z-65 | 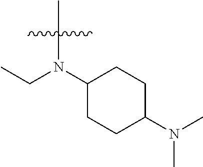 [SMILES: *N(CC)C1CCC(N(C)C)CC1] | 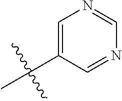 [SMILES: *c1cncnc1] | Me | 526 |
| 66 | z-66 | 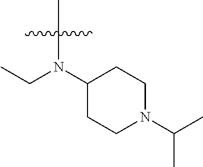 [SMILES: *N(CC)C1CCN(C(C)C)CC1] | 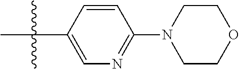 [SMILES: *c1ccc(N2CCOCC2)nc1] | Me | 610 |
| 67 | z-67 | 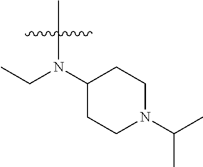 [SMILES: *N(CC)C1CCN(C(C)C)CC1] | 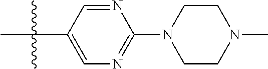 [SMILES: *c1cnc(N2CCN(C)CC2)nc1] | Me | 624 |
| 68 | z-68 | 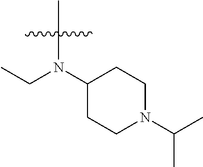 [SMILES: *N(CC)C1CCN(C(C)C)CC1] | 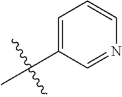 [SMILES: *c1cccnc1] | Me | 525 |
| 69 | z-69 | 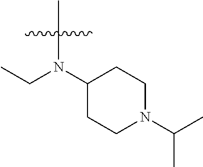 [SMILES: *N(CC)C1CCN(C(C)C)CC1] | 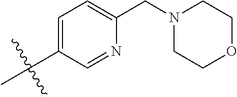 [SMILES: *c1ccc(CN2CCOCC2)nc1] | Me | 624 |
| 70 | z-70 | 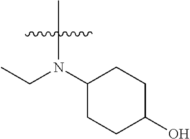 [SMILES: *N(CC)C1CCC(O)CC1] | 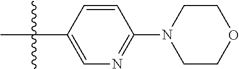 [SMILES: *c1ccc(N2CCOCC2)nc1] | Me | 583.4 |
| 71 | z-71 | 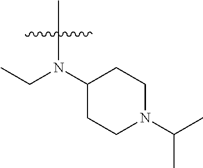 [SMILES: *N(CC)C1CCN(C(C)C)CC1] | 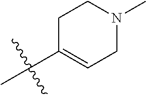 [SMILES: *C1=CCN(C)CC1] | Me | 543.4 |
| 72 | z-72 | 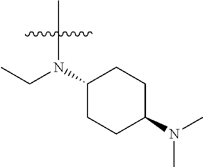 [SMILES: *N(CC)[C@H]1CC[C@H](N(C)C)CC1] | 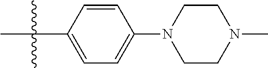 [SMILES: *c1ccc(N2CCN(C)CC2)cc1] | Me | 622.5 |
| 73 | z-73 | 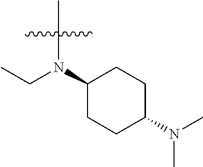 [SMILES: *N(CC)[C@H]1CC[C@H](N(C)C)CC1] | 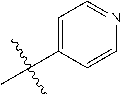 [SMILES: *c1ccncc1] | Me | 525.4 |
| 74 | z-74 | 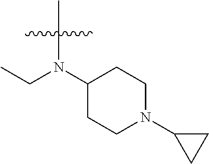 [SMILES: *N(CC)C1CCN(C2CC2)CC1] | 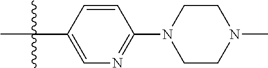 [SMILES: *c1ccc(N2CCN(C)CC2)nc1] | Me | 621.5 |
| 75 | z-75 | 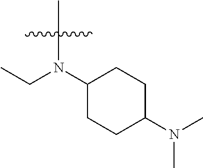 [SMILES: *N(CC)C1CCC(N(C)C)CC1] | 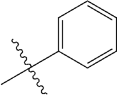 [SMILES: *c1ccccc1] | Me | 524.0 |
| 76 | z-76 | 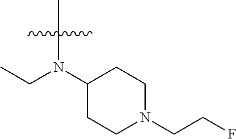 [SMILES: *N(CC)C1CCN(CCF)CC1] | 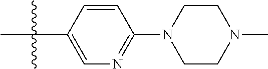 [SMILES: *c1ccc(N2CCN(C)CC2)nc1] | Me | 627 |
| 77 | z-77 | 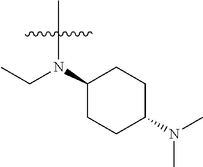 [SMILES: *N(CC)[C@H]1CC[C@H](N(C)C)CC1] | 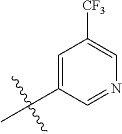 [SMILES: *c1cncc(C(F)(F)F)c1] | Me | 593 |
| 78 | z-78 | 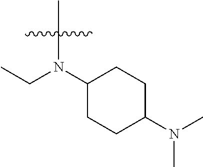 [SMILES: *N(CC)C1CCC(N(C)C)CC1] | 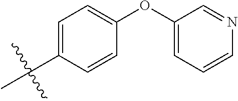 [SMILES: *c1ccc(Oc2cccnc2)cc1] | Me | 617 |
| 79 | z-79 | 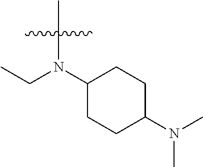 [SMILES: *N(CC)C1CCC(N(C)C)CC1] | 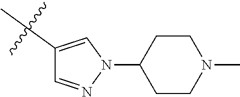 [SMILES: *c1cnn(C2CCN(C)CC2)c1] | Me | 611 |
| 80 | z-80 | 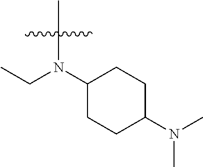 [SMILES: *N(CC)C1CCC(N(C)C)CC1] | 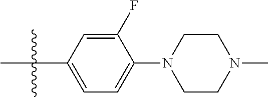 [SMILES: *c1ccc(N2CCN(C)CC2)c(F)c1] | Me | 640.4 |
| 81 | z-81 | 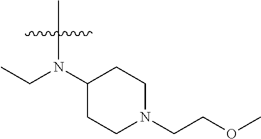 [SMILES: *N(CC)C1CCN(CCOC)CC1] | 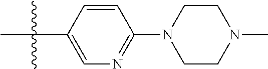 [SMILES: *c1ccc(N2CCN(C)CC2)nc1] | Me | 639 |
| 82 | z-82 | 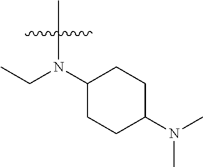 [SMILES: *N(CC)C1CCC(N(C)C)CC1] | 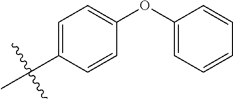 [SMILES: *c1ccc(Oc2ccccc2)cc1] | Me | 616 |
| 83 | z-83 | 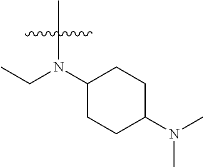 [SMILES: *N(CC)C1CCC(N(C)C)CC1] | 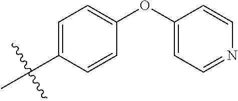 [SMILES: *c1ccc(Oc2ccncc2)cc1] | Me | 617 |
| 84 | z-84 | 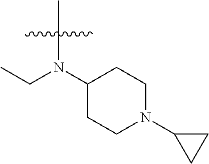 [SMILES: *N(CC)C1CCN(C2CC2)CC1] | 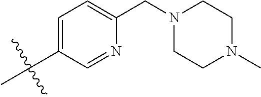 [SMILES: *c1ccc(CN2CCN(C)CC2)nc1] | Me | 635 |
| 85 | z-85 | 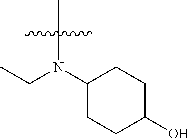 [SMILES: *N(CC)C1CCC(O)CC1] | 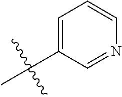 [SMILES: *c1cccnc1] | Me | 498.3 |
| 86 | z-86 | 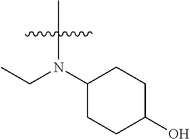 [SMILES: *N(CC)C1CCC(O)CC1] | 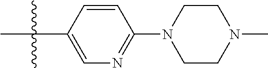 [SMILES: *c1ccc(N2CCN(C)CC2)nc1] | Me | 596 |
| 87 | z-87 | 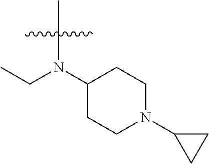 [SMILES: *N(CC)C1CCN(C2CC2)CC1] | 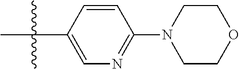 [SMILES: *c1ccc(N2CCOCC2)nc1] | Me | 608.4 |
| 88 | z-88 | 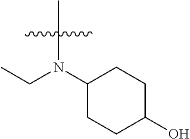 [SMILES: *N(CC)C1CCC(O)CC1] | 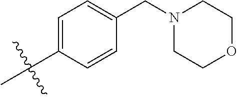 [SMILES: *c1ccc(CN2CCOCC2)cc1] | Me | 596.0 |
| 89 | z-89 | 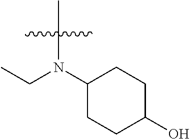 [SMILES: *N(CC)C1CCC(O)CC1] | 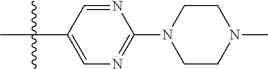 [SMILES: *c1cnc(N2CCN(C)CC2)nc1] | Me | 597 |
| 90 | z-90 | 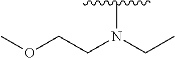 [SMILES: *N(CC)CCOC] | 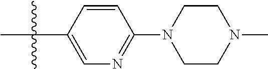 [SMILES: *c1ccc(N2CCN(C)CC2)nc1] | Me | 556 |
| 91 | z-91 | 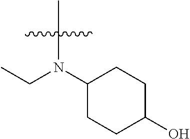 [SMILES: *N(CC)C1CCC(O)CC1] | 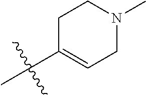 [SMILES: *C1=CCN(C)CC1] | Me | 516.4 |
| 92 | z-92 | 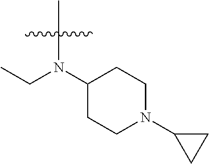 [SMILES: *N(CC)C1CCN(C2CC2)CC1] | 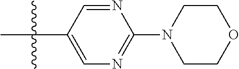 [SMILES: *c1cnc(N2CCOCC2)nc1] | Me | 609.4 |
| 93 | z-93 | 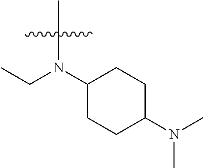 [SMILES: *N(CC)C1CCC(N(C)C)CC1] | 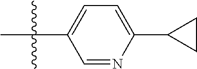 [SMILES: *c1ccc(C2CC2)nc1] | Me | 565.4 |
| 94 | z-94 | 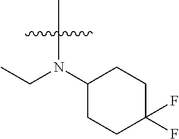 [SMILES: *N(CC)C1CCC(F)(F)CC1] | 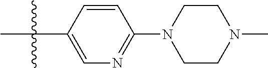 [SMILES: *c1ccc(N2CCN(C)CC2)nc1] | Me | 616.4 |
| 95 | z-95 | 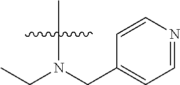 [SMILES: *N(CC)Cc1ccncc1] | 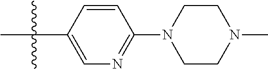 [SMILES: *c1ccc(N2CCN(C)CC2)nc1] | Me | 589 |
| 96 | z-96 | 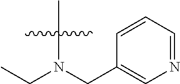 [SMILES: *N(CC)Cc1cccnc1] | 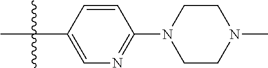 [SMILES: *c1ccc(N2CCN(C)CC2)nc1] | Me | 589 |

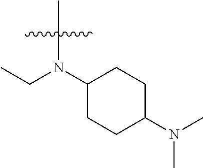 [SMILES: *N(CC)C1CCC(N(C)C)CC1]

| | | | | | |
|---|---|---|---|---|---|
| 97 | z-97 | 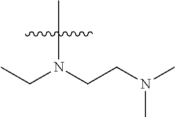 [SMILES: *N(CC)CCN(C)C] | 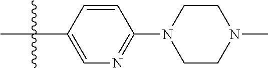 [SMILES: *c1ccc(N2CCN(C)CC2)nc1] | Me | 569 |
| 98 | z-98 | 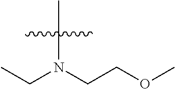 [SMILES: *N(CC)CCOC] | 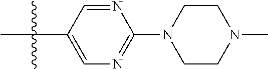 [SMILES: *c1cnc(N2CCN(C)CC2)nc1] | Me | 557.4 |
| 99 | z-99 | 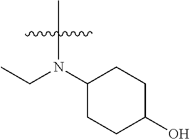 [SMILES: *N(CC)C1CCC(O)CC1] | 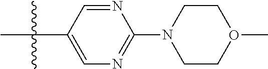 | Me | 584.4 |
| 100 | z-100 | 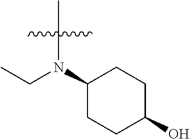 [SMILES: *N(CC)[C@H]1CC[C@@H](O)CC1] | 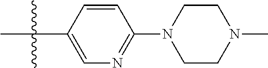 [SMILES: *c1ccc(N2CCN(C)CC2)nc1] | Me | 596 |
| 101 | z-101 | 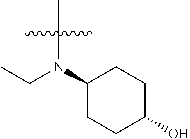 [SMILES: *N(CC)[C@H]1CC[C@H](O)CC1] | 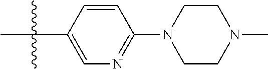 [SMILES: *c1ccc(N2CCN(C)CC2)nc1] | Me | 596 |
| 102 | z-102 | 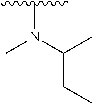 [SMILES: *N(C)C(C)CC] | 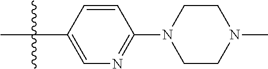 [SMILES: *c1ccc(N2CCN(C)CC2)nc1] | Me | 540 |
| 103 | z-103 | 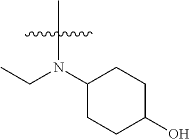 [SMILES: *N(CC)C1CCC(O)CC1] | 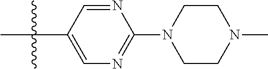 [SMILES: *c1cnc(N2CCN(C)CC2)nc1] | OMe | 613.4 |
| 104 | z-104 | 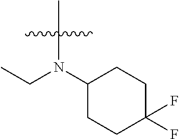 [SMILES: *N(CC)C1CCC(F)(F)CC1] | 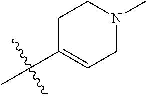 [SMILES: *C1=CCN(C)CC1] | Me | 536.4 |
| 105 | z-105 | 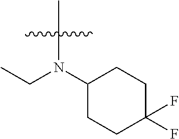 [SMILES: *N(CC)C1CCC(F)(F)CC1] | 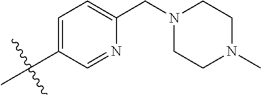 [SMILES: *c1ccc(CN2CCN(C)CC2)nc1] | Me | 630.4 |
| 106 | z-106 | 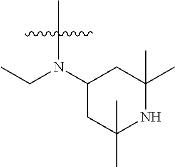 | 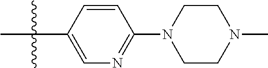 [SMILES: *c1ccc(N2CCN(C)CC2)nc1] | Me | 637 |
| 107 | z-107 | 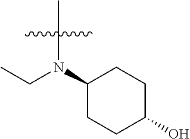 [SMILES: *N(CC)[C@H]1CC[C@H](O)CC1] | 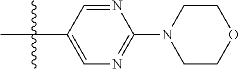 [SMILES: *c1cnc(N2CCOCC2)nc1] | Me | 584.4 |
| 108 | z-108 | 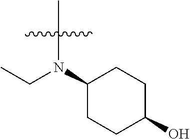 [SMILES: *N(CC)[C@H]1CC[C@@H](O)CC1] | 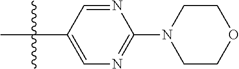 [SMILES: *c1cnc(N2CCOCC2)nc1] | Me | 584.4 |
| 109 | z-109 | 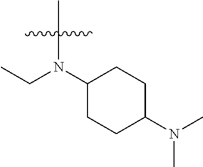 [SMILES: *N(CC)C1CCC(N(C)C)CC1] | 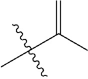 [SMILES: *C(=C)C] | Me | 488.4 |
| 110 | z-110 | 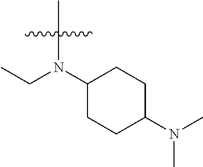 [SMILES: *N(CC)C1CCC(N(C)C)CC1] | 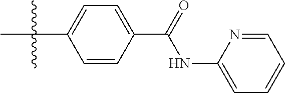 [SMILES: *c1ccc(C(=O)Nc2ccccn2)cc1] | Me | 644 |
| 111 | z-111 | 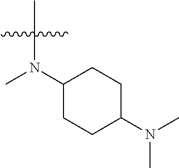 | 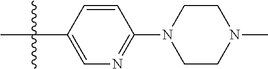 [SMILES: *c1ccc(N2CCN(C)CC2)nc1] | Me | 609.5 |
| 112 | z-112 | 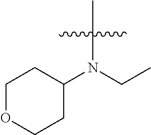 [SMILES: *N(CC)C1CCOCC1] | 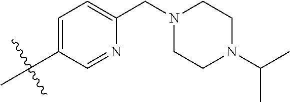 [SMILES: *c1ccc(CN2CCN(C(C)C)CC2)nc1] | Me | 624 |
| 113 | z-113 | 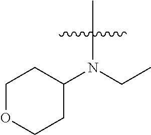 [SMILES: *N(CC)C1CCOCC1] | 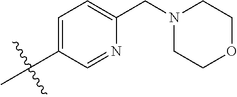 [SMILES: *c1ccc(CN2CCOCC2)nc1] | Me | 583 |
| 114 | z-114 | 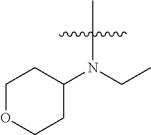 [SMILES: *N(CC)C1CCOCC1] | 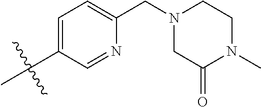 [SMILES: *c1ccc(CN2CCN(C)C(=O)C2)nc1] | Me | 610 |
| 115 | z-115 | 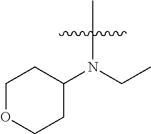 [SMILES: *N(CC)C1CCOCC1] | 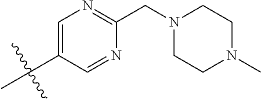 [SMILES: *c1cnc(CN2CCN(C)CC2)nc1] | Me | 597 |
| 116 | z-116 | 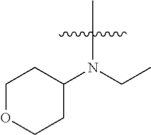 [SMILES: *N(CC)C1CCOCC1] | 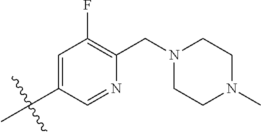 [SMILES: *c1cnc(CN2CCN(C)CC2)c(F)c1] | Me | 614 |
| 117 | z-117 | 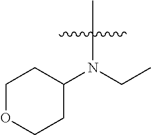 [SMILES: *N(CC)C1CCOCC1] | 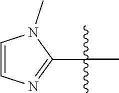 [SMILES: *c1nccn1C] | Me | 487 |
| 118 | z-118 | 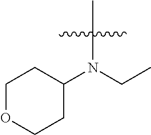 [SMILES: *N(CC)C1CCOCC1] | 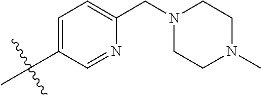 [SMILES: *c1ccc(CN2CCN(C)CC2)nc1] | Me | 596.4 |
| 119 | z-119 | 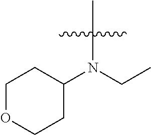 [SMILES: *N(CC)C1CCOCC1] | 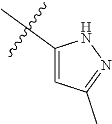 [SMILES: *c1cc(C)n[nH]1] | Me | 487.3 |
| 120 | z-120 | 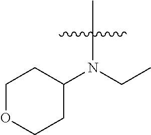 [SMILES: *N(CC)C1CCOCC1] | 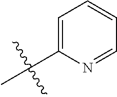 [SMILES: *c1ccccn1] | Me | 484.3 |
| 121 | z-121 | 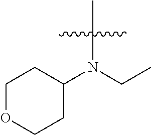 [SMILES: *N(CC)C1CCOCC1] | 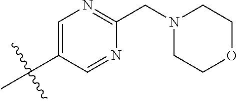 [SMILES: *c1cnc(CN2CCOCC2)nc1] | Me | 584.3 |
| 122 | z-122 | 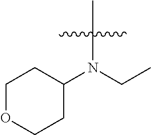 [SMILES: *N(CC)C1CCOCC1] | 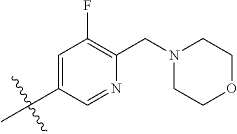 [SMILES: *c1cnc(CN2CCOCC2)c(F)c1] | Me | 601.3 |
| 123 | z-123 | 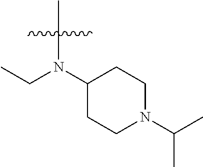 [SMILES: *N(CC)C1CCN(C(C)C)CC1] | 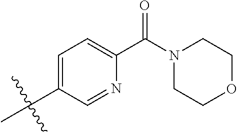 [SMILES: *c1ccc(C(=O)N2CCOCC2)nc1] | Me | 638.4 |
| 124 | z-124 | 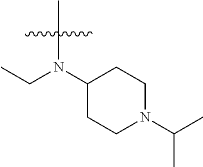 [SMILES: *N(CC)C1CCN(C(C)C)CC1] | 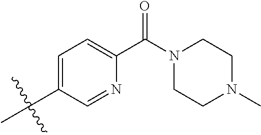 [SMILES: *c1ccc(C(=O)N2CCN(C)CC2)nc1] | Me | 651.5 |
| 125 | z-125 | 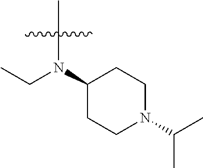 | 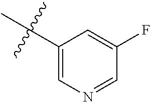 [SMILES: *c1cncc(F)c1] | Me | 543.4 |
| 126 | z-126 | 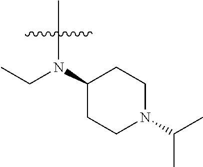 | 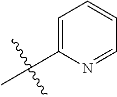 [SMILES: *c1ccccn1] | Me | 525.4 |
| 127 | z-127 | 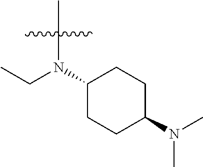 [SMILES: *N(CC)[C@H]1CC[C@H](N(C)C)CC1] | 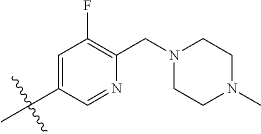 [SMILES: *c1cnc(CN2CCN(C)CC2)c(F)c1] | Me | 655.5 |
| 128 | z-128 | 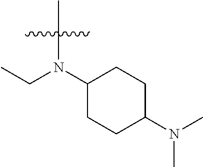 [SMILES: *N(CC)C1CCC(N(C)C)CC1] | 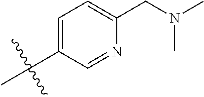 [SMILES: *c1ccc(CN(C)C)nc1] | Me | 582.3 |
| 129 | z-129 | 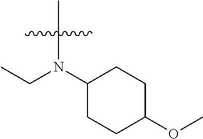 [SMILES: *N(CC)C1CCC(OC)CC1] | 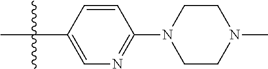 [SMILES: *c1ccc(N2CCN(C)CC2)nc1] | Me | 610.4 |
| 130 | z-130 | 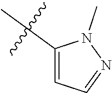 [SMILES: *c1ccnn1C] | Cl | Me | 394 |
| 131 | z-131 | 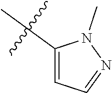 [SMILES: *c1ccnn1C] | 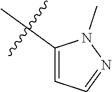 [SMILES: *c1ccnn1C] | Me | 440 |
| 132 | z-132 | 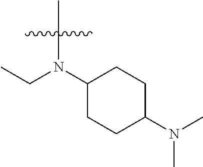 [SMILES: *N(CC)C1CCC(N(C)C)CC1] | Cl | Me | 482 |
| 133 | z-133 | 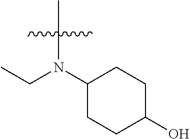 [SMILES: *N(CC)C1CCC(O)CC1] | Cl | Me | 455 |
| 134 | z-134 | 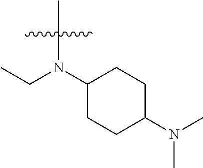 [SMILES: *N(CC)C1CCC(N(C)C)CC1] | CF₃ | Me | 516 |
| 135 | z-135 | 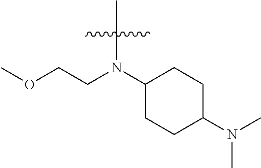 | Cl | Me | 512.3 |
| 136 | z-136 | 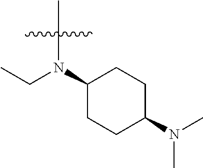 [SMILES: *N(CC)[C@H]1CC[C@@H](N(C)C)CC1] | Cl | Me | 482 |
| 137 | z-137 | 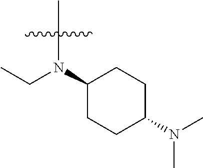 [SMILES: *N(CC)[C@H]1CC[C@H](N(C)C)CC1] | Cl | Me | 482 |
| 138 | z-138 | 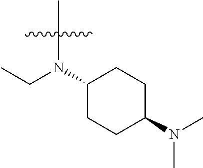 [SMILES: *N(CC)[C@H]1CC[C@H](N(C)C)CC1] | Cl | OMe | 498 |
| 139 | z-139 | 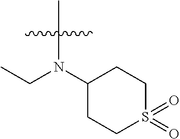 [SMILES: *N(CC)C1CCS(=O)(=O)CC1] | Cl | Me | 488.9 |
| 140 | z-140 | 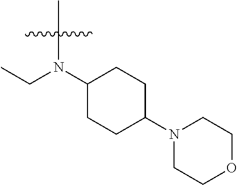 [SMILES: *N(CC)C1CCC(N2CCOCC2)CC1] | Cl | Me | 524 |
| 141 | z-141 | 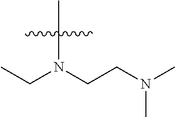 [SMILES: *N(CC)CCN(C)C] | Cl | Me | 428 |
| 142 | z-142 | 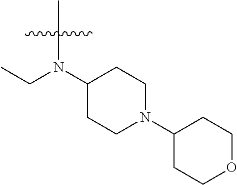 [SMILES: *N(CC)C1CCN(C2CCOCC2)CC1] | Cl | Me | 524 |
| 143 | z-143 | 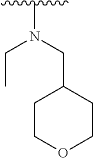 [SMILES: *N(CC)CC1CCOCC1] | Cl | Me | 455 |
| 144 | z-144 | 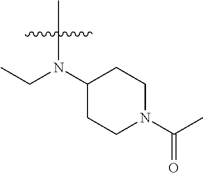 [SMILES: *N(CC)C1CCN(C(C)=O)CC1] | Cl | Me | 482 |
| 145 | z-145 | 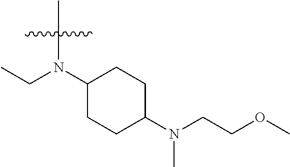 [SMILES: *N(CC)C1CCC(N(C)CCOC)CC1] | Cl | Me | 526 |
| 146 | z-146 | 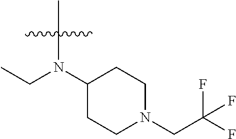 [SMILES: *N(CC)C1CCN(CC(F)(F)F)CC1] | Cl | Me | 522 |
| 147 | z-147 | 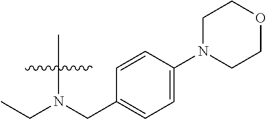 [SMILES: *N(CC)Cc1ccc(N2CCOCC2)cc1] | Cl | Me | 532 |
| 148 | z-148 | 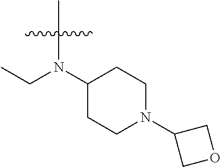 [SMILES: *N(CC)C1CCN(C2COC2)CC1] | C1 | Me | 496.3 |
| 149 | z-149 | 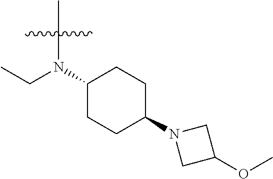 [SMILES: *N(CC)[C@H]1CC[C@H](N2CC(OC)C2)CC1] | C1 | Me | 524 |
| 150 | z-150 | 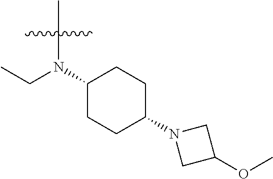 [SMILES: *N(CC)[C@H]1CC[C@@H](N2CC(OC)C2)CC1] | Cl | Me | 524 |
| 151 | z-151 | 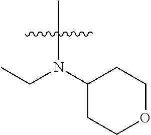 [SMILES: *N(CC)C1CCOCC1] | 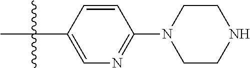 [SMILES: *c1ccc(N2CCNCC2)nc1] | Me | 568 |
| 152 | z-152 | 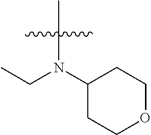 [SMILES: *N(CC)C1CCOCC1] | 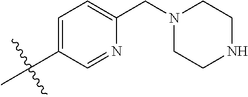 [SMILES: *c1ccc(CN2CCNCC2)nc1] | Me | 582.4 |
| 153 | z-153 | 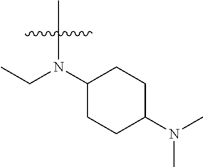 [SMILES: *N(CC)C1CCC(N(C)C)CC1] | 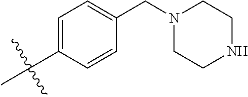 [SMILES: *c1ccc(CN2CCNCC2)cc1] | Me | 622 |
| 154 | z-154 | 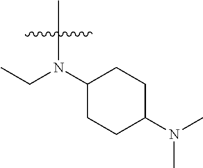 [SMILES: *N(CC)C1CCC(N(C)C)CC1] | 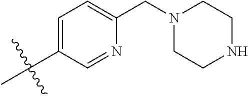 [SMILES: *c1ccc(CN2CCNCC2)nc1] | Me | 623 |
| 155 | z-155 | 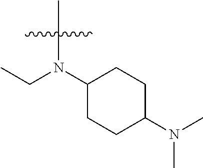 [SMILES: *N(CC)C1CCC(N(C)C)CC1] | 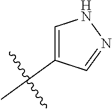 [SMILES: *c1cn[nH]c1] | Me | 514 |
| 156 | z-156 | 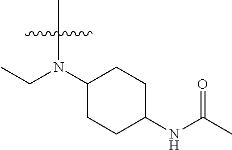 [SMILES: *N(CC)C1CCC(NC(C)=O)CC1] | 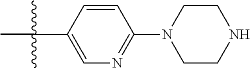 [SMILES: *c1ccc(N2CCNCC2)nc1] | Me | 623 |
| 157 | z-157 | 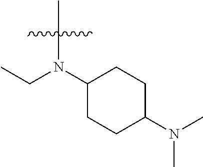 [SMILES: *N(CC)C1CCC(N(C)C)CC1] | 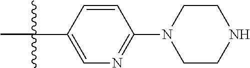 [SMILES: *c1ccc(N2CCNCC2)nc1] | Me | 609 |
| 158 | z-158 | 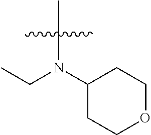 [SMILES: *N(CC)C1CCOCC1] | 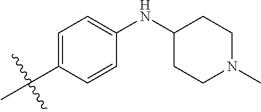 [SMILES: *c1ccc(NC2CCN(C)CC2)cc1] | Me | 595 |
| 159 | z-159 | 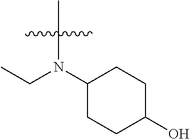 [SMILES: *N(CC)C1CCC(O)CC1] | 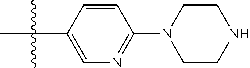 [SMILES: *c1ccc(N2CCNCC2)nc1] | Me | 582 |
| 160 | z-160 | 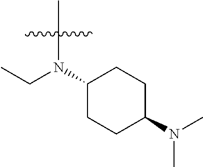 [SMILES: *N(CC)[C@H]1CC[C@H](N(C)C)CC1] | 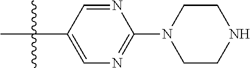 [SMILES: *c1cnc(N2CCNCC2)nc1] | Me | 610 |
| 161 | z-161 | 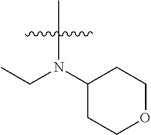 [SMILES: *N(CC)C1CCOCC1] | 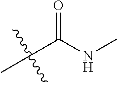 [SMILES: *C(=O)NC] | Me | 464 |
| 162 | z-162 | 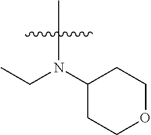 [SMILES: *N(CC)C1CCOCC1] | 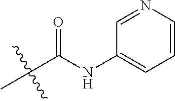 [SMILES: *C(=O)Nc1cccnc1] | Me | 527 |
| 163 | z-163 | 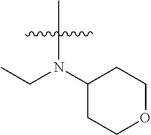 [SMILES: *N(CC)C1CCOCC1] | 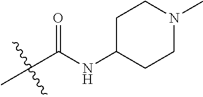 [SMILES: *C(=O)NC1CCN(C)CC1] | Me | 547 |
| 164 | z-164 | 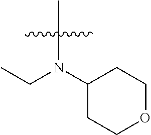 [SMILES: *N(CC)C1CCOCC1] | 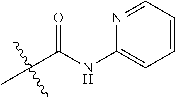 [SMILES: *C(=O)Nc1ccccn1] | Me | 527 |
| 165 | z-165 | 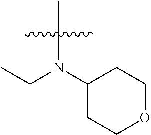 [SMILES: *N(CC)C1CCOCC1] | 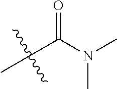 [SMILES: *C(=O)N(C)C] | Me | 478 |
| 166 | z-166 | 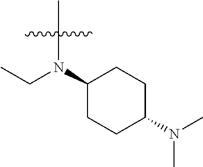 [SMILES: *N(CC)[C@H]1CC[C@H](N(C)C)CC1] | 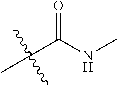 [SMILES: *C(=O)NC] | Me | 505 |
| 167 | z-167 | 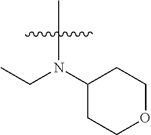 [SMILES: *N(CC)C1CCOCC1] | 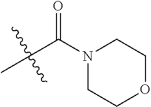 [SMILES: *C(=O)N1CCOCC1] | Me | 520 |
| 168 | z-168 | 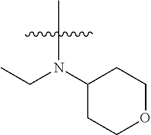 [SMILES: *N(CC)C1CCOCC1] | 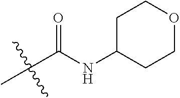 [SMILES: *C(=O)NC1CCOCC1] | Me | 534 |
| 169 | z-169 | 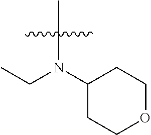 [SMILES: *N(CC)C1CCOCC1] | 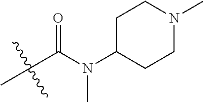 [SMILES: *C(=O)N(C)C1CCN(C)CC1] | Me | 561 |
| 170 | z-170 | 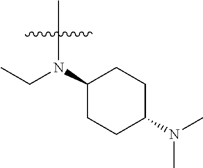 [SMILES: *N(CC)[C@H]1CC[C@H](N(C)C)CC1] | 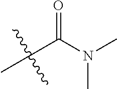 [SMILES: *C(=O)N(C)C] | Me | 519 |
| 171 | z-171 | 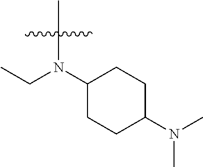 [SMILES: *N(CC)C1CCC(N(C)C)CC1] | 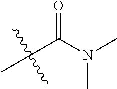 [SMILES: *C(=O)N(C)C] | Me | 519 |
| 172 | z-172 | 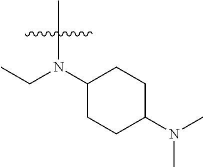 [SMILES: *N(CC)C1CCC(N(C)C)CC1] | 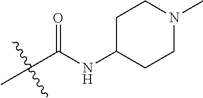 [SMILES: *C(=O)NC1CCN(C)CC1] | Me | 588 |
| 173 | z-173 | 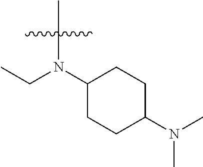 [SMILES: *N(CC)C1CCC(N(C)C)CC1] | 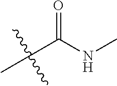 [SMILES: *C(=O)NC] | Me | 505 |
| 174 | z-174 | 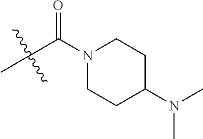 [SMILES: *C(=O)N1CCC(N(C)C)CC1] | 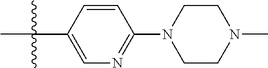 [SMILES: *c1ccc(N2CCN(C)CC2)nc1] | Me | 609 |
| 175 | z-175 | 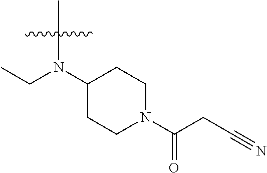 [SMILES: *N(CC)C1CCN(C(=O)CC#N)CC1] | Cl | Me | 507 |
| 176 | z-176 | 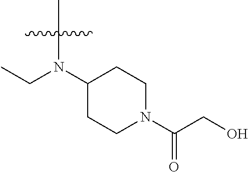 [SMILES: *N(CC)C1CCN(C(=O)CO)CC1] | Cl | Me | 498 |
| 177 | z-177 | 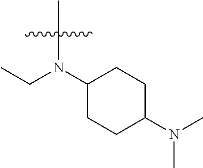 [SMILES: *N(CC)C1CCC(N(C)C)CC1] | 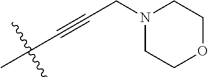 [SMILES: *C#CCN1CCOCC1] | Me | 571.4 |
| 178 | z-178 | 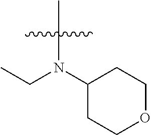 [SMILES: *N(CC)C1CCOCC1] | 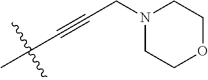 [SMILES: *C#CCN1CCOCC1] | Me | 530.4 |
| 179 | z-179 | 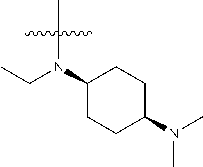 [SMILES: *N(CC)[C@H]1CC[C@@H](N(C)C)CC1] | 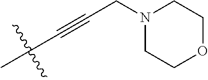 [SMILES: *C#CCN1CCOCC1] | Me | 571 |
| 180 | z-180 | 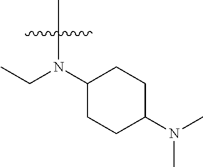 [SMILES: *N(CC)C1CCC(N(C)C)CC1] | 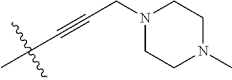 [SMILES: *C#CCN1CCN(C)CC1] | Me | 584 |
| 181 | z-181 | 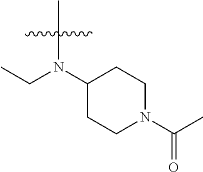 [SMILES: *N(CC)C1CCN(C(C)=O)CC1] | 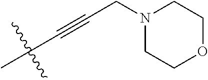 [SMILES: *C#CCN1CCOCC1] | Me | 571 |
| 182 | z-182 | 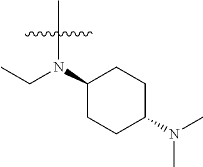 [SMILES: *N(CC)[C@H]1CC[C@H](N(C)C)CC1] | 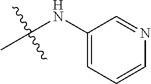 [SMILES: *Nc1cccnc1] | Me | 540.4 |
| 183 | z-183 | 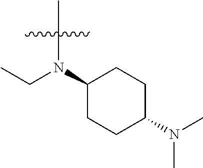 [SMILES: *N(CC)[C@H]1CC[C@H](N(C)C)CC1] | 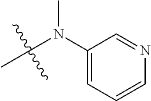 [SMILES: *N(C)c1cccnc1] | Me | 554.4 |
| 184 | z-184 | 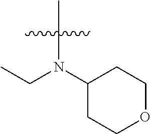 [SMILES: *N(CC)C1CCOCC1] | 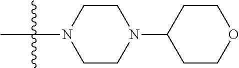 [SMILES: *N1CCN(C2CCOCC2)CC1] | Me | 575 |
| 185 | z-185 | 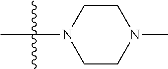 [SMILES: *N1CCN(C)CC1] | 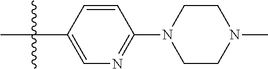 [SMILES: *c1ccc(N2CCN(C)CC2)nc1] | Me | 553 |
| 186 | z-186 | 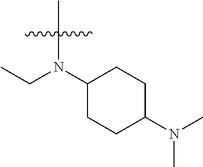 [SMILES: *N(CC)C1CCC(N(C)C)CC1] | 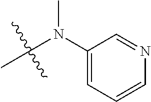 [SMILES: *N(C)c1cccnc1] | Me | 554.4 |
| 187 | z-187 | 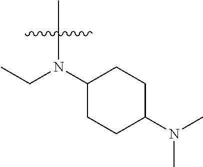 [SMILES: *N(CC)C1CCC(N(C)C)CC1] | 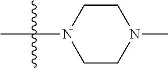 [SMILES: *N1CCN(C)CC1] | Me | 546 |
| 188 | z-188 | 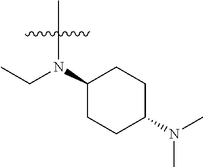 [SMILES: *N(CC)[C@H]1CC[C@H](N(C)C)CC1] | 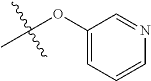 [SMILES: *Oc1cccnc1] | Me | 539 |
| 189 | z-189 | 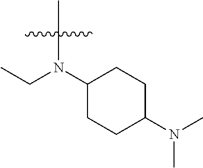 [SMILES: *N(CC)C1CCC(N(C)C)CC1] | 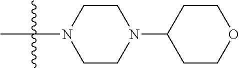 [SMILES: *N1CCN(C2CCOCC2)CC1] | Me | 616 |
| 190 | z-190 | 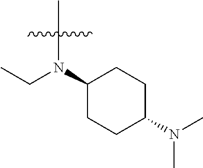 [SMILES: *N(CC)[C@H]1CC[C@H](N(C)C)CC1] | 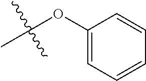 [SMILES: *Oc1ccccc1] | Me | 540 |
| 191 | z-191 | 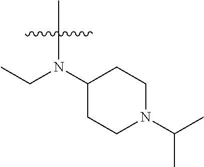 [SMILES: *N(CC)C1CCN(C(C)C)CC1] | 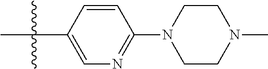 [SMILES: *c1ccc(N2CCN(C)CC2)nc1] | OMe | 639 |
| 192 | z-192 | 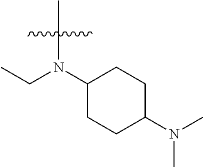 [SMILES: *N(CC)C1CCC(N(C)C)CC1] | 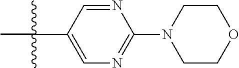 [SMILES: *c1cnc(N2CCOCC2)nc1] | OMe | 627.4 |
| 193 | z-193 | 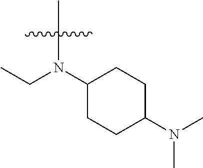 [SMILES: *N(CC)C1CCC(N(C)C)CC1] | 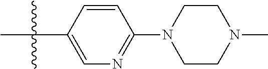 [SMILES: *c1ccc(N2CCN(C)CC2)nc1] | OMe | 639.4 |
| 195 | z-195 | 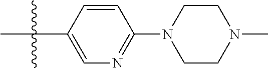 [SMILES: *c1ccc(N2CCN(C)CC2)nc1] | 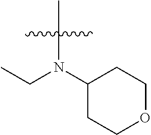 [SMILES: *N(CC)C1CCOCC1] | OMe | 598 |
| 196 | z-196 | 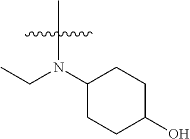 [SMILES: *N(CC)C1CCC(O)CC1] | 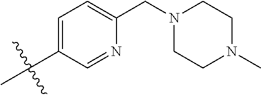 [SMILES: *c1ccc(CN2CCN(C)CC2)nc1] | Me | 610.5 |
| 197 | z-197 | 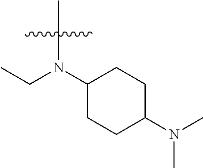 [SMILES: *N(CC)C1CCC(N(C)C)CC1] | Cl | OMe | 498 |
| 198 | z-198 | 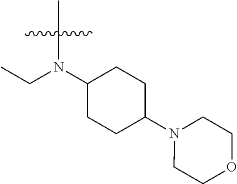 [SMILES: *N(CC)C1CCC(N2CCOCC2)CC1] | Cl | OMe | 540 |
| 199 | z-199 | 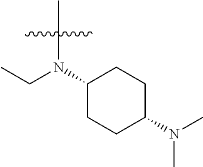 [SMILES: *N(CC)[C@H]1CC[C@@H](N(C)C)CC1] | Cl | OMe | 498 |
| 200 | z-200 | 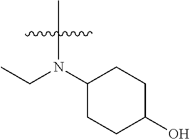 [SMILES: *N(CC)C1CCC(O)CC1] | CF₃ | Me | 489 |
| 201 | z-201 | 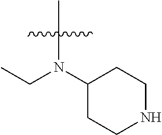 [SMILES: *N(CC)C1CCNCC1] | Cl | Me | 440 |
| 202 | z-202 | 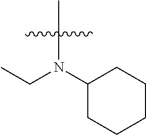 [SMILES: *N(CC)C1CCCCC1] | Cl | Me | 439 |
| 203 | z-203 | 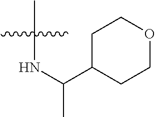 [SMILES: *NC(C)C1CCOCC1] | Cl | Me | 441 |
| 204 | z-204 | 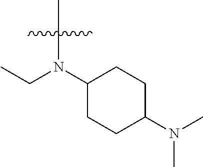 [SMILES: *N(CC)C1CCC(N(C)C)CC1] | Cl | CH₂CH₃ | 496 |
| 205 | z-205 | 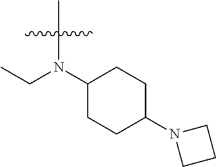 [SMILES: *N(CC)C1CCC(N2CCC2)CC1] | Cl | Me | 494 |
| 207 | z-207 | 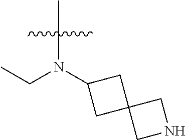 [SMILES: *N(CC)C1CC2(CNC2)C1] | Cl | Me | 452 |
| 208 | z-208 | 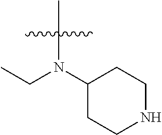 [SMILES: *N(CC)C1CCNCC1] | Cl | OMe | 456 |
| 209 | z-209 | 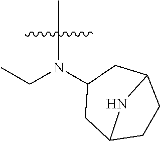 [SMILES: *N(CC)C1CC2CCC(C1)N2] | Cl | Me | 466 |
| 210 | z-210 | 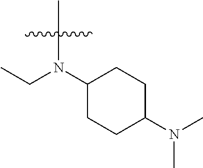 [SMILES: *N(CC)C1CCC(N(C)C)CC1] | 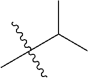 [SMILES: *C(C)C] | Me | 490.4 |
| 211 | z-211 | 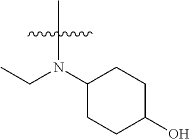 [SMILES: *N(CC)C1CCC(O)CC1] | 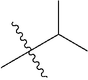 [SMILES: *C(C)C] | Me | 463.4 |
| 212 | z-212 | 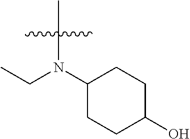 [SMILES: *N(CC)C1CCC(O)CC1] | 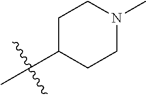 [SMILES: *C1CCN(C)CC1] | Me | 518.4 |
| 213 | z-213 | 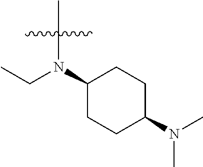 [SMILES: *N(CC)[C@H]1CC[C@@H](N(C)C)CC1] | 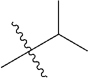 [SMILES: *C(C)C] | Me | 490.4 |
| 214 | z-214 | 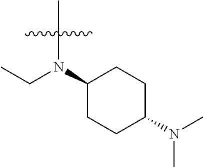 [SMILES: *N(CC)[C@H]1CC[C@H](N(C)C)CC1] | 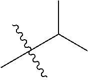 [SMILES: *C(C)C] | Me | 490.4 |
| 215 | z-215 | 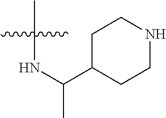 [SMILES: *NC(C)C1CCNCC1] | Cl | Me | 440 |

| | |
|---|---|
| z-23 | ¹H NMR (400 MHz, DMSO-d₆): δ 11.49 (br. s., 1H), 8.28 (s, 1H), 7.88 (d, 1H), 7.72 (d, 2H), 7.63 (s, 1H), 7.39 (d, 3H), 7.17 (d, 1H), 5.88 (s, 1H), 4.49 (d, 2H), 3.82 (d, 2H), 3.48 (s, 2H), 3.25-3.21 (m, 5H), 2.35 (br. s., 4H), 2.22 (s, 3H), 2.12 (s, 3H), 1.74-1.70 (m, 2H), 1.64-1.56 (m, 2H), 1.52-1.48 (m, 4H), 0.89-0.83 (m, 5H). |
| z-37 | ¹H NMR (400 MHz, DMSO-d₆): δ 11.47 (br. s., 1H), 8.14 (d, 2H), 7.84 (d, 1H), 7.68 (d, 1H), 7.54 (s, 1H), 7.41-7.36 (m, 3H), 7.09 (s, 1H), 5.85 (s, 1H), 4.46 (s, 2H), 3.44 (s, 2H), 3.26-3.20 (m, 2H), 3.90 (m, 1H), 2.31 (s, 4H), 2.21 (s, 3H), 2.18 (s, 3H), 2.08 (s, 3H), 1.80-1.75 (m, 2H), 1.75-1.70 (m, 2H), 1.50-1.25 (m, 8H), 1.25-1.00 (m, 6H), 1.85-1.75 (m, 3H). |
| z-39 | ¹H NMR (400 MHz, DMSO-d₆): δ 8.63 (d, 1H), 8.21-8.14 (m, 2H), 8.02 (dd, 1H), 7.88-7.84 (m, 1H), 7.61-7.57 (m, 1H), 7.30 (br. s., 1H), 7.09 (d, 1H), 6.94 (d, 1H), 5.89 (s, 1H), 4.48 (d, 2H), 3.74-3.71 (m, 4H), 3.51-3.48 (m, 4H), 3.23 (q, 2H), 3.19-3.15 (m, 1H), 2.98-2.89 (m, 2H), 2.36 (s, 1H), 2.33 (s, 3H), 2.21 (s, 3H), 2.12 (s, 3H), 1.97-1.81 (m, 5H), 1.50-1.40 (m, 2H), 1.28-1.15 (m, 2H), 0.90-0.85 (m, 3H). |
| z-40 | ¹H NMR (400 MHz, DMSO-d₆): δ 8.61 (d, 1H), 8.30-8.21 (m, 2H), 8.13 (s, 1H), 8.01 (dd, 1H), 7.83 (d, 1H), 7.56 (s, 1H), 7.28 (br. s., 1H), 7.07 (d, 1H), 6.92 (d, 1H), 5.87 (s, 1H), 4.46 (s, 2H), 3.72-3.69 (m, 4H), 3.49-3.46 (m, 4H), 3.21 (q, 2H), 2.96-2.88 (m, 1H), 2.66-2.58 (m, 1H), 2.36 (s, 6H), 2.19 (s, 3H), 2.10 (s, 3H), 1.93-1.83 (m, 4H), 1.50-1.38 (m, 2H), 1.30-1.18 (m, 2H), 0.85 (t, 3H). |
| z-43 | ¹H NMR (400 MHz, DMSO-d₆): δ 8.62 (d, 1H), 8.25 (br. s., 2H), 8.17 (s, 1H), 8.01 (dd, 1H), 7.86 (d, 1H), 7.59 (s, 1H), 7.32-7.28 (m, 1H), 7.16 (d, 1H), 6.94 (s, 1H), 5.89 (s, 1H), 4.48 (d, 2H), 3.74-3.71 (m, 4H), 3.51-3.48 (m, 4H), 3.17 (q, 2H), 2.26 (s, 6H), 2.22 (s, 3H), 2.12 (s, 3H), 1.92-1.84 (m, 2H), 1.78-1.68 (m, 2H), 1.50-1.41 (m, 2H), 1.40-1.30 (m, 2H), 0.85 (t, 3H). |
| z-44 | ¹H NMR (400 MHz, DMSO-d₆): δ 11.46 (br. s., 1H), 8.82 (s, 2H), 8.14 (s, 1H), 7.84 (d, 1H), 7.55 (s, 1H), 7.19-7.16 (m, 1H), 7.06 (d, 1H), 5.87 (s, 1H), 4.46 (d, 2H), 3.79-3.75 (m, 4H), 3.20 (q, 2H), 2.93-2.85 (m, 1H), 2.37-2.34 (m, 4H), 2.19 (d, 6H), 2.10 (s, 10H), 1.89-1.84 (m, 2H), 1.76-1.73 (m, 2H), 1.47-1.36 (m, 2H), 1.12-1.01 (m, 2H), 0.85 (t, 3H). |
| z-46 | ¹H NMR (400 MHz, DMSO-d₆): δ 11.47 (br. s., 1H), 8.88 (s, 2H), 8.17 (s, 1H), 7.86 (d, 1H), 7.58 (s, 1H), 7.22-7.19 (m, 1H), 7.08 (d, 1H), 5.89 (s, 1H), 4.48 (d, 2H), 3.78-3.74 (m, 4H), 3.70-3.67 (m, 4H), 3.22 (q, 2H), 2.96-2.87 (m, 1H), 2.21 (s, 3H), 2.11 (s, 10H), 2.90-1.86 (m, 2H), 1.78-1.74 (m, 2H), 1.45-1.40 (m, 2H), 1.12-1.07 (m, 2H), 0.87 (t, 3H). |
| z-49 | ¹H NMR (400 MHz, DMSO-d₆): δ 8.57 (d, 1H), 8.17 (s, 2H), 8.12 (s, 1H), 7.97 (dd, 1H), 7.83 (d, 1H), 7.55 (s, 1H), 7.28-7.27 (m, 1H), 7.07 (d, 1H), 6.91 (d, 1H), 5.87 (s, 1H), 4.46 (d, 2H), 3.54-3.51 (m, 6H), 3.21 (q, 2H), 2.95-2.85 (m, 1H), 2.66-2.56 (m, 1H), 2.47-2.44 (m, 1H), 2.42-2.38 (m, 4H), 2.36 (s, 4H), 2.20 (d, 6H), 2.10 (s, 3H), 1.93-1.81 (m, 2H), 1.47-1.41 (m, 2H), 1.25-1.20 (m, 2H), 0.86 (t, 3H). |
| z-51 | ¹H NMR (400 MHz, DMSO-d₆): δ 8.84 (s, 2H), 8.20 (s, 1H), 7.87 (d, 1H), 7.62 (s, 1H), 7.23-7.20 (m, 1H), 7.16 (d, 1H), 5.89 (s, 1H), 4.47 (d, 2H), 3.81-3.77 (m, 4H), 3.47-3.44 (m, 1H), 3.17 (q, 2H), 2.39-2.36 (m, 4H), 2.29 (s, 6H), 2.21 (d, 6H), 2.21-2.19 (m, 1H), 2.12 (s, 3H), 1.92-1.86 (m, 2H), 1.76-1.70 (m, 2H), 1.49-1.33 (m, 4H), 0.85 (t, 3H). |
| z-52 | ¹H NMR (400 MHz, DMSO-d₆): δ 11.45 (br. s., 1H), 8.88 (d, 1H), 8.22 (s, 1H), 8.11 (dd, 1H), 7.86 (d, 1H), 7.58 (s, 1H), 7.48 (d, 1H), 7.32-7.30 (m, 1H), 7.07 (d, 1H), 5.86 (s, 1H), 4.45 (d, 2H), 3.60 (s, 2H), 3.22 (q, 2H), 2.95-2.85 (m, 1H), 2.42-2.38 (m, 2H), 2.34-2.28 (m, 3H), 2.18 (s, 3H), 2.13-2.08 (m, 15H), 1.90-1.82 (m, 2H), 1.77-1.71 (m, 2H), 1.44-1.40 (m, 2H), 1.09-1.04 (m, 2H), 0.85 (t, 3H). |
| z-53 | ¹H NMR (400 MHz, DMSO-d₆): δ 11.45 (br. s., 1H), 8.56 (d, 1H), 8.13 (s, 1H), 7.95 (d, 1H), 7.83 (d, 1H), 7.56 (s, 1H), 7.27 (s, 1H), 7.13 (d, 1H), 6.90 (d, 1H), 5.86 (s, 1H), 4.45 (d, 2H), 3.53-3.49 (m, 4H), 3.49-3.47 (m, 1H), 3.27 (s, 2H), 3.13 (q, 2H), 2.40-2.37 (m, 5H), 2.32-2.24 (m, 4H), 2.19 (d, 6H), 2.09 (s, 3H), 1.92-1.82 (m, 2H), 1.76-1.64 (m, 2H), 1.50-1.30 (m, 4H), 0.81 (t, 3H). |
| z-54 | ¹H NMR (400 MHz, DMSO-d₆): δ 8.25 (s, 2H), 8.18 (s, 1H), 7.88 (s, 1H), 7.73 (d, 2H), 7.58 (s, 1H), 7.39 (d, 3H), 7.11 (d, 1H), 5.89 (s, 1H), 4.49 (d, 2H), 3.50 (s, 2H), 3.23 (q, 2H), 2.99-2.92 (m, 1H), 2.69-2.63 (m, 1H), 2.44-2.39 (m, 13H), 2.20 (d, 6H), 2.12 (s, 3H), 1.95-1.86 (m, 4H), 1.50-1.42 (m, 2H), 1.28-1.23 (m, 2H), 0.88 (t, 3H). |
| z-56 | ¹H NMR (400 MHz, DMSO-d₆): δ 8.58 (s, 1H), 8.17 (s, 1H), 7.98 (d, 1H), 7.83 (d, 1H), 7.61 (s, 1H), 7.30-7.28 (m, 1H), 7.10 (d, 1H), 6.89 (d, 1H), 5.86 (s, 1H), 4.45 (d, 2H), 3.51-3.50 (m, 4H), 3.19-3.17 (m, 4H), 2.98-2.93 (m, 1H), 2.73-2.70 (m, 2H), 2.38-2.36 (m, 4H), 2.19 (s, 3H), 2.18 (s, 3H), 2.09 (s, 3H), 1.90-1.85 (m, 2H), 1.73-1.69 (m, 2H), 0.82 (t, 3H). |
| z-64 | ¹H NMR (400 MHz, DMSO-d₆): δ 11.49 (br. s., 1H), 9.04 (s, 1H), 8.58 (d, 1H), 8.31 (s, 1H), 8.18 (d, 1H), 7.91 (d, 1H), 7.66 (s, 1H), 7.52-7.48 (m, 1H), 7.37 (s, 1H), 7.16 (d, 1H), 5.89 (s, 1H), 4.49 (d, 2H), 3.40-3.32 (m, 1H), 3.19 (q, 2H), 2.21 (s, 3H), 2.16 (s, 6H), 2.12 (s, 3H), 2.05-2.03 (m, 1H), 1.88-1.86 (m, 2H), 1.75-1.72 (m, 2H), 1.50-1.44 (m, 2H), 1.31-1.28 (m, 2H), 0.86 (t, 3H). |
| z-72 | ¹H NMR (400 MHz, DMSO-d₆): δ 11.50 (br. s., 1H), 8.08 (s, 1H), 7.83 (d, 1H), 7.65 (d, 2H), 7.52 (s, 1H), 7.35-7.31 (m, 1H), 7.08 (d, 1H), 7.03 (d, 2H), 5.88 (s, 1H), 4.48 (d, 2H), 3.23-3.16 (m, 6H), 2.94-2.87 (m, 1H), 2.48-2.45 (m, 4H), 2.23 (s, 3H), 2.21 (s, 3H), 2.13 (s, 6H), 2.12 (s, 3H), 1.91-1.86 (m, 2H), 1.79-1.75 (m, 2H), 1.49-1.37 (m, 2H), 1.16-1.03 (m, 2H), 0.87 (t, 3H). |
| z-74 | ¹H NMR (400 MHz, DMSO-d₆): δ 11.46 (br. s., 1H), 8.56 (d, 1H), 8.12 (s, 1H), 7.95 (dd, 1H), 7.81 (d, 1H), 7.54 (s, 1H), 7.27-7.24 (m, 1H), 7.08 (d, 1H), 6.89 (d, 1H), 5.86 (s, 1H), 4.45 (d, 2H), 3.52-3.49 (m, 4H), 3.17 (q, 2H), 2.98-2.92 (m, 1H), 2.87-2.83 (m, 2H), 2.39-2.36 (m, 4H), 2.19 (s, 3H), 2.18 (s, 3H), 2.09 (s, 3H), 2.01 (t, 2H), 1.71-1.64 (m, 2H), 1.53-1.42 (m, 3H), 0.82 (t, 3H), 0.32-0.30 (m, 2H), 0.20-0.18 (m, 2H). |
| z-80 | ¹H NMR (400 MHz, DMSO-d₆): δ 11.50 (br. s., 1H), 8.27 (s, 1H), 8.14 (s, 1H), 7.86 (d, 1H), 7.65 (dd, 1H), 7.56-7.53 (m, 2H), 7.34-7.31 (m, 1H), 7.13-7.07 (m, 2H), 5.89 (s, 1H), 4.48 (d, 2H), 3.25-3.22 (m, 2H), 3.07-3.04 (m, 4H), 2.96-2.86 (m, 1H), 2.50-2.47 (m, 4H), 2.24 (s, 3H), 2.21 (s, 3H), 2.16 (s, 6H), 2.12 (s, 3H), 1.91-1.86 (m, 2H), 1.81-1.76 (m, 2H), 1.46-1.41 (m, 2H), 1.14-1.09 (m, 2H), 0.87 (t, 3H). |
| z-86 | ¹H-NMR(400M, DMSO-d₆): δ11.45(s, 1H), 8.55(s, 1H), 8.08(s, 1H), 7.95(dd, 1H), 7.81(d, 1H), 7.59-7.51(m, 2H), 7.24(s, 1H), 7.09-7.04(m, 1H), 6.90(d, 1H ), 5.86(s, 1H), 4.45-4.41(m, 2H), 4.27(d, 1H), 3.61(br, 1H), 3.50(t, 4H), 3.27-3.21(m, 2H), 308-3.02(m, 1H), 2.38(t, 4H), 2.19-2.18(m, 6H), 2.09(s, 3H), 1.84-1.75(m, 3H), 1.61-1.58(m, 2H), 1.49-1.46(m, 2H), 1.31-1.24(m, 2H), 0.84(t, 3H). |
| z-89 | ¹H-NMR(400M, DMSO-d₆): δ11.45(s, 1H), 8.80(d, 1H), 8.12(s, 1H), 7.83(d, 1H), 7.55(d, 1H), 7.16(s, 1H), 7.09-7.04(m, 1H), 5.86(s, 1H), 4.45-4.41(m, 4H), 4.28(s, 1H), 3.77-3.74(m, 4H), 3.61(s, 1H), 3.21-3.17(m, 2H), 3.13-3.10(m, 1H), 2.35-2.33(m, 4H), 2.17(s, 6H), 2.09(s, 3H), 1.81-1.75(m, 2H), 1.61-1.58(m, 2H), 1.49-1.46(m, 2H), 1.27-1.25(m, 2H), 0.87-0.84(m, 3H). |
| z-91 | ¹H NMR (400 MHz, dmso) δ 11.46 (s, 1H), 7.89-7.67 (m, 2H), 7.37 (s, 1H), 7.23 (s, 1H), 7.08-7.02 (m, 1H), 6.20 (s, 1H), 5.85 (s, 1H), 4.43 (s, 2H), 4.28 (m, 1H), 3.60 (m, 1H), 3.27-3.23 (m, 2H), 3.15-3.14 (m, 2H), 3.04-2.91 (m, 3H), 2.56 (s, 3H), 2.26 (s, 3H), 2.17 (s, 2H), 2.09 (s, 3H), 1.83-1.52 (m, 4H), 1.50-1.33 (m, 2H), 1.32-1.13 (m, 2H), 0.80 (t, 3H). |
| z-93 | ¹H NMR (400 MHz, DMSO-d₆): δ 8.84 (d, 1H), 8.24 (s, 1H), 8.22 (s, 1H), 8.04 (dd, 1H), 7.88 (d, 1H), 7.59 (s, 1H), 7.39 (d, 1H), 7.32-7.30 (m, 1H), 7.10 (d, 1H), 5.89 (s, 1H), 4.48 (d, 2H), 3.23 (q, 2H), 2.97-2.90 (m, 1H), 2.21 (s, 3H), 2.18 (s, 6H), 2.12 (s, 3H), 1.93-1.87 (m, 2H), 1.82-1.76 (m, 2H), 1.47-1.41 (m, 2H), 1.16-1.11 (m, 2H), 1.00-0.95 (m, 4H), 0.88 (t, 3H). |
| z-94 | ¹H NMR (400 MHz, DMSO-d₆): δ 8.58 (s, 1H), 8.16 (s, 1H), 7.97 (d, 1H), 7.83 (d, 1H), 7.60 (s, 1H), 7.31 (br. s., 1H), 7.08 (d, 1H), 6.90 (s, 1H), 5.86 (s, 1H), 4.45 (s, 2H), 3.52-3.50 (m, 4H), 3.30-3.20 (m, 1H), 3.17 (q, 2H), 2.40-2.38 (m, 4H), 2.20 (s, 3H), 2.18 (s, 3H), 2.09 (s, 3H), 1.97-1.95 (m, 2H), 1.78-1.65 (m, 6H), 0.83 (t, 3H). |
| z-99 | ¹H NMR (400 MHz, DMSO) δ 11.62 (br. s, 1H), 8.86 (s, 2H), 8.22 (s, 1H), 8.10 (s, 1H), 7.80 (d, 1H), 7.64 (s, 1H), 7.14 (s, 1H), 5.94 (s, 1H), 4.46 (d, 2H), 4.30 (s, 1H), 3.78-3.58 (m, 9H), 3.24-3.13 (m, 2H), 3.08-2.99 (m, 1H), 2.22 (s, 3H), 2.11 (s, 3H), 1.82-1.72 (m, 2H), 1.66-1.55 (m, 2H), 1.51-1.41 (m, 2H), 1.32-1.19 (m, 2H), 0.84 (t, 3H). |
| z-100 | ¹H-NMR(400M, DMSO-d₆): δ11.47(s, 1H), 8.56(s, 1H), 8.10(s, 1H), 7.95(d, 1H), 7.82(d, 1H), 7.52(s, 1H), 7.26-7.24(m, 1H), 7.08(d, 1H), 6.90(d, 1H), 5.86(s, 1H), 4.45 (s, 2H), 4.30(s, 1H), 3.61(s, 1H), 3.52-3.49(m, 4H), 3.22-3.19(m, 2H), 3.13-3.10(m, 1H), 3.06-3.04(m, 1H), 2.39-2.37(m, 4H), 2.19-2.18(m, 6H) 2.09(s, 3H), 1.82-1.79(m, 2H), 1.62-1.59(m, 2H), 1.50-1.46(m, 2H), 1.30-1.27(m, 2H), 0.85(t, 3H). |
| z-101 | ¹H-NMR(400M, DMSO-d₆): δ11.48(s, 1H), 8.56(s, 1H), 8.11(s, 1H), 7.98-7.95(m, 1H), 7.82(d, 1H), 7.53(s, 1H), 7.29(br, 1H), 7.06(d, 1H), 6.91(d, 1H), 5.88(s, 1H), 4.46-4.43(s, 3H), 4.54-3.51(m, 4H), 3.21-3.18(m, 2H), 2.94-2.88(m, 1H), 2.43-2.47(m, 4H), 2.22-2.17(m, 6H), 2.10(s, 3H), 1.78-1.75(m, 3H), 1.46-1.41(m, 3H), 1.38-1.32(m, 2H), 1.04-1.01(m, 2H), 0.85-0.82(m, 3H). |
| z-127 | ¹H NMR (400 MHz, CD₃OD): δ 8.75 (s, 1H), 8.17 (s, 1H), 8.01 (dd, 1H), 7.88 (d, 1H), 7.65 (d, 1H), 7.28 (d, 1H), 6.14 (s, 1H), 4.64 (s, 2H), 3.80 (d, 2H), 3.34-3.30 (m, 2H), 3.07-3.00 (m, 1H), 2.66-2.46 (m, 8H), 2.36 (s, 3H), 2.26 (d, 6H), 2.25 (s, 6H), 2.24-2.20 (m, 1H), 1.98 (dd, 4H), 1.59-1.49 (m, 2H), 1.26-1.16 (m, 2H), 0.97 (s, 3H). |
| z-129 | ¹H NMR (400 MHz, DMSO-d₆): δ 8.56 (s, 1H), 8.10 (s, 1H), 7.95 (dd, 1H), 7.81 (d, 1H), 7.52 (d, 1H), 7.29 (br. s., 1H), 7.07 (d, 1H), 6.90 (d, 1H), 5.86 (s, 1H), 4.44 (d, 2H), 3.52-3.49 (m, 4H), 3.22-3.13 (m, 6H), 2.41-2.38 (m, 4H), 2.20 (s, 3H), 2.18 (s, 3H), 2.09 (s, 3H), 1.95-1.90 (m, 1H), 1.82-1.60 (m, 4H), 1.53-1.49 (m, 2H), 1.30-1.19 (m, 2H), 0.83 (t, 3H). |
| z- 132 | ¹H-NMR (400M, DMSO-d₆): δ11.43(s, 1H), 8.08-8.05(m, 1H), 7.87-7.85(m, 1H), 7.27-7.24(m, 1H), 7.19-7.16(m, 1H), 7.06-6.99(m, 1H ), 5.85(s, 1H), 4.38 (s, 2H), 3.28-3.24(m, 1H), 3.13-3.09(m, 4H), 2.89-2.85(m, 1H), 2.17-2.09(m, 11H), 1.81-1.78(m, 4H), 1.42-1.39(m, 2H), 1.26-1.24(m, 1H), 1.09-1.07(m, 2H), 0.87-0.77(m, 3H). |
| z-135 | ¹H NMR (400 MHz, DMSO-d₆) δ 8.05 (s, 1H), 7.86 (d, 1H), 7.35 (s, 1H), 7.17 (s, 1H), 6.98 (d, 1H), 5.85 (s, 1H), 4.38 (d, 2H), 3.18-3.12 (m, 2H), 3.08 (s, 2H), 2.89-2.73 (m, 2H), 2.18-2.12 (m, 3H), 2.09 (s, 3H), 2.05 (s, 4H), 1.76 (dd, 4H), 1.39 (dd, 3H), 1.20 (s, 3H), 1.02 (dd, 2H), 0.81 (t, 1H). |
| z-136 | ¹H NMR (400 MHz, DMSO-d₆)δ 11.41 (s, 1H), 8.07 (s, 1H), 7.86 (d, 1H), 7.27 (s, 1H), 7.17 s, 1H), 7.06 (d, 1H), 5.85 (s, 1H), 4.39 (d, 2H), 3.26-3.24(m, 1H), 3.10 (m, 2H), 2.82-2.79(m, 1H), 2.19-2.07 (m, 10H), 2.02 (s, 1H), 1.68-1.67 (m, 4H), 1.42-1.40 (m, 2H), 1.28-1.22 (m, 2H), 1.11-1.08 (m, 1H), 0.81-0.78 (m, 3H). |
| z-137 | ¹H-NMR(400M, DMSO-d₆): δ11.43(br, 1H), 8.04(s, 1H), 7.86 (d, 1H), 7.24(d, 1H), 7.17-7.15(m, 1H), 6.99(d, 1H ), 5.85(s, 1H), 4.38 (s, 2H), 3.39-3.38(d, 2H), 3.15-3.10(m, 2H), 2.86-2.80(m, 2H), 2.16-2.10(m, 11H), 1.82-1.73(m, 4H), 1.45-1.42(m, 2H), 1.20-1.10(m, 2H), 0.83-0.79(m, 3H). |
| z-138 | ¹H-NMR(400M, DMSO-d₆): δ 11.41 (s, 1H), 7.98 (d, 1H), 7.85 (d, 1H), 7.23 (d, 1H), 7.14-7.12 (m, 1H), 6.99 (d, 1H), 6.10 (s, 1H), 4.30 (d, 2H), 3.77 (s, 3H), 3.13 (q, 2H), 2.89-2.83(m, 1H), 2.14 (d, 9H), 1.77 (m, 4H), 1.40 (q, 2H), 1.06 (q, 2H), 0.81 (t, 3H). |
| z-139 | ¹H NMR (400 MHz, DMSO-d₆): δ 8.13 (s, 1H), 7.90 (d, 1H), 7.36 (d, 1H), 7.21 (t, 1H), 7.04 (d, 1H), 5.85 (s, 1H), 4.40 (d, 2H), 3.41-3.49 (m, 1H), 3.12 (q, 2H), 3.08-2.94 (m, 4H), 2.16 (s, 3H), 2.09-2.04 (m, 7H), 0.81 (t, 3H). |
| z-144 | ¹H NMR (400 MHz, DMSO-d₆) δ 11.42 (s, 1H), 8.10 (s, 1H), 7.87 (d, 1H), 7.31 (s, 1H), 7.18 (s, 1H), 7.05 (d, 1H), 5.85 (s, 1H), 4.39 (d, 2H), 4.24 (d, 1H), 3.71 (d, 1H), 3.21-3.05 (m, 3H), 2.90 (t, 1H), 2.16 (s, 3H), 2.09 (s, 3H), 1.92 (s, 3H), 1.71 (s, 2H), 1.49 (d, 1H), 1.36 (d, 1H), 0.80 (t, 3H). |
| z-145 | ¹H NMR (400 MHz, DMSO-d₆)δ 11.42 (s, 1H), 8.04 (d, 1H), 7.85 (d, 1H), 7.23 (d, 1H), 7.15 (s, 1H), 6.99 (d, 1H), 5.85 (s, 1H), 4.39 (d, 2H), 3.29-3.25 (m, 2H), 3.16 (s, 3H), 3.12-3.10 (m, 2H), 2.85-2.82 (m, 1H), 2.45-2.42 (m, 2H), 2.28-2.25(m, 1H), 2.16 (s, 3H), 2.09-2.06 (m, 6H) 1.80-1.78 (m, 3H), 1.66-1.64 (m, 2H), 1.42-1.40 (m, 2H), 1.10-1.08 (m, 2H), 0.81 (t, 3H). |
| z-146 | ¹H NMR (400 MHz, DMSO-d₆) δ 11.42 (s, 1H), 8.08 (d, 1H), 7.86 (d, 1H), 7.28 (d, 1H), 7.17 (t, 1H), 7.03 (d, 1H), 5.85 (s, 1H), 4.39 (d, 2H), 3.18-2.99 (m, 4H), 2.92-2.89 (m, 3H), 2.23-2.21 (m, 2H), 2.16 (s, 3H), 2.09 (s, 3H), 1.68-1.47 (m, 5H), 0.79 (t, 3H). |
| z-149 | ¹H NMR (400 MHz, DMSO-d₆) δ 11.43 (s, 1H), 8.05 (s, 1H), 7.87 (d, 1H), 7.23 (d, 1H), 7.16 (t, 1H), 7.01 (d, 1H), 5.85 (s, 1H), 4.39 (d, 2H), 3.90-3.88( m, 1H), 3.42-3.40 (m, 2H), 3.13-3.08 (m, 6H), 2.62-2.60 (m, 1H), 2.17 (s, 3H), 2.11-2.09 (m, 4H), 1.73-1.71 (m, 2H), 1.50-1.48 (m, 2H), 1.45-1.33 (m, 4H), 1.23-1.13 (m, 2H), 0.80 (t, 3H). |
| z-150 | ¹H NMR (400 MHz, DMSO-d₆)δ 11.43 (s, 1H), 8.05 (d, 1H), 7.86 (d, 1H), 7.25 (d, 1H), 7.16 (d, 1H), 6.99 (d, 1H), 5.86 (s, 1H), 4.39 (d, 2H), 3.84-3.81 (m, 1H), 3.38-3.35 (m, 2H), 3.16-3.08 (m, 5H), 2.88-2.86 (m, 1H), 2.66-2.64 (m, 2H), 2.17 (s, 3H), 2.10 (s, 3H), 1.85-1.64 (m, 5H), 1.50-1.32 (m, 4H), 0.85-0.69 (m, 3H). |
| z-152 | ¹H NMR (400 MHz, DMSO) δ 11.44 (br. s, 1H), 8.89 (d, 1H), 8.27 (s, 1H), 8.12 (dd, 1H), 7.88 (d, 1H), 7.65 (s, 1H), 7.49 (d, 1H), 7.32 (t, 1H), 7.14 (d, 1H), 5.86 (s, 1H), 4.45 (d, 2H), 3.81-3.78 (m, 2H), 3.57 (s, 2H), 3.25-3.12 (m, 5H), 2.69-2.66 (m, 4H), 2.38-2.34 (m, 4H), 2.18 (s, 3H), 2.09 (s, 3H), 1.73-1.67 (m, 2H), 1.61-1.51 (m, 2H), 0.84 (t, 3H). |
| z-175 | ¹H NMR (400 MHz, DMSO-d₆) δ 11.42 (s, 1H), 8.11 (d, 1H), 7.87 (d, 1H), 7.33 (d, 1H), 7.19 (s, 1H), 7.06 (d, 1H), 5.86 (s, 1H), 4.39 (d, 2H), 4.19 (d, 1H), 3.97 (d, 2H), 3.56 (d, 1H), 3.20-3.18 (m, 1H), 3.13-3.10 (m, 2H), 2.92 (t, 1H), 2.59-2.57 (m, 1H), 2.17 (s, 3H), 2.10 (s, 3H), 1.74-1.72 (m, 2H), 1.62-1.49 (m, 1H), 1.42-1.40 (m, 1H), 0.80 (t, 3H). |
| z-176 | ¹H NMR (400 MHz, DMSO-d₆) δ 11.42 (s, 1H), 8.10 (t, 1H), 7.87 (d, 1H), 7.32 (d, 1H), 7.18 (s, 1H), 7.05 (d, 1H), 5.85 (s, 1H), 4.45-4.36 (m, 3H), 4.24-4.21 (m, 1H), 4.10-3.94 (m, 2H), 3.60 (m, 1H), 3.24-3.07 (m, 3H), 2.85-2.83 (m, 1H), 2.58-2.55 (m, 1H), 2.17 (s, 3H), 2.09 (s, 3H), 1.73-1.71(m, 2H), 1.55-1.37 (m, 2H), 0.80 (t, 3H). |
| z-177 | ¹H NMR (400 MHz, DMSO-d₆): δ 11.44 (br. s., 1H), 8.08 (s, 1H), 7.89 (d, 1H), 7.30-7.27 (m, 1H), 7.24 (s, 1H), 7.05 (d, 1H), 5.88 (s, 1H), 4.43 (d, 2H), 3.63-3.60 (m, 4H), 3.51 (s, 2H), 3.14 (q, 2H), 2.88-2.80 (m, 1H), 2.53-2.50 (m, 4H), 2.22 (s, 3H), 2.13-2.12 (m, 9H), 1.85-1.74 (m, 4H), 1.44-1.39 (m, 2H), 1.11-1.06 (m, 2H), 0.82 (t, 3H). |
| z-178 | ¹H NMR (400 MHz, DMSO-d₆): δ 11.45 (br. s., 1H), 8.13 (s, 1H), 7.90 (d, 1H), 7.31 (s, 2H), 7.11 (d, 1H), 5.88 (s, 1H), 4.43 (d, 2H), 3.81 (d, 2H), 3.63-3.60 (m, 4H), 3.52 (s, 2H), 3.23-3.12 (m, 5H), 2.54-2.50 (m, 4H), 2.20 (s, 3H), 2.12 (s, 3H), 1.68-1.64 (m, 2H), 1.60-1.52 (m, 2H), 0.81 (t, 3H). |
| z-192 | ¹H NMR (400 MHz, DMSO-d₆): δ 8.85 (s, 2H), 8.09 (s, 1H), 7.82 (d, 1H), 7.56 (s, 1H), 7.16-7.14 (m, 1H), 7.04 (d, 1H), 6.12 (s, 1H), 4.33 (d, 2H), 3.77 (s, 3H), 3.74-3.71 (m, 4H), 3.67-3.64 (m, 4H), 3.23-3.18 (q, 2H), 2.92-2.89 (m, 1H), 2.49-2.30 (m, 1H), 2.21 (s, 6H), 2.18 (s, 3H), 1.90-1.86 (m, 2H), 1.82-1.77 (m, 2H), 1.44-1.39 (m, 2H), 1.16-1.11 (m, 2H), 0.84 (t, 3H). |
| z-193 | ¹H NMR (400 MHz, DMSO-d₆): δ 11.51 (br. s., 1H), 8.58 (d, 1H), 8.06 (s, 1H), 7.97 (dd, 1H), 7.84 (d, 1H), 7.54 (s, 1H), 7.29 (br. s., 1H), 7.07 (d, 1H), 6.93 (d, 1H), 6.15 (s, 1H), 4.37 (d, 2H), 3.81 (s, 3H), 3.55-3.52 (m, 4H), 3.23 (q, 2H), 2.96-2.88 (m, 1H), 2.43-2.39 (m, 4H), 2.21-2.12 (m, 13H), 1.91-1.87 (m, 2H), 1.79-1.75 (m, 2H), 1.48-1.38 (m, 2H), 1.15-1.08 (m, 2H), 0.87 (t, 3H). |
| z-196 | ¹H NMR (400 MHz, dmso) δ 11.45 (s, 1H), 8.88 (s, 1H), 8.22 (s, 1H), 8.11 (d, 1H), 7.87 (d, 1H), 7.58 (s, 1H), 7.48 (d, 1H), 7.30 (s, 1H), 7.11 (d, 1H), 5.86 (s, 1H), 4.45 (d, 2H), 4.28 (d, 1H), 3.61 (s, 3H), 3.29-3.26 (m, 4H), 3.22-3.19 (m, 2H), 3.07 (s, 1H), 2.43-2.34 (m, 4H), 2.18 (s, 5H), 2.09 (s, 3H), 1.82-1.75 (m, 2H), 1.64-1.55 (m, 2H), 1.52-1.48 (m, 2H), 1.31-1.20(m, 2H), 1.08-0.97 (m, 1H), 0.86 (t, 3H). |
| z-197 | ¹H NMR (400 MHz, DMSO-d₆)δ 11.42 (s, 1H), 8.04-7.96 (m, 1H), 7.85 (dd, 1H), 7.24 (dd, 1H), 7.12 (t, 1H), 7.01 (dd, 1H), 6.10 (s, 1H), 4.29 (d, 2H), 3.77 (s, 3H), 3.12 (m, 2H), 2.85 (m, 1H), 2.18 (s, 3H), 2.10 (s, 2H), 2.06 (s, 5H), 1.76 (m, 4H), 1.43-1.40 (m, 2H), 1.26-1.24(m, 1H), 1.11-0.96 (m, 2H), 0.80 (m, 3H). |
| z-199 | ¹H NMR (400 MHz, CDCl₃): δ 7.99 (d, 1H), 7.71 (dd, 1H), 7.56-7.47 (m, 1H), 7.33 (s, 1H), 7.18 (dd, 2H), 5.99 (s, 1H), 4.71 (d, 2H), 3.88 (s, 3H), 3.39 (s, 1H), 3.18 (q, 2H), 2.44 (s, 3H), 2.29 (d, 6H), 2.01 (dd, 5H), 1.75 (d, 4H), 1.45 (dd, 5H), 1.24 (s, 8H), 0.96 (t, 2H), 0.88 (dd, 4H). |
| z-201 | ¹H NMR (400 MHz, DMSO-d₆)δ 8.30 (s, 1H), 8.16 (s, 1H), 7.91 (d, 1H), 7.39 (s, 1H), 7.08 (d, 1H), 5.89 (s, 1H), 4.42 (d, 2H), 3.17-3.14 (m, 5H), 2.70 (t, 2H), 2.20 (s, 3H), 2.13 (s, 3H), 1.86-1.84 (m, 2H), 1.71-1.68(m, 2H), 0.84 (t, 3H). |
| z-204 | ¹H NMR (400 MHz, DMSO-d₆) δ 11.43 (s, 1H), 8.04 (dd, 1.8 Hz, 1H), 7.86 (dd, 1H), 7.25 (dd, 1H) 7.17 (t, 1H), 7.03 (dd, 1H), 5.90 (s, 1H), 4.40 (d, 2H), 3.15-3.12 (m, 2H), 2.87-2.85 (m, 1H), 2.56-2.47 (m, 2H), 2.20-2.07 (m, 10H), 1.82-1.83 (m, 4H), 1.48-1.34 (m, 2H), 1.32-1.18 (m, 1H), 1.08-1.06 (m, 4H), 0.80 (q, 3H). |
| z-207 | ¹H NMR (400 MHz, DMSO-d₆) δ 8.04 (d, 1H), 7.86 (d, 1H), 7.15 (s, 1H), 7.03 (dd, 2H), 5.85 (s, 1H), 4.39 (d, 2H), 3.76-374 (m, 2H), 3.48-3.46(m, 2H), 3.45-3.42 (m, 3H), 3.09-3.07 (m, 2H), 2.31-2.29 (m, 2H), 2.16 (s, 3H), 2.10 (s, 3H), 1.76-1.74 (m, 2H), 0.73 (t, 3H). |
| z-208 | ¹H NMR (400 MHz, DMSO-d₆): 8.00 (s, 1H), 7.85 (d, 1H), 7.25 (d, 1H), 7.13 (t, 1H), 7.01 (d, 1H), 6.15 (s, 1H), 4.29 (d, 2H), 3.77 (s, 3H), 3.13 (q, 2H), 3.00-2.84 (m, 3H), 2.26 (t, 2H), 2.18 (s, 3H), 1.64 (d, 2H), 1.48-1.36 (m, 2H), 0.82 (t, 3H). |
| z-209 | ¹H NMR (400 MHz, DMSO-d₆) δ 12.46 (s, 1H), 9.86 (s, 1H), 8.63 (br, 1H), 8.46 (d, 1H), 7.80 (d, 1H), 7.70 (d, 1H), 7.14 (d, 1H), 6.25 (s, 1H), 4.52 (d, 2H), 3.90-3.81 (m, 6H), 3.28-3.25 (m, 2H), 2.37 (s, 3H), 2.21 (s, 3H), 2.05-2.02 (m, 3H), 1.86-1.83 (m, 2H), 0.70 (t, 3H). |
| z-213 | ¹H NMR (400 MHz, DMSO-d₆): δ 11.45 (br. s., 1H), 7.75 (d, 1H), 7.61 (s, 1H), 7.18 (s, 1H), 7.06 (d, 2H), 5.82 (s, 1H), 4.40 (d, 2H), 3.05 (q, 2H), 2.96-2.88 (m, 1H), 2.16 (s, 3H), 2.14 (s, 6H), 2.11-2.06 (m, 1H), 2.06 (s, 3H), 1.79-1.62 (m, 4H), 1.40-1.30 (m, 2H), 1.28-1.17 (m, 9H), 0.75 (t, 3H). |
| z-214 | ¹H NMR (400 MHz, DMSO-d₆): δ 11.49 (br. s., 1H), 7.80 (d, 1H), 7.64 (s, 1H), 7.20 (s, 1H), 7.10-7.04 (m, 2H), 5.88 (s, 1H), 4.46 (d, 2H), 3.16 (q, 2H), 3.00-2.95 (m, 1H), 2.22 (s, 3H), 2.20-2.11 (m, 9H), 1.87-1.74 (m, 4H), 1.44-1.39 (m, 2H), 1.27-1.23 (m, 10H), 0.83 (t, 3H). |

### Example 194: Preparation of 4-((4,6-dimethyl-2-oxo-1,2-dihydropyridin-3-yl) methyl amino)-8-(ethyl(tetrahydro-2H-pyran-4-yl)amino)quinoline-6-carbonitrile (compound z-194)

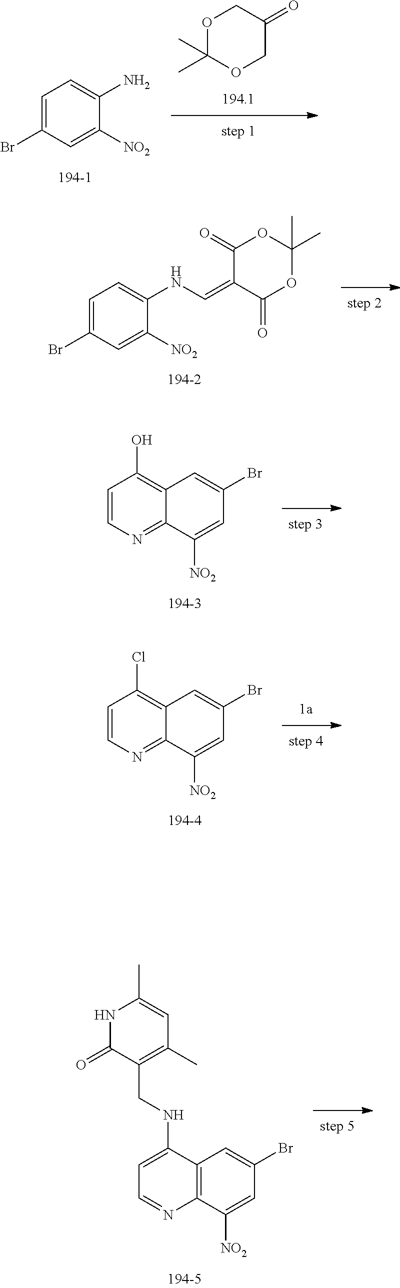

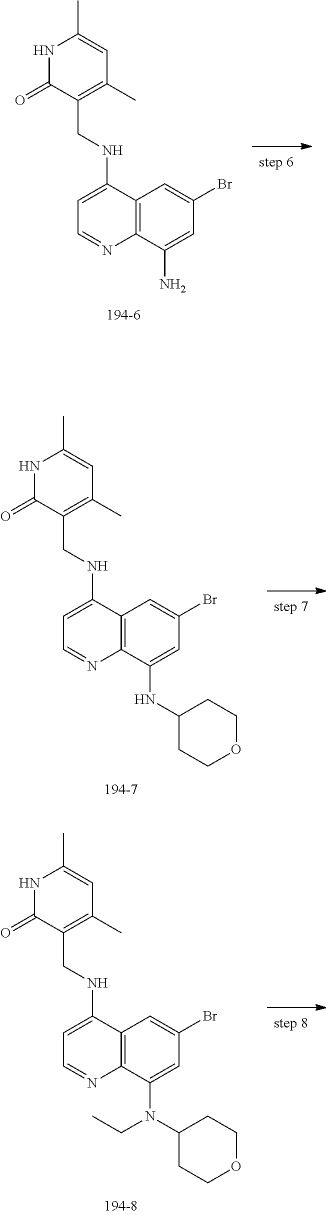 [SMILES: Cc1cc(C)c(CNc2ccnc3c(N)cc(Br)cc23)c(=O)[nH]1 ; Cc1cc(C)c(CNc2ccnc3c(NC4CCOCC4)cc(Br)cc23)c(=O)[nH]1 ; CCN(c1cc(Br)cc2c(NCc3c(C)cc(C)[nH]c3=O)ccnc12)C1CCOCC1]

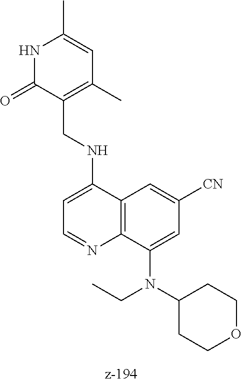 [SMILES: CCN(c1cc(C#N)cc2c(NCc3c(C)cc(C)[nH]c3=O)ccnc12)C1CCOCC1]

Step 1: A solution of compound 194-1 (2.1 g, 10 mmol) in CH(OEt)₃ (20 mL) was added compound 194.1 (2.1 g, 15 mmol) and the mixture was stirred at 80° C. for 1 hour. LC-MS was used to trace the reaction until the reaction was complete. The reaction solution was cooled to room temperature and filtered. The filter cake was washed with n-hexane and the washed cake was exactly compound 194-2. MS m/z (ESI): 369 [MH]⁺.

Step 2: A solution of compound 194-2 (1.85 g, 5 mmol) in diphenyl ether (15 mL) was stirred at 250° C. for 1 h. LC-MS was used to trace the reaction until the reaction was complete. The reaction solution was cooled to room temperature, filtered, and the filtrate was dried and concentrated, purified by combiflash to give compound 194-3. MS m/z (ESI): 269 [MH]⁺.

Step 3: The preparation method was the same as that of compound 2a-4, except that compound 2a-3 in the preparation method of compound 2a-4 was replaced by compound 194-3. MS m/z (ESI): 287 [M+H]⁺.

Step 4: The preparation method was the same as that of compound 2a, except that compound 2a-4 in the preparation method of compound 2a was replaced by compound 194-4. MS m/z (ESI): 403 [M+H]⁺.

Step 5: The preparation method was the same as that of compound 11a-1, except that compound 2a-4 in the preparation method of compound 11a-1 was replaced by compound 194-5. MS m/z (ESI): 373 [M+H]⁺.

Step 6: The preparation method was the same as that of compound 11a-3, except that compound 11a-2 in the preparation method of compound 11a-3 was replaced by compound 194-6. MS m/z (ESI): 457 [M+H]⁺.

Step 7: The preparation method was the same as that of compound 11a-4, except that compound 11a-3 in the preparation method of compound 11a-4 was replaced by compound 194-7. MS m/z (ESI): 485 [M+H]⁺.

Step 8: The preparation method was the same as that of compound z-7, except that compounds z-3 and 7.1 in the preparation method of compound z-7 were replaced by compound 194-8 and sodium cyanoborohydride. MS m/z (ESI): 432[M+H]⁺. ¹H NMR (400 MHz, DMSO) δ11.50 (s, 1H), 8.42 (s, 1H), 8.40 (d, 1H), 7.39 (t, 1H), 7.27 (s, 1H), 6.74 (d, 1H), 5.90 (s, 1H), 4.27 (d, 2H), 4.00-3.94 (m, 1H), 3.86-3.83 (m, 2H), 3.39 (q, 2H), 3.22 (t, 2H), 2.19 (s, 3H), 2.13 (s, 3H), 1.74-1.60 (m, 4H), 0.87 (t, 3H).

### Example 206: Preparation of 3-((5-(ethyl(4-isopropylcyclohexyl) amino)-7-(2-methoxy ethoxy) isoquinolin-1-ylamino) methyl)-4,6-dimethyl-2(1H)-one (compound z-206)

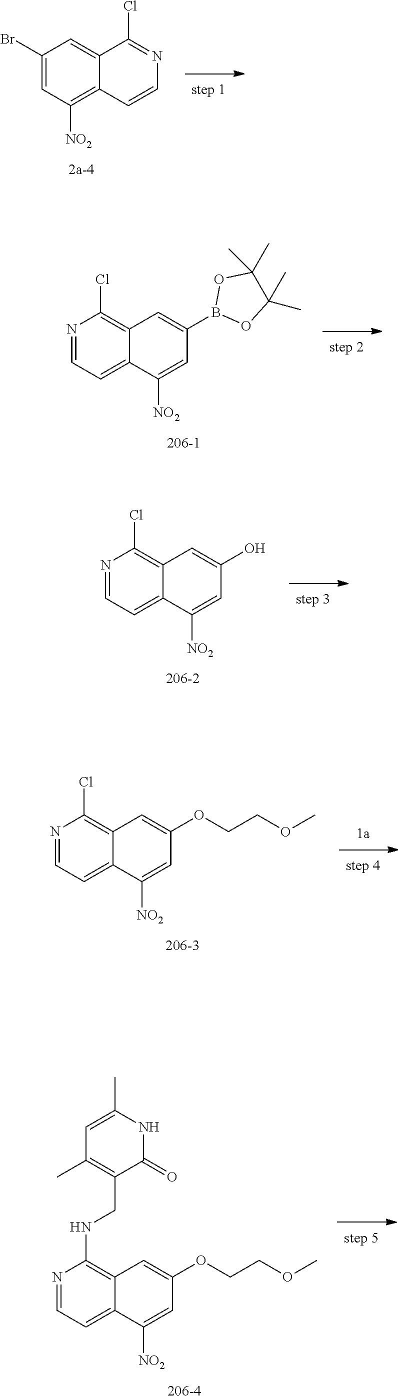 [SMILES: O=[N+]([O-])c1cc(Br)cc2c(Cl)nccc12 ; CC1(C)OB(c2cc([N+](=O)[O-])c3ccnc(Cl)c3c2)OC1(C)C ; O=[N+]([O-])c1cc(O)cc2c(Cl)nccc12 ; COCCOc1cc([N+](=O)[O-])c2ccnc(Cl)c2c1 ; COCCOc1cc([N+](=O)[O-])c2ccnc(NCc3c(C)cc(C)[nH]c3=O)c2c1]

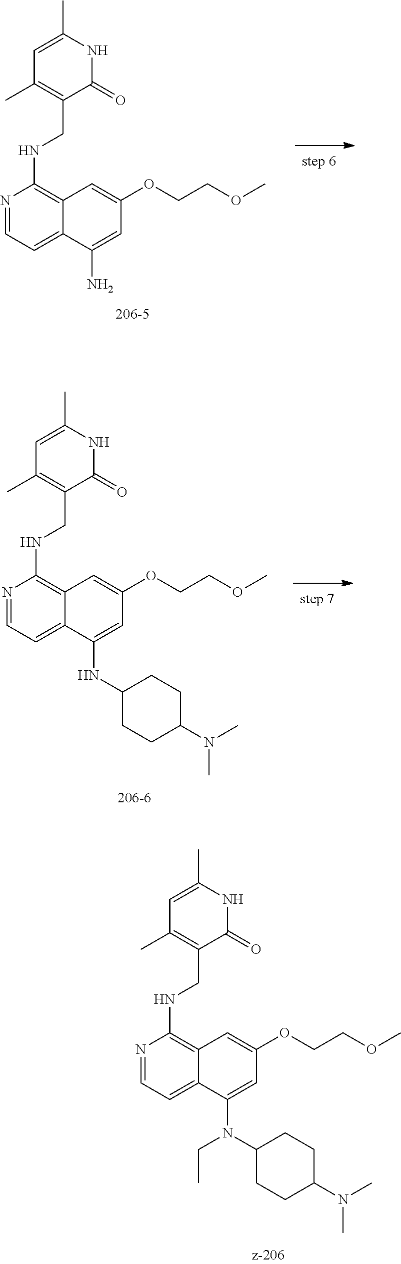 [SMILES: COCCOc1cc(N)c2ccnc(NCc3c(C)cc(C)[nH]c3=O)c2c1 ; COCCOc1cc(NC2CCC(N(C)C)CC2)c2ccnc(NCc3c(C)cc(C)[nH]c3=O)c2c1 ; CCN(c1cc(OCCOC)cc2c(NCc3c(C)cc(C)[nH]c3=O)nccc12)C1CCC(N(C)C)CC1]

Step 1: The preparation method was the same as that of compound 1b, except that compound 1b-2 in the preparation method of compound 1b was replaced by compound 2a-4. MS m/z (ESI): 253 [M+H]⁺.

Step 2: To a solution of compound 206-1 (1 g, 2.99 mmol) in acetone (20 mL) was added dropwise a solution of H₃K₅O₁₈S₄(2.02 g, 3.29 mmol) in water (4 mL) and the mixture was stirred at room temperature for 15 minutes. LC-MS was used to trace the reaction until the reaction was complete. The system was quenched with ammonium chloride solution, extracted with dichloromethane, and the organic layer was dried and concentrated to obtain compound 206-2. MS m/z (ESI): 223[M+H]⁺.

Step 3: The preparation method was the same as that of compound 2a, except that compound 2a-4 and 1a in the preparation method of compound 2a were replaced by compound 206-2 and 2-bromoethyl methyl ether. MS m/z (ESI): 283.1[M+H]⁺.

Step 4: The preparation method was the same as that of compound 2a, except that compound 2a-4 in the preparation method of compound 2a was replaced by compound 206-3. MS m/z (ESI): 399.3[M+H]⁺.

Step 5: The preparation method was the same as that of compound 3a, except that compound 3a-5 in the preparation method of compound 3a was replaced by compound 206-4. MS m/z (ESI): 369.3[M+H]⁺.

Step 6: The preparation method was the same as that of compound 15-1, except that compound 4a in the preparation method of compound 15-1 was replaced by compound 206-5. MS m/z (ESI): 494.3 [M+H]⁺.

Step 7: The preparation method was the same as that of compound z-15, except that compound 15-1 in the preparation method of compound z-15 was replaced by compound 206-6. After purification by Prep-HPLC, a solid compound z-206 (38 mg, 18%) was obtained. MS m/z (ESI): 522.4[M+H]⁺.

### Example 216: Preparation of 4-((5-((4-(dimethylamino)cyclohexyl)(ethyl) amino)-7-(6-(4-methylpiperazin-1-yl)pyridine-3-yl)isoquinolin-1-ylamino) methyl)-1,5-dimethyl-H-pyrazol-3-(2H)-one (compound z-216)

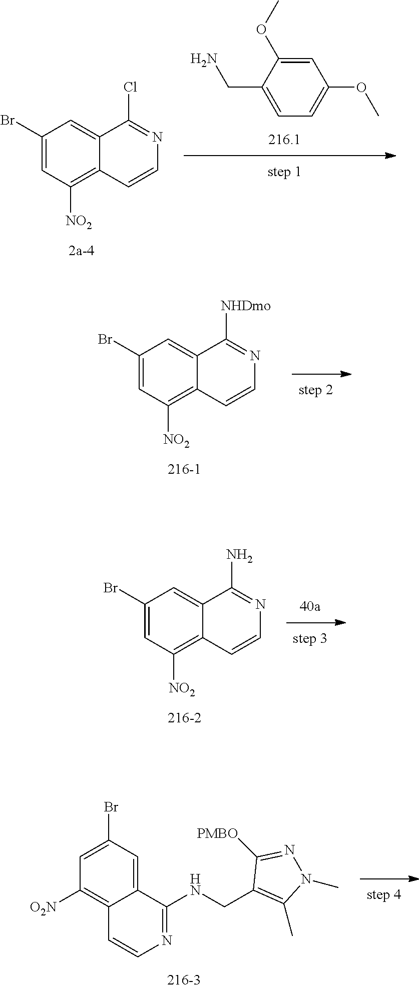

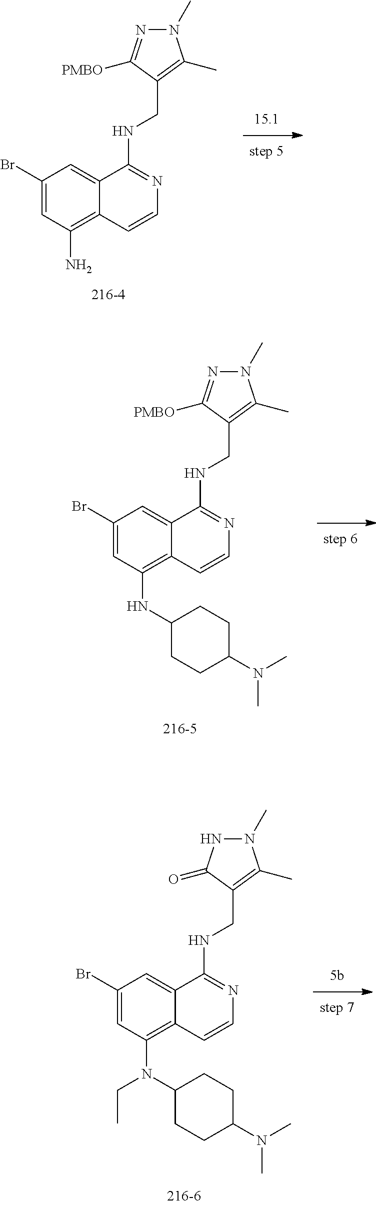

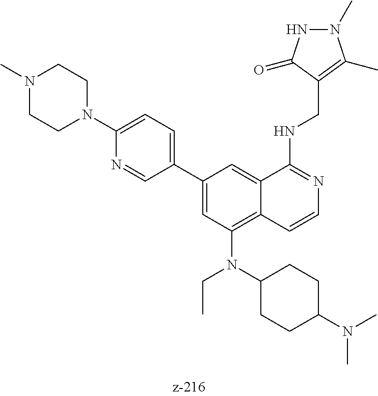

Step 1: A solution of compound 2a-4 (100 mg, 0.34 mmol) in DMF (2 mL) was added compound 216.1 (87 mg, 0.52 mmol) and potassium carbonate (140 mg, 1 mmol) under argon atmosphere and the mixture was stirred at 80° C. for 16 hours. Water was added to the reaction solution, filtered, and the filter cake was washed with water and dried under reduced pressure at a temperature of less than 50° C. to give compound 216-1. MS m/z (ESI): 418 [M+H]⁺.

Step 2: To a solution of compound 216-1 (3 g, 7.1 mmol) in dichloromethane (18 mL) was added triethylamine (18 mL) under argon atmosphere, and the mixture was stirred at room temperature for 2 hours. The reaction solution was concentrated to obtain compound 216-2. MS m/z (ESI): 267.9 [M+H]⁺.

Step 3: To a solution of compound 216-2 (470 mg, 1.7 mmol) in DMSO (10 mL) was added compound 40a (600 mg, 1.7 mmol) and DIPEA (450 mg, 3.5 mmol) under argon atmosphere, and the mixture was stirred at room temperature for 2 hours. LC-MS was used to trace the reaction until the reaction was complete. The reaction solution was extracted with dichloromethane/water, the organic layer was dried and concentrated, and purified by combiflash to give compound 216-3. MS m/z (ESI): 512[M+H]⁺.

Step 4: The preparation method was the same as that of compound 11a-1, except that compound 2a-4 in the preparation method of compound 11a-1 was replaced by compound 216-3. MS m/z (ESI): 482.1[M+H]⁺.

Step 5: The preparation method was the same as that of compound 15-1, except that compound 4a in the preparation method of compound 15-1 was replaced by compound 216-4. MS m/z (ESI): 607 [M+H]⁺.

Step 6: The preparation method was the same as that of compound z-15, except that compound 15-1 in the preparation method of compound z-15 was replaced by compound 216-5. MS m/z (ESI): 515 [M+H]⁺.

Step 7: The preparation method was the same as that of compound z-7, except that compounds z-3 and 7.1 in the preparation method of compound z-7 were replaced by compounds 216-6 and 5b. MS m/z (ESI): 612[M+H]⁺.

### Example 217: Preparation of 1-((4,6-dimethyl-2-oxo-1,2-dihydropyridin-3-yl) methyl amino)-7-(6-(4-methylpiperazine-1-yl)pyridin-3-yl)isoquinoline-5-carbonitrile (compound z-217)

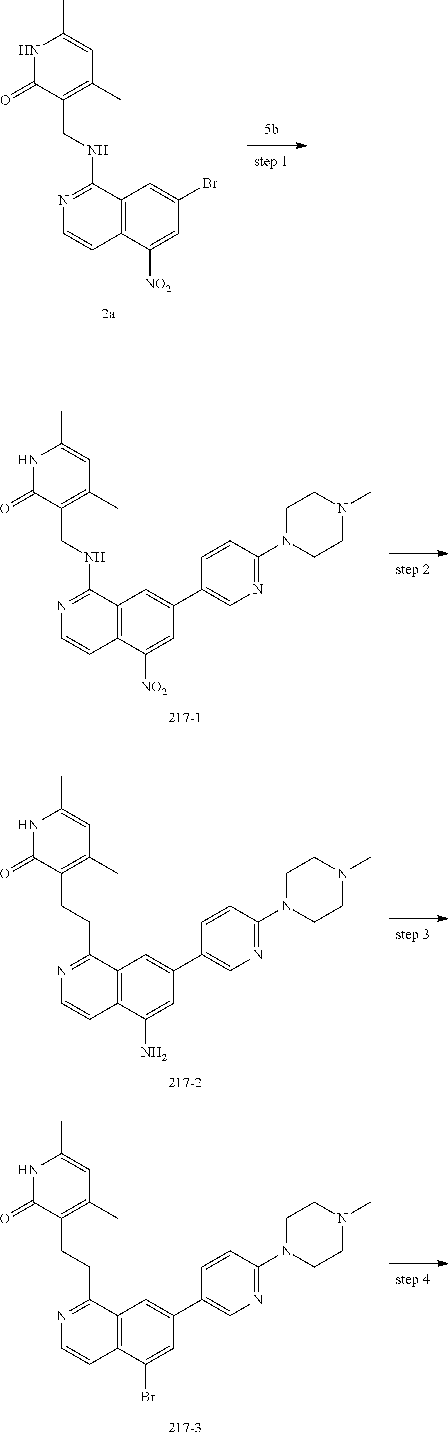 [SMILES: Cc1cc(C)c(CNc2nccc3c([N+](=O)[O-])cc(Br)cc23)c(=O)[nH]1 ; Cc1cc(C)c(CNc2nccc3c([N+](=O)[O-])cc(-c4ccc(N5CCN(C)CC5)nc4)cc23)c(=O)[nH]1 ; Cc1cc(C)c(CCc2nccc3c(N)cc(-c4ccc(N5CCN(C)CC5)nc4)cc23)c(=O)[nH]1 ; Cc1cc(C)c(CCc2nccc3c(Br)cc(-c4ccc(N5CCN(C)CC5)nc4)cc23)c(=O)[nH]1]

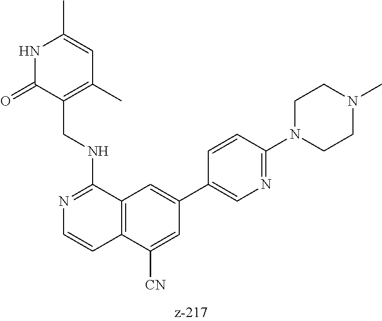 [SMILES: Cc1cc(C)c(CNc2nccc3c(C#N)cc(-c4ccc(N5CCN(C)CC5)nc4)cc23)c(=O)[nH]1]

Step 1: The preparation method was the same as that of compound a-17, except that compounds 2-3 and 7.1 in the preparation method of compound z-7 were replaced by compounds 2a and 5b. MS m/z (ESI): 500[M+H]⁺.

Step 2: The preparation method was the same as that of compound 11a-1, except that compound 2a-4 in the preparation method of compound 11a-1 was replaced by compound 217-1. MS m/z (ESI): 470.1 [M+H]⁺.

Step 3: The preparation method was the same as that of compound 30b-4, except that compound 30b-3 in the preparation method of compound 30b-4 was replaced by compound 217-2. MS m/z (ESI): 535 [M+H]⁺.

Step 4: The preparation method was the same as that of compound z-6, except that compound z-3 in the preparation method of compound z-6 was replaced by compound 217-3. After purification by Prep-HPLC, a white solid compound z-217 (3 mg, 3.4%) was obtained. MS m/z (ESI): 480[M+H]⁺.

### Example 218: Preparation of 3-((5-((4-(dimethylamino) cyclohexyl) (ethyl) amino)-6-fluoro-7-(6-(4-methylpiperazin-1-yl)pyridin-3-yl)isoquinolin-1-ylamino) methyl)-4,6-dimethyl-2(1H)-one (compound z-218)

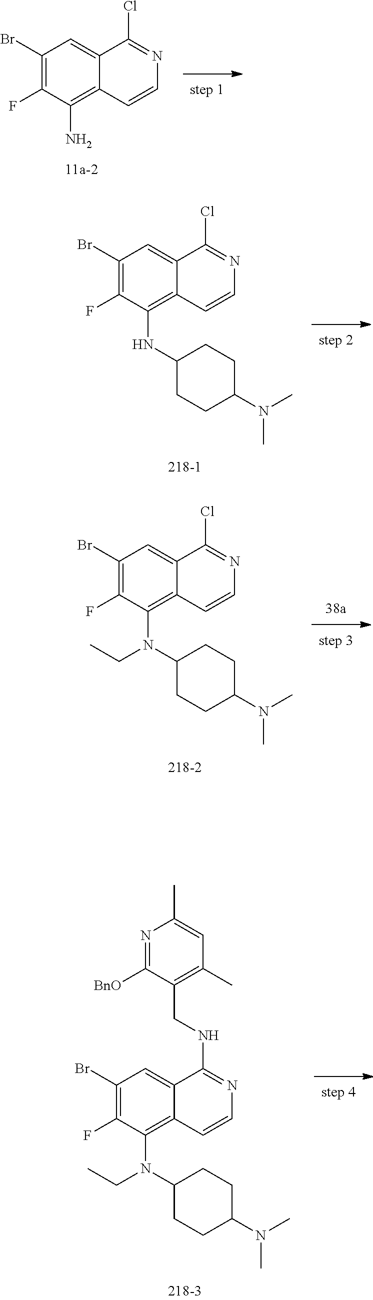 [SMILES: Nc1c(F)c(Br)cc2c(Cl)nccc12 ; CN(C)C1CCC(Nc2c(F)c(Br)cc3c(Cl)nccc23)CC1 ; CCN(c1c(F)c(Br)cc2c(Cl)nccc12)C1CCC(N(C)C)CC1 ; CCN(c1c(F)c(Br)cc2c(NCc3c(C)cc(C)nc3OCc3ccccc3)nccc12)C1CCC(N(C)C)CC1]

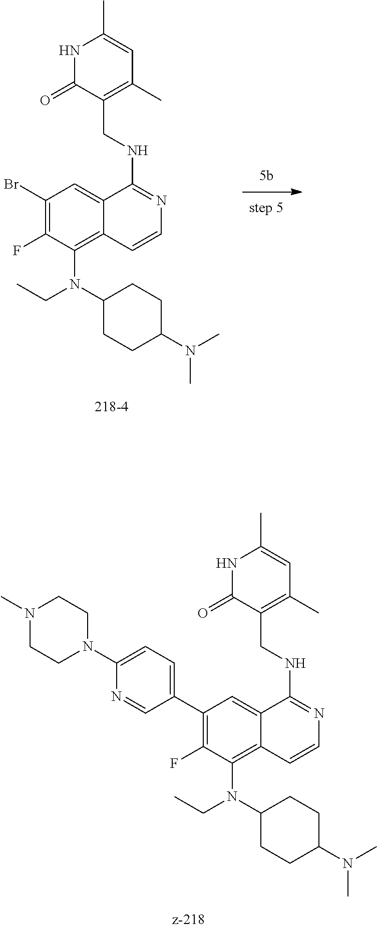

Step 1: The preparation method was the same as that of compound 15-1, except that compound 4a in the preparation method of compound 15-1 was replaced by compound 11a-2. MS m/z (ESI): 408 [M+H]⁺.

Step 2: The preparation method was the same as that of compound z-15, except that compound 15-1 in the preparation method of compound z-15 was replaced by compound 218-1. MS m/z (ESI): 428[M+H]⁺.

Step 3: The preparation method was the same as that of compound 2a, except that compounds 2a-4 and 1a in the preparation method of compound 2a were replaced by compounds 218-2 and 38a. MS m/z (ESI): 634[M+H]⁺.

Step 4: The preparation method was the same as that of compound z-8, except that compound 8-1 in the preparation method of compound z-8 was replaced by compound 218-3. MS m/z (ESI): 544[M+H]⁺.

Step 5: The preparation method was the same as that of compound z-7, except that compounds z-3 and 7.1 in the preparation method of compound z-7 were replaced by compounds 218-4 and 5b. After purification by Prep-HPLC, a white solid compound z-218 (1 mg, 3.4%) was obtained. MS m/z (ESI): 641.5[M+H]⁺. ¹H NMR (400 MHz, dmso) δ 11.43 (s, 1H), 8.34 (s, 1H), 8.19 (d, 1H), 7.86 (d, 1H), 7.75 (d, 1H), 7.28 (s, 1H), 7.18 (d, 1H), 6.90 (d, 1H), 5.85 (s, 1H), 4.42 (s, 2H), 3.51 (s, 4H), 3.00-2.91 (m, 2H), 2.64 (s, 1H), 2.38 (s, 4H), 2.28-2.22 (m, 6H), 2.19-2.06 (m, 7H), 2.08 (s, 3H), 1.99-1.92 (m, 4H), 1.83-1.71 (m, 2H), 1.45-1.38 (m, 2H), 0.82 (t, 3H).

### Example 219: Preparation of 3-((5-((4-(dimethylamino) cyclohexyl)(ethyl)amino)-3-methoxy-7-(6-(4-methylpiperazine-1-yl)pyridin-3-yl) isoquinolin-1-ylamino) methyl)-4,6-dimethyl-2(1H)-one (compound z-219)

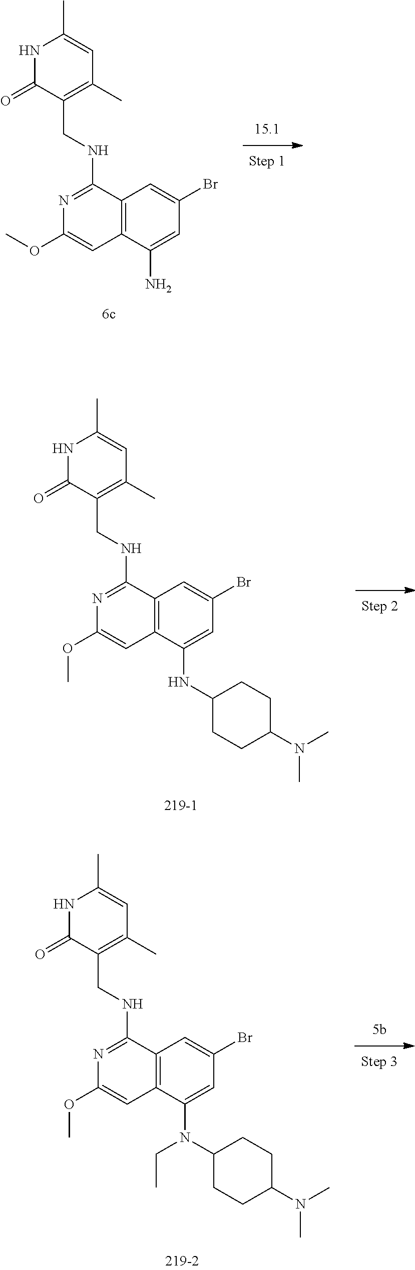 [SMILES: COc1cc2c(N)cc(Br)cc2c(NCc2c(C)cc(C)[nH]c2=O)n1 ; COc1cc2c(NC3CCC(N(C)C)CC3)cc(Br)cc2c(NCc2c(C)cc(C)[nH]c2=O)n1 ; CCN(c1cc(Br)cc2c(NCc3c(C)cc(C)[nH]c3=O)nc(OC)cc12)C1CCC(N(C)C)CC1]

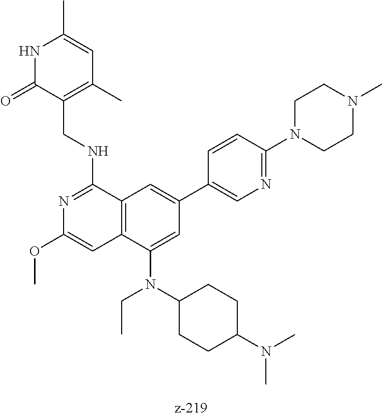 [SMILES: CCN(c1cc(-c2ccc(N3CCN(C)CC3)nc2)cc2c(NCc3c(C)cc(C)[nH]c3=O)nc(OC)cc12)C1CCC(N(C)C)CC1]

Step 1: The preparation method was the same as that of compound 15-1, except that compound 4a in the preparation method of compound 15-1 was replaced by compound 6c. MS m/z (ESI): 530.3[M+H]⁺.

Step 2: The preparation method was the same as that of compound z-15, except that compound 15-1 in the preparation method of compound z-15 was replaced by compound 219-1. MS m/z (ESI): 558.3 [M+H]⁺.

Step 3: The preparation method was the same as that of compound z-7, except that compounds z-3 and 7.1 in the preparation method of compound z-7 were replaced by compounds 219-2 and 5b. After purification by Prep-HPLC, a white solid compound z-219 (400 mg, 97%) was obtained. MS m/z (ESI): 653.5[M+H]⁺.

### Example 220: Preparation of 3-((5-(ethyl (2-methylpiperidin-4-yl)amino)-7-(6-(4-methyl piperazin-1-yl)pyridin-3-yl)isoquinolin-1-ylamino) methyl)-4,6-dimethylpyridine-2(1H)-one (compound z-220)

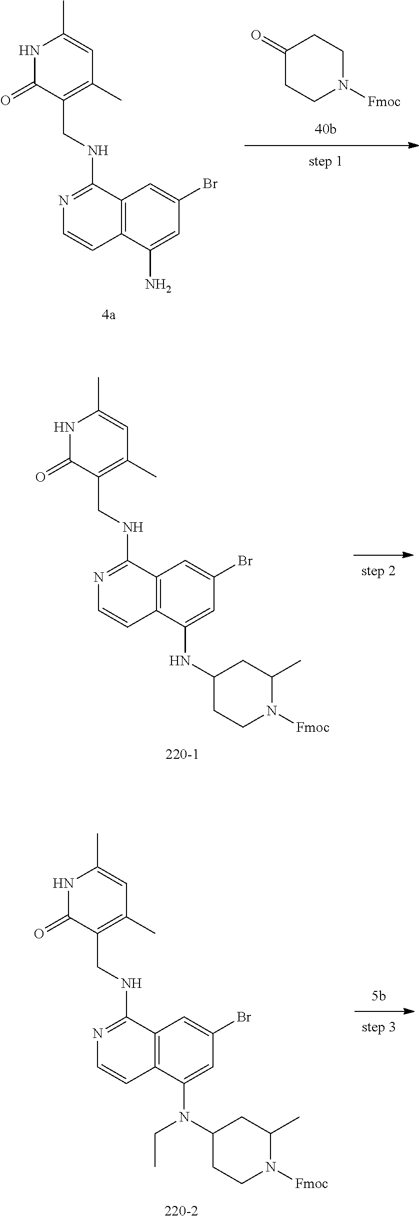

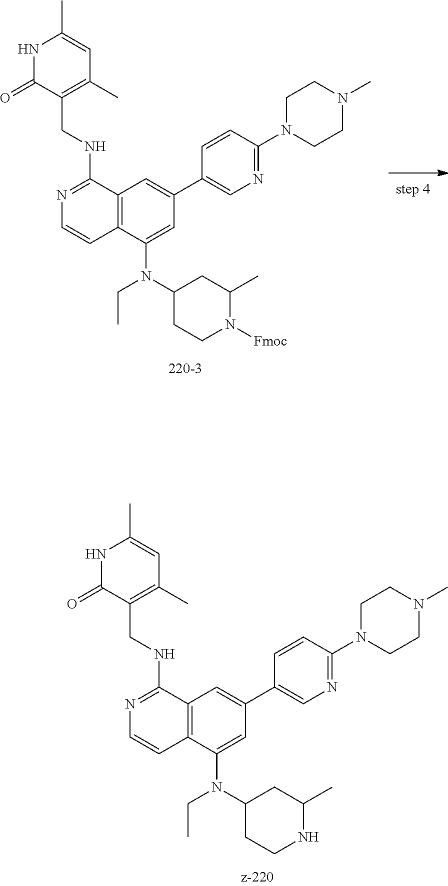

Step 1: The preparation method was the same as that of compound 15-1, except that compound 15.1 in the preparation method of compound 15-1 was replaced by compound 40b. MS m/z (ESI): 692 [M+H]⁺.

Step 2: The preparation method was the same as that of compound z-15, except that compound 15-1 in the preparation method of compound z-15 was replaced by compound 220-1. MS m/z (ESI): 720 [M+H]⁺.

Step 3: The preparation method was the same as that of compound z-7, except that compounds z-3 and 7.1 in the preparation method of compound z-7 were replaced by compounds 220-2 and 5b. MS m/z (ESI): 818[M+H]⁺.

Step 4: To a solution of compound 220-3 (20 mg, 0.025 mmol) in 10 mL of dichloromethane was added piperidine (1 mL) and the mixture was stirred at room temperature for 2 hours. LC-MS was used to trace the reaction until the reaction was complete. The reaction solution was concentrated and purified by Prep-HPLC to give a solid compound z-220 (8 mg, 44%). MS m/z (ESI): 595 [M+H]⁺.

### Example 221: Preparation of 3-((5-(ethyl(4-hydroxy-4-methylcyclohexyl) amino)-7-(2-(4-methylpiperazin-1-yl) pyrimidin-5-yl)isoquinolin-1-ylamino) methyl)-4,6-dimethyl pyridin-2(1H)-one (compound z-221)

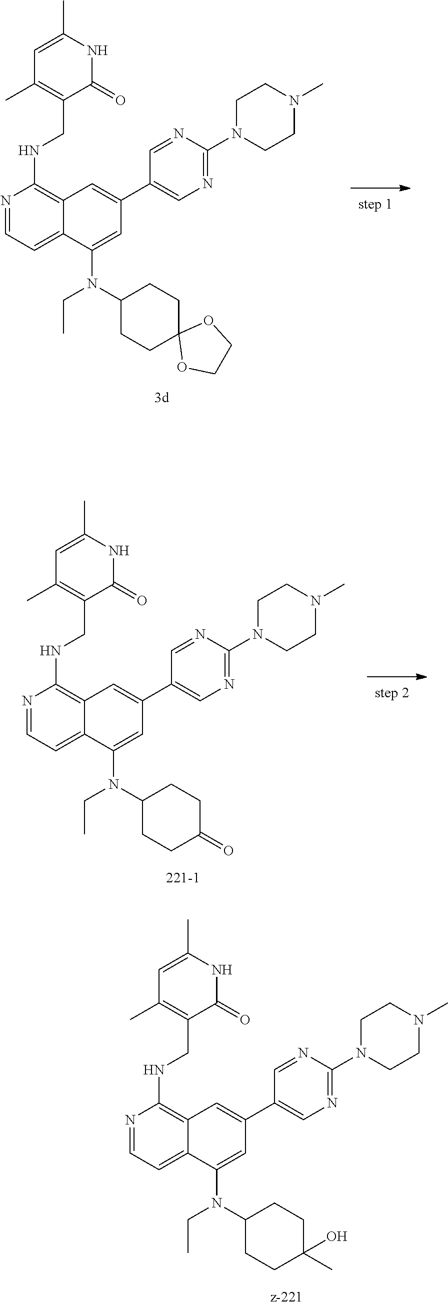

Step 1: A solution of compound 3d (100 mg, 1.1 mmol) and TsOH (755 mg, 4.38 mmol) in tetrahydrofuran (15 mL) and water (15 mL) was heated to 75° C. and stirred overnight. LC-MS was used to trace the reaction until the reaction was complete. The reaction solution was cooled to room temperature, poured into water, extracted with ethyl acetate, concentrated, and purified by combiflash to give 300 mg solid compound 221-1. MS m/z (ESI): 595.4 [M+H]⁺.

Step 2: To a solution of compound 221-1 (280 mg, 0.47 mmol) in tetrahydrofuran (15 mL) was added dropwise MeMgBr (0.94 mL, 2.82 mmol) under an ice bath and the mixture was stirred under an ice bath for 2 hours. LC-MS was used to trace the reaction until the reaction was complete. The reaction solution was quenched with ammonium chloride, extracted with ethyl acetate, concentrated, and purified by Prep-HPLC to get a white solid compound z-221 (6 mg, 1.8%). MS m/z (ESI): 612.5 [M+H]⁺.

### Example 222: Preparation of 3-((4-((4-(dimethylamino) cyclohexyl) (ethyl) amino)-2-ethoxy-1,7-naphthyridin-8-ylamino) methyl)-4,6-dimethyl-2(1H)-one (compound z-222)

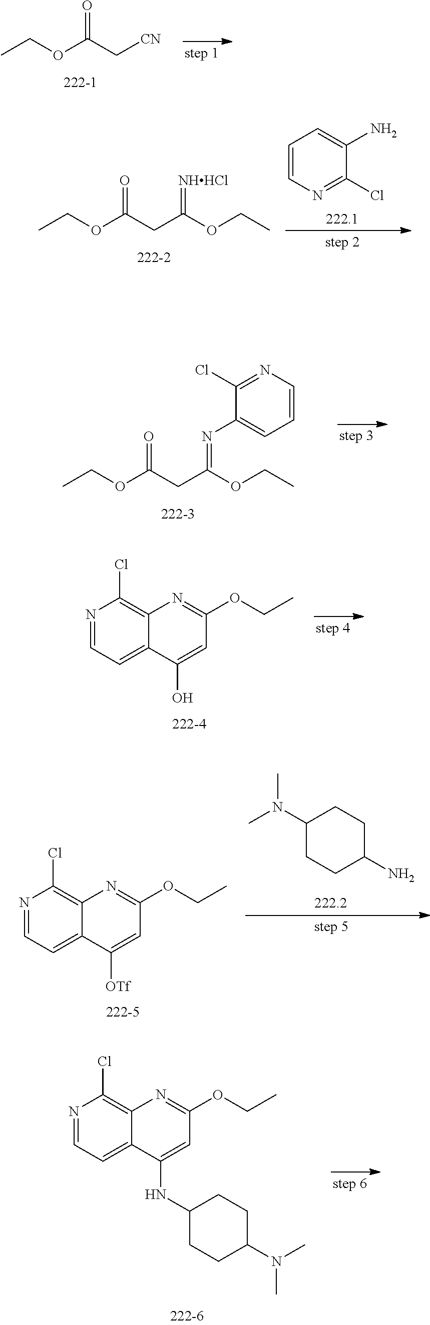

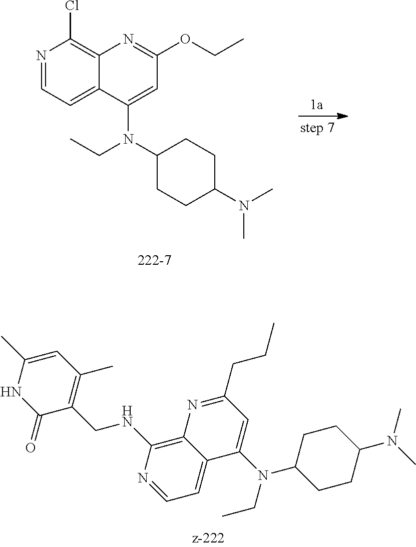

Step 1: A solution of compound 222-1 (46 g, 407 mmol), dry ethanol (20 g, 435 mmol) in methyl tert-butyl ether (40 mL) was passed through hydrochloric acid gas (24 g) under an ice bath, the mixture was reacted under an ice bath for 6 hours, and then stirred at room temperature for 16 hours. The solids in the system were crushed, washed with ethyl acetate, and dried under reduced pressure to give compound 222-2. MS m/z (ESI): 160 [M+H]⁺.

Step 2: To compound 222.1 (25.4 g, 277 mmol) was added a solution of compound 222-2 (54 g, 277 mmol) in ethanol (350 mL) under a nitrogen atmosphere. The mixture was reacted at room temperature for 24 hours and then raised to 50° C. and stirred for 6 hours. The reaction solution was concentrated and purified by combiflash to give 28 g compound 222-3. MS m/z (ESI): 271[M+H]⁺.

Step 3: A solution of compound 222-3 (13 g, 48 mmol) in diphenyl ether (100 mL) was heated to 210-230° C. and stirred for 10 minutes. The reaction solution was purified by combiflash to give compound 222-4. MS m/z (ESI): 225[M+H]⁺.

Step 4: Compound N-phenyl bis(trifluoromethanesulfonyl)imide (4.4 g, 12.28 mmol) was added to a solution of compound 222-4 (1.1 g, 4.91 mmol) and triethylamine (2 g, 19.64 mmol) in dichloromethane (30 mL). The mixture was stirred at 30° C. for 4 hours. LC-MS was used to trace the reaction until the reaction was complete. The reaction solution was added water, extracted with dichloromethane, the organic layer was dried, concentrated, and purified by combiflash to give 3.4 g of compound 222-5. MS m/z (ESI): 357[M+H]⁺.

Step 5: A solution of compound 222-5 (4 g, 2.8 mmol), 222.2 (478 mg, 3.4 mmol), Xantphos (162 mg, 0.28 mmol), Pd₂ (dba)₃ (258 mg, 0.28 mmol), sodium tert-butoxide (444 mg, 4.64 mmol) in toluene (6 mL) was microwaved to react at 100° C. for 12 minutes. LC-MS was used to trace the reaction until the reaction was complete. The reaction solution was filtered, concentrated, and purified by combiflash to give 1.07 g of compound 222-6. MS m/z (ESI): 349 [M+H]⁺.

Step 6: To a solution of compound 222-6 (1.07 g, 3.07 mmol) in DMF (25 mL) was added sodium hydride (129 mg, 3.23 mmol). The mixture was stirred at room temperature for 30 minutes, and iodoethane (504 mg, 3.23 mmol) in DMF (5 mL) was added dropwise. The mixture was stirred at room temperature for 3 hours. LC-MS was used to trace the reaction until the reaction was complete. The reaction solution was poured into water and extracted with dichloromethane. The organic layer was dried and concentrated to give compound 222-7. MS m/z (ESI): 377 [M+H]⁺.

Step 7: The preparation method was the same as that of compound 226-6, except that compounds 225-5 and 222.2 in the preparation method of 222-6 were replaced by compounds 222-7 and 1a. After purification by Prep-HPLC, compound z-222 (2.9 mg, 1%) was obtained as a white solid. MS m/z (ESI): 493[M+H]⁺. ¹H-NMR (400 MHz, CDCl₃): δ 10.51 (bs, 1H), 7.83 (d, 1H), 6.83 (d, 1H), 6.46 (s, 1H), 5.85 (s, 1H), 4.71 (d, 2H), 4.40 (q, 2H), 3.50 (s, 1H), 3.26 (q, 2H), 2.41 (s, 3H), 2.31 (s, 5H), 2.21 (s, 3H), 2.02 (s, 1H), 1.81 (s, 2H), 1.59 (s, 4H), 1.48 (t, 3H), 1.37 (t, 3H), 0.91 (t, 3H).

### Example 223: Preparation of 3-((5-((4-(dimethylamino) cyclohexyl)(ethyl)amino)-4-fluoro-7-(6-(4-methylpiperazin-1-yl)pyridin-3-yl)isoquinolin-1-ylamino) methyl)-4,6-dimethyl-2(1H)-one (compound z-223)

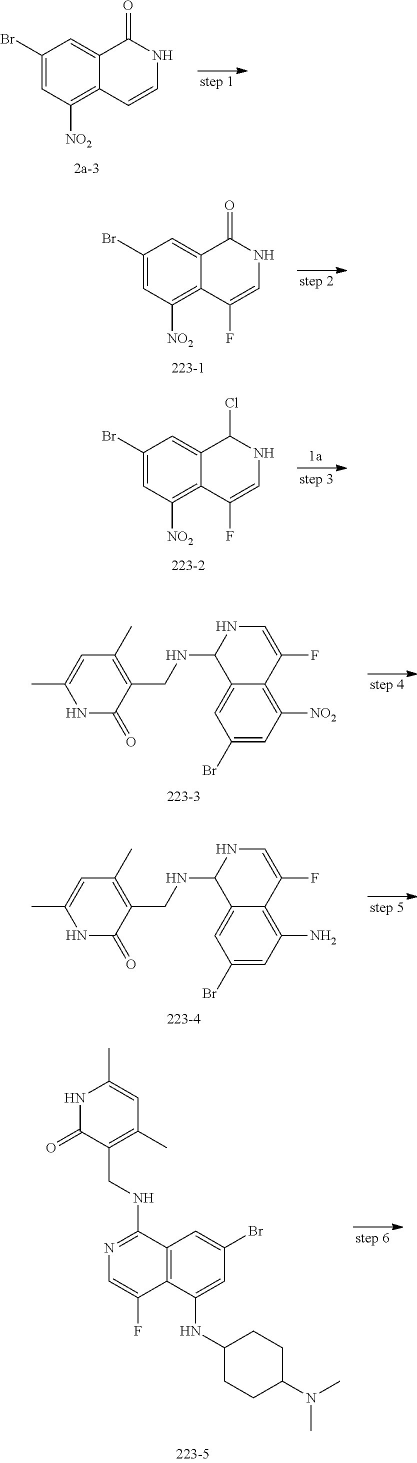 [SMILES: O=c1[nH]ccc2c([N+](=O)[O-])cc(Br)cc12 ; O=c1[nH]cc(F)c2c([N+](=O)[O-])cc(Br)cc12 ; O=[N+]([O-])c1cc(Br)cc2c1C(F)=CNC2Cl ; Cc1cc(C)c(CNC2NC=C(F)c3c2cc(Br)cc3[N+](=O)[O-])c(=O)[nH]1 ; Cc1cc(C)c(CNC2NC=C(F)c3c(N)cc(Br)cc32)c(=O)[nH]1 ; Cc1cc(C)c(CNc2ncc(F)c3c(NC4CCC(N(C)C)CC4)cc(Br)cc23)c(=O)[nH]1]

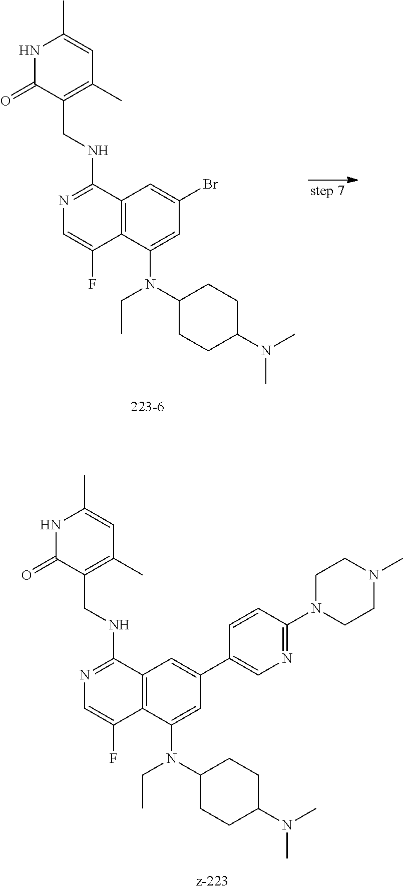 [SMILES: CCN(c1cc(Br)cc2c(NCc3c(C)cc(C)[nH]c3=O)ncc(F)c12)C1CCC(N(C)C)CC1 ; CCN(c1cc(-c2ccc(N3CCN(C)CC3)nc2)cc2c(NCc3c(C)cc(C)[nH]c3=O)ncc(F)c12)C1CCC(N(C)C)CC1]

Step 1: The preparation method was the same as that of compound 11a-2, except that compound 11a-1 in the preparation method of compound 11a-2 was replaced by compound 2a-3. MS m/z (ESI): 285[M−H]⁺.

Step 2: The preparation method was the same as that of compound 2a-4, except that compound 2a-3 in the preparation method of compound 2a-4 was replaced by compound 223-1. MS m/z (ESI): 307[M+H]⁺.

Step 3: The preparation method was the same as that of compound 2a, except that compound 2a-4 in the preparation method of compound 2a was replaced by compound 223-2. MS m/z (ESI): 421[M+H]⁺.

Step 4: The preparation method was the same as that of compound 3a, except that compound 3a-5 in the preparation method of compound 3a was replaced by compound 223-3. MS m/z (ESI): 391 [M+H]⁺.

Step 5: The preparation method was the same as that of compound z-4, except that compound 4-1 in the preparation method of compound z-4 was replaced by compound 223-4. MS m/z (ESI): 516[M+H]⁺.

Step 6: The preparation method was the same as that of compound z-15, except that compound 15-1 in the preparation method of compound z-15 was replaced by compound 223-5. MS m/z (ESI): 544[M+H]⁺.

Step 7: The preparation method was the same as that of compound z-7, except that compounds z-3 and 7.1 in the preparation method of compound z-7 were replaced by compounds 223-6 and 5b. After purification by Prep-HPLC, a white solid compound z-223 (0.5 mg, 22%) was obtained. MS m/z (ESI): 641 [M+H]⁺.

### Example 224: Preparation of 3-((5-(ethyl(tetrahydro-2H-pyran-4-yl) amino)-6-fluoro-7-(6-(4-methylpiperazin-1-yl)pyridin-3-yl) isoquinolin-1-ylamino)methyl)-4,6-dimethyl-2(1H)-one (compound z-224)

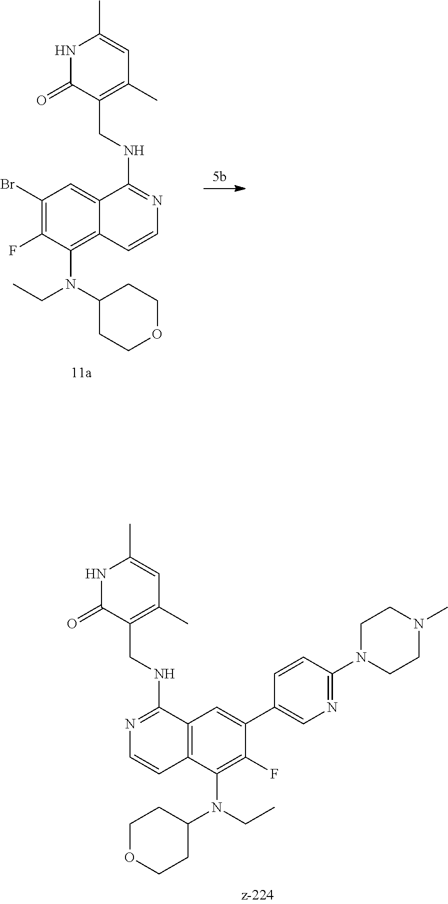 [SMILES: CCN(c1c(F)c(Br)cc2c(NCc3c(C)cc(C)[nH]c3=O)nccc12)C1CCOCC1 ; CCN(c1c(F)c(-c2ccc(N3CCN(C)CC3)nc2)cc2c(NCc3c(C)cc(C)[nH]c3=O)nccc12)C1CCOCC1]

The preparation method was the same as that of compound-7, except that compounds z-3 and 7.1 in the preparation method of z-7 were replaced by compounds 11a and 5b. After purification by Prep-HPLC, a white solid compound z-224 (13 mg, 12.7%) was obtained. MS m/z (ESI): 600[M+H]⁺. ¹H NMR (400 MHz, DMSO) δ11.64-11.17 (m, 1H), 8.33 (s, 1H), 8.20 (d, 1H), 8.16 (s, 2H), 7.86 (d, 1H), 7.74 (d, 1H), 7.31-7.24 (m, 1H), 7.21 (d, 1H), 6.89 (d, 1H), 5.84 (s, 1H), 4.41 (d, 2H), 3.83 (d, 2H), 3.68 (d, 2H), 3.52-3.49 (m, 5.0H), 2.41-2.33 (m, 4H), 2.18 (s, 3H), 2.16 (s, 3H), 2.07 (s, 3H), 1.90 (d, 2H), 1.50-1.40 (m, 2H), 1.46-1.43 (m, 2H), 0.81 (t, 3H).

### Example 225: Preparation of N-(4-((1-((4,6-dimethyl-2-oxo-1,2-dihydropyridin-3-yl) methylamino)-7-(6-(4-methyl piperazin-1-yl) pyridin-3-yl) isoquinolin-5-yl) (ethyl) amino) cyclohexyl) acetamide (compound z-225)

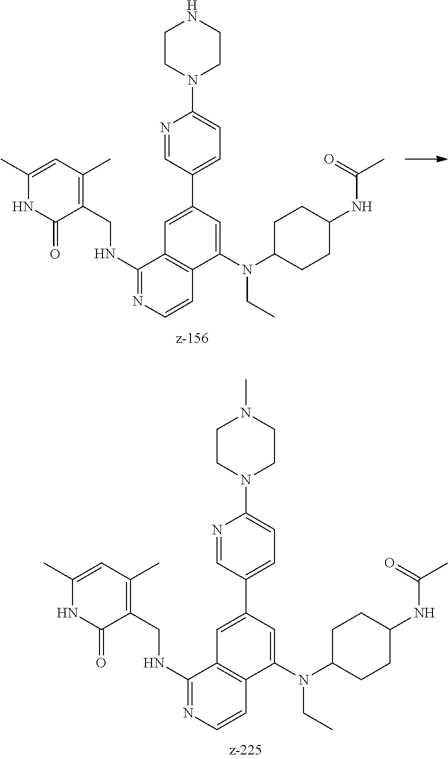

To a solution of compound z-156 (40 mg, 0.064 mmol) in tetrahydrofuran (8 mL) was added a solution of triethylamine (13 mg, 0.128 mmol) and methyl iodide (9 mg, 0.064 mmol) in tetrahydrofuran (0.3 mL) and the mixture was stirred at room temperature for 1 h. LC-MS was used to trace the reaction until the reaction was complete. The reaction solution was poured into water, extracted with ethyl acetate, the organic layer was concentrated and purified by Prep-HPLC to give a white solid compound z-225 (5 mg, 9.8%). MS m/z (ESI): 637.5[M+H]⁺.

### Example 226: Preparation of 3-((7-chloro-5-(ethyl (1-methylpiperidin-4-yl)amino) isoquinolin-1-ylamino)methyl)-4,6-dimethyl-2(1H)-one (compound z-226)

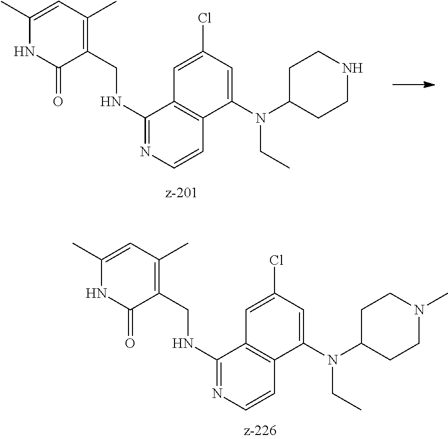

To a solution of compound z-201 (100 mg, 0.23 mmol) and formaldehyde (5 mL) in 1,4-dioxane (10 mL) and trifluoroacetic acid (0.5 mL) was added sodium triacetoxyborohydride (300 mg, 1.37 mmol) and the mixture was stirred at room temperature overnight. LC-MS was used to trace the reaction until the reaction was complete. The reaction solution was added saturated sodium bicarbonate solution, extracted with ethyl acetate; the organic layer was concentrated, and purified by Prep-HPLC to give a white solid compound z-226 (16 mg, 15.2%). MS m/z (ESI): 454[M+H]⁺. ¹H NMR (400 MHz, DMSO-d₆) δ 11.42 (s, 1H), 8.07 (d, 1H), 7.86 (d, 1H), 7.27 (d, 1H), 7.17 (t, 1H), 7.02 (d, 1H), 5.85 (s, 1H), 4.39 (d, 2H), 3.13-3.11 (m, 2H), 2.90-2.88 (m, 1H), 2.69-2.67 (m, 2H), 2.16 (s, 3H), 2.08-2.06 (m, 6H), 1.76-1.53 (m, 6H), 0.80 (t, 3H).

### Example 227: Preparation of 1-((4,6-dimethyl-2-oxo-1,2-dihydropyridin-3-yl) methyl amino)-5-(((1R,4R)-4-(dimethylamino) cyclohexyl) (ethyl)amino) isoquinoline-7-carboxamide (compound z-227)

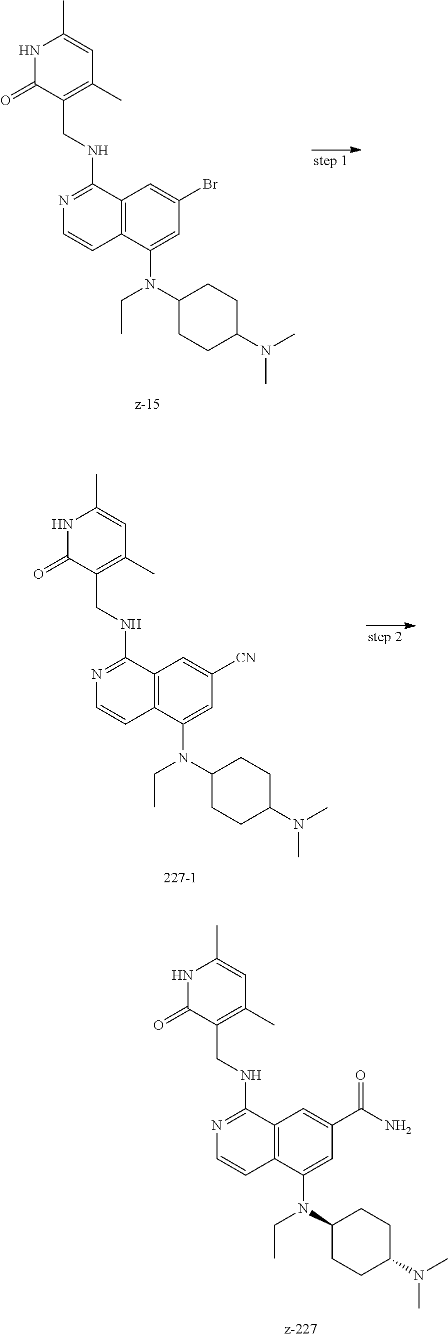 [SMILES: CCN(c1cc(Br)cc2c(NCc3c(C)cc(C)[nH]c3=O)nccc12)C1CCC(N(C)C)CC1 ; CCN(c1cc(C#N)cc2c(NCc3c(C)cc(C)[nH]c3=O)nccc12)C1CCC(N(C)C)CC1 ; CCN(c1cc(C(N)=O)cc2c(NCc3c(C)cc(C)[nH]c3=O)nccc12)[C@H]1CC[C@H](N(C)C)CC1]

Step 1: The preparation method is the same as that of compound z-6, except that compound z-3 in the preparation method of z-6 is replaced by compound z-15. MS m/z (ESI): 473.3 [M+H]⁺.

Step 2: A mixture of compound 227-1 (70 mg, 0.148 mmol) and sodium hydroxide (4 mL, 1 M) in ethanol (4 mL) was stirred under reflux for 2 hours. LC-MS was used to trace the reaction until the reaction was complete. The reaction solution was poured into water. The pH was adjusted to 3, and the mixture was extracted with ethyl acetate. The aqueous layer was concentrated and extracted with ethyl acetate. The organic layer was concentrated and purified by Prep-HPLC to give a white solid compound z-227 (1 mg, 1.4%). MS m/z (ESI): 491.4[M+H]⁺.

### Example 228: Preparation of 3-((5-(ethyl(tetrahydro-2H-pyran-4-yl) amino)-7-((1-methyl piperidin-4-ylamino)methyl)isoquine-1-ylamino) methyl)-4,6-dimethyl-2(1H)-one (compound z-228)

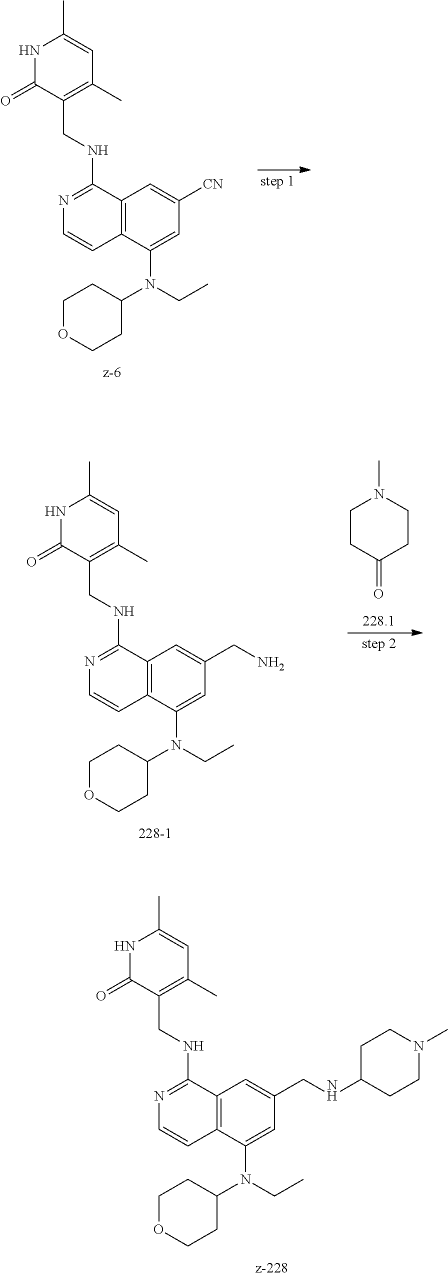

Step 1: The preparation method was the same as that of compound 1a, except that compound 1a-1 in the preparation method of compound 1a was replaced by compound z-6. MS m/z (ESI): 436.3 [M+H]⁺.

Step 2: The preparation method was the same as that of compound z-2, except that compound 4a and tetrahydropyrone in the preparation method of compound z-2 were replaced by compound 228-1 and compound 228.1. After purification by Prep-HPLC, a white solid compound z-228 (3.6 mg, 1.6%) was obtained. MS m/z (ESI): 533.4 [M+H]⁺.

### Example 229: Preparation of 3-((5-(ethyl(1-(methylsulfonyl) piperidin-4-yl)amino) isoquinolin-1-ylamino)methyl)-4,6-dimethyl-2(1H)-one (compound z-229)

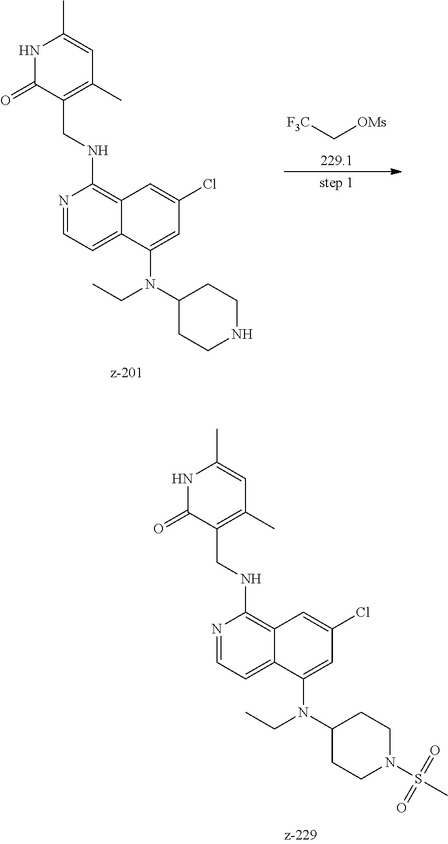 [SMILES: CCN(c1cc(Cl)cc2c(NCc3c(C)cc(C)[nH]c3=O)nccc12)C1CCNCC1 ; CS(=O)(=O)OCC(F)(F)F ; CCN(c1cc(Cl)cc2c(NCc3c(C)cc(C)[nH]c3=O)nccc12)C1CCN(S(C)(=O)=O)CC1]

To a solution of compound z-201 (150 mg, 0.34 mmol), compound 229.1 (242 mg, 1.36 mmol) in acetonitrile (5 mL) was added potassium carbonate (235 mg, 1.7 mmol), and the mixture was stirred at 50° C. overnight. LC-MS was used to trace the reaction until the reaction was complete. The mixture was cooled to room temperature, filtered, and the filtrate was concentrated and purified by Prep-HPLC to give compound z-229 (14.9 mg, 8.5%) as a white solid. MS m/z (ESI): 518[M+H]⁺. ¹H NMR (400 MHz, DMSO-d₆) δ 11.42 (s, 1H), 8.10 (s, 1H), 7.87 (d, 1H), 7.32 (s, 1H), 7.19 (s, 1H), 7.03 (d, 1H), 5.85 (s, 1H), 4.39 (d, 2H), 3.48-3.46 (m, 2H), 3.19-3.04 (m, 2H), 2.76 (s, 3H), 2.65-2.63 (m, 2H), 2.16 (s, 3H), 2.09 (s, 3H), 1.81-1.78 (m, 2H), 1.65-1.63 (m, 2H), 0.81 (t, 3H).

### Example 230: Preparation of 3-((5-((4-(dimethylamino) cyclohexyl)(ethyl)amino) isoquinolin-1-ylamino)methyl)-4,6-dimethyl-2(1H)-one (compound z-230)

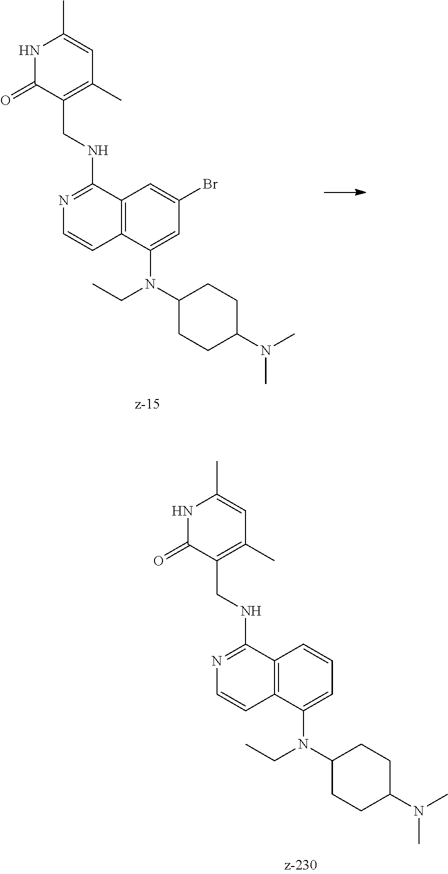 [SMILES: CCN(c1cc(Br)cc2c(NCc3c(C)cc(C)[nH]c3=O)nccc12)C1CCC(N(C)C)CC1 ; CCN(c1cccc2c(NCc3c(C)cc(C)[nH]c3=O)nccc12)C1CCC(N(C)C)CC1]

The preparation method was the same as that of compound z-5, except that compound z-3 in the preparation method of compound z-5 was replaced by compound z-15. After purification by Prep-HPLC, a white solid compound z-230 (5 mg, 3%) was obtained. MS m/z (ESI): 448.3 [M+H]⁺.

### Example 231: Preparation of 3-((5-(((1S,4S)-4-(dimethylamino) cyclohexyl) (ethyl) amino)-6-fluoro-7-(6-(4-methylpiperazin-1-yl)pyridin-3-yl)isoquinolin-1-ylamino) methyl)-4,6-dimethyl-2(1H)-one (compound z-231)

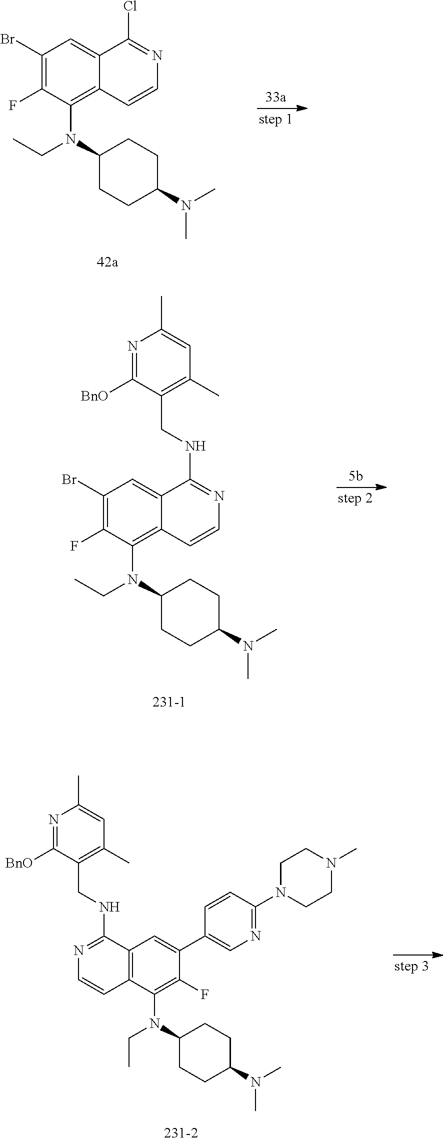 [SMILES: CCN(c1c(F)c(Br)cc2c(Cl)nccc12)[C@H]1CC[C@@H](N(C)C)CC1 ; CCN(c1c(F)c(Br)cc2c(NCc3c(C)cc(C)nc3OCc3ccccc3)nccc12)[C@H]1CC[C@@H](N(C)C)CC1 ; CCN(c1c(F)c(-c2ccc(N3CCN(C)CC3)nc2)cc2c(NCc3c(C)cc(C)nc3OCc3ccccc3)nccc12)[C@H]1CC[C@@H](N(C)C)CC1]

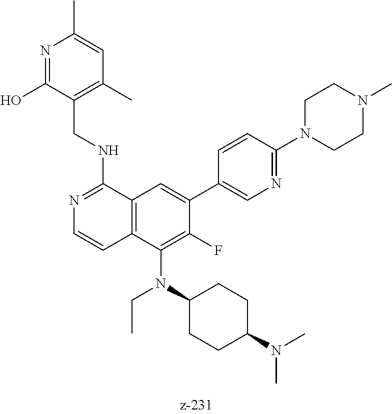 [SMILES: CCN(c1c(F)c(-c2ccc(N3CCN(C)CC3)nc2)cc2c(NCc3c(C)cc(C)nc3O)nccc12)[C@H]1CC[C@@H](N(C)C)CC1]

Step 1: The preparation method was the same as that of compound 218-3, except that compound 218-2 in the preparation method of compound 218-3 was replaced by compound 42a. MS m/z (ESI): 634 [M+H]⁺.

Step 2: The preparation method was the same as that of compound z-7, except that compounds z-3 and 7.1 in the preparation method of compound z-7 were replaced by compounds 231-1 and 5b. MS m/z (ESI): 731[M+H]⁺.

Step 3: The preparation method was the same as that of compound z-8, except that compound 8-1 in the preparation method of compound z-8 was replaced by compound 231-2. After purification by Prep-HPLC, compound z-231 (0.7 mg, 1%) was obtained as a white solid. MS m/z (ESI): 641.4 [M+H]⁺.

### Example 232: Preparation of 3-((5-(((1R,4R)-4-(dimethylamino) cyclohexyl) (ethyl) amino)-6-fluoro-7-(6-(4-methylpiperazin-1-yl) pyridin-3-yl) isoquinolin-1-ylamino) methyl)-4,6-dimethyl-2(1H)-one (compound z-232)

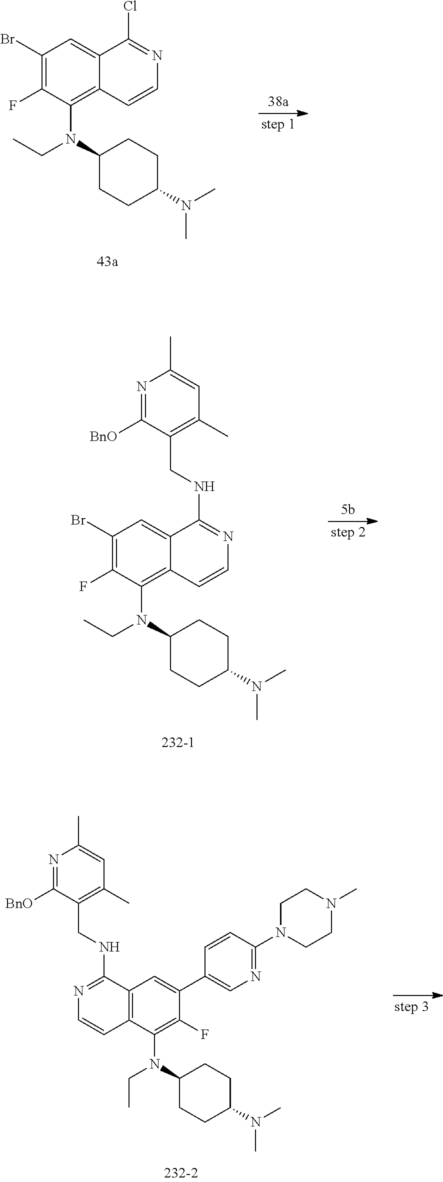 [SMILES: CCN(c1c(F)c(Br)cc2c(Cl)nccc12)[C@H]1CC[C@H](N(C)C)CC1 ; CCN(c1c(F)c(Br)cc2c(NCc3c(C)cc(C)nc3OCc3ccccc3)nccc12)[C@H]1CC[C@H](N(C)C)CC1 ; CCN(c1c(F)c(-c2ccc(N3CCN(C)CC3)nc2)cc2c(NCc3c(C)cc(C)nc3OCc3ccccc3)nccc12)[C@H]1CC[C@H](N(C)C)CC1]

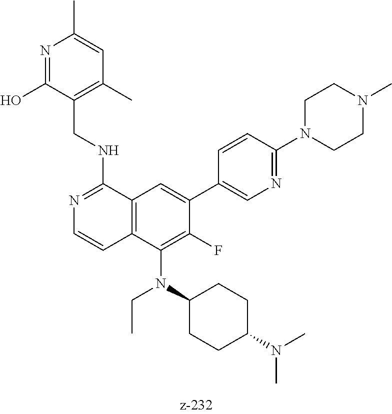 [SMILES: CCN(c1c(F)c(-c2ccc(N3CCN(C)CC3)nc2)cc2c(NCc3c(C)cc(C)nc3O)nccc12)[C@H]1CC[C@H](N(C)C)CC1]

Step 1: The preparation method was the same as that of compound 218-3, except that compound 218-2 in the preparation method of compound 218-3 was replaced by compound 43a. MS m/z (ESI): 634 [M+H]⁺.

Step 2: The preparation method was the same as that of compound z-7, except that compounds z-3 and 7.1 in the preparation method of compound z-7 were replaced by compounds 232-1 and 5b. MS m/z (ESI): 731 [M+H]⁺.

Step 3: The preparation method was the same as that of compound z-8, except that compound 8-1 in the preparation method of compound z-8 was replaced by compound 232-2. After purification by Prep-HPLC, compound z-232 (23 mg, 24%) was obtained as a white solid. MS m/z (ESI): 641.4[M+H]⁺. ¹H NMR (400 MHz, DMSO) δ 11.48 (s, 1H), 8.34 (s, 1H), 8.27-8.14 (m, 2H), 7.86 (d, 1H), 7.74 (d, 1H), 7.29 (s, 1H), 7.18 (d, 1H), 6.90 (d, 1H), 5.85 (s, 1H), 4.42 (d, 2H), 3.59-3.40 (m, 4H), 3.28-3.05 (m, 3H), 2.98-2.95 (m, 2H), 2.41-2.34 (m, 4H), 2.27 (s, 5H), 2.19-2.16 (m, 5H), 2.08 (s, 3H), 1.86-1.67 (m, 4H), 1.30-1.17 (m, 4H), 0.82 (t, 3H).

### Example 233: Preparation of 3-((7-chloro-5-(((1S,4S)-4-(dimethylamino) cyclohexyl) (ethyl)amino) isoquinolin-1-yloxy)methyl)-4,6-dimethylpyridine-2(1H)-one (compound z-233)

### Example 234: Preparation of 3-((7-chloro-5-(((1R,4R)-4-(dimethylamino) cyclohexyl) (ethyl) amino) isoquinolin-1-yloxy)methyl)-4,6-dimethylpyridine-2(1H)-one (compound z-234)

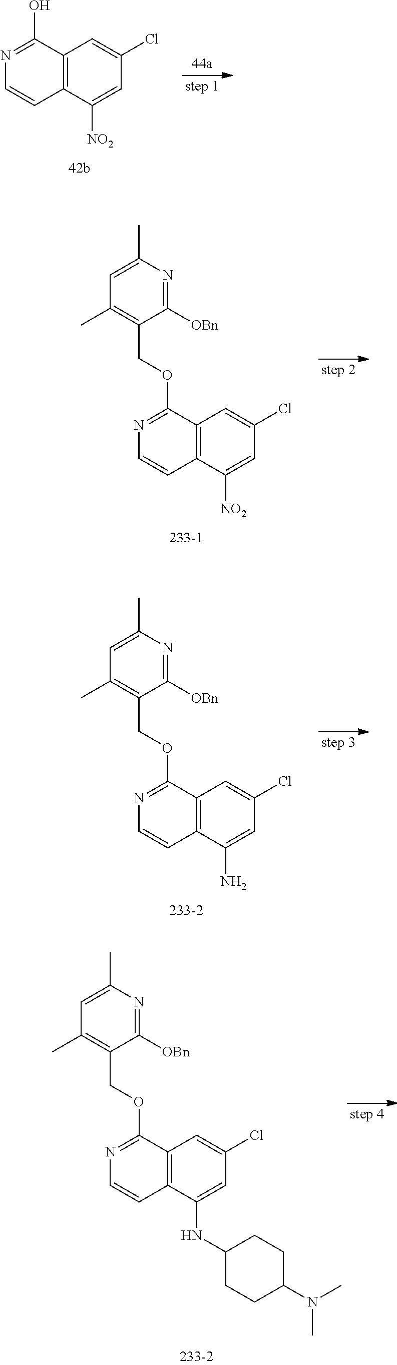

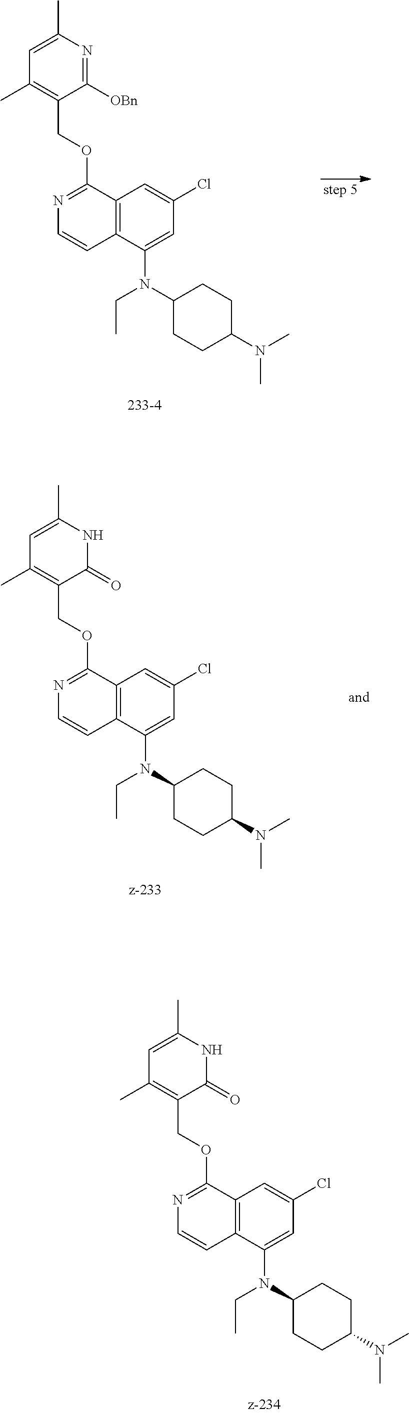

Step 1: A solution of compound 42b (277 mg, 1.233 mmol), compound 41a (300 mg, 1.233 mmol), PPh₃ (647 mg, 2.466 mmol) and DIAD (499 mg, 2.466 mmol) in tetrahydrofuran (15 mL) was stirred at room temperature for 3 hours. LC-MS was used to trace the reaction until the reaction was complete. The reaction solution was concentrated and purified by combiflash to give 450 mg of compound 233-1. MS m/z (ESI): 449.9 [M+H]⁺.

Step 2: The preparation method was the same as that of compound 38b-1, except that compound 3a-4 in the preparation method of compound 38b-1 was replaced by compound 233-1. MS m/z (ESI): 419.9 [M+H]⁺.

Step 3: The preparation method was the same as that of compound 15-1, except that compound 4a in the preparation method of compound 15-1 was replaced by compound 233-2. MS m/z (ESI): 544.9 [M+H]⁺.

Step 4: The preparation method was the same as that of compound z-15, except that compound 15-1 in the preparation method of compound z-15 was replaced by compound 233-3. MS m/z (ESI): 573 [M+H]⁺.

Step 5: The preparation method was the same as that of compound z-8, except that compound 8-1 in the preparation method of compound z-8 was replaced by compound 233-4. After Prep-HPLC purification, solid compounds z-233 (2.2 mg, 1.4) and z-234 (4.5 mg, 3%) were obtained. MS m/z (ESI): 483[M+H]⁺. ¹H NMR (400 MHz, DMSO-d₆): 11.65 (s, 1H), 7.95 (d, 1H), 7.59 (d, 1H), 7.53 (d, 1H), 6.87 (d, 1H), 5.93 (s, 1H), 4.96 (s, 2H), 3.07 (q, 2H), 2.69 (d, 6H), 2.27 (3H), 2.13 (s, 3H), 2.00-1.86 (m, 3H), 1.72-1.60 (m, 4H), 1.52-1.38 (m, 3H), 0.79 (t, 3H).

### Example 235: Preparation of 3-((5-(ethyl (piperidin-4-yl)amino)-7-isopropyl isoquinolin-1-ylamino)methyl)-4,6-dimethyl-2(1H)-one (compound z-235)

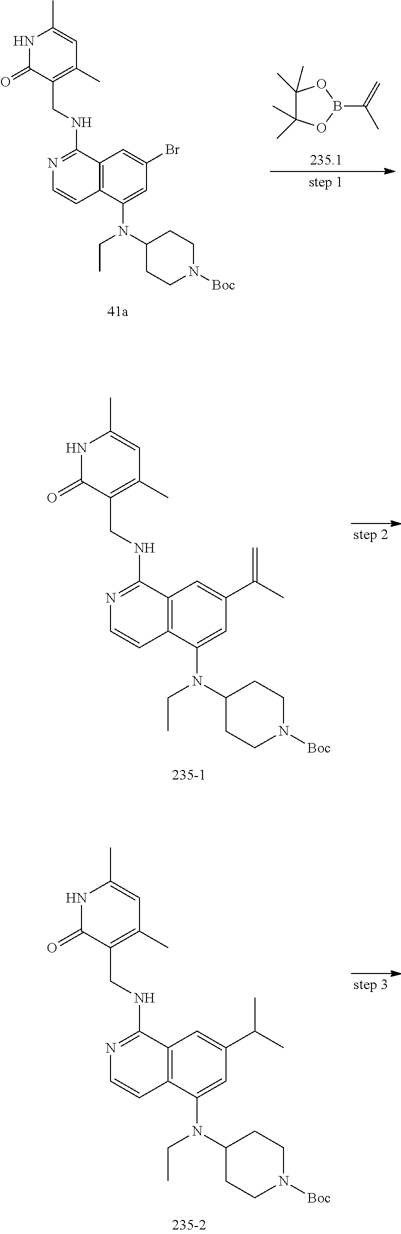 [SMILES: CCN(c1cc(Br)cc2c(NCc3c(C)cc(C)[nH]c3=O)nccc12)C1CCN(C(=O)OC(C)(C)C)CC1 ; C=C(C)B1OC(C)(C)C(C)(C)O1 ; C=C(C)c1cc(N(CC)C2CCN(C(=O)OC(C)(C)C)CC2)c2ccnc(NCc3c(C)cc(C)[nH]c3=O)c2c1 ; CCN(c1cc(C(C)C)cc2c(NCc3c(C)cc(C)[nH]c3=O)nccc12)C1CCN(C(=O)OC(C)(C)C)CC1]

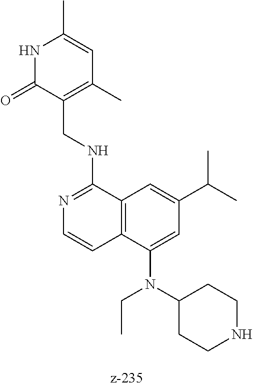 [SMILES: CCN(c1cc(C(C)C)cc2c(NCc3c(C)cc(C)[nH]c3=O)nccc12)C1CCNCC1]

Step 1: The preparation method was the same as that of compound z-7, except that compounds z-3 and 7.1 in the preparation method of compound z-7 were replaced by compounds 31a and 235.1. MS m/z (ESI): 546 [M+H]⁺.

Step 2: The preparation method was the same as that of compound z-10, except that compound 10-1 in the preparation method of compound z-10 was replaced by compound 235-1. MS m/z (ESI): 548 [M+H]⁺.

Step 3: The preparation method was the same as that of compound z-8, except that compound 8-1 in the preparation method of compound z-8 was replaced by compound 235-2. After Prep-HPLC purification, a white solid compound z-235 (1.3 mg, 1.8%) was obtained. MS m/z (ESI): 448 [M+H]⁺.

### Biological Test

### [Test Example 1] In Vitro Methyltransferase Activity Test

Recombinant PRC2 (EZH2-Y641F) was purchased from Active motif, S-adenosyl-methionine (SAM) and Poly-L-lysine (PLL) were purchased from Sigma-Aldrich, H3(1-50)K27mel polypeptide was purchased from Cisbio. LANCEUltra system (Perkinelmer) was used for detection. In the enzyme activity test, a compound to be tested was 1:3 diluted for 8 gradient points, added into a reaction plate, and then added 100 ng recombinant enzyme. And then a buffer [20 mM Tris pH8.5, 2 mM MgCl2, 0.01% Tween-20, 1 mM TCEP] containing 2.5 M SAM/250 nM H3(1-50)K27mel premixture was added to conduct an enzyme reaction at room tempreture. After reacted for 3 hours, a dection solution containing premixed PLL, detection antibody and Ulight was added, reacted for 1 h at RT, then fluorescence was read on Tecan infinite pro. IC50s were calculated by four-factor model fitting in a XLfit software. The results are shown in Table 1:

TABLE 1
Inhibitory Activity of Compounds against EZH2 Y641F

| | EZH2 | | EZH2 | | EZH2 |
|---|---|---|---|---|---|
| com- | Y641F | com- | Y641F | com- | Y641F |
| pound | IC50 (μM) | pound | IC50 (μM) | pound | IC50 (μM) |
| Z-1 | 0.422 | Z-8 | 0.086 | Z-3 | 0.1855 |
| Z-4 | 0.295 | Z-14 | 0.426 | Z-6 | 0.611 |
| Z-7 | 0.481 | Z-17 | 0.013 | Z-9 | 0.057 |
| Z-13 | 0.092 | Z-23 | 0.212 | Z-15 | 0.091 |
| Z-16 | 0.428 | Z-29 | 0.157 | Z-18 | 0.284 |
| Z-28 | 0.341 | Z-32 | 0.488 | Z-21 | 0.205 |
| Z-31 | 0.328 | Z-35 | 0.221 | Z-24 | 0.152 |
| Z-37 | 0.028 | Z-38 | 0.138 | Z-27 | 0.096 |
| Z-40 | 0.035 | Z-41 | 0.038 | Z-30 | 0.298 |
| Z-43 | <0.014 | Z-44 | 0.035 | Z-33 | 0.145 |
| Z-46 | 0.014 | Z-47 | 0.092 | Z-36 | <0.014 |
| Z-49 | 0.010 | Z-50 | 0.026 | Z-39 | 0.016 |
| Z-52 | <0.005 | Z-53 | 0.011 | Z-42 | 0.058 |
| Z-55 | 0.017 | Z-56 | 0.007 | Z-45 | 0.038 |
| Z-58 | 0.022 | Z-59 | 0.277 | Z-48 | 0.303 |
| Z-61 | 0.012 | Z-62 | 0.080 | Z-51 | <0.005 |
| Z-64 | 0.009 | Z-65 | 0.052 | Z-54 | 0.019 |
| Z-67 | 0.070 | Z-68 | 0.150 | Z-57 | 0.086 |
| Z-73 | 0.150 | Z-74 | 0.326 | Z-60 | 0.321 |
| Z-76 | 0.060 | Z-77 | 0.177 | Z-63 | 0.012 |
| Z-79 | 0.045 | Z-80 | 0.026 | Z-66 | 0.159 |
| Z-85 | 0.269 | Z-83 | 0.244 | Z-69 | 0.100 |
| Z-88 | 0.164 | Z-86 | 0.128 | Z-72 | 0.068 |
| Z-91 | 0.075 | Z-89 | 0.043 | Z-75 | 0.128 |
| Z-94 | 0.159 | Z-101 | 0.095 | Z-78 | 0.198 |
| Z-97 | 0.283 | Z-110 | 0.067 | Z-81 | 0.053 |
| Z-100 | 0.032 | Z-113 | 0.153 | Z-84 | 0.083 |
| Z-109 | 0.182 | Z-116 | 0.041 | Z-87 | 0.474 |
| Z-112 | 0.096 | Z-122 | 0.439 | Z-93 | 0.074 |
| Z-115 | 0.047 | Z-125 | 0.022 | Z-99 | 0.251 |
| Z-118 | 0.085 | Z-128 | 0.225 | Z-105 | 0.197 |
| Z-121 | 0.474 | Z-134 | 0.117 | Z-108 | 0.283 |
| Z-124 | 0.051 | Z-137 | 0.074 | Z-111 | 0.037 |
| Z-127 | 0.010 | Z-149 | 0.159 | Z-114 | 0.084 |
| Z-136 | 0.049 | Z-152 | 0.096 | Z-117 | 0.348 |
| Z-139 | 0.418 | Z-155 | 0.159 | Z-123 | 0.075 |
| Z-145 | 0.203 | Z-158 | 0.367 | Z-126 | 0.156 |
| Z-151 | 0.050 | Z-161 | 0.331 | Z-132 | 0.107 |
| Z-154 | 0.019 | Z-164 | 0.371 | Z-138 | 0.037 |
| Z-157 | 0.016 | Z-167 | 0.453 | Z-144 | 0.152 |
| Z-160 | 0.006 | Z-170 | 0.374 | Z-150 | 0.061 |
| Z-163 | 0.033 | Z-176 | 0.139 | Z-153 | 0.054 |
| Z-166 | 0.015 | Z-179 | 0.015 | Z-156 | 0.037 |
| Z-169 | 0.451 | Z-182 | 0.504 | Z-159 | 0.021 |
| Z-172 | 0.081 | Z-188 | 0.112 | Z-162 | 0.253 |
| Z-175 | 0.182 | Z-191 | 0.206 | Z-177 | 0.031 |
| Z-178 | 0.348 | Z-197 | 0.051 | Z-180 | 0.013 |
| Z-181 | 0.226 | Z-206 | 0.115 | Z-183 | 0.128 |
| Z-187 | 0.213 | Z-209 | 0.010 | Z-186 | 0.067 |
| Z-190 | 0.472 | Z-212 | 0.185 | Z-189 | 0.307 |
| Z-193 | 0.021 | Z-218 | 0.028 | Z-192 | 0.042 |
| Z-196 | 0.016 | Z-221 | 0.194 | Z-201 | 0.059 |
| Z-199 | 0.008 | Z-224 | 0.360 | Z-204 | 0.024 |
| Z-205 | 0.035 | Z-227 | 0.064 | Z-207 | 0.032 |
| Z-208 | 0.009 | Z-223 | 0.045 | Z-210 | 0.193 |
| Z-214 | 0.373 | Z-229 | 0.429 | Z-213 | 0.114 |
| Z-220 | 0.031 | Z-232 | 0.026 | Z-216 | 0.365 |
| Z-225 | 0.068 | Z-235 | 0.129 | Z-222 | 0.143 |
| Z-231 | 0.174 | | | | |

### [Test Example 2] Cell Proliferation Test

Cell Proliferation Test

Cell lines Pfeiffer (CRL-2632), suDHL-6 (CRL-2959) and suDHL-10 (CRL-2963) were obtained from ATCC. All cell lines were cultured in RPMI-1640 media (Gibco) plus 10% FBS (Gibco). Cells were harvested by centrifuge and densities were determined using a CounterStar cell counter. Appropriate number of cells were plated in a 96-well culture plate and incubated overnight. Compounds to be tested were 1:3 diluted for 8 gradient points and added into corresponding wells. After 6 days' growth, cell counting kit-8 (Dojindo) was used to detect cell viability and absorbance values were read on Tecan infinite pro. IC50s were calculated by four-factor model fitting in a XLfit software. The results are shown in Table 2:

TABLE 2
Inhibitory Activity of Compounds against Pfeiffer cells

| com- | Pfeiffer | com- | Pfeiffer | com- | Pfeiffer |
|---|---|---|---|---|---|
| pound | IC50 (μM) | pound | IC50 (μM) | pound | IC50 (μM) |
| Z-37 | 0.991 | Z-17 | 0.768 | Z-36 | 0.410 |
| Z-40 | 0.634 | Z-35 | 0.611 | Z-39 | 0.507 |
| Z-43 | 0.691 | Z-44 | 0.422 | Z-45 | 0.599 |
| Z-46 | 0.362 | Z-53 | 0.317 | Z-51 | 0.249 |
| Z-49 | 0.770 | Z-65 | 0.972 | Z-54 | 0.614 |
| Z-52 | 0.383 | Z-74 | 0.799 | Z-63 | 0.800 |
| Z-55 | 0.481 | Z-86 | 0.483 | Z-66 | 0.766 |
| Z-58 | 0.359 | Z-89 | 0.401 | Z-72 | 0.581 |
| Z-64 | 0.357 | Z-92 | 0.688 | Z-93 | 0.279 |
| Z-70 | 0.516 | Z-101 | 0.743 | Z-96 | 0.926 |
| Z-73 | 0.593 | Z-113 | 0.713 | Z-99 | 0.201 |
| Z-82 | 0.484 | Z-116 | 0.525 | Z-105 | 0.988 |
| Z-85 | 0.521 | Z-122 | 0.833 | Z-108 | 0.612 |
| Z-91 | 0.291 | Z-125 | 0.772 | Z-111 | 0.478 |
| Z-94 | 0.384 | Z-137 | 0.584 | Z-114 | 0.788 |
| Z-97 | 0.326 | Z-179 | 0.540 | Z-126 | 0.765 |
| Z-100 | 0.103 | Z-197 | 0.371 | Z-129 | 0.940 |
| Z-112 | 0.595 | Z-206 | 0.234 | Z-132 | 0.301 |
| Z-115 | 0.706 | Z-209 | 0.250 | Z-138 | 0.096 |
| Z-118 | 0.973 | Z-218 | 0.212 | Z-150 | 0.354 |
| Z-121 | 0.646 | Z-224 | 0.605 | Z-177 | 0.444 |
| Z-127 | 0.442 | Z-208 | 0.155 | Z-180 | 0.380 |
| Z-136 | 0.465 | Z-220 | 0.651 | Z-183 | 0.395 |
| Z-193 | 0.620 | Z-232 | 0.188 | Z-186 | 0.251 |
| Z-196 | 0.583 | Z-213 | 0.694 | Z-192 | 0.393 |
| Z-199 | 0.599 | Z-222 | 0.484 | Z-201 | 0.133 |
| Z-210 | 0.340 | | | | |

As can be seen from Table 1 and Table 2, the representative compounds of the present disclosure have high inhibitory activity against EZH2 enzymes and cells.

All publications mentioned herein are incorporated by reference as if each individual document was cited as a reference, as in the present application. It should also be understood that, after reading the above teachings of the present disclosure, those skilled in the art can make various changes or modifications, equivalents of which falls in the scope of claims as defined in the appended claims.

1. A compound of formula (I), or a pharmaceutically acceptable salt, stereoisomer, solvate or prodrug thereof: 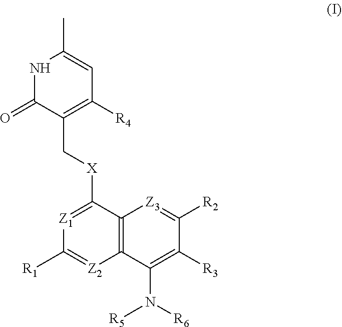
wherein, X is NH or O;
R₁, R₃ are each independently hydrogen, halogen, C₁₋₈ alkyl, halogenated C₁₋₈ alkyl, C₁₋₈ alkoxy, C₃₋₈ cycloalkyl or C₃₋₈ cycloalkoxy;
R₄ is a hydrogen, halogen, hydroxy, CN, C₁₋₈ alkyl, halogenated C₁₋₈ alkyl, C₃₋₈ cycloalkyl, C₁₋₈ alkoxy, halogenated C₁₋₈ alkoxy, C₃₋₈ cycloalkoxy, C₆₋₁₀ aryl, C(O)C₁₋₈ alkyl, or C(O)OC₁₋₈ alkyl;
Z₁ is N or CR₈; Z₂ is N or CR₉; Z₃ is N or CR₁₀;
R₈, R₉, R₁₀ are each independently hydrogen, halogen, or C₁₋₈ alkyl;
R₂ is a hydrogen, halogen, CN, C₁₋₈ alkyl, C₂₋₈ alkynyl, halogenated C₁₋₈ alkyl, C₃₋₈ cycloalkyl, C₁₋₈ alkoxy, C(O)NRₐ₁R_{b1}, NRₐ₂R_{b2}, NCORₐ₃, ORₐ₄, C₆₋₁₀ aryl, 4 to 6 membered saturated or unsaturated single heterocycle, or 5 to 6 membered monocyclic heteroaryl ring;
Rₐ₁, R_{b1}, Rₐ₂, R_{b2}, Rₐ₃, Rₐ₄ are each independently hydrogen, C₁₋₈ alkyl, 4 to 6 membered saturated single heterocycle, 5 to 6 membered monocyclic heteroaryl ring, or C₆₋₁₀ aryl;
Or, Rₐ₁, R_{b1} and a nitrogen atom attached thereto form a 5 to 6 membered saturated single heterocycle;
R₅ is a hydrogen, C₁₋₈ alkyl, or C₁₋₈ alkyl substituted with C₁₋₈ alkoxy;
R₆ is a C₁₋₈ alkyl, C₃₋₈ cycloalkyl, 4 to 6 membered saturated single heterocycle, spiro, spiroheterocycle, bridged ring, or bridged heterocycle;
the alkyl, cycloalkyl, alkoxy, alkynyl, aryl, 4 to 6 membered saturated or unsaturated single heterocycle, 5 to 6 membered monocyclic heteroaryl ring, spiro, spiroheterocycle, bridged ring, and bridged heterocycle each is unsubstituted or substituted with 1, 2 or 3 substituents selected from the group consisting of NRₐ₀R_{b0}, hydroxymethyl, hydroxyethyl, carboxyl, —C(O)OC₁₋₆ alkyl, halogen, acetyl, hydroxy, C₁₋₈ alkyl, C₁₋₈ alkoxy, halogenated C₁₋₈ alkyl, C₃₋₈ cycloalkyl, halogenated C₁₋₈ alkoxy, —SO₂C₁₋₈ alkyl, C₆₋₁₀ aryl, 4 to 6 membered saturated single heterocycle, 5 to 6 membered monocyclic heteroaryl ring, O(CH₂)ₙOC₁₋₈ alkyl or —Y-L; wherein Y is a bond, CH₂, NH, O, CON or C(O); L is a C₆₋₁₀ aryl, 5 to 6 membered monocyclic heteroaryl ring, 4 to 6 membered saturated single heterocycle or NRₐ₀R_{b0}; Rₐ₀, R_{b0} are each independently hydrogen, acetyl, C₁₋₈ alkyl, or C₁₋₈ alkyl substituted with C₁₋₈ alkoxy.

2. The compound of claim 1, or a pharmaceutically acceptable salt, stereoisomer, solvate or prodrug thereof, wherein X is NH.

3. The compound of claim 1, or a pharmaceutically acceptable salt, stereoisomer, solvate or prodrug thereof, wherein R₂ is halogen, CN, C₁₋₈ alkyl, halogenated C₁₋₈ alkyl, C₃₋₈ cycloalkyl, or C₁₋₈ alkoxy.

4.-8. (canceled)

9. The compound of claim 1, or a pharmaceutically acceptable salt, stereoisomer, solvate or prodrug thereof, wherein R₂ is phenyl; the phenyl is unsubstituted or substituted with 1, 2 or 3 groups selected from the group consisting of C₁₋₈ alkyl, halogenated C₁₋₈ alkyl, C₁₋₈ alkoxy, halogen, —O(CH₂)ₙOC₁₋₈ alkyl or —Y₁-L₁; wherein Y₁ is a bond, CH₂, NH, O or CON; L₁ is C₆₋₁₀ aryl, 5 to 6 membered monocyclic heteroaryl ring, 4 to 6 membered saturated single heterocycle or NRₐ₀R_{b0}; Rₐ₀, R_{b0} are each independently hydrogen or C₁₋₈ alkyl;
the aryl, 4 to 6 membered saturated single heterocycle, 5 to 6 membered monocyclic heteroaryl ring is unsubstituted or substituted with 1, 2 or 3 groups selected from the group consisting of NRₐ₀R_{b0}, hydroxymethyl, hydroxyethyl, carboxyl, —C(O)OC₁₋₆ alkyl, acetyl, hydroxy, C₁₋₃ alkyl, halogenated C₁₋₃ alkyl, C₃₋₆ cycloalkyl, azetidine, oxetane, tetrahydrofuran, tetrahydrothiophene, pyrrolidine, piperidine, oxazolidine, piperazine, dioxolane, dioxane, morpholine, thiomorpholine, thiomorpholine-1,1-dioxide or tetrahydropyrane; Rₐ₀, R_{b0} are each independently hydrogen or C₁₋₃ alkyl.

10. The compound of claim 1, or a pharmaceutically acceptable salt, stereoisomer, solvate or prodrug thereof, wherein R₂ is a structure shown by formula A: 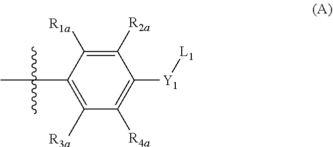
wherein R₁ₐ, R₂ₐ, R₃ₐ, R₄ₐ are each independently hydrogen, C₁₋₈ alkyl, halogenated C₁₋₈ alkyl, C₁₋₈ alkoxy or halogen; Y₁, L₁ are as defined above.

11. The compound of claim 1, or a pharmaceutically acceptable salt, stereoisomer, solvate or prodrug thereof, wherein R₂ is 5 to 6 membered monocyclic heteroaryl ring; the 5 to 6 membered monocyclic heteroaryl ring is unsubstituted or substituted with 1, 2 or 3 groups selected from the group consisting of halogen, C₁₋₈ alkyl, halogenated C₁₋₈ alkyl, C₃₋₈ cycloalkyl or —Y₂-L₂; wherein Y₂ is a bond, CH₂ or C(O); L₂ is a hydrogen, C₃₋₆ cycloalkyl, 4 to 6 membered saturated single heterocycle or NRₐ₀R_{b0}; Rₐ₀, R_{b0} are each independently hydrogen or C₁₋₈ alkyl;
the 4 to 6 membered saturated single heterocycle is unsubstituted or substituted with 1, 2 or 3 groups selected from the group consisting of NRₐ₀R_{b0}, hydroxymethyl, hydroxyethyl, carboxyl, —C(O)OC₁₋₆ alkyl, acetyl, hydroxy, C₁₋₃ alkyl, halogenated C₁₋₃ alkyl, C₃₋₆ cycloalkyl, azetidine, oxetane, tetrahydrofuran, tetrahydrothiophene, pyrrolidine, piperidine, oxazolidine, piperazine, dioxolane, dioxane, morpholine, thiomorpholine, thiomorpholine-1,1-dioxide or tetrahydropyrane; Rₐ₀, R_{b0} are each independently hydrogen or C₁₋₃ alkyl.

12. The compound of claim 1, or a pharmaceutically acceptable salt, stereoisomer, solvate or prodrug thereof, wherein R₂ is a structure shown by formula B or C: 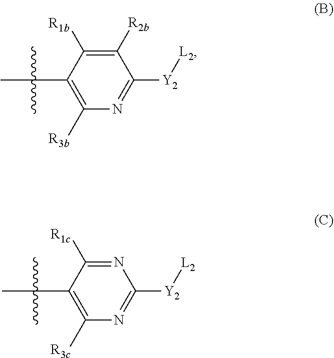
wherein R_{1b}, R_{2b}, R_{3b}, R_{1c}, R_{3c} are each independently hydrogen, halogen, C₁₋₈ alkyl, halogenated C₁₋₈ alkyl, or C₃₋₈ cycloalkyl; Y₂, L₂ are as defined above.

13. (canceled)

14. The compound of claim 1, or a pharmaceutically acceptable salt, stereoisomer, solvate or prodrug thereof, wherein R₂ is fluorine, chlorine, bromine, CN, methyl, ethyl, n-propyl, isopropyl, cyclopropyl, methoxy, ethoxy, or trifluoromethyl, or R₂ is the following structure:

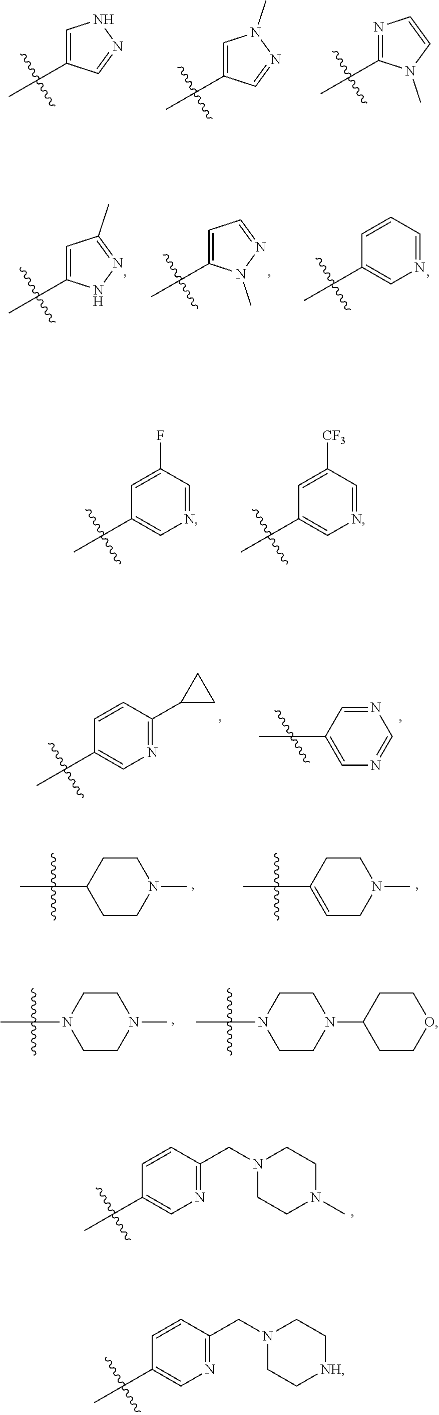

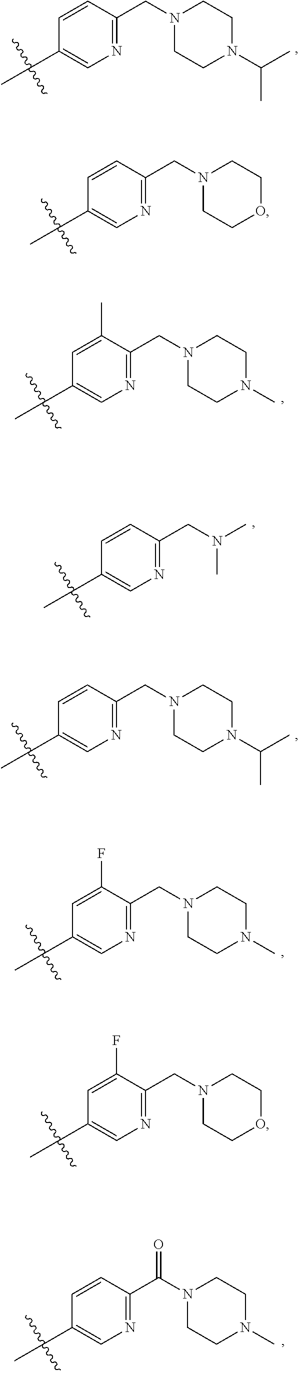

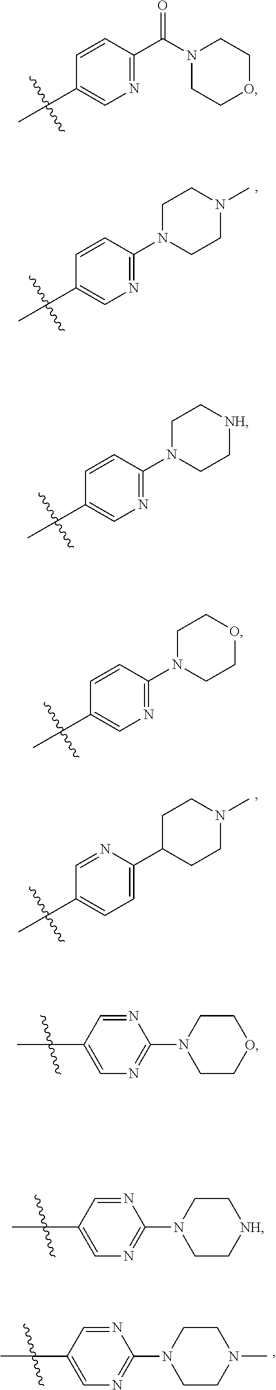

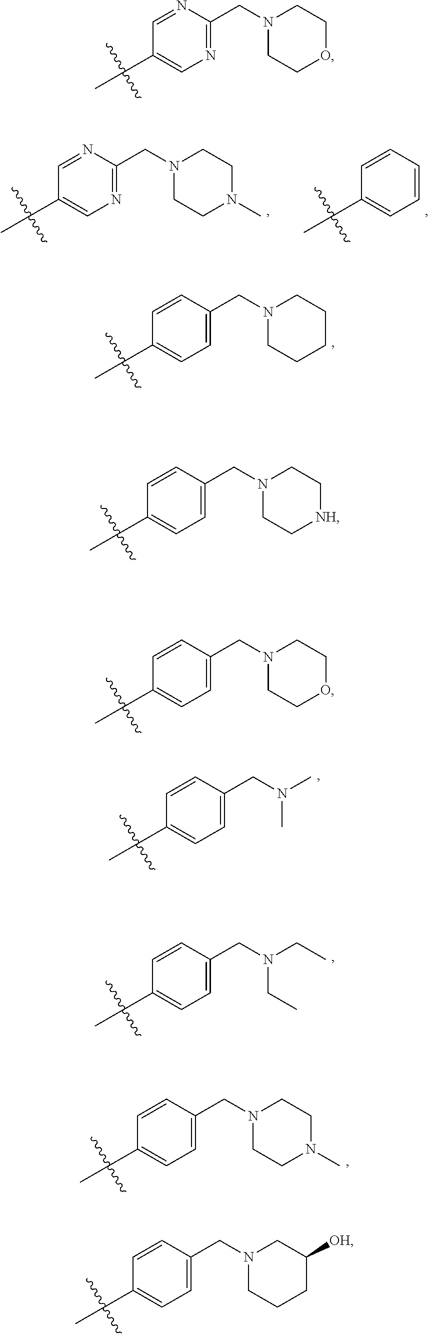

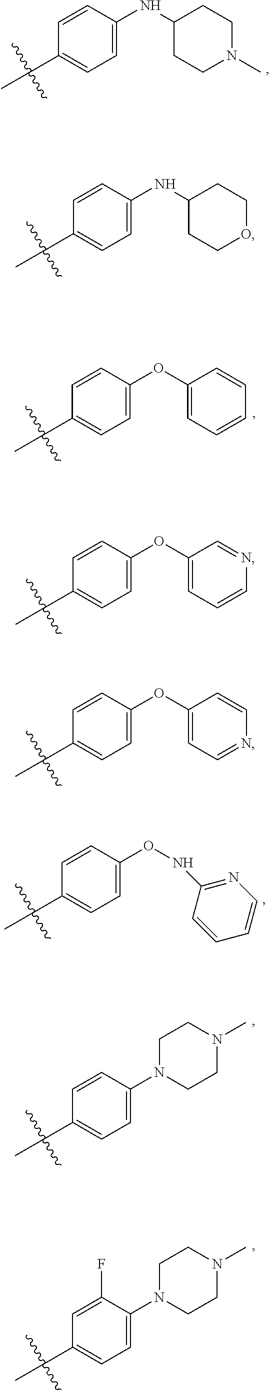

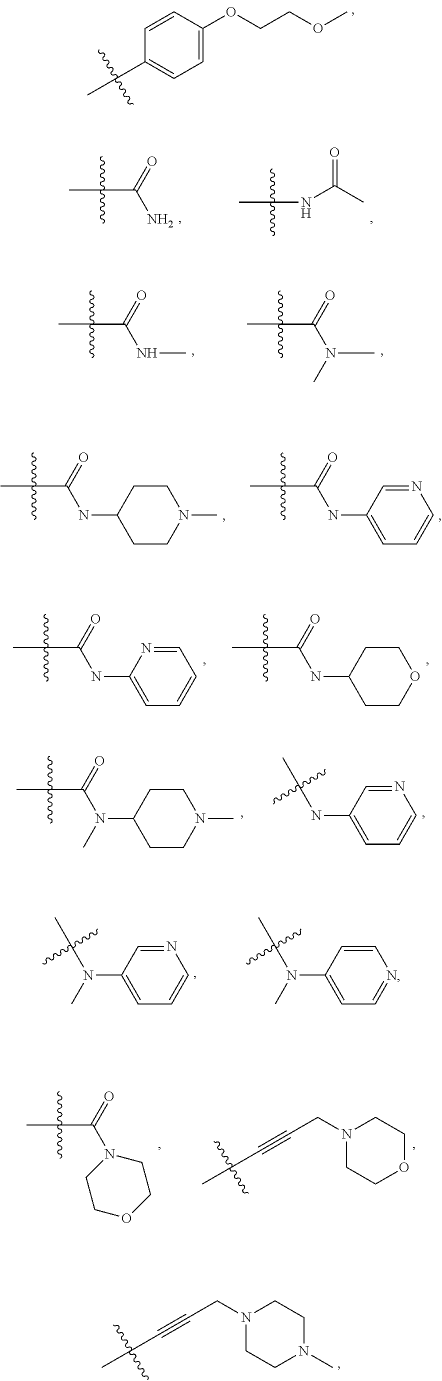

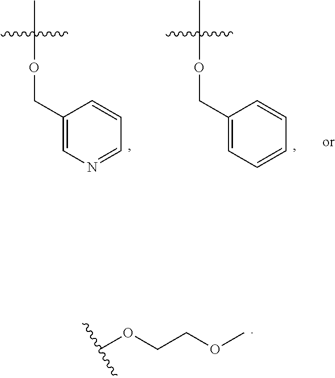

15. The compound of claim 1, or a pharmaceutically acceptable salt, stereoisomer, solvate or prodrug thereof, wherein R₅ is hydrogen, methyl, ethyl or ethyl substituted with methoxy.

16. (canceled)

17. The compound of claim 1, or a pharmaceutically acceptable salt, stereoisomer, solvate or prodrug thereof, wherein R₆ is C₃₋₆ cycloalkyl; the cycloalkyl is unsubstituted or substituted with 1, 2 or 3 groups selected from the group consisting of NRₐ₀R_{b0}, halogen, hydroxy, C₁₋₈ alkyl, C₁₋₈ alkoxy, halogenated C₁₋₈ alkyl, C₃₋₈ cycloalkyl, or 4 to 6 membered saturated single heterocycle; Rₐ₀, R_{b0} are each independently hydrogen, acetyl, C₁₋₈ alkyl, or C₁₋₈ alkyl substituted with C₁₋₈ alkoxy; the 4 to 6 membered saturated single heterocycle is unsubstituted or substituted with C₁₋₈ alkoxy.

18. (canceled)

19. The compound of claim 1, or a pharmaceutically acceptable salt, stereoisomer, solvate or prodrug thereof, wherein R₆ is 4 to 6 membered saturated single heterocycle; the 4 to 6 membered saturated single heterocycle is unsubstituted or substituted with 1, 2 or 3 groups selected from the group consisting of acetyl, C₁₋₈ alkyl, C₁₋₈ alkyl substituted with C₁₋₈ alkoxy, —SO₂C₁₋₈ alkyl, C₃₋₈ cycloalkyl, or 4 to 6 membered saturated single heterocycle; the acetyl is unsubstituted or substituted with CN or hydroxy.

20.-21. (canceled)

22. The compound of claim 1, or a pharmaceutically acceptable salt, stereoisomer, solvate or prodrug thereof, wherein R₆ is the following structure:

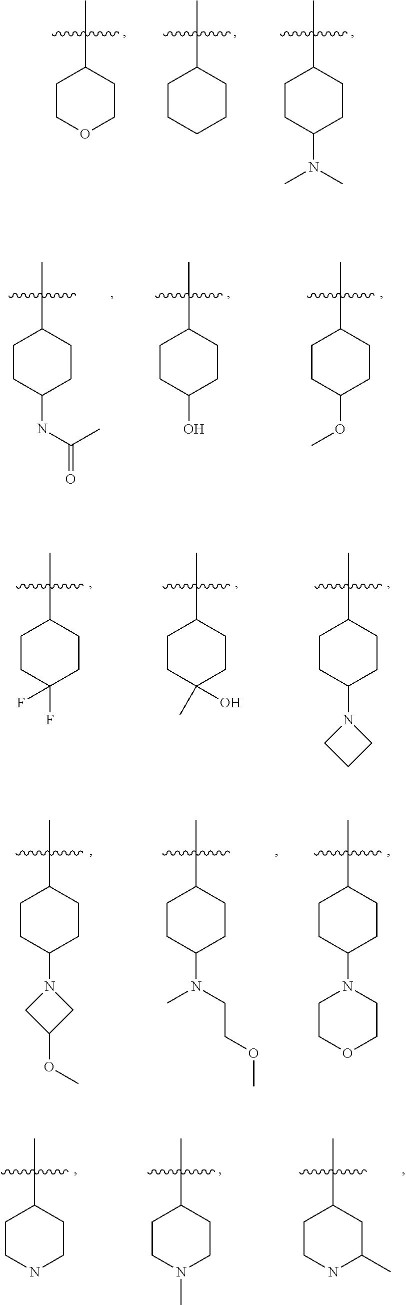

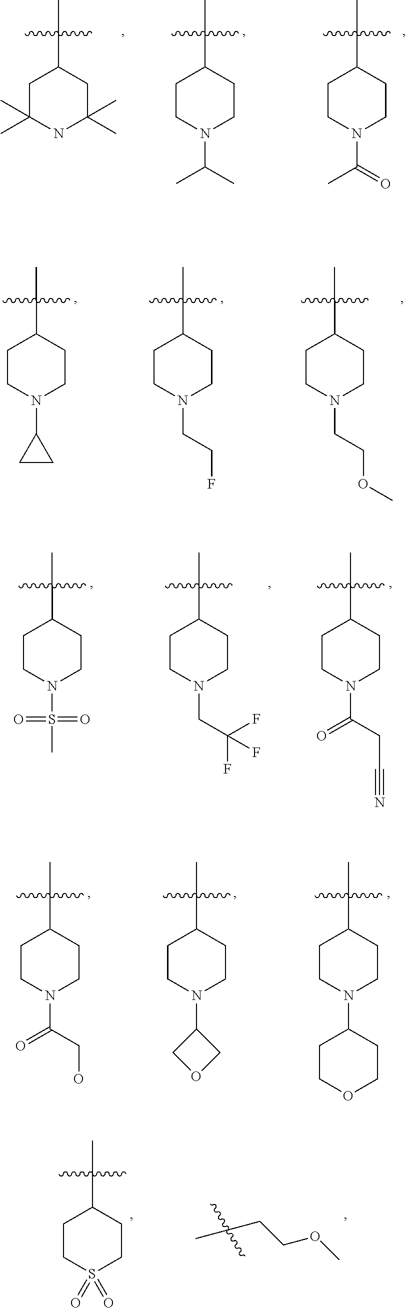

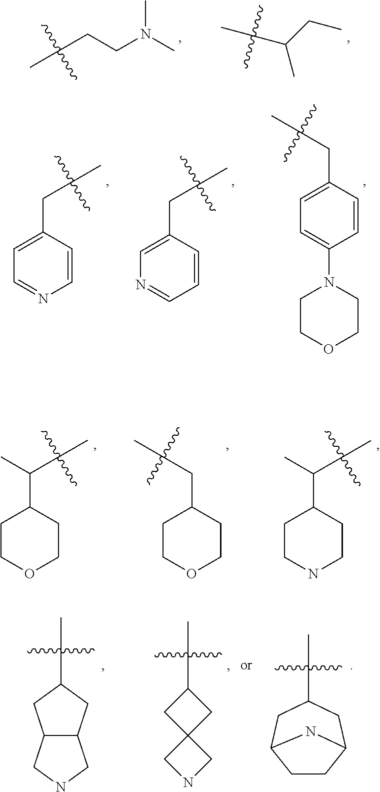

23. The compound of claim 1, or a pharmaceutically acceptable salt, stereoisomer, solvate or prodrug thereof, wherein R₁ is hydrogen or halogen.

24. The compound of claim 1, or a pharmaceutically acceptable salt, stereoisomer, solvate or prodrug thereof, wherein R₃ is hydrogen or halogen.

25. The compound of claim 1, or a pharmaceutically acceptable salt, stereoisomer, solvate or prodrug thereof, wherein R₄ is hydroxy, C₁₋₃ alkyl or C₁₋₃ alkoxy.

26. The compound of claim 1, or a pharmaceutically acceptable salt, stereoisomer, solvate or prodrug thereof, wherein Z₁ is N; Z₂ is CR₉; Z₃ is CR₁₀; R₉, R₁₀ are each independently hydrogen, halogen or C₁₋₈ alkyl, preferably R₉, R₁₀ are hydrogen.

27. The compound of claim 1, or a pharmaceutically acceptable salt, stereoisomer, solvate or prodrug thereof, wherein Z₁ is N; Z₂ is N; Z₃ is CR₁₀; R₁₀ is hydrogen, halogen or C₁₋₈ alkyl, preferably R₁₀ is hydrogen.

28.-30. (canceled)

31. A pharmaceutical composition comprising a compound of claim 1, or a pharmaceutically acceptable salt, solvate, stereoisomer or prodrug thereof; and a pharmaceutically acceptable carrier.

32.-33. (canceled)

34. A method for treating a disease or condition mediated by EZH2, comprising administering to a patient in need a therapeutically effective amount of the compound of claim 1, or a pharmaceutically acceptable salt, solvate, stereoisomer or prodrug thereof.

35. The method for treating a disease or condition mediated by EZH2 of claim 34, further comprising administering to a patient in need another therapeutically active agent.
